(19) United States
(12) Patent Application Publication
(10) Pub. No.: US 2025/0179492 A1
(43) Pub. Date: Jun. 5, 2025

(54) COMPOSITIONS OF MODIFIED TREMS AND USES THEREOF

(71) Applicant: Flagship Pioneering Innovations VI, LLC, Cambridge, MA (US)

(72) Inventors: Theonie Anastassiadis, Somerville, MA (US); David Charles Donnell Butler, Medford, MA (US); Neil Kubica, Swampscott, MA (US); Qingyi Li, Somerville, MA (US); Armand Gatien Ngounou Wetie, Woburn, MA (US); Hongchuan Yu, Chestnut Hill, MA (US); William F. Kiesman, Wayland, MA (US); Guangliang Wang, Lexington, MA (US)

(21) Appl. No.: 18/876,922

(22) PCT Filed: Jun. 22, 2023
(86) PCT No.: PCT/US2023/026027

§ 371 Date: Dec. 19, 2024

Related U.S. Application Data
(60) Provisional application No. 63/354,602, filed on Jun. 22, 2022.
(60) Provisional application No. 63/354,604, filed on Jun. 22, 2022.

(51) Int. Cl. C12N 15/113

(52) U.S. Cl. CPC C12N 15/113; C12N 2310/315; C12N 2310/321; C12N 2310/322; C12N 2310/351

(57) ABSTRACT

The invention relates generally to tRNA-based effector molecules (TREMs) comprising an asialoglycoprotein receptor (ASGPR) binding moiety, as well as compositions and methods relating thereto.

### CLAIM OF PRIORITY

This application claims priority to U.S. Provisional Application No. 63/354,602, filed Jun. 22, 2022; and U.S. Provisional Application No. 63/354,604, filed Jun. 22, 2022. The entire contents of each of the foregoing applications are incorporated herein by reference in their entirety.

### BACKGROUND

tRNAs are complex RNA molecules that possess a number of functions including the ability to initiate and elongate proteins.

### SUMMARY

The present disclosure features, inter alia, a tRNA-based effector molecule (TREM) entity comprising an asialoglycoprotein receptor (ASGPR) binding moiety, as well as compositions and methods of use thereof. The ASGPR binding moiety may be conjugated to a sugar moiety (e.g., ribose moiety) of a nucleotide, to a nucleobase of a nucleotide, within an internucleotide linkage (e.g., the phosphate backbone), or at a terminus (e.g., the 5′ or 3′ terminus) of the TREM entity. In an embodiment, the TREM entity comprises a TREM, a TREM Core Fragment, or a TREM Fragment. In an embodiment, the ASGPR binding moiety is bound to a purine nucleobase or a pyrimidine nucleobase. In an embodiment, the nucleobase comprises adenine, thymine, cytosine, guanosine, or uracil, or a variant or modified form thereof.

In one aspect, the TREM entity (e.g., TREM) described herein comprises the sequence of Formula A: [L1]-[ASt Domain1]-[L2]-[DH Domain]-[L3]-[ACH Domain]-[VL Domain]-[TH Domain]-[L4]-[ASt Domain2] (A), wherein, independently, the TREM comprises an ASGPR binding moiety. In an embodiment, the ASGPR binding moiety comprises an ASGPR carbohydrate and an ASGPR linker. In an embodiment, the ASGPR binding moiety comprises a galactose (Gal) and/or N-acetylgalactosamine (GalNAc) moiety. In an embodiment, the ASGPR binding moiety comprises a plurality of Gal and/or GalNAc moieties (e.g., 2, 3, 4, 5, 6, 7, 8, or more Gal and/or GalNAc moieties). In an embodiment, the ASGPR binding moiety comprises a triantennary GalNAc moiety. In an embodiment, the TREM further comprises a chemical modification (e.g., a phosphothiorate internucleotide linkage, or a 2′-modification on a ribose moiety within the TREM).

In an embodiment, the ASGPR binding moiety is present on a sugar moiety (e.g., a ribose moiety) within the TREM. In an embodiment, the ASGPR binding moiety is present on a sugar moiety (e.g., a ribose moiety) at the 2′ position of the sugar moiety. In an embodiment, the ASGPR binding moiety is present on the sugar moiety (e.g., a ribose moiety) at the 2′ oxygen or carbon of the sugar moiety. In an embodiment, the ASGPR binding moiety is present on a sugar moiety (e.g., a ribose moiety) at the 4′ position of the sugar moiety. In an embodiment, the ASGPR binding moiety is present on the sugar moiety (e.g., a ribose moiety) at the 4′ carbon of the sugar moiety. In an embodiment, the ASGPR binding moiety is bound to the phosphate backbone at any nucleotide position within the TREM.

In an embodiment, the ASGPR binding moiety is present on a sugar moiety (e.g., ribose moiety) of a nucleotide within a TREM domain selected from L1, ASt Domain1, L2, DH Domain, L3, ACH Domain, VL Domain, TH Domain, L4, and ASt Domain2. In an embodiment, the ASGPR binding moiety is present on a sugar moiety (e.g., ribose moiety) of a nucleotide within the L1 region. In an embodiment, the ASGPR binding moiety is present on a sugar moiety (e.g., ribose moiety) of a nucleotide within the ASt Domain1. In an embodiment, the ASGPR binding moiety is present on a sugar moiety (e.g., ribose moiety) of a nucleotide within the L2 region. In an embodiment, the ASGPR binding moiety is present on a sugar moiety (e.g., ribose moiety) of a nucleotide within the DH Domain. In an embodiment, the ASGPR binding moiety is present on a sugar moiety (e.g., ribose moiety) of a nucleotide within the L3 region. In an embodiment, the ASGPR binding moiety is present on a sugar moiety (e.g., ribose moiety) of a nucleotide within the ACH Domain. In an embodiment, the ASGPR binding moiety is present on a sugar moiety (e.g., ribose moiety) of a nucleotide within the VL Domain. In an embodiment, the ASGPR binding moiety is present on a sugar moiety (e.g., ribose moiety) of a nucleotide within the TH Domain. In an embodiment, the ASGPR binding moiety is present on a sugar moiety (e.g., ribose moiety) of a nucleotide within the L4 region. In an embodiment, the ASGPR binding moiety is present on a sugar moiety (e.g., ribose moiety) of a nucleotide within the ASt Domain2.

In an embodiment, the ASGPR binding moiety is bound to the phosphate backbone at any nucleotide position within a TREM domain selected from L1, ASt Domain1, L2, DH Domain, L3, ACH Domain, VL Domain, TH Domain, L4, and ASt Domain2. In an embodiment, the ASGPR binding moiety is bound to the phosphate backbone at any nucleotide position within the L1 region. In an embodiment, the ASGPR binding moiety is bound to the phosphate backbone at any nucleotide position within the ASt Domain1. In an embodiment, the ASGPR binding moiety is bound to the phosphate backbone at any nucleotide position within the L2 region. In an embodiment, the ASGPR binding moiety is bound to the phosphate backbone at any nucleotide position within the DH Domain. In an embodiment, the ASGPR binding moiety is bound to the phosphate backbone at any nucleotide position within the LD3 region. In an embodiment, the ASGPR binding moiety is bound to the phosphate backbone at any nucleotide position within the ACH Domain. In an embodiment, the ASGPR binding moiety is bound to the phosphate backbone at any nucleotide position within the VL Domain. In an embodiment, the ASGPR binding moiety is bound to the phosphate backbone at any nucleotide position within the TH Domain. In an embodiment, the ASGPR binding moiety is bound to the phosphate backbone at any nucleotide position within the L4 region. In an embodiment, the ASGPR binding moiety is bound to the phosphate backbone at any nucleotide position within the ASt Domain2.

In an embodiment, the ASGPR binding moiety is present on a nucleobase within a nucleotide in the TREM. In an embodiment, the ASGPR binding moiety is present on the 5′ terminus of the TREM. In an embodiment, the ASGPR binding moiety is present on the 3′ terminus of the TREM.

In an embodiment, the ASGPR binding moiety is present in a TREM domain selected from L1, ASt Domain1, L2, DH Domain, L3, ACH Domain, VL Domain, TH Domain, L4, and ASt Domain2. In an embodiment, the ASGPR binding moiety is present in the L1 region. In an embodiment, the ASGPR binding moiety is present in the AST Domain1. In an embodiment, the ASGPR binding moiety is present in the L2 region. In an embodiment, the ASGPR binding moiety is present in the DH Domain. In an embodiment, the ASGPR binding moiety is present in the L3 region. In an embodiment, the ASGPR binding moiety is present in the ACH Domain. In an embodiment, the ASGPR binding moiety is present in the VL Domain. In an embodiment, the ASGPR binding moiety is present in the TH Domain. In an embodiment, the ASGPR binding moiety is present in the L4 region. In an embodiment, the ASGPR binding moiety is present in the AST Domain2.

In an embodiment, the ASGPR binding moiety is bound to an adenine nucleobase at a carbon atom or a nitrogen atom. In an embodiment, the ASGPR binding moiety is bound to an adenine at the C2 position, N9 position, or C8 position. In an embodiment, the ASGPR binding moiety is bound to the adenine at the C2 position. In an embodiment, the ASGPR binding moiety is bound to the adenine at the N9 position. In an embodiment, the ASGPR binding moiety is bound to the adenine at the C8 position. In an embodiment, the ASGPR binding moiety is bound to a substituent on the adenosine nucleobase, e.g., an amine on the adenine nucleobase (e.g., amine off the C6 position).

In an embodiment, the ASGPR binding moiety is bound to a guanine nucleobase at a carbon or nitrogen atom. In an embodiment, the ASGPR binding moiety is bound to the guanine at the N1, C2, N9, or C8 position. In an embodiment, the ASGPR binding moiety is bound to the guanine at the NI position. In an embodiment, the ASGPR binding moiety is bound to the guanine at the C2 position. In an embodiment, the ASGPR binding moiety is bound to the guanine at the N9 position. In an embodiment, the ASGPR binding moiety is bound to the guanine at the C8 position. In an embodiment, the ASGPR binding moiety is bound to a substituent on the guanosine nucleobase, e.g., an amine on the guanosine nucleobase (e.g., amine off the C2 position).

In an embodiment, the ASGPR binding moiety is bound to a cytosine nucleobase at a carbon atom. In an embodiment, the ASGPR binding moiety is bound to the cytosine at the C4, C5, or C6 position. In an embodiment, the ASGPR binding moiety is bound to the cytosine at the C4 position. In an embodiment, the ASGPR binding moiety is bound to the cytosine at the C5 position. In an embodiment, the ASGPR binding moiety is bound to the cytosine at the C6 position. In an embodiment, the ASGPR binding moiety is bound to a substituent on the cytosine nucleobase, e.g., an amine on the cytosine nucleobase (e.g., amine off the C4 position).

In an embodiment, the ASGPR binding moiety is bound to a uracil nucleobase at a carbon or nitrogen atom. In an embodiment, the ASGPR binding moiety is bound to the uracil at the N3, C5, or C6 position. In an embodiment, the ASGPR binding moiety is bound to the uracil at the N3 position. In an embodiment, the ASGPR binding moiety is bound to the uracil at the C5 position. In an embodiment, the ASGPR binding moiety is bound to the uracil at the C6 position.

In an embodiment, the ASGPR binding moiety is bound to a thymine nucleobase at a carbon or a nitrogen atom. In an embodiment, the ASGPR binding moiety is bound to the thymine at the N3, C5, or C6 position. In an embodiment, the ASGPR binding moiety is bound to the thymine at the N3 position. In an embodiment, the ASGPR binding moiety is bound to the thymine at the C5 position. In an embodiment, the ASGPR binding moiety is bound to the thymine at the C6 position. In an embodiment, the ASGPR binding moiety is bound to a substituent on the thymine nucleobase, e.g., a methyl on the thymine nucleobase (e.g., a methyl off the C5 position).

In an embodiment, the ASGPR binding moiety is bound to the terminal nucleotide of a TREM molecule. In an embodiment, the terminal nucleotide is an adenine, a guanine, a cytosine, thymine, a uracil, or a variant thereof. In an embodiment, the ASGPR binding moiety is bound to the 5′ and/or 3′ terminal nucleotide of the TREM molecule. In an embodiment, the ASGPR binding moiety is bound to the 5′ terminal nucleotide. In an embodiment, the ASGPR binding moiety is bound to the 3′ terminal nucleotide. In an embodiment, the ASGPR binding moiety is bound to the 5′ terminal nucleotide and the 3′ terminal nucleotide. In an embodiment, the ASGPR binding moiety is bound to the 5′ terminal nucleotide at the C5′ hydroxyl group of the sugar moiety (e.g., ribose moiety). In an embodiment, the ASGPR binding moiety is bound to the 3′ terminal nucleotide at the C3′ ribose position.

In an embodiment, the TREM comprising an ASGPR binding moiety retains the ability to support protein synthesis, be charged by a synthetase, be bound by an elongation factor, introduce an amino acid into a peptide chain, support elongation, and/or support initiation. In an embodiment, the TREM comprising an ASGPR binding moiety comprises at least X contiguous nucleotides without a chemical modification, wherein X is greater than 10. In an embodiment, the TREM comprising an ASGPR binding moiety comprises no more than 5, 10, or 15 nucleotides of a type (e.g., A, T, C, G or U) that do not comprise chemical modification, and is further modified at a TREM domain (e.g., L1, ASt Domain1, L2, DH Domain, L3, ACH Domain, VL Domain, TH Domain, L4, and/or ASt Domain2.). In an embodiment, the TREM comprising an ASGPR binding moiety comprises no more than 5, 10, or 15 nucleotides of a type (e.g., A, T, C, G or U) that do not comprise chemical modification. In an embodiment, the TREM comprising an ASGPR binding moiety comprises no more than 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that do not comprise a chemical modification. In an embodiment, the TREM comprising an ASGPR binding moiety comprises at least X contiguous nucleotides comprising a chemical modification, wherein X is greater than 10. In an embodiment, the TREM comprising an ASGPR binding moiety comprises more than 5, 10, or 15 nucleotides of a type (e.g., A, T, C, G or U) that comprise a chemical modification, and is further modified at a TREM domain (e.g., L1, ASt Domain1, L2, DH Domain, L3, ACH Domain, VL Domain, TH Domain, L4, and ASt Domain2.). In an embodiment, the TREM comprising an ASGPR binding moiety comprises 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 80 nucleotides of a type (e.g., A, T, C, G or U) that comprise a chemical modification. In an embodiment, the chemical modification is a naturally occurring chemical modification or a non-naturally occurring chemical modification (e.g., a phosphothiorate internucleotide linkage or a 2′-modification on a ribose moiety within the TREM). In an embodiment, the chemical modification comprises a fluorophore.

In another aspect, a TREM comprising an ASGPR binding moiety, or a composition thereof, described herein may be used to modulate a production parameter (e.g., an expression parameter and/or a signaling parameter) of an RNA corresponding to, or a polypeptide encoded by, a nucleic acid sequence comprising an endogenous open reading frame (ORF) having a premature termination codon (PTC).

In another aspect, a TREM comprising an ASGPR binding moiety, or a composition thereof, described herein may be used in a method of modulating a production parameter of an mRNA corresponding to, or polypeptide encoded by, an endogenous open reading frame (ORF) in a subject, which ORF comprises a premature termination codon (PTC), contacting the subject with a TREM comprising an ASGPR binding moiety or a composition thereof in an amount and/or for a time sufficient to modulate the production parameter of the mRNA or polypeptide, wherein the TREM comprising an ASGPR binding moiety has an anticodon that pairs with the codon having the first sequence, thereby modulating the production parameter in the subject. In an embodiment, the production parameter comprises a signaling parameter and/or an expression parameter, e.g., as described herein.

In another aspect, a TREM comprising an ASGPR binding moiety, or a composition thereof, described herein may be used in a method of treating a subject having an endogenous open reading frame (ORF) which comprises a premature termination codon (PTC), comprising providing a TREM comprising an ASGPR binding moiety, or a composition thereof, wherein the TREM comprising an ASGPR binding moiety comprises an anticodon that pairs with the PTC in the ORF; contacting the subject with the TREM comprising an ASGPR binding moiety or a composition thereof in an amount and/or for a time sufficient to treat the subject, thereby treating the subject. In an embodiment, the PTC comprises UAA, UGA or UAG.

In another aspect, a TREM comprising an ASGPR binding moiety, or a composition thereof, described herein may be used in a method of treating a subject having an disease or disorder associated with a premature termination codon (PTC), comprising providing a TREM comprising an ASGPR binding moiety or a composition described herein; contacting the subject with the TREM comprising an ASGPR binding moiety or a composition thereof in an amount and/or for a time sufficient to treat the subject, thereby treating the subject. In an embodiment, the PTC comprises UAA, UGA or UAG. In an embodiment, the disease or disorder associated with a PTC is a disease or disorder described herein, e.g., a cancer or a monogenic disease.

Additional features of any of the aforesaid TREM entities (e.g., TREMs, TREM core fragments, TREM Fragments, TREM compositions, preparations, methods of making TREM compositions and preparations, and methods of using TREM compositions and preparations include one or more of the following enumerated embodiments).

Those skilled in the art will recognize or be able to ascertain using no more than routine experimentation, many equivalents to the specific embodiments of the invention described herein, including in the Drawings, Description, Examples, and Claims.

### BRIEF DESCRIPTION OF DRAWINGS

FIG. 1 is a table listing exemplary TREMs. The sequences of each of these TREMs are provided in the table, wherein r: ribonucleotide and the modifications are annotated as follows, for example: m: 2′-OMe; *: PS linkage; f: 2′-fluoro; moe: 2′-moe; d: deoxyribonucleotide; 5MeC: 5-methylcytosine; Cy3: a exemplary fluorophore; 5-LC-N: a linker; GalNAc: triantennary GalNAc as described herein. Thus, for example, mA represents 2′-O-methyl adenosine, moe5MeC represents 2′-MOE nucleotide with 5-methylcytosine nucleobase, and dA represents an adenosine deoxyribonucleotide.

### DETAILED DESCRIPTION OF CERTAIN EMBODIMENTS

The present disclosure features tRNA-based effector molecule (TREM) entities (e.g., TREMs, TREM Core Fragments, and TREM Fragments) comprising an asialoglycoprotein receptor (ASGPR) binding moiety bound to a sugar, nucleobase, and/or to the phosphate backbone at any nucleotide position, including a terminus, as well as related methods of use thereof. As disclosed herein, TREM entities (e.g., TREMs) are complex molecules which can mediate a variety of cellular processes. Pharmaceutical TREM compositions, e.g., TREMs comprising an ASGPR binding moiety, can be administered to a cell, a tissue, or to a subject to modulate these functions.

### Definitions

An “acceptor stem domain (AStD),” as that term is used herein, refers to a domain that binds an amino acid. In an embodiment, an AStD comprises an ASt Domain1 and an ASt Domain2. For example, the ASt Domain 1 is at or near the 5′ end of the TREM and the ASt Domain2 is at or near the 3′ end of the TREM. An AStD comprises sufficient RNA sequence to mediate, e.g., when present in an otherwise wildtype tRNA, acceptance of an amino acid, e.g., its cognate amino acid or a non-cognate amino acid, and transfer of the amino acid (AA) in the initiation or elongation of a polypeptide chain. Typically, the AStD comprises a 3′-end adenosine (CCA) for acceptor stem charging which is part of synthetase recognition. In an embodiment the ASID has at least 75, 80, 85, 85, 90, 95, or 100% identity with a naturally occurring AStD, e.g., an AStD encoded by a nucleic acid in Table 1. In an embodiment, the TREM can comprise a fragment or analog of an AStD, e.g., an AStD encoded by a nucleic acid in Table 1, which fragment in embodiments that has AStD activity and in other embodiments do not have AStD activity. One of ordinary skill can determine the relevant corresponding sequence for any of the domains, stems, loops, or other sequence features mentioned herein from a sequence encoded by a nucleic acid in Table 1. For example, one of ordinary skill can determine the sequence which corresponds to an AStD from a tRNA sequence encoded by a nucleic acid in Table 1. In an embodiment, the ASGPR binding moiety is present within the AStD (e.g., is bound to a nucleotide, for example, to a sugar moiety, a nucleobase, the internucleotide region, and/or a terminus within the AStD). In an embodiment, the ASGPR binding moiety is bound to a sugar moiety (e.g., ribose moiety) of a nucleotide in the AStD. In an embodiment, the ASGPR binding moiety is present within the internucleotide linkage (e.g., the phosphate backbone) in the AStD. In an embodiment, the ASGPR binding moiety is bound to a nucleobase in the AStD. In an embodiment, the ASGPR binding moiety is present on a terminus (e.g., the 5′ or 3′ terminus) within the AStD.

In an embodiment, the ASt Domain1 comprises positions 1-9 within the TREM sequence. In an embodiment, the ASGPR binding moiety is present within the ASt Domain1 (e.g., positions 1-9) within the TREM sequence. In an embodiment, the ASGPR binding moiety is bound to a sugar moiety (e.g., ribose moiety) of a nucleotide at positions 1-9 within a TREM sequence. In an embodiment, the ASGPR binding moiety is present within the internucleotide linkage (e.g., the phosphate backbone) at positions 1-9 within a TREM sequence. In an embodiment, the ASGPR binding moiety is bound to a nucleobase within a nucleotide at positions 1-9 within the TREM sequence. In an embodiment, the ASGPR binding moiety is bound to the 5′ terminus of the ASt Domain1 (e.g., position 1 of the ASt Domain1).

In an embodiment, the ASt Domain2 comprises positions 65-76 within the TREM sequence. In an embodiment, the ASGPR binding moiety is present within ASt Domain2 (e.g., positions 65-76) within the TREM sequence. In an embodiment, the ASGPR binding moiety is bound to a sugar moiety (e.g., ribose moiety) of a nucleotide at positions 65-76 within a TREM sequence. In an embodiment, the ASGPR binding moiety is present within the internucleotide linkage (e.g., the phosphate backbone) at positions 65-76 within a TREM sequence. In an embodiment, the ASGPR binding moiety is bound to a nucleobase within a nucleotide at positions 65-76 within the TREM sequence. In an embodiment, the ASGPR binding moiety is bound to the 3′ terminus of the ASt Domain2 (e.g., position 76 of the ASt Domain2).

In an embodiment the AStD falls under the corresponding sequence of a consensus sequence provided in the “Consensus Sequence” section or differs from the consensus sequence by no more than 1, 2, 5, or 10 positions. In an embodiment, the ASGPR binding moiety is present within the AStD which falls under the corresponding sequence of a consensus sequence provided in the “Consensus Sequence” section or differs from the consensus sequence by no more than 1, 2, 5, or 10 positions.

In an embodiment, the AStD comprises residues R₁-R₂-R₃-R₄-R₅-R₆-R₇ (an exemplary ASt Domain1) and residues R₆₅-R₆₆-R₆₇-R₆₈-R₆₉-R₇₀-R₇₁ (an exemplary ASt Domain2) of Formula I _{ZZZ}, wherein ZZZ indicates any of the twenty amino acids. In some embodiments, Formula I _{ZZZ} refers to all species.

In an embodiment, the AStD comprises residues R₁-R₂-R₃-R₄-R₅-R₆-R₇ and residues R₆₅-R₆₆-R₆₇-R₆₈-R₆₉-R₇₀-R₇₁ of Formula II _{ZZZ}, wherein ZZZ indicates any of the twenty amino acids. In some embodiments, Formula II _{ZZZ} refers to mammals.

In an embodiment, the ASID comprises residues R₁-R₂-R₃-R₄-R₅-R₆-R₇ and residues R₆₅-R₆₆-R₆₇-R₆₈-R₆₉-R₇₀-R₇₁ of Formula III _{ZZZ}, wherein ZZZ indicates any of the twenty amino acids. In some embodiments, Formula III _{ZZZ} refers to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

An “anticodon hairpin domain (ACHD)”, as that term is used herein, refers to a domain comprising an anticodon that binds a respective codon in an mRNA, and comprises sufficient sequence, e.g., an anticodon triplet, to mediate, e.g., when present in an otherwise wildtype tRNA, pairing (with or without wobble) with a codon. In an embodiment the ACHD has at least 75, 80, 85, 85, 90, 95, or 100% identity with a naturally occurring ACHD, e.g., an ACHD encoded by a nucleic acid in Table 1. In an embodiment, the TREM can comprise a fragment or analog of an ACHD, e.g., an ACHD encoded by a nucleic acid in Table 1, which fragment in embodiments has ACHD activity and in other embodiments does not have ACHD activity. In an embodiment, the ASGPR binding moiety is present within the ACHD (e.g., is bound to a nucleotide, for example, to a sugar moiety, a nucleobase, and/or the internucleotide region within the ACHD). In an embodiment, the ASGPR binding moiety is bound to a sugar moiety (e.g., ribose moiety) of a nucleotide in the ACHD. In an embodiment, the ASGPR binding moiety is presenting within the internucleotide linkage (e.g., the phosphate backbone) in the ACHD. In an embodiment, the ASGPR binding moiety is bound to a nucleobase within a nucleotide in the ACHD.

In an embodiment, the ACHD comprises positions 27-43 within the TREM sequence. In an embodiment, the ASGPR binding moiety is present within the ACHD (e.g., positions 27-43) within the TREM sequence. In an embodiment, the ASGPR binding moiety is present within the ACHD (e.g., positions 27-43). In an embodiment, the ASGPR binding moiety is bound to a sugar moiety (e.g., ribose moiety) of a nucleotide at positions 27-43) within a TREM sequence. In an embodiment, the ASGPR binding moiety is present within the internucleotide linkage (e.g., the phosphate backbone) at positions 27-43 within a TREM sequence. In an embodiment, the ASGPR binding moiety is bound to a nucleobase within a nucleotide at positions 27-43 within the TREM sequence.

In an embodiment the ACHD falls under the corresponding sequence of a consensus sequence provided in the “Consensus Sequence” section or differs from the consensus sequence by no more than 1, 2, 5, or 10 positions. In an embodiment, the ASGPR binding moiety is present within the ACHD which falls under the corresponding sequence of a consensus sequence provided in the “Consensus sequence” section or a sequence that differs from the consensus sequence by no more than 1, 2, 5, or 10 positions.

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula I _{ZZZ}, wherein ZZZ indicates any of the twenty amino acids. In some embodiments, Formula I _{ZZZ} refers to all species.

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula II 777, wherein ZZZ indicates any of the twenty amino acids. In some embodiments, Formula II _{ZZZ} refers to mammals.

In an embodiment, the ACHD comprises residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ of Formula III _{ZZZ}, wherein ZZZ indicates any of the twenty amino acids. In some embodiments, Formula III _{ZZZ} refers to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

In an embodiment, the anticodon of a TREM entity comprises three nucleotide residues and pairs with a three nucleotide codon. In an embodiment, the anticodon of a TREM entity consists of three nucleotide residues and pairs with an anticodon which consists of three nucleotide residues. In an embodiment the anticodon of the TREM entity does not pair with a codon having four, five or a larger number of nucleotide residues but pairs only with three codon nucleotide residues.

In an embodiment, the TREM entity does not alter the reading frame of an mRNA. In an embodiment, the anti-codon of a TREM entity pairs with a triplet codon of an mRNA, and docs not pair with an adjacent nucleotide.

In an embodiment, use of the TREM entity does not alter the length of the polypeptide transcribed from the mRNA, e.g., it does not suppress a termination codon, e.g., a premature termination codon. In an embodiment, the TREM does not alter the length of the ORF of an mRNA.

An “asialoglycoprotein receptor (ASGPR) binding moiety,” as that term is used herein, refers to a moiety which binds an asialoglycoprotein receptor. In an embodiment, the ASGPR binding moiety as described herein refers to structure comprising: (i) an ASGPR carbohydrate and (ii) a ASGPR linker (e.g., a linker connecting the carbohydrate to the TREM). Exemplary ASGPR moieties include galactose (Gal), galactosamine (GalNH₂), or an N-acetylgalactosamine (GalNAc) moiety, for example, a Gal, GalNH₂, or GalNAc, or an analog thereof. The ASGPR binding moieties may comprise functional groups (e.g., hydroxyl groups, carboxylate groups, amines) that may be protected by a chemical protecting group, e.g., an acetyl group or methyl group. In an embodiment, the ASGPR binding moiety comprises a triantennary GalNAc moiety. ASGPR binding moieties are described in further detail herein.

A “cognate adaptor function TREM,” as that term is used herein, refers to a TREM which mediates initiation or elongation with the AA (the cognate AA) associated in nature with the anti-codon of the TREM.

“Decreased expression,” as that term is used herein, refers to a decrease in comparison to a reference, e.g., in the case where altered control region, or addition of an agent, results in a decreased expression of the subject product, it is decreased relative to an otherwise similar cell without the alteration or addition.

A dihydrouridine hairpin domain (DHD), as that term is used herein, refers to a domain which comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a DHD mediates the stabilization of the TREM's tertiary structure. In an embodiment the DHD has at least 75, 80, 85, 85, 90, 95, or 100% identity with a naturally occurring DHD, e.g., a DHD encoded by a nucleic acid in Table 1. In an embodiment, the TREM can comprise a fragment or analog of a DHD, e.g., a DHD encoded by a nucleic acid in Table 1, which fragment in embodiments has DHD activity and in other embodiments does not have DHD activity. In an embodiment, the ASGPR binding moiety is present within the DHD (e.g., is bound to a nucleotide, for example, to a sugar moiety, a nucleobase, and/or the internucleotide region within the DHD). In an embodiment, the ASGPR binding moiety is bound to a sugar moiety (e.g., ribose moiety) of a nucleotide in the DHD. In an embodiment, the ASGPR binding moiety is present within the internucleotide linkage (e.g., the phosphate backbone) in the DHD. In an embodiment, the ASGPR binding moiety is bound to a nucleobase within a nucleotide in the DHD.

In an embodiment, the DHD comprises positions 10-26 within the TREM sequence. In an embodiment, the ASGPR binding moiety is present within the DHD (e.g., positions 10-26) within the TREM sequence. In an embodiment, the ASGPR binding moiety is bound to a sugar moiety (e.g., ribose moiety) of a nucleotide at positions 10-26 within a TREM sequence. In an embodiment, the ASGPR binding moiety is present within the internucleotide linkage (e.g., the phosphate backbone) at positions 10-26 within a TREM sequence. In an embodiment, the ASGPR binding moiety is bound to a nucleobase within a nucleotide at positions 10-26 within the TREM sequence.

In an embodiment the DHD falls under the corresponding sequence of a consensus sequence provided in the “Consensus Sequence” section, or differs from the consensus sequence by no more than 1, 2, 5, or 10 positions. In an embodiment, the ASGPR binding moiety is present within the DHD which falls under the corresponding sequence of a consensus sequence provided in the “Consensus Sequence” section or a sequence that differs from the consensus sequence by no more than 1, 2, 5, or 10 positions.

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula I _{ZZZ}, wherein ZZZ indicates any of the twenty amino acids. In some embodiments, Formula I _{ZZZ} refers to all species.

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula II _{ZZZ}, wherein ZZZ indicates any of the twenty amino acids. In some embodiments, Formula II _{ZZZ} refers to mammals.

In an embodiment, the DHD comprises residues R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ of Formula III _{ZZZ}, wherein ZZZ indicates any of the twenty amino acids. In some embodiments, Formula III _{ZZZ} refers to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

An “exogenous nucleic acid,” as that term is used herein, refers to a nucleic acid sequence that is not present in or differs by at least one nucleotide from the closest sequence in a reference cell, e.g., a cell into which the exogenous nucleic acid is introduced. In an embodiment, an exogenous nucleic acid comprises a nucleic acid that encodes a TREM.

An “exogenous TREM,” as that term is used herein, refers to a TREM that:
(a) differs by at least one nucleotide or one post transcriptional modification from the closest sequence tRNA in a reference cell, e.g., a cell into which the exogenous nucleic acid is introduced;
(b) has been introduced into a cell other than the cell in which it was transcribed;
(c) is present in a cell other than one in which it naturally occurs; or
(d) has an expression profile, e.g., level or distribution, that is non-wildtype, e.g., it is expressed at a higher level than wildtype. In an embodiment, the expression profile can be mediated by a change introduced into a nucleic acid that modulates expression or by addition of an agent that modulates expression of the RNA molecule. In an embodiment an exogenous TREM comprises 1, 2, 3 or 4 of properties (a)-(d).

A “GMP-grade composition,” as that term is used herein, refers to a composition in compliance with current good manufacturing practice (cGMP) guidelines, or other similar requirements. In an embodiment, a GMP-grade composition can be used as a pharmaceutical product.

As used herein, the terms “increasing” and “decreasing” refer to modulating that results in, respectively, greater or lesser amounts of function, expression, or activity of a particular metric relative to a reference. For example, subsequent to administration to a cell, tissue or subject of a TREM described herein, the amount of a marker of a metric (e.g., protein translation, mRNA stability, protein folding) as described herein may be increased or decreased by at least 5%, 10%, 15%, 20%, 25%, 30%, 35%, 40%, 45%, 50%, 55%, 60%, 65%, 70%, 75%, 80%, 85%, 90%, 95% or 98%, 2×, 3×, 5×, 10× or more relative to the amount of the marker prior to administration or relative to the effect of a negative control agent. The metric may be measured subsequent to administration at a time that the administration has had the recited effect, e.g., at least 12 hours, 24 hours, one week, one month, 3 months, or 6 months, after a treatment has begun.

“Increased expression,” as that term is used herein, refers to an increase in comparison to a reference, e.g., in the case where altered control region, or addition of an agent, results in an increased expression of the subject product, it is increased relative to an otherwise similar cell without the alteration or addition.

A Linker 2 region (L2), as that term is used herein, refers to a linker comprising residues R₈-R₉ of a consensus sequence provided in the “Consensus Sequence” section.

A Linker 3 region (L3), that term is used herein, refers to a linker comprising residue R₂₉ of a consensus sequence provided in the “Consensus Sequence” section.

A “Linker 4 region (L4), as that term is used herein, refers to a domain comprising residue R₇₂ of a consensus sequence provided in the “Consensus Sequence” section.

A “modification,” as that term is used herein with reference to a nucleotide, refers to a modification of the chemical structure, e.g., a covalent modification, of the subject nucleotide. The modification can be naturally occurring or non-naturally occurring. In an embodiment, the modification is present within the nucleobase, nucleotide sugar, or internucleotide linkage of a nucleotide of the TREM. In an embodiment, the modification is non-naturally occurring. In an embodiment, the modification is naturally occurring. In an embodiment, the modification is a synthetic modification. In an embodiment, the modification is a modification provided in Table 5.

A “naturally occurring nucleotide,” as that term is used herein, refers to a nucleotide that does not comprise a non-naturally occurring modification. In an embodiment, it includes a naturally occurring modification.

A “nucleotide,” as that term is used herein, refers to an entity comprising a sugar, typically a pentameric sugar; a nucleobase; and a phosphate linking group (e.g., internucleotide linkage). In an embodiment, a nucleotide comprises a naturally occurring, e.g., naturally occurring in a human cell, nucleotide, e.g., an adenine, thymine, guanine, cytosine, or uracil nucleotide.

A “thymine hairpin domain (THD), as that term is used herein, refers to a domain which comprises sufficient RNA sequence, to mediate, e.g., when present in an otherwise wildtype tRNA, recognition of the ribosome, e.g., acts as a recognition site for the ribosome to form a TREM-ribosome complex during translation. In an embodiment the THD has at least 75, 80, 85, 85, 90, 95, or 100% identity with a naturally occurring THD, e.g., a THD encoded by a nucleic acid in Table 1. In an embodiment, the TREM can comprise a fragment or analog of a THD, e.g., a THD encoded by a nucleic acid in Table 1, which fragment in embodiments has THD activity and in other embodiments does not have THD activity. In an embodiment, the ASGPR binding moiety is present within the THD (e.g., is bound to a nucleotide, for example, to a sugar moiety, a nucleobase, and/or the internucleotide region within the THD). In an embodiment, the ASGPR binding moiety is bound to a sugar moiety (e.g., ribose moiety) of a nucleotide in the THD. In an embodiment, the ASGPR binding moiety is present within the internucleotide linkage (e.g., the phosphate backbone) in the THD. In an embodiment, the ASGPR binding moiety is bound to a nucleobase within a nucleotide in the THD.

In an embodiment, the THD comprises positions 50-64 within the TREM sequence. In an embodiment, the ASGPR binding moiety is present within the THD (e.g., positions 50-64) within the TREM sequence. In an embodiment, the ASGPR binding moiety is bound to a sugar moiety (e.g., ribose moiety) of a nucleotide at positions 50-64 within a TREM sequence. In an embodiment, the ASGPR binding moiety is present within the internucleotide linkage (e.g., the phosphate backbone) at positions 50-64 within a TREM sequence. In an embodiment, the ASGPR binding moiety is bound to a nucleobase within a nucleotide at positions 50-64 within the TREM sequence.

In an embodiment the THD falls under the corresponding sequence of a consensus sequence provided in the “Consensus Sequence” section, or differs from the consensus sequence by no more than 1, 2, 5, or 10 positions. In an embodiment, the ASGPR binding moiety is present within the THD which falls under the corresponding sequence of a consensus sequence provided in the “Consensus Sequence” section or a sequence that differs from the consensus sequence by no more than 1, 2, 5, or 10 positions.

In an embodiment, the THD comprises residues -R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula I _{ZZZ}, wherein ZZZ indicates any of the twenty amino acids. In some embodiments, Formula I _{ZZZ} refers to all species.

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II _{ZZZ}, wherein ZZZ indicates any of the twenty amino acids. In some embodiments, Formula II _{ZZZ} refers to mammals.

In an embodiment, the THD comprises residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ of Formula II _{ZZZ}, wherein ZZZ indicates any of the twenty amino acids. In some embodiments, Formula III _{ZZZ} refers to humans.

In an embodiment, ZZZ indicates any of the amino acids: Alanine, Arginine, Asparagine, Aspartate, Cysteine, Glutamine, Glutamate, Glycine, Histidine, Isoleucine, Methionine, Leucine, Lysine, Phenylalanine, Proline, Serine, Threonine, Tryptophan, Tyrosine, or Valine.

A “tRNA-based effector molecule” or “TREM,” as that term is used herein, refers to an RNA molecule comprising a structure or property from (a)-(v) below, and which is a recombinant TREM, a synthetic TREM, or a TREM expressed from a heterologous cell. The TREMs described in the present invention are synthetic molecules and are made, e.g., in a cell free reaction, e.g., in a solid state or liquid phase synthetic reaction. TREMs are chemically distinct, e.g., in terms of primary sequence, type or location of modifications from the endogenous tRNA molecules made in cells, e.g., in mammalian cells, e.g., in human cells. A TREM can have a plurality (e.g., 2, 3, 4, 5, 6, 7, 8, 9) of the structures and functions of (a)-(v).

In an embodiment, a TREM is non-native, as evaluated by structure or the way in which it was made.

In an embodiment, a TREM comprises one or more of the following structures or properties:
(a′) an optional linker region of a consensus sequence provided in the “Consensus Sequence” section, e.g., a Linker 1 region;
(a) an acceptor stem domain (an AStD), which typically comprises an ASt Domain1 and an ASt Domain2.
(a′-1) a Linker 2 region (L2) a linker comprising residues R₈-R₉ of a consensus sequence provided in the “Consensus Sequence” section, e.g., a Linker 2 region;
(b) a DHD or dihydrouridine hairpin domain (DHD);
(b′-1) a Linker 3 region, or L3;
(c) an ACHD or anticodon hairpin domain;
(d) a VLD, or variable loop domain (VLD);
(e) a THD or thymine hairpin domain (THD);
(c′1) an L4 linker comprising residue R₇₂ of a consensus sequence provided in the “Consensus Sequence” section;
(f) under physiological conditions, it comprises a stem structure and one or a plurality of loop structures, e.g., 1, 2, or 3 loops. A loop can comprise a domain described herein, e.g., a domain selected from (a)-(e). A loop can comprise one or a plurality of domains. In an embodiment, a stem or loop structure has at least 75, 80, 85, 85, 90, 95, or 100% identity with a naturally occurring stem or loop structure, e.g., a stem or loop structure encoded by a nucleic acid in Table 1. In an embodiment, the TREM can comprise a fragment or analog of a stem or loop structure, e.g., a stem or loop structure encoded by a nucleic acid in Table 1, which fragment in embodiments has activity of a stem or loop structure, and in other embodiments does not have activity of a stem or loop structure;
(g) a tertiary structure, e.g., an L-shaped tertiary structure;
(h) adaptor function, i.e., the TREM mediates acceptance of an amino acid, e.g., its cognate amino acid and transfer of the AA in the initiation or elongation of a polypeptide chain;
(i) cognate adaptor function wherein the TREM mediates acceptance and incorporation of an amino acid (e.g., cognate amino acid) associated in nature with the anti-codon of the TREM to initiate or elongate a polypeptide chain;
(j) non-cognate adaptor function, wherein the TREM mediates acceptance and incorporation of an amino acid (e.g., non-cognate amino acid) other than the amino acid associated in nature with the anti-codon of the TREM in the initiation or elongation of a polypeptide chain;
(k) a regulatory function, e.g., an epigenetic function (e.g., gene silencing function or signaling pathway modulation function), cell fate modulation function, mRNA stability modulation function, protein stability modulation function, protein transduction modulation function, or protein compartmentalization function;
(l) a structure which allows for ribosome binding;
(m) a post-transcriptional modification, e.g., a naturally occurring post-transcriptional modification;
(n) the ability to inhibit a functional property of a tRNA, e.g., any of properties (h)-(k) possessed by a tRNA;
(o) the ability to modulate cell fate;
(p) the ability to modulate ribosome occupancy;
(q) the ability to modulate protein translation;
(r) the ability to modulate mRNA stability;
(s) the ability to modulate protein folding and structure;
(t) the ability to modulate protein transduction or compartmentalization;
(u) the ability to modulate protein stability; or
(v) the ability to modulate a signaling pathway, e.g., a cellular signaling pathway.

In an embodiment, a TREM comprises a full-length tRNA molecule or a fragment thereof.

In an embodiment, a TREM comprises the following properties: (a)-(e).

In an embodiment, a TREM comprises the following properties: (a) and (c).

In an embodiment, a TREM comprises the following properties: (a), (c) and (h).

In an embodiment, a TREM comprises the following properties: (a), (c), (h) and (b).

In an embodiment, a TREM comprises the following properties: (a), (c), (h) and (e).

In an embodiment, a TREM comprises the following properties: (a), (c), (h), (b) and (e).

In an embodiment, a TREM comprises the following properties: (a), (c), (h), (b), (e) and (g).

In an embodiment, a TREM comprises the following properties: (a), (c), (h) and (m).

In an embodiment, a TREM comprises the following properties: (a), (c), (h), (m), and (g).

In an embodiment, a TREM comprises the following properties: (a), (c), (h), (m) and (b).

In an embodiment, a TREM comprises the following properties: (a), (c), (h), (m) and (e).

In an embodiment, a TREM comprises the following properties: (a), (c), (h), (m), (g), (b) and (e).

In an embodiment, a TREM comprises the following properties: (a), (c), (h), (m), (g), (b), (e) and (q).

In an embodiment, a TREM comprises:
(i) an amino acid attachment domain that binds an amino acid (e.g., an AStD, as described in (a) herein; and
(ii) an anticodon that binds a respective codon in an mRNA (e.g., an ACHD, as described in (c) herein).

In an embodiment the TREM comprises a flexible RNA linker which provides for covalent linkage of (i) to (ii).

In an embodiment, the TREM mediates protein translation.

In an embodiment a TREM comprises a linker, e.g., an RNA linker, e.g., a flexible RNA linker, which provides for covalent linkage between a first and a second structure or domain. In an embodiment, an RNA linker comprises at least 1, 2, 3, 4, 5, 6, 7, 8, 9, 10, 11, 12, 13, 14 or 15 ribonucleotides. A TREM can comprise one or a plurality of linkers, e.g., in embodiments a TREM comprising (a), (b), (c), (d) and (e) can have a first linker between a first and second domain, and a second linker between a third domain and another domain.

In an embodiment, the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

In an embodiment, a TREM comprises an RNA sequence at least 60, 65, 70, 75, 80, 85, 90, 95, 96, 97, 98 or 99% identical with, or which differs by no more than 1, 2, 3, 4, 5, 10, 15, 20, 25, or 30 ribonucleotides from, an RNA sequence encoded by a DNA sequence listed in Table 1, or a fragment or functional fragment thereof. In an embodiment, a TREM comprises an RNA sequence encoded by a DNA sequence listed in Table 1, or a fragment or functional fragment thereof. In an embodiment, a TREM comprises an RNA sequence encoded by a DNA sequence at least 60, 65, 70, 75, 80, 85, 90, 95, 96, 97, 98 or 99% identical with a DNA sequence listed in Table 1, or a fragment or functional fragment thereof. In an embodiment, a TREM comprises a TREM domain, e.g., a domain described herein, comprising at least 60, 65, 70, 75, 80, 85, 90, 95, 96, 97, 98, or 99% identical with, or which differs by no more than 1, 2, 3, 4, 5, 10, or 15, ribonucleotides from, an RNA encoded by a DNA sequence listed in Table 1, or a fragment or a functional fragment thereof. In an embodiment, a TREM comprises a TREM domain, e.g., a domain described herein, comprising an RNA sequence encoded by DNA sequence listed in Table 1, or a fragment or functional fragment thereof. In an embodiment, a TREM comprises a TREM domain, e.g., a domain described herein, comprising an RNA sequence encoded by DNA sequence at least 60, 65, 70, 75, 80, 85, 90, 95, 96, 97, 98 or 99% identical with a DNA sequence listed in Table 1, or a fragment or functional fragment thereof.

In an embodiment, a TREM is 76-90 nucleotides in length. In embodiments, a TREM or a fragment or functional fragment thereof is between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 20-90 nucleotides, between 20-80 nucleotides, 20-70 nucleotides, between 20-60 nucleotides, between 20-50 nucleotides, between 20-40 nucleotides, between 30-90 nucleotides, between 30-80 nucleotides, between 30-70 nucleotides, between 30-60 nucleotides, or between 30-50 nucleotides.

In an embodiment, a TREM is aminoacylated, e.g., charged, with an amino acid by an aminoacyl tRNA synthetase.

In an embodiment, a TREM is not charged with an amino acid, e.g., an uncharged TREM (uTREM).

In an embodiment, a TREM comprises less than a full length tRNA. In embodiments, a TREM can correspond to a naturally occurring fragment of a tRNA, or to a non-naturally occurring fragment. Exemplary fragments include: TREM halves (e.g., from a cleavage in the ACHD, e.g., in the anticodon sequence, e.g., 5′halves or 3′ halves); a 5′ fragment (e.g., a fragment comprising the 5′ end, e.g., from a cleavage in a DHD or the ACHD); a 3′ fragment (e.g., a fragment comprising the 3′ end, e.g., from a cleavage in the THD); or an internal fragment (e.g., from a cleavage in one or more of the ACHD, DHD or THD).

A “TREM core fragment,” as that term is used herein, refers to a portion of the sequence of Formula B: [L1]_{y}-[ASt Domain1]ₓ-[L2]_{y}-[DH Domain]_{y}-[L3]_{y}-[ACH Domain]ₓ-[VL Domain] y-[TH Domain] y-[L4] y-[ASt Domain2]ₓ, wherein: x=1 and y=0 or 1.

A “TREM fragment,” as used herein, refers to a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2].

A “non-cognate adaptor function TREM,” as that term is used herein, refers to a TREM which mediates initiation or elongation with an AA (a non-cognate AA) other than the AA associated in nature with the anti-codon of the TREM. In an embodiment, a non-cognate adaptor function TREM is also referred to as a mischarged TREM (mTREM).

A “non-naturally occurring sequence,” as that term is used herein, refers to a sequence wherein an Adenine is replaced by a residue other than an analog of adenine, a cytosine is replaced by a residue other than an analog of cytosine, a guanine is replaced by a residue other than an analog of guanine, and a uracil is replaced by a residue other than an analog of uracil. An analog refers to any possible derivative of the ribonucleotides, A, G, C or U. In an embodiment, a sequence having a derivative of any one of ribonucleotides A, G, C or U is a non-naturally occurring sequence.

A “pharmaceutical TREM composition,” as that term is used herein, refers to a TREM composition that is suitable for pharmaceutical use. Typically, a pharmaceutical TREM composition comprises a pharmaceutical excipient. In an embodiment the TREM will be the only active ingredient in the pharmaceutical TREM composition. In embodiments the pharmaceutical TREM composition is free, substantially free, or has less than a pharmaceutically acceptable amount, of host cell proteins, DNA, e.g., host cell DNA, endotoxins, and bacteria.

A “post-transcriptional processing,” as that term is used herein, with respect to a subject molecule, e.g., a TREM, RNA or tRNAs, refers to a covalent modification of the subject molecule. In an embodiment, the covalent modification occurs post-transcriptionally. In an embodiment, the covalent modification occurs co-transcriptionally. In an embodiment the modification is made in vivo, e.g., in a cell used to produce a TREM. In an embodiment the modification is made ex vivo, e.g., it is made on a TREM isolated or obtained from the cell which produced the TREM. In an embodiment, the post-transcriptional modification is selected from a post-transcriptional modification listed in Table 2.

A “subject,” as this term is used herein, includes any organism, such as a human or other animal. In embodiments, the subject is a vertebrate animal (e.g., mammal, bird, fish, reptile, or amphibian). In embodiments, the subject is a mammal, e.g., a human. In embodiments, the method subject is a non-human mammal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). The subject may be a male or female of any age group, e.g., a pediatric subject (e.g., infant, child, adolescent) or adult subject (e.g., young adult, middle-aged adult, or senior adult)). A non-human subject may be a transgenic animal.

A “synthetic TREM,” as that term is used herein, refers to a TREM which was synthesized other than in or by a cell having an endogenous nucleic acid encoding the TREM, e.g., a synthetic TREM is synthetized by cell-free solid phase synthesis. A synthetic TREM can have the same, or a different, sequence, or tertiary structure, as a native tRNA.

A “recombinant TREM,” as that term is used herein, refers to a TREM that was expressed in a cell modified by human intervention, having a modification that mediates the production of the TREM, e.g., the cell comprises an exogenous sequence encoding the TREM, or a modification that mediates expression, e.g., transcriptional expression or post-transcriptional modification, of the TREM. A recombinant TREM can have the same, or a different, sequence, set of post-transcriptional modifications, or tertiary structure, as a reference tRNA, e.g., a native tRNA.

A “tRNA”, as that term is used herein, refers to a naturally occurring transfer ribonucleic acid in its native state.

A “TREM composition,” as that term is used herein, refers to a composition comprising a plurality of TREMs, a plurality of TREM core fragments and/or a plurality of TREM fragments. A TREM composition can comprise one or more species of TREMs, TREM core fragments or TREM fragments. In an embodiment, the composition comprises only a single species of TREM, TREM core fragment or TREM fragment. In an embodiment, the TREM composition comprises a first TREM, TREM core fragment or TREM fragment species; and a second TREM, TREM core fragment or TREM fragment species. In an embodiment, the TREM composition comprises X TREM, TREM core fragment or TREM fragment species, wherein X=2, 3, 4, 5, 6, 7, 8, 9, or 10. In an embodiment, the TREM, TREM core fragment or TREM fragment has at least 70, 75, 80, 85, 90, or 95, or has 100%, identity with a sequence encoded by a nucleic acid in Table 1. A TREM composition can comprise one or more species of TREMs, TREM core fragments or TREM fragments. In an embodiment, the TREM composition is at least 10, 20, 30, 40, 50, 60, 70, 80, 90, 95 or 99% dry weight TREMs (for a liquid composition dry weight refers to the weight after removal of substantially all liquid, e.g., after lyophilization). In an embodiment, the composition is a liquid. In an embodiment, the composition is dry, e.g., a lyophilized material. In an embodiment, the composition is a frozen composition. In an embodiment, the composition is sterile. In an embodiment, the composition comprises at least 0.5 g, 1.0 g, 5.0 g, 10 g, 15 g, 25 g, 50 g, 100 g, 200 g, 400 g, or 500 g (e.g., as determined by dry weight) of TREM.

In an embodiment, at least X % of the TREMs in a TREM composition comprises a chemical modification at a selected position, and X is 80, 90, 95, 96, 97, 98, 99, or 99.5.

In an embodiment, at least X % of the TREMs in a TREM composition comprises a chemical modification at a first position and a chemical modification at a second position, and X, independently, is 80, 90, 95, 96, 97, 98, 99, or 99.5. In embodiments, the modification at the first and second position is the same. In embodiments, the modification at the first and second position are different. In embodiments, the nucleotide at the first and second position is the same, e.g., both are adenine. In embodiments, the nucleotide at the first and second position are different, e.g., one is adenine and one is thymine.

In an embodiment, at least X % of the TREMs in a TREM composition comprises a chemical modification at a first position and less than Y % have a chemical modification at a second position, wherein X is 80, 90, 95, 96, 97, 98, 99, or 99.5 and Y is 20, 20, 5, 2, 1, 0.1, or 0.01. In embodiments, the nucleotide at the first and second position is the same, e.g., both are adenine. In embodiments the nucleotide at the first and second position are different, e.g., one is adenine and one is thymine.

A “variable loop domain (VLD),” as that term is used herein refers to a domain which comprises sufficient RNA sequence to mediate, e.g., when present in an otherwise wildtype tRNA, recognition of aminoacyl-tRNA synthetase, e.g., acts as a recognition site for aminoacyl-tRNA synthetase for amino acid charging of the TREM. In embodiments, a VLD mediates the stabilization of the TREM's tertiary structure. In an embodiment, a VLD modulates, e.g., increases, the specificity of the TREM, e.g., for its cognate amino acid, e.g., the VLD modulates the TREM's cognate adaptor function. In an embodiment the VLD has at least 75, 80, 85, 85, 90, 95, or 100% identity with a naturally occurring VLD, e.g., a VLD encoded by a nucleic acid in Table 1. In an embodiment, the TREM can comprise a fragment or analog of a VLD, e.g., a VLD encoded by a nucleic acid in Table 1, which fragment in embodiments has VLD activity and in other embodiments does not have VLD activity. In an embodiment, the ASGPR binding moiety is present within the VLD (e.g., is bound to a nucleotide, for example, to a sugar moiety, a nucleobase, and/or the internucleotide region within the VLD). In an embodiment, the ASGPR binding moiety is bound to a sugar moiety (e.g., ribose moiety) of a nucleotide in the VLD. In an embodiment, the ASGPR binding moiety is presenting within the internucleotide linkage (e.g., the phosphate backbone) in the VLD. In an embodiment, the ASGPR binding moiety is bound to a nucleobase within a nucleotide in the VLD.

In an embodiment, the VLD comprises positions 44-49 within the TREM sequence. In an embodiment, the ASGPR binding moiety is present within the VLD (e.g., positions 44-49) within the TREM sequence. In an embodiment, the ASGPR binding moiety is bound to a sugar moiety (e.g., ribose moiety) of a nucleotide at positions 44-49 within a TREM sequence. In an embodiment, the ASGPR binding moiety is present within the internucleotide linkage (e.g., the phosphate backbone) at positions 44-49 within a TREM sequence. In an embodiment, the ASGPR binding moiety is bound to a nucleobase within a nucleotide at positions 44-49 within the TREM sequence.

In an embodiment the VLD falls under the corresponding sequence of a consensus sequence provided in the “Consensus Sequence” section. In an embodiment, the ASGPR binding moiety is present within the VLD which falls under the corresponding sequence of a consensus sequence provided in the “Consensus Sequence” section or differs from the consensus sequence by no more than 1, 2, 5, or 10 positions.

In an embodiment, the VLD comprises residue -[R₄₇]ₓ of a consensus sequence provided in the “Consensus Sequence” section, wherein x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271).

### TREM Entities

Described herein are TREM entities, e.g., a TREM, a TREM Core Fragment, or a TREM Fragment, modified with an asialoglycoprotein receptor (ASGPR) binding moiety, as well as compositions and methods of use thereof. A TREM entity (e.g., a TREM) refers to an RNA molecule comprising one or more of the properties described herein. A TREM entity (e.g., a TREM) can comprise a chemical modification, e.g., as provided in Table 5.

In an embodiment, the ASGPR binding moiety is bound to an adenine nucleobase at a carbon atom or a nitrogen atom. In an embodiment, the ASGPR binding moiety is bound to an adenine at the C2 position, N9 position, or C8 position. In an embodiment, the ASGPR binding moiety is bound to the adenine at the C2 position. In an embodiment, the ASGPR binding moiety is bound to the adenine at the N9 position. In an embodiment, the ASGPR binding moiety is bound to the adenine at the C8 position. In an embodiment, the ASGPR binding moiety is bound to a substituent on the adenosine nucleobase, e.g., an amine on the adenine nucleobase (e.g., amine off the C6 position).

In an embodiment, the ASGPR binding moiety is bound to a guanine nucleobase at a carbon or nitrogen atom. In an embodiment, the ASGPR binding moiety is bound to the guanine at the N1, C2, N9, or C8 position. In an embodiment, the ASGPR binding moiety is bound to the guanine at the NI position. In an embodiment, the ASGPR binding moiety is bound to the guanine at the C2 position. In an embodiment, the ASGPR binding moiety is bound to the guanine at the N9 position. In an embodiment, the ASGPR binding moiety is bound to the guanine at the C8 position. In an embodiment, the ASGPR binding moiety is bound to a substituent on the guanosine nucleobase, e.g., an amine on the guanosine nucleobase (e.g., amine off the C2 position).

In an embodiment, the ASGPR binding moiety is bound to a cytosine nucleobase at a carbon atom. In an embodiment, the ASGPR binding moiety is bound to the cytosine at the C4, C5, or C6 position. In an embodiment, the ASGPR binding moiety is bound to the cytosine at the C4 position. In an embodiment, the ASGPR binding moiety is bound to the cytosine at the C5 position. In an embodiment, the ASGPR binding moiety is bound to the cytosine at the C6 position. In an embodiment, the ASGPR binding moiety is bound to a substituent on the cytosine nucleobase, e.g., an amine on the cytosine nucleobase (e.g., amine off the C4 position).

In an embodiment, the ASGPR binding moiety is bound to a uracil nucleobase at a carbon or nitrogen atom. In an embodiment, the ASGPR binding moiety is bound to the uracil at the N3, C5, or C6 position. In an embodiment, the ASGPR binding moiety is bound to the uracil at the N3 position. In an embodiment, the ASGPR binding moiety is bound to the uracil at the C5 position. In an embodiment, the ASGPR binding moiety is bound to the uracil at the C6 position.

In an embodiment, the ASGPR binding moiety is bound to a thymine nucleobase at a carbon or a nitrogen atom. In an embodiment, the ASGPR binding moiety is bound to the thymine at the N3, C5, or C6 position. In an embodiment, the ASGPR binding moiety is bound to the thymine at the N3 position. In an embodiment, the ASGPR binding moiety is bound to the thymine at the C5 position. In an embodiment, the ASGPR binding moiety is bound to the thymine at the C6 position. In an embodiment, the ASGPR binding moiety is bound to a substituent on the thymine nucleobase, e.g., a methyl on the thymine nucleobase (e.g., a methyl off the C5 position).

In an embodiment, the ASGPR binding moiety is bound to the terminal nucleotide of a TREM molecule. In an embodiment, the terminal nucleotide is an adenine, a guanine, a cytosine, thymine, a uracil, or a variant thereof. In an embodiment, the ASGPR binding moiety is bound to the 5′ and/or 3′ terminal nucleotide of the TREM molecule. In an embodiment, the ASGPR binding moiety is bound to the 5′ terminal nucleotide. In an embodiment, the ASGPR binding moiety is bound to the 3′ terminal nucleotide. In an embodiment, the ASGPR binding moiety is bound to the 5′ terminal nucleotide and the 3′ terminal nucleotide. In an embodiment, the ASGPR binding moiety is bound to the 5′ terminal nucleotide at the C5 hydroxyl group of the sugar moiety (e.g., ribose moiety). In an embodiment, the ASGPR binding moiety is bound to the terminal nucleotide at the 5′ hydroxyl group. In an embodiment, the ASGPR binding moiety is bound to the 3′ terminal nucleotide at the C3′ ribose position.

In an embodiment, a TREM entity includes a TREM comprising a sequence of Formula A; a TREM core fragment comprising a sequence of Formula B; or a TREM fragment comprising a portion of a TREM which TREM comprises a sequence of Formula A.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the ASt Domain 1 (e.g., on a sugar moiety (e.g., ribose moiety), on the phosphate backbone, on a nucleobase, or at a terminus (e.g., the 5′ terminus) within the ASt Domain 1). In an embodiment, the ASGPR binding moiety is bound to a sugar moiety (e.g., ribose moiety) within the ASt Domain1. In an embodiment, the ASGPR binding moiety is bound to the phosphate backbone at any nucleotide position within the ASt Domain 1. In an embodiment, the ASGPR binding moiety is present on a nucleobase of a nucleotide within ASt Domain1. In an embodiment, the ASGPR binding moiety is present at the 5′ terminus within ASt Domain1 or at [L1]. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the ASt Domain 2 (e.g., on a sugar moiety (e.g., ribose moiety), on the phosphate backbone, on a nucleobase, or at a terminus (e.g., the 3′ terminus) within the ASt Domain 2). In an embodiment, the ASGPR binding moiety is bound to a sugar (e.g., ribose moiety) within the ASt Domain2. In an embodiment, the ASGPR binding moiety is bound to the phosphate backbone at any nucleotide position within the ASt Domain 2. In an embodiment, the ASGPR binding moiety is present on a nucleobase of a nucleotide within ASt Domain2. In an embodiment, the ASGPR binding moiety is present at the 3′ terminus within ASt Domain2. In an embodiment, the ASGPR binding moiety is present within an internucleotide linkage of ASt Domain2. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the either one or both of the ASt Domain 1 and ASt Domain 2 (e.g., on a sugar moiety (e.g., ribose moiety), on the phosphate backbone, on a nucleobase, or at a terminus (e.g., 5′ or 3′ terminus) within the ASt Domain 1 and/or ASt Domain 2). In an embodiment, the ASGPR binding moiety is bound to a sugar moiety (e.g., ribose moiety) within either one or both of the ASt Domain1 and ASt Domain 2. In an embodiment, the ASGPR binding moiety is bound to the phosphate backbone at any nucleotide position within either one or both of the ASt Domain 1 and ASt Domain 2. In an embodiment, the ASGPR binding moiety is present on a nucleobase of a nucleotide within ASt Domain1 or ASt Domain2. In an embodiment, the ASGPR binding moiety is present at the 5′ terminus within ASt Domain1 or [L1] or the 3′ terminus within ASt Domain2. In an embodiment, the ASGPR binding moiety is present within an internucleotide linkage of ASt Domain1 or ASt Domain2. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the DH Domain (e.g., on a sugar moiety (e.g., ribose moiety), on the phosphate backbone, or on a nucleobase of a nucleotide within the DH Domain). In an embodiment, the ASGPR binding moiety is bound to a sugar moiety (e.g., ribose moiety) within the DH Domain. In an embodiment, the ASGPR binding moiety is bound to the phosphate backbone at any nucleotide position within the DH Domain. In an embodiment, the ASGPR binding moiety is present on a nucleobase of a nucleotide within the DH Domain. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the ACH Domain (e.g., on a sugar moiety (e.g., ribose moiety), on the phosphate backbone, or on a nucleobase of a nucleotide within the ACH Domain). In an embodiment, the ASGPR binding moiety is bound to a sugar moiety (e.g., ribose moiety) within the ACH Domain. In an embodiment, the ASGPR binding moiety is bound to the phosphate backbone at any nucleotide position within the ACH Domain. In an embodiment, the ASGPR binding moiety is present on a nucleobase of a nucleotide within the ACH Domain. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the VL Domain (e.g., on a sugar moiety (e.g., ribose moiety), on the phosphate backbone, or on a nucleobase of a nucleotide within the VL Domain). In an embodiment, the ASGPR binding moiety is bound to a sugar moiety (e.g., ribose moiety) within the VL Domain. In an embodiment, the ASGPR binding moiety is bound to the phosphate backbone at any nucleotide position within the VL Domain. In an embodiment, the ASGPR binding moiety is present on a nucleobase of a nucleotide within the VL Domain. In an embodiment, [L1] is optional. In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is present within the TH Domain (e.g., on a sugar moiety (e.g., ribose moiety), on the phosphate backbone, or on a nucleobase of a nucleotide within the TH Domain). In an embodiment, the ASGPR binding moiety is bound to a sugar moiety (e.g., ribose moiety) within the TH Domain. In an embodiment, the ASGPR binding moiety is bound to the phosphate backbone at any nucleotide position within the TDH Domain. In an embodiment, the ASGPR binding moiety is present on a nucleobase of a nucleotide within the TH Domain. In an embodiment, [VL Domain] is optional. In an embodiment, [L1] is optional.

In an embodiment, a TREM comprises a sequence of Formula A: [L1]-[ASt Domain1]-[L2]-[DH Domain]-[L3]-[ACH Domain]-[VL Domain]-[TH Domain]-[L4]-[ASt Domain2], wherein the ASGPR binding moiety is bound to a nucleobase within one or more domains selected from [ASt Domain1], [DH Domain], [ACH Domain], [TH Domain], and/or [ASt Domain2]. In an embodiment, [VL Domain] is optional. In an embodiment, [L1] is optional.

In an embodiment, a TREM core fragment comprises a sequence of Formula B: [L1] y-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is present within either one or both of the ASt Domain 1 and ASt Domain 2 (e.g., on a sugar moiety (e.g., ribose moiety), on the phosphate backbone, or on a nucleobase within the ASt Domain 1 and/or ASt Domain 2). In an embodiment, the ASGPR binding moiety is bound to a sugar moiety (e.g., ribose moiety) within either one or both of the ASt Domain1 and AST Domain 2. In an embodiment, the ASGPR binding moiety is bound to the phosphate backbone at any nucleotide position within either one or both of the ASt Domain 1 and ASt Domain 2. In an embodiment, the ASGPR binding moiety is bound to a nucleobase within a nucleotide within one or both of ASt Domain1 and ASt Domain2. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is present within the DH Domain (e.g., on a sugar moiety (e.g., ribose moiety), on the phosphate backbone, or on a nucleobase within the DH Domain). In an embodiment, the ASGPR binding moiety is bound to a sugar moiety (e.g., ribose moiety) within the DH Domain. In an embodiment, the ASGPR binding moiety is bound to the phosphate backbone at any nucleotide position within the DH Domain. In an embodiment, the ASGPR binding moiety is bound to a nucleobase within a nucleotide within the DH Domain. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is present within the ACH Domain (e.g., on a sugar moiety (e.g., ribose moiety), on the phosphate backbone, or on a nucleobase within the ACH Domain). In an embodiment, the ASGPR binding moiety is bound to a sugar moiety (e.g., ribose moiety) within the ACH Domain. In an embodiment, the ASGPR binding moiety is bound to the phosphate backbone at any nucleotide position within the ACH Domain. the ASGPR binding moiety is bound to a nucleobase within a nucleotide within the ACH Domain. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is present within the VL Domain (e.g., on a sugar moiety (e.g., ribose moiety) or on the phosphate backbone within the VL Domain). In an embodiment, the ASGPR binding moiety is bound to a sugar moiety (e.g., ribose moiety) within the VL Domain. In an embodiment, the ASGPR binding moiety is bound to the phosphate backbone at any nucleotide position within the VL Domain. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is present within the TH Domain (e.g., on a sugar moiety (e.g., ribose moiety), on the phosphate backbone, or on a nucleobase within the TH Domain). In an embodiment, the ASGPR binding moiety is bound to a sugar moiety (e.g., ribose moiety) within the TH Domain. In an embodiment, the ASGPR binding moiety is bound to the phosphate backbone at any nucleotide position within the TH Domain. In an embodiment, the ASGPR binding moiety is bound to a nucleobase within a nucleotide within the TH Domain. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is bound to a sugar moiety (e.g., ribose moiety) of a nucleotide within one or more domains selected from [ASt Domain1], [DH Domain], [ACH Domain], [TH Domain], and/or [ASt Domain2]. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is bound to a phosphate backbone of a nucleotide within one or more domains selected from [ASt Domain1], [DH Domain], [ACH Domain], [TH Domain], and/or [ASt Domain2]. In an embodiment, y=0. In an embodiment, y=1.

In an embodiment, a TREM core fragment comprises a sequence of Formula B: [L1]_{y}-[ASt Domain1]ₓ-[L2]_{y}-[DH Domain]_{y}-[L3]_{y}-[ACH Domain]ₓ-[VL Domain]_{y}-[TH Domain]_{y}-[L4]_{y}-[ASt Domain2]ₓ, wherein: x=1 and y=0 or 1, and the ASGPR binding moiety is bound to a nucleobase within one or more domains selected from [ASt Domain1], [DH Domain], [ACH Domain], [TH Domain], and/or [ASt Domain2]. In an embodiment, y=0. In an embodiment, y=1.

In an embodiment, a TREM fragment comprises a portion of a TREM, wherein the TREM comprises a sequence of Formula A: [L1]-[ASt Domain1]-[L2]-[DH Domain]-[L3]-[ACH Domain]-[VL Domain]-[TH Domain]-[L4]-[ASt Domain2], and wherein the TREM fragment comprises: one, two, three or all or any combination of the following: a TREM half (e.g., from a cleavage in the ACH Domain, e.g., in the anticodon sequence, e.g., a 5′half or a 3′ half); a 5′ fragment (e.g., a fragment comprising the 5′ end, e.g., from a cleavage in a DH Domain or the ACH Domain); a 3′ fragment (e.g., a fragment comprising the 3′ end, e.g., from a cleavage in the TH Domain); or an internal fragment (e.g., from a cleavage in any one of the ACH Domain, DH Domain or TH Domain). Exemplary TREM fragments include TREM halves (e.g., from a cleavage in the ACHD, e.g., 5′TREM halves or 3′ TREM halves), a 5′ fragment (e.g., a fragment comprising the 5′ end, e.g., from a cleavage in a DHD or the ACHD), a 3′ fragment (e.g., a fragment comprising the 3′ end of a TREM, e.g., from a cleavage in the THD), or an internal fragment (e.g., from a cleavage in one or more of the ACHD, DHD or THD).

In an embodiment, a TREM, a TREM core fragment or a TREM fragment can be charged with an amino acid (e.g., a cognate amino acid); charged with a non-cognate amino acid (e.g., a mischarged TREM (mTREM)); or not charged with an amino acid (e.g., an uncharged TREM (uTREM)). In an embodiment, a TREM, a TREM core fragment or a TREM fragment can be charged with an amino acid selected from alanine, arginine, asparagine, aspartate, cysteine, glutaminc, glutamate, glycinc, histidine, isolcucine, methionine, leucinc, lysinc, phenylalanine, proline, serine, threonine, tryptophan, tyrosine, or valine.

In an embodiment, the TREM, TREM core fragment or TREM fragment is a cognate TREM. In an embodiment, the TREM, TREM core fragment or TREM fragment is a non-cognate TREM. In an embodiment, the TREM, TREM core fragment or TREM fragment recognizes a codon provided in Table 2 or Table 3.

TABLE 2
List of codons
AAA
AAC
AAG
AAU
ACA
ACC
ACG
ACU
AGA
AGC
AGG
AGU
AUA
AUC
AUG
AUU
CAA
CAC
CAG
CAU
CCA
CCC
CCG
CCU
CGA
CGC
CGG
CGU
CUA
CUC
CUG
CUU
GAA
GAC
GAG
GAU
GCA
GCC
GCG
GCU
GGA
GGC
GGG
GGU
GUA
GUC
GUG
GUU
UAA
UAC
UAG
UAU
UCA
UCC
UCG
UCU
UGA
UGC
UGG
UGU
UUA
UUC
UUG
UUU

TABLE 3
Amino acids and corresponding codons

| Amino Acid | mRNA codons |
|---|---|
| Alanine | GCU, GCC, GCA, GCG |
| Arginine | CGU, CGC, CGA, CGG, AGA, AGG |
| Asparagine | AAU, AAC |
| Aspartate | GAU, GAC |
| Cysteine | UGU, UGC |
| Glutamate | GAA, GAG |
| Glutamine | CAA, CAG |
| Glycine | GGU, GGC, GGA, GGG |
| Histidine | CAU, CAC |
| Isoleucine | AUU, AUC, AUA |
| Leucine | UUA, UUG, CUU, CUC, CUA, CUG |
| Lysine | AAA, AAG |
| Methionine | AUG |
| Phenylalanine | UUU, UUC |
| Proline | CCU, CCC, CCA, CCG |
| Serine | UCU, UCC, UCA, UCG, AGU, AGC |
| Stop | UAA, UAG, UGA |
| Threonine | ACU, ACC, ACA, ACG |
| Tryptophan | UGG |
| Tyrosine | UAU, UAC |
| Valine | GUU, GUC, GUA, GUG |

In an embodiment, a TREM comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment, a TREM comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM, a TREM core fragment, or TREM fragment comprises at least 5, 10, 15, 20, 25, or 30 consecutive nucleotides of an RNA sequence encoded by a DNA sequence disclosed in Table 1, e.g., at least 5, 10, 15, 20, 25, or 30 consecutive nucleotides of an RNA sequence encoded by any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM, a TREM core fragment, or TREM fragment comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, a TREM core fragment or a TREM fragment comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, a TREM core fragment or a TREM fragment comprises a sequence of a length of between 10-90 ribonucleotides (rnt), between 10-80 rnt, between 10-70 rnt, between 10-60 rnt, between 10-50 rnt, between 10-40 rnt, between 10-30 rnt, between 10-20 rnt, between 20-90 rnt, between 20-80 rnt, 20-70 rnt, between 20-60 rnt, between 20-50 rnt, between 20-40 rnt, between 30-90 rnt, between 30-80 rnt, between 30-70 rnt, between 30-60 rnt, or between 30-50 rnt.

In any and all embodiments, the TREM described herein comprises a consensus sequence of Formula I _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula I corresponds to all species; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a sugar within one or more of R₀-R₁-R₂-R₃-R₄-R₅-R₆-R₇-R₈ or (v) an ASGPR binding moiety is bound to a sugar within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula I ZZZ,
R0-R1-R2-R3-R4-R5-R6-R7-R8-R9-R10-R11-R12-R13-R14-R15-R16-R17-R18-R19-R20-R21-R22-R23-R24-R25-R26-R27-R28-R29-R30-R31-R32-R33-R34-R35-R36-R37-R38-R39-R40-R41-R42-R43-R44-R45-R46-[R47]x1-R48-R49-R50-R51-R52-R53-R54-R55-R56-R57-R58-R59-R60-R61-R62-R63-R64-R65-R66-R67-R68-R69-R70-R71-R72
wherein (i) ZZZ indicates any of the twenty amino acids; (ii) Formula I corresponds to all species; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to the phosphate backbone at any nucleotide position within one or more of R0-R1-R2-R3-R4-R5-R6-R7-R8 or (v) an ASGPR binding moiety is bound to the phosphate backbone at any nucleotide position within one or more of R61-R62-R63-R64-R65-R66-R67-R68-R69-R70-R71-R72.

In any and all embodiments, the TREM described herein comprises a consensus sequence of Formula I _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula I corresponds to all species; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a nucleobase within one or more of R₀-R₁-R₂-R₃-R₄-R₅-R₆-R₇-R₈ or (v) an ASGPR binding moiety is bound to a nucleobase within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula II _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula II corresponds to mammals; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a sugar within one or more of R₀-R₁-R₂-R₃-R₄-R₅-R₆-R₇-R₈ or (v) an ASGPR binding moiety is bound to a sugar within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula II ZZZ,
R0-R1-R2-R3-R4-R5-R6-R7-R8-R9-R10-R11-R12-R13-R14-R15-R16-R17-R18-R19-R20-R21-R22-R23-R24-R25-R26-R27-R28-R29-R30-R31-R32-R33-R34-R35-R36-R37-R38-R39-R40-R41-R42-R43-R44-R45-R46-[R47]x1-R48-R49-R50-R51-R52-R53-R54-R55-R56-R57-R58-R59-R60-R61-R62-R63-R64-R65-R66-R67-R68-R69-R70-R71-R72
wherein (i) ZZZ indicates any of the twenty amino acids; (ii) Formula II corresponds to mammals; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to the phosphate backbone at any nucleotide position within one or more of R0-R1-R2-R3-R4-R5-R6-R7-R8 or (v) an ASGPR binding moiety is bound to the phosphate backbone at any nucleotide position within one or more of R61-R62-R63-R64-R65-R66-R67-R68-R69-R70-R71-R72.

In any and all embodiments, the TREM described herein comprises a consensus sequence of Formula II _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula II corresponds to mammals; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1- 24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a nucleobase within one or more of R₀-R₁-R₂-R₃-R₄-R₅-R₆-R₇-R₈ or (v) an ASGPR binding moiety is bound to a nucleobase within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula IIII _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula III corresponds to humans; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a sugar within one or more of R₀-R₁-R₂-R₃-R₄-R₅-R₆-R₇-R₈ or (v) an ASGPR binding moiety is bound to a sugar within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula IIII _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula III corresponds to humans; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to the phosphate backbone at any nucleotide position within one or more of R₀-R₁-R₂-R₃-R₄-R₅-R₆-R₇-R₈ or (v) an ASGPR binding moiety is bound to the phosphate backbone at any nucleotide position within one or more of R₆₁-R₆₂-R₆₃-R₆₄-R₆₅-R₆₆-R₆₇-R₆₈-R₆₉-R₇₀-R₇₁-R₇₂.

In any and all embodiments, the TREM described herein comprises a consensus sequence of Formula IIII _{ZZZ},
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ₁-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅-R₆₆-R₆₇-R₆₈-R₆₉-R₇₀-R₇₁-R₇₂
wherein (i) _{ZZZ} indicates any of the twenty amino acids; (ii) Formula III corresponds to humans; (iii) x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271); and (iv) an ASGPR binding moiety is bound to a nucleobase within one or more of R₀-R₁-R₂-R₃-R₄-R₅-R₆-R₇-Rx or (v) an ASGPR binding moiety is bound to a nucleobase within one or more of R₆₁-R₆₂-R₆₃-R₆₄-R₆₅-R₆₆-R₆₇-R₆₈-R₆₉-R₇₀-R₇₁-R₇₂.

TABLE 1

| SEQ ID NO | tRNA name | tRNA sequence |
|---|---|---|
| 1 | Ala_AGC_chr6: 28763741- | GGGGGTATAGCTCAGTGGTAGAGCGCGTG |
| | 28763812 (−) | CTTAGCATGCACGAGGTCCTGGGTTCGAT |
| | | CCCCAGTACCTCCA |
| 2 | Ala_AGC_chr6: 26687485- | GGGGAATTAGCTCAAGTGGTAGAGCGCTT |
| | 26687557 (+) | GCTTAGCACGCAAGAGGTAGTGGGATCG |
| | | ATGCCCACATTCTCCA |
| 3 | Ala_AGC_chr6: 26572092- | GGGGAATTAGCTCAAATGGTAGAGCGCTC |
| | 26572164 (−) | GCTTAGCATGCGAGAGGTAGCGGGATCG |
| | | ATGCCCGCATTCTCCA |
| 4 | Ala_AGC_chr6: 26682715- | GGGGAATTAGCTCAAGTGGTAGAGCGCTT |
| | 26682787 (+) | GCTTAGCATGCAAGAGGTAGTGGGATCGA |
| | | TGCCCACATTCTCCA |
| 5 | Ala_AGC_chr6: 26705606- | GGGGAATTAGCTCAAGCGGTAGAGCGCTT |
| | 26705678 (+) | GCTTAGCATGCAAGAGGTAGTGGGATCGA |
| | | TGCCCACATTCTCCA |
| 6 | Ala_AGC_chr6: 26673590- | GGGGAATTAGCTCAAGTGGTAGAGCGCTT |
| | 26673662 (+) | GCTTAGCATGCAAGAGGTAGTGGGATCAA |
| | | TGCCCACATTCTCCA |
| 7 | Ala_AGC_chr14: 89445442- | GGGGAATTAGCTCAAGTGGTAGAGCGCTC |
| | 89445514 (+) | GCTTAGCATGCGAGAGGTAGTGGGATCGA |
| | | TGCCCGCATTCTCCA |
| 8 | Ala_AGC_chr6: 58196623- | GGGGAATTAGCCCAAGTGGTAGAGCGCTT |
| | 58196695 (−) | GCTTAGCATGCAAGAGGTAGTGGGATCGA |
| | | TGCCCACATTCTCCA |
| 9 | Ala_AGC_chr6: 28806221- | GGGGGTGTAGCTCAGTGGTAGAGCGCGTG |
| | 28806292 (−) | CTTAGCATGCACGAGGCCCCGGGTTCAAT |
| | | CCCCGGCACCTCCA |
| 10 | Ala_AGC_chr6: 28574933- | GGGGGTGTAGCTCAGTGGTAGAGCGCGTG |
| | 28575004 (+) | CTTAGCATGTACGAGGTCCCGGGTTCAAT |
| | | CCCCGGCACCTCCA |
| 11 | Ala_AGC_chr6: 28626014- | GGGGATGTAGCTCAGTGGTAGAGCGCATG |
| | 28626085 (−) | CTTAGCATGCATGAGGTCCCGGGTTCGAT |
| | | CCCCAGCATCTCCA |
| 12 | Ala_AGC_chr6: 28678366- | GGGGGTGTAGCTCAGTGGTAGAGCGCGTG |
| | 28678437 (+) | CTTAGCATGCACGAGGCCCTGGGTTCAAT |
| | | CCCCAGCACCTCCA |
| 13 | Ala_AGC_chr6: 28779849- | GGGGGTATAGCTCAGCGGTAGAGCGCGT |
| | 28779920 (−) | GCTTAGCATGCACGAGGTCCTGGGTTCAA |
| | | TCCCCAATACCTCCA |
| 14 | Ala_AGC_chr6: 28687481- | GGGGGTGTAGCTCAGTGGTAGAGCGCGTG |
| | 28687552 (+) | CTTAGCATGCACGAGGCCCCGGGTTCAAT |
| | | CCCTGGCACCTCCA |
| 15 | Ala_AGC_chr2: 27274082- | GGGGGATTAGCTCAAATGGTAGAGCGCTC |
| | 27274154 (+) | GCTTAGCATGCGAGAGGTAGCGGGATCG |
| | | ATGCCCGCATCCTCCA |
| 16 | Ala_AGC_chr6: 26730737- | GGGGAATTAGCTCAGGCGGTAGAGCGCTC |
| | 26730809 (+) | GCTTAGCATGCGAGAGGTAGCGGGATCG |
| | | ACGCCCGCATTCTCCA |
| 17 | Ala_CGC_chr6: 26553731- | GGGGATGTAGCTCAGTGGTAGAGCGCATG |
| | 26553802 (+) | CTTCGCATGTATGAGGTCCCGGGTTCGAT |
| | | CCCCGGCATCTCCA |
| 18 | Ala_CGC_chr6: 28641613- | GGGGATGTAGCTCAGTGGTAGAGCGCATG |
| | 28641684 (−) | CTTCGCATGTATGAGGCCCCGGGTTCGAT |
| | | CCCCGGCATCTCCA |
| 19 | Ala_CGC_chr2: 157257281- | GGGGATGTAGCTCAGTGGTAGAGCGCGC |
| | 157257352 (+) | GCTTCGCATGTGTGAGGTCCCGGGTTCAA |
| | | TCCCCGGCATCTCCA |
| 20 | Ala_CGC_chr6: 28697092- | GGGGGTGTAGCTCAGTGGTAGAGCGCGTG |
| | 28697163 (+) | CTTCGCATGTACGAGGCCCCGGGTTCGAC |
| | | CCCCGGCTCCTCCA |
| 21 | Ala_TGC_chr6: 28757547- | GGGGGTGTAGCTCAGTGGTAGAGCGCATG |
| | 28757618 (−) | CTTTGCATGTATGAGGTCCCGGGTTCGAT |
| | | CCCCGGCACCTCCA |
| 22 | Ala_TGC_chr6: 28611222- | GGGGATGTAGCTCAGTGGTAGAGCGCATG |
| | 28611293 (+) | CTTTGCATGTATGAGGTCCCGGGTTCGAT |
| | | CCCCGGCATCTCCA |
| 23 | Ala_TGC_chr5: 180633868- | GGGGATGTAGCTCAGTGGTAGAGCGCATG |
| | 180633939 (+) | CTTTGCATGTATGAGGCCCCGGGTTCGAT |
| | | CCCCGGCATCTCCA |
| 24 | Ala_TGC_chr12: 125424512- | GGGGATGTAGCTCAGTGGTAGAGCGCATG |
| | 125424583 (+) | CTTTGCACGTATGAGGCCCCGGGTTCAAT |
| | | CCCCGGCATCTCCA |
| 25 | Ala_TGC_chr6: 28785012- | GGGGGTGTAGCTCAGTGGTAGAGCGCATG |
| | 28785083 (−) | CTTTGCATGTATGAGGCCTCGGGTTCGAT |
| | | CCCCGACACCTCCA |
| 26 | Ala_TGC_chr6: 28726141- | GGGGGTGTAGCTCAGTGGTAGAGCACATG |
| | 28726212 (−) | CTTTGCATGTGTGAGGCCCCGGGTTCGAT |
| | | CCCCGGCACCTCCA |
| 27 | Ala_TGC_chr6: 28770577- | GGGGGTGTAGCTCAGTGGTAGAGCGCATG |
| | 28770647 (−) | CTTTGCATGTATGAGGCCTCGGTTCGATC |
| | | CCCGACACCTCCA |
| 28 | Arg_ACG_chr6: 26328368- | GGGCCAGTGGCGCAATGGATAACGCGTCT |
| | 26328440 (+) | GACTACGGATCAGAAGATTCCAGGTTCGA |
| | | CTCCTGGCTGGCTCG |
| 29 | Arg_ACG_chr3: 45730491- | GGGCCAGTGGCGCAATGGATAACGCGTCT |
| | 45730563 (−) | GACTACGGATCAGAAGATTCTAGGTTCGA |
| | | CTCCTGGCTGGCTCG |
| 30 | Arg_CCG_chr6: 28710729- | GGCCGCGTGGCCTAATGGATAAGGCGTCT |
| | 28710801 (−) | GATTCCGGATCAGAAGATTGAGGGTTCGA |
| | | GTCCCTTCGTGGTCG |
| 31 | Arg_CCG_chr17: 66016013- | GACCCAGTGGCCTAATGGATAAGGCATCA |
| | 66016085 (−) | GCCTCCGGAGCTGGGGATTGTGGGTTCGA |
| | | GTCCCATCTGGGTCG |
| 32 | Arg_CCT_chr17: 73030001- | GCCCCAGTGGCCTAATGGATAAGGCACTG |
| | 73030073 (+) | GCCTCCTAAGCCAGGGATTGTGGGTTCGA |
| | | GTCCCACCTGGGGTA |
| 33 | Arg_CCT_chr17: 73030526- | GCCCCAGTGGCCTAATGGATAAGGCACTG |
| | 73030598 (−) | GCCTCCTAAGCCAGGGATTGTGGGTTCGA |
| | | GTCCCACCTGGGGTG |
| 34 | Arg_CCT_chr16: 3202901- | GCCCCGGTGGCCTAATGGATAAGGCATTG |
| | 3202973 (+) | GCCTCCTAAGCCAGGGATTGTGGGTTCGA |
| | | GTCCCACCCGGGGTA |
| 35 | Arg_CCT_chr7: 139025446- | GCCCCAGTGGCCTAATGGATAAGGCATTG |
| | 139025518 (+) | GCCTCCTAAGCCAGGGATTGTGGGTTCGA |
| | | GTCCCATCTGGGGTG |
| 36 | Arg_CCT_chr16: 3243918- | GCCCCAGTGGCCTGATGGATAAGGTACTG |
| | 3243990 (+) | GCCTCCTAAGCCAGGGATTGTGGGTTCGA |
| | | GTTCCACCTGGGGTA |
| 37 | Arg_TCG_chr15: 89878304- | GGCCGCGTGGCCTAATGGATAAGGCGTCT |
| | 89878376 (+) | GACTTCGGATCAGAAGATTGCAGGTTCGA |
| | | GTCCTGCCGCGGTCG |
| 38 | Arg_TCG_chr6: 26323046- | GACCACGTGGCCTAATGGATAAGGCGTCT |
| | 26323118 (+) | GACTTCGGATCAGAAGATTGAGGGTTCGA |
| | | ATCCCTCCGTGGTTA |
| 39 | Arg_TCG_chr17: 73031208- | GACCGCGTGGCCTAATGGATAAGGCGTCT |
| | 73031280 (+) | GACTTCGGATCAGAAGATTGAGGGTTCGA |
| | | GTCCCTTCGTGGTCG |
| 40 | Arg_TCG_chr6: 26299905- | GACCACGTGGCCTAATGGATAAGGCGTCT |
| | 26299977 (+) | GACTTCGGATCAGAAGATTGAGGGTTCGA |
| | | ATCCCTTCGTGGTTA |
| 41 | Arg_TCG_chr6: 28510891- | GACCACGTGGCCTAATGGATAAGGCGTCT |
| | 28510963 (−) | GACTTCGGATCAGAAGATTGAGGGTTCGA |
| | | ATCCCTTCGTGGTTG |
| 42 | Arg_TCG_chr9: 112960803- | GGCCGTGTGGCCTAATGGATAAGGCGTCT |
| | 112960875 (+) | GACTTCGGATCAAAAGATTGCAGGTTTGA |
| | | GTTCTGCCACGGTCG |
| 43 | Arg_TCT_chr1: 94313129- | GGCTCCGTGGCGCAATGGATAGCGCATTG |
| | 94313213 (+) | GACTTCTAGAGGCTGAAGGCATTCAAAGG |
| | | TTCCGGGTTCGAGTCCCGGCGGAGTCG |
| 44 | Arg_TCT_chr17: 8024243- | GGCTCTGTGGCGCAATGGATAGCGCATTG |
| | 8024330 (+) | GACTTCTAGTGACGAATAGAGCAATTCAA |
| | | AGGTTGTGGGTTCGAATCCCACCAGAGTC |
| | | G |
| 45 | Arg_TCT_chr9: 131102355- | GGCTCTGTGGCGCAATGGATAGCGCATTG |
| | 131102445 (−) | GACTTCTAGCTGAGCCTAGTGTGGTCATT |
| | | CAAAGGTTGTGGGTTCGAGTCCCACCAGA |
| | | GTCG |
| 46 | Arg_TCT_chr11: 59318767- | GGCTCTGTGGCGCAATGGATAGCGCATTG |
| | 59318852 (+) | GACTTCTAGATAGTTAGAGAAATTCAAAG |
| | | GTTGTGGGTTCGAGTCCCACCAGAGTCG |
| 47 | Arg_TCT_chr1: 159111401- | GTCTCTGTGGCGCAATGGACGAGCGCGCT |
| | 159111474 (−) | GGACTTCTAATCCAGAGGTTCCGGGTTCG |
| | | AGTCCCGGCAGAGATG |
| 48 | Arg_TCT_chr6: 27529963- | GGCTCTGTGGCGCAATGGATAGCGCATTG |
| | 27530049 (+) | GACTTCTAGCCTAAATCAAGAGATTCAAA |
| | | GGTTGCGGGTTCGAGTCCCTCCAGAGTCG |
| 49 | Asn_GTT_chr1: 161510031- | GTCTCTGTGGCGCAATCGGTTAGCGCGTT |
| | 161510104 (+) | CGGCTGTTAACCGAAAGGTTGGTGGTTCG |
| | | ATCCCACCCAGGGACG |
| 50 | Asn_GTT_chr1: 143879832- | GTCTCTGTGGCGCAATCGGCTAGCGCGTT |
| | 143879905 (−) | TGGCTGTTAACTAAAAGGTTGGCGGTTCG |
| | | AACCCACCCAGAGGCG |
| 51 | Asn_GTT_chr1: 144301611- | GTCTCTGTGGTGCAATCGGTTAGCGCGTT |
| | 144301684 (+) | CCGCTGTTAACCGAAAGCTTGGTGGTTCG |
| | | AGCCCACCCAGGGATG |
| 52 | Asn_GTT_chr1: 149326272- | GTCTCTGTGGCGCAATCGGCTAGCGCGTT |
| | 149326345 (−) | TGGCTGTTAACTAAAAAGTTGGTGGTTCG |
| | | AACACACCCAGAGGCG |
| 53 | Asn_GTT_chr1: 148248115- | GTCTCTGTGGCGCAATCGGTTAGCGCGTT |
| | 148248188 (+) | CGGCTGTTAACCGAAAGGTTGGTGGTTCG |
| | | AGCCCACCCAGGGACG |
| 54 | Asn_GTT_chr1: 148598314- | GTCTCTGTGGCGCAATCGGTTAGCGCATT |
| | 148598387 (−) | CGGCTGTTAACCGAAAGGTTGGTGGTTCG |
| | | AGCCCACCCAGGGACG |
| 55 | Asn_GTT_chr1: 17216172- | GTCTCTGTGGCGCAATCGGTTAGCGCGTT |
| | 17216245 (+) | CGGCTGTTAACCGAAAGATTGGTGGTTCG |
| | | AGCCCACCCAGGGACG |
| 56 | Asn_GTT_chr1: 16847080- | GTCTCTGTGGCGCAATCGGTTAGCGCGTT |
| | 16847153 (−) | CGGCTGTTAACTGAAAGGTTGGTGGTTCG |
| | | AGCCCACCCAGGGACG |
| 57 | Asn_GTT_chr1: 149230570- | GTCTCTGTGGCGCAATGGGTTAGCGCGTT |
| | 149230643 (−) | CGGCTGTTAACCGAAAGGTTGGTGGTTCG |
| | | AGCCCATCCAGGGACG |
| 58 | Asn_GTT_chr1: 148000805- | GTCTCTGTGGCGTAGTCGGTTAGCGCGTT |
| | 148000878 (+) | CGGCTGTTAACCGAAAAGTTGGTGGTTCG |
| | | AGCCCACCCAGGAACG |
| 59 | Asn_GTT_chr1: 149711798- | GTCTCTGTGGCGCAATCGGCTAGCGCGTT |
| | 149711871 (−) | TGGCTGTTAACTAAAAGGTTGGTGGTTCG |
| | | AACCCACCCAGAGGCG |
| 60 | Asn_GTT_chr1: 145979034- | GTCTCTGTGGCGCAATCGGTTAGCGCGTT |
| | 145979107 (−) | CGGCTGTTAACTGAAAGGTTAGTGGTTCG |
| | | AGCCCACCCGGGGACG |
| 61 | Asp_GTC_chr12: 98897281- | TCCTCGTTAGTATAGTGGTTAGTATCCCCG |
| | 98897352 (+) | CCTGTCACGCGGGAGACCGGGGTTCAATT |
| | | CCCCGACGGGGAG |
| 62 | Asp_GTC_chr1: 161410615- | TCCTCGTTAGTATAGTGGTGAGTATCCCC |
| | 161410686 (−) | GCCTGTCACGCGGGAGACCGGGGTTCGAT |
| | | TCCCCGACGGGGAG |
| 63 | Asp_GTC_chr6: 27551236- | TCCTCGTTAGTATAGTGGTGAGTGTCCCC |
| | 27551307 (−) | GTCTGTCACGCGGGAGACCGGGGTTCGAT |
| | | TCCCCGACGGGGAG |
| 64 | Cys_GCA_chr7: 149007281- | GGGGGCATAGCTCAGTGGTAGAGCATTTG |
| | 149007352 (+) | ACTGCAGATCAAGAGGTCCCTGGTTCAAA |
| | | TCCAGGTGCCCCCT |
| 65 | Cys_GCA_chr7: 149074601- | GGGGGTATAGCTCAGGGGTAGAGCATTTG |
| | 149074672 (−) | ACTGCAGATCAAGAGGTCCCTGGTTCAAA |
| | | TCCAGGTGCCCCCC |
| 66 | Cys_GCA_chr7: 149112229- | GGGGGTATAGCTTAGCGGTAGAGCATTTG |
| | 149112300 (−) | ACTGCAGATCAAGAGGTCCCCGGTTCAAA |
| | | TCCGGGTGCCCCCT |
| 67 | Cys_GCA_chr7: 149344046- | GGGGGTATAGCTTAGGGGTAGAGCATTTG |
| | 149344117 (−) | ACTGCAGATCAAAAGGTCCCTGGTTCAAA |
| | | TCCAGGTGCCCCTT |
| 68 | Cys_GCA_chr7: 149052766- | GGGGGTATAGCTCAGGGGTAGAGCATTTG |
| | 149052837 (−) | ACTGCAGATCAAGAGGTCCCCAGTTCAAA |
| | | TCTGGGTGCCCCCT |
| 69 | Cys_GCA_chr17: 37017937- | GGGGGTATAGCTCAGGGGTAGAGCATTTG |
| | 37018008 (−) | ACTGCAGATCAAGAAGTCCCCGGTTCAAA |
| | | TCCGGGTGCCCCCT |
| 70 | Cys_GCA_chr7: 149281816- | GGGGGTATAGCTCAGGGGTAGAGCATTTG |
| | 149281887 (+) | ACTGCAGATCAAGAGGTCTCTGGTTCAAA |
| | | TCCAGGTGCCCCCT |
| 71 | Cys_GCA_chr7: 149243631- | GGGGGTATAGCTCAGGGGTAGAGCACTTG |
| | 149243702 (+) | ACTGCAGATCAAGAAGTCCTTGGTTCAAA |
| | | TCCAGGTGCCCCCT |
| 72 | Cys_GCA_chr7: 149388272- | GGGGATATAGCTCAGGGGTAGAGCATTTG |
| | 149388343 (−) | ACTGCAGATCAAGAGGTCCCCGGTTCAAA |
| | | TCCGGGTGCCCCCC |
| 73 | Cys_GCA_chr7: 149072850- | GGGGGTATAGTTCAGGGGTAGAGCATTTG |
| | 149072921 (−) | ACTGCAGATCAAGAGGTCCCTGGTTCAAA |
| | | TCCAGGTGCCCCCT |
| 74 | Cys_GCA_chr7: 149310156- | GGGGGTATAGCTCAGGGGTAGAGCATTTG |
| | 149310227 (−) | ACTGCAAATCAAGAGGTCCCTGATTCAAA |
| | | TCCAGGTGCCCCCT |
| 75 | Cys_GCA_chr4: 124430005- | GGGGGTATAGCTCAGTGGTAGAGCATTTG |
| | 124430076 (−) | ACTGCAGATCAAGAGGTCCCCGGTTCAAA |
| | | TCCGGGTGCCCCCT |
| 76 | Cys_GCA_chr7: 149295046- | GGGCGTATAGCTCAGGGGTAGAGCATTTG |
| | 149295117 (+) | ACTGCAGATCAAGAGGTCCCCAGTTCAAA |
| | | TCTGGGTGCCCCCT |
| 77 | Cys_GCA_chr7: 149361915- | GGGGGTATAGCTCACAGGTAGAGCATTTG |
| | 149361986 (+) | ACTGCAGATCAAGAGGTCCCCGGTTCAAA |
| | | TCTGGGTGCCCCCT |
| 78 | Cys_GCA_chr7: 149253802- | GGGCGTATAGCTCAGGGGTAGAGCATTTG |
| | 149253871 (+) | ACTGCAGATCAAGAGGTCCCCAGTTCAAA |
| | | TCTGGGTGCCCA |
| 79 | Cys_GCA_chr7: 149292305- | GGGGGTATAGCTCACAGGTAGAGCATTTG |
| | 149292376 (−) | ACTGCAGATCAAGAGGTCCCCGGTTCAAA |
| | | TCCGGTTACTCCCT |
| 80 | Cys_GCA_chr7: 149286164- | GGGGGTATAGCTCAGGGGTAGAGCACTTG |
| | 149286235 (−) | ACTGCAGATCAAGAGGTCCCTGGTTCAAA |
| | | TCCAGGTGCCCCCT |
| 81 | Cys_GCA_chr17: 37025545- | GGGGGTATAGCTCAGTGGTAGAGCATTTG |
| | 37025616 (−) | ACTGCAGATCAAGAGGTCCCTGGTTCAAA |
| | | TCCGGGTGCCCCCT |
| 82 | Cys_GCA_chr15: 80036997- | GGGGGTATAGCTCAGTGGGTAGAGCATTT |
| | 80037069 (+) | GACTGCAGATCAAGAGGTCCCCGGTTCAA |
| | | ATCCGGGTGCCCCCT |
| 83 | Cys_GCA_chr3: 131947944- | GGGGGTGTAGCTCAGTGGTAGAGCATTTG |
| | 131948015 (−) | ACTGCAGATCAAGAGGTCCCTGGTTCAAA |
| | | TCCAGGTGCCCCCT |
| 84 | Cys_GCA_chr1: 93981834- | GGGGGTATAGCTCAGGTGGTAGAGCATTT |
| | 93981906 (−) | GACTGCAGATCAAGAGGTCCCCGGTTCAA |
| | | ATCCGGGTGCCCCCT |
| 85 | Cys_GCA_chr14: 73429679- | GGGGGTATAGCTCAGGGGTAGAGCATTTG |
| | 73429750 (+) | ACTGCAGATCAAGAGGTCCCCGGTTCAAA |
| | | TCCGGGTGCCCCCT |
| 86 | Cys_GCA_chr3: 131950642- | GGGGGTATAGCTCAGGGGTAGAGCATTTG |
| | 131950713 (−) | ACTGCAGATCAAGAGGTCCCTGGTTCAAA |
| | | TCCAGGTGCCCCCT |
| 87 | Gln_CTG_chr6: 18836402- | GGTTCCATGGTGTAATGGTTAGCACTCTG |
| | 18836473 (+) | GACTCTGAATCCAGCGATCCGAGTTCAAA |
| | | TCTCGGTGGAACCT |
| 88 | Gln_CTG_chr6: 27515531- | GGTTCCATGGTGTAATGGTTAGCACTCTG |
| | 27515602 (−) | GACTCTGAATCCAGCGATCCGAGTTCAAG |
| | | TCTCGGTGGAACCT |
| 89 | Gln_CTG_chr1: 145963304- | GGTTCCATGGTGTAATGGTGAGCACTCTG |
| | 145963375 (+) | GACTCTGAATCCAGCGATCCGAGTTCGAG |
| | | TCTCGGTGGAACCT |
| 90 | Gln_CTG_chr1: 147737382- | GGTTCCATGGTGTAATGGTAAGCACTCTG |
| | 147737453 (−) | GACTCTGAATCCAGCGATCCGAGTTCGAG |
| | | TCTCGGTGGAACCT |
| 91 | Gln_CTG_chr6: 27263212- | GGTTCCATGGTGTAATGGTTAGCACTCTG |
| | 27263283 (+) | GACTCTGAATCCGGTAATCCGAGTTCAAA |
| | | TCTCGGTGGAACCT |
| 92 | Gln_CTG_chr6: 27759135- | GGCCCCATGGTGTAATGGTCAGCACTCTG |
| | 27759206 (−) | GACTCTGAATCCAGCGATCCGAGTTCAAA |
| | | TCTCGGTGGGACCC |
| 93 | Gln_CTG_chr1: 147800937- | GGTTCCATGGTGTAATGGTAAGCACTCTG |
| | 147801008 (+) | GACTCTGAATCCAGCCATCTGAGTTCGAG |
| | | TCTCTGTGGAACCT |
| 94 | Gln_TTG_chr17: 47269890- | GGTCCCATGGTGTAATGGTTAGCACTCTG |
| | 47269961 (+) | GACTTTGAATCCAGCGATCCGAGTTCAAA |
| | | TCTCGGTGGGACCT |
| 95 | Gln_TTG_chr6: 28557156- | GGTCCCATGGTGTAATGGTTAGCACTCTG |
| | 28557227 (+) | GACTTTGAATCCAGCAATCCGAGTTCGAA |
| | | TCTCGGTGGGACCT |
| 96 | Gln_TTG_chr6: 26311424- | GGCCCCATGGTGTAATGGTTAGCACTCTG |
| | 26311495 (−) | GACTTTGAATCCAGCGATCCGAGTTCAAA |
| | | TCTCGGTGGGACCT |
| 97 | Gln_TTG_chr6: 145503859- | GGTCCCATGGTGTAATGGTTAGCACTCTG |
| | 145503930 (+) | GGCTTTGAATCCAGCAATCCGAGTTCGAA |
| | | TCTTGGTGGGACCT |
| 98 | Glu_CTC_chr1: 145399233- | TCCCTGGTGGTCTAGTGGTTAGGATTCGG |
| | 145399304 (−) | CGCTCTCACCGCCGCGGCCCGGGTTCGAT |
| | | TCCCGGTCAGGGAA |
| 99 | Glu_CTC_chr1: 249168447- | TCCCTGGTGGTCTAGTGGTTAGGATTCGG |
| | 249168518 (+) | CGCTCTCACCGCCGCGGCCCGGGTTCGAT |
| | | TCCCGGTCAGGAAA |
| 100 | Glu_TTC_chr2: 131094701- | TCCCATATGGTCTAGCGGTTAGGATTCCT |
| | 131094772 (−) | GGTTTTCACCCAGGTGGCCCGGGTTCGAC |
| | | TCCCGGTATGGGAA |
| 101 | Glu_TTC_chr13: 45492062- | TCCCACATGGTCTAGCGGTTAGGATTCCT |
| | 45492133 (−) | GGTTTTCACCCAGGCGGCCCGGGTTCGAC |
| | | TCCCGGTGTGGGAA |
| 102 | Glu_TTC_chr1: 17199078- | TCCCTGGTGGTCTAGTGGCTAGGATTCGG |
| | 17199149 (+) | CGCTTTCACCGCCGCGGCCCGGGTTCGAT |
| | | TCCCGGCCAGGGAA |
| 103 | Glu_TTC_chr1: 16861774- | TCCCTGGTGGTCTAGTGGCTAGGATTCGG |
| | 16861845 (−) | CGCTTTCACCGCCGCGGCCCGGGTTCGAT |
| | | TCCCGGTCAGGGAA |
| 104 | Gly_CCC_chr1: 16872434- | GCATTGGTGGTTCAGTGGTAGAATTCTCG |
| | 16872504 (−) | CCTCCCACGCGGGAGACCCGGGTTCAATT |
| | | CCCGGCCAATGCA |
| 105 | Gly_CCC_chr2: 70476123- | GCGCCGCTGGTGTAGTGGTATCATGCAAG |
| | 70476193 (−) | ATTCCCATTCTTGCGACCCGGGTTCGATTC |
| | | CCGGGCGGCGCA |
| 106 | Gly_CCC_chr17: 19764175- | GCATTGGTGGTTCAATGGTAGAATTCTCG |
| | 19764245 (+) | CCTCCCACGCAGGAGACCCAGGTTCGATT |
| | | CCTGGCCAATGCA |
| 107 | Gly_GCC_chr1: 161413094- | GCATGGGTGGTTCAGTGGTAGAATTCTCG |
| | 161413164 (+) | CCTGCCACGCGGGAGGCCCGGGTTCGATT |
| | | CCCGGCCCATGCA |
| 108 | Gly_GCC_chr1: 161493637- | GCATTGGTGGTTCAGTGGTAGAATTCTCG |
| | 161493707 (−) | CCTGCCACGCGGGAGGCCCGGGTTCGATT |
| | | CCCGGCCAATGCA |
| 109 | Gly_GCC_chr16: 70812114- | GCATTGGTGGTTCAGTGGTAGAATTCTCG |
| | 70812184 (−) | CCTGCCACGCGGGAGGCCCGGGTTTGATT |
| | | CCCGGCCAGTGCA |
| 110 | Gly_GCC_chr1: 161450356- | GCATAGGTGGTTCAGTGGTAGAATTCTTG |
| | 161450426 (+) | CCTGCCACGCAGGAGGCCCAGGTTTGATT |
| | | CCTGGCCCATGCA |
| 111 | Gly_GCC_chr16: 70822597- | GCATTGGTGGTTCAGTGGTAGAATTCTCG |
| | 70822667 (+) | CCTGCCATGCGGGCGGCCGGGCTTCGATT |
| | | CCTGGCCAATGCA |
| 112 | Gly_TCC_chr19: 4724082- | GCGTTGGTGGTATAGTGGTTAGCATAGCT |
| | 4724153 (+) | GCCTTCCAAGCAGTTGACCCGGGTTCGAT |
| | | TCCCGGCCAACGCA |
| 113 | Gly_TCC_chr1: 145397864- | GCGTTGGTGGTATAGTGGTGAGCATAGCT |
| | 145397935 (−) | GCCTTCCAAGCAGTTGACCCGGGTTCGAT |
| | | TCCCGGCCAACGCA |
| 114 | Gly_TCC_chr17: 8124866- | GCGTTGGTGGTATAGTGGTAAGCATAGCT |
| | 8124937 (+) | GCCTTCCAAGCAGTTGACCCGGGTTCGAT |
| | | TCCCGGCCAACGCA |
| 115 | Gly_TCC_chr1: 161409961- | GCGTTGGTGGTATAGTGGTGAGCATAGTT |
| | 161410032 (−) | GCCTTCCAAGCAGTTGACCCGGGCTCGAT |
| | | TCCCGCCCAACGCA |
| 116 | His_GTG_chr1: 145396881- | GCCGTGATCGTATAGTGGTTAGTACTCTG |
| | 145396952 (−) | CGTTGTGGCCGCAGCAACCTCGGTTCGAA |
| | | TCCGAGTCACGGCA |
| 117 | His_GTG_chr1: 149155828- | GCCATGATCGTATAGTGGTTAGTACTCTG |
| | 149155899 (−) | CGCTGTGGCCGCAGCAACCTCGGTTCGAA |
| | | TCCGAGTCACGGCA |
| 118 | Ile_AAT_chr6: 58149254- | GGCCGGTTAGCTCAGTTGGTTAGAGCGTG |
| | 58149327 (+) | GCGCTAATAACGCCAAGGTCGCGGGTTCG |
| | | ATCCCCGTACGGGCCA |
| 119 | Ile_AAT_chr6: 27655967- | GGCCGGTTAGCTCAGTTGGTTAGAGCGTG |
| | 27656040 (+) | GTGCTAATAACGCCAAGGTCGCGGGTTCG |
| | | ATCCCCGTACTGGCCA |
| 120 | Ile_AAT_chr6: 27242990- | GGCTGGTTAGCTCAGTTGGTTAGAGCGTG |
| | 27243063 (−) | GTGCTAATAACGCCAAGGTCGCGGGTTCG |
| | | ATCCCCGTACTGGCCA |
| 121 | Ile_AAT_chr17: 8130309- | GGCCGGTTAGCTCAGTTGGTTAGAGCGTG |
| | 8130382 (−) | GTGCTAATAACGCCAAGGTCGCGGGTTCG |
| | | AACCCCGTACGGGCCA |
| 122 | Ile_AAT_chr6: 26554350- | GGCCGGTTAGCTCAGTTGGTTAGAGCGTG |
| | 26554423 (+) | GTGCTAATAACGCCAAGGTCGCGGGTTCG |
| | | ATCCCCGTACGGGCCA |
| 123 | Ile_AAT_chr6: 26745255- | GGCCGGTTAGCTCAGTTGGTTAGAGCGTG |
| | 26745328 (−) | GTGCTAATAACGCTAAGGTCGCGGGTTCG |
| | | ATCCCCGTACTGGCCA |
| 124 | Ile_AAT_chr6: 26721221- | GGCCGGTTAGCTCAGTTGGTCAGAGCGTG |
| | 26721294 (−) | GTGCTAATAACGCCAAGGTCGCGGGTTCG |
| | | ATCCCCGTACGGGCCA |
| 125 | lle_AAT_chr6: 27636362- | GGCCGGTTAGCTCAGTCGGCTAGAGCGTG |
| | 27636435 (+) | GTGCTAATAACGCCAAGGTCGCGGGTTCG |
| | | ATCCCCGTACGGGCCA |
| 126 | Ile_AAT_chr6: 27241739- | GGCTGGTTAGTTCAGTTGGTTAGAGCGTG |
| | 27241812 (+) | GTGCTAATAACGCCAAGGTCGTGGGTTCG |
| | | ATCCCCATATCGGCCA |
| 127 | Ile_GAT_chrX: 3756418- | GGCCGGTTAGCTCAGTTGGTAAGAGCGTG |
| | 3756491 (−) | GTGCTGATAACACCAAGGTCGCGGGCTCG |
| | | ACTCCCGCACCGGCCA |
| 128 | Ilc_TAT_chr19: 39902808- | GCTCCAGTGGCGCAATCGGTTAGCGCGCG |
| | 39902900 (−) | GTACTTATATGACAGTGCGAGCGGAGCAA |
| | | TGCCGAGGTTGTGAGTTCGATCCTCACCT |
| | | GGAGCA |
| 129 | Ile_TAT_chr2: 43037676- | GCTCCAGTGGCGCAATCGGTTAGCGCGCG |
| | 43037768 (+) | GTACTTATACAGCAGTACATGCAGAGCAA |
| | | TGCCGAGGTTGTGAGTTCGAGCCTCACCT |
| | | GGAGCA |
| 130 | Ile_TAT_chr6: 26988125- | GCTCCAGTGGCGCAATCGGTTAGCGCGCG |
| | 26988218 (+) | GTACTTATATGGCAGTATGTGTGCGAGTG |
| | | ATGCCGAGGTTGTGAGTTCGAGCCTCACC |
| | | TGGAGCA |
| 131 | Ile_TAT_chr6: 27599200- | GCTCCAGTGGCGCAATCGGTTAGCGCGCG |
| | 27599293 (+) | GTACTTATACAACAGTATATGTGCGGGTG |
| | | ATGCCGAGGTTGTGAGTTCGAGCCTCACC |
| | | TGGAGCA |
| 132 | Ile_TAT_chr6: 28505367- | GCTCCAGTGGCGCAATCGGTTAGCGCGCG |
| | 28505460 (+) | GTACTTATAAGACAGTGCACCTGTGAGCA |
| | | ATGCCGAGGTTGTGAGTTCAAGCCTCACC |
| | | TGGAGCA |
| 133 | Leu_AAG_chr5: 180524474- | GGTAGCGTGGCCGAGCGGTCTAAGGCGCT |
| | 180524555 (−) | GGATTAAGGCTCCAGTCTCTTCGGAGGCG |
| | | TGGGTTCGAATCCCACCGCTGCCA |
| 134 | Leu_AAG_chr5: 180614701- | GGTAGCGTGGCCGAGCGGTCTAAGGCGCT |
| | 180614782 (+) | GGATTAAGGCTCCAGTCTCTTCGGGGGCG |
| | | TGGGTTCGAATCCCACCGCTGCCA |
| 135 | Leu_AAG_chr6: 28956779- | GGTAGCGTGGCCGAGCGGTCTAAGGCGCT |
| | 28956860 (+) | GGATTAAGGCTCCAGTCTCTTCGGGGGCG |
| | | TGGGTTCAAATCCCACCGCTGCCA |
| 136 | Leu_AAG_chr6: 28446400- | GGTAGCGTGGCCGAGTGGTCTAAGACGCT |
| | 28446481 (−) | GGATTAAGGCTCCAGTCTCTTCGGGGGCG |
| | | TGGGTTTGAATCCCACCGCTGCCA |
| 137 | Leu_CAA_chr6: 28864000- | GTCAGGATGGCCGAGTGGTCTAAGGCGCC |
| | 28864105 (−) | AGACTCAAGCTAAGCTTCCTCCGCGGTGG |
| | | GGATTCTGGTCTCCAATGGAGGCGTGGGT |
| | | TCGAATCCCACTTCTGACA |
| 138 | Leu_CAA_chr6: 28908830- | GTCAGGATGGCCGAGTGGTCTAAGGCGCC |
| | 28908934 (+) | AGACTCAAGCTTGGCTTCCTCGTGTTGAG |
| | | GATTCTGGTCTCCAATGGAGGCGTGGGTT |
| | | CGAATCCCACTTCTGACA |
| 139 | Leu_CAA_chr6: 27573417- | GTCAGGATGGCCGAGTGGTCTAAGGCGCC |
| | 27573524 (−) | AGACTCAAGCTTACTGCTTCCTGTGTTCG |
| | | GGTCTTCTGGTCTCCGTATGGAGGCGTGG |
| | | GTTCGAATCCCACTTCTGACA |
| 140 | Leu_CAA_chr6: 27570348- | GTCAGGATGGCCGAGTGGTCTAAGGCGCC |
| | 27570454 (−) | AGACTCAAGTTGCTACTTCCCAGGTTTGG |
| | | GGCTTCTGGTCTCCGCATGGAGGCGTGGG |
| | | TTCGAATCCCACTTCTGACA |
| 141 | Leu_CAA_chr1: 249168054- | GTCAGGATGGCCGAGTGGTCTAAGGCGCC |
| | 249168159 (+) | AGACTCAAGGTAAGCACCTTGCCTGCGGG |
| | | CTTTCTGGTCTCCGGATGGAGGCGTGGGT |
| | | TCGAATCCCACTTCTGACA |
| 142 | Leu_CAA_chr11: 9296790- | GCCTCCTTAGTGCAGTAGGTAGCGCATCA |
| | 9296863 (+) | GTCTCAAAATCTGAATGGTCCTGAGTTCA |
| | | AGCCTCAGAGGGGGCA |
| 143 | Leu_CAA_chr1: 161581736- | GTCAGGATGGCCGAGCAGTCTTAAGGCGC |
| | 161581819 (−) | TGCGTTCAAATCGCACCCTCCGCTGGAGG |
| | | CGTGGGTTCGAATCCCACTTTTGACA |
| 144 | Leu_CAG_chr1: 161411323- | GTCAGGATGGCCGAGCGGTCTAAGGCGCT |
| | 161411405 (+) | GCGTTCAGGTCGCAGTCTCCCCTGGAGGC |
| | | GTGGGTTCGAATCCCACTCCTGACA |
| 145 | Leu_CAG_chr16: 57333863- | GTCAGGATGGCCGAGCGGTCTAAGGCGCT |
| | 57333945 (+) | GCGTTCAGGTCGCAGTCTCCCCTGGAGGC |
| | | GTGGGTTCGAATCCCACTTCTGACA |
| 146 | Leu_TAA_chr6: 144537684- | ACCAGGATGGCCGAGTGGTTAAGGCGTTG |
| | 144537766 (+) | GACTTAAGATCCAATGGACATATGTCCGC |
| | | GTGGGTTCGAACCCCACTCCTGGTA |
| 147 | Leu_TAA_chr6: 27688898- | ACCGGGATGGCCGAGTGGTTAAGGCGTTG |
| | 27688980 (−) | GACTTAAGATCCAATGGGCTGGTGCCCGC |
| | | GTGGGTTCGAACCCCACTCTCGGTA |
| 148 | Leu_TAA_chr11: 59319228- | ACCAGAATGGCCGAGTGGTTAAGGCGTTG |
| | 59319310 (+) | GACTTAAGATCCAATGGATTCATATCCGC |
| | | GTGGGTTCGAACCCCACTTCTGGTA |
| 149 | Leu_TAA_chr6: 27198334- | ACCGGGATGGCTGAGTGGTTAAGGCGTTG |
| | 27198416 (−) | GACTTAAGATCCAATGGACAGGTGTCCGC |
| | | GTGGGTTCGAGCCCCACTCCCGGTA |
| 150 | Leu_TAG_chr17: 8023632- | GGTAGCGTGGCCGAGCGGTCTAAGGCGCT |
| | 8023713 (−) | GGATTTAGGCTCCAGTCTCTTCGGAGGCG |
| | | TGGGTTCGAATCCCACCGCTGCCA |
| 151 | Leu_TAG_chr14: 21093529- | GGTAGTGTGGCCGAGCGGTCTAAGGCGCT |
| | 21093610 (+) | GGATTTAGGCTCCAGTCTCTTCGGGGGCG |
| | | TGGGTTCGAATCCCACCACTGCCA |
| 152 | Leu_TAG_chr16: 22207032- | GGTAGCGTGGCCGAGTGGTCTAAGGCGCT |
| | 22207113 (−) | GGATTTAGGCTCCAGTCATTTCGATGGCG |
| | | TGGGTTCGAATCCCACCGCTGCCA |
| 153 | Lys_CTT_chr14: 58706613- | GCCCGGCTAGCTCAGTCGGTAGAGCATGG |
| | 58706685 (−) | GACTCTTAATCCCAGGGTCGTGGGTTCGA |
| | | GCCCCACGTTGGGCG |
| 154 | Lys_CTT_chr19: 36066750- | GCCCAGCTAGCTCAGTCGGTAGAGCATAA |
| | 36066822 (+) | GACTCTTAATCTCAGGGTTGTGGATTCGT |
| | | GCCCCATGCTGGGTG |
| 155 | Lys_CTT_chr19: 52425393- | GCAGCTAGCTCAGTCGGTAGAGCATGAGA |
| | 52425466 (−) | CTCTTAATCTCAGGGTCATGGGTTCGTGC |
| | | CCCATGTTGGGTGCCA |
| 156 | Lys_CTT_chr1: 145395522- | GCCCGGCTAGCTCAGTCGGTAGAGCATGA |
| | 145395594 (−) | GACTCTTAATCTCAGGGTCGTGGGTTCGA |
| | | GCCCCACGTTGGGCG |
| 157 | Lys_CTT_chr16: 3207406- | GCCCGGCTAGCTCAGTCGGTAGAGCATGA |
| | 3207478 (−) | GACCCTTAATCTCAGGGTCGTGGGTTCGA |
| | | GCCCCACGTTGGGCG |
| 158 | Lys_CTT_chr16: 3241501- | GCCCGGCTAGCTCAGTCGGTAGAGCATGG |
| | 3241573 (+) | GACTCTTAATCTCAGGGTCGTGGGTTCGA |
| | | GCCCCACGTTGGGCG |
| 159 | Lys_CTT_chr16: 3230555- | GCCCGGCTAGCTCAGTCGATAGAGCATGA |
| | 3230627 (−) | GACTCTTAATCTCAGGGTCGTGGGTTCGA |
| | | GCCGCACGTTGGGCG |
| 160 | Lys_CTT_chr1: 55423542- | GCCCAGCTAGCTCAGTCGGTAGAGCATGA |
| | 55423614 (−) | GACTCTTAATCTCAGGGTCATGGGTTTGA |
| | | GCCCCACGTTTGGTG |
| 161 | Lys_CTT_chr16: 3214939- | GCCTGGCTAGCTCAGTCGGCAAAGCATGA |
| | 3215011 (+) | GACTCTTAATCTCAGGGTCGTGGGCTCGA |
| | | GCTCCATGTTGGGCG |
| 162 | Lys_CTT_chr5: 26198539- | GCCCGACTACCTCAGTCGGTGGAGCATGG |
| | 26198611 (−) | GACTCTTCATCCCAGGGTTGTGGGTTCGA |
| | | GCCCCACATTGGGCA |
| 163 | Lys_TTT_chr16: 73512216- | GCCTGGATAGCTCAGTTGGTAGAGCATCA |
| | 73512288 (−) | GACTTTTAATCTGAGGGTCCAGGGTTCAA |
| | | GTCCCTGTTCAGGCA |
| 164 | Lys_TTT_chr12: 27843306- | ACCCAGATAGCTCAGTCAGTAGAGCATCA |
| | 27843378 (+) | GACTTTTAATCTGAGGGTCCAAGGTTCAT |
| | | GTCCCTTTTTGGGTG |
| 165 | Lys_TTT_chr11: 122430655- | GCCTGGATAGCTCAGTTGGTAGAGCATCA |
| | 122430727 (+) | GACTTTTAATCTGAGGGTCCAGGGTTCAA |
| | | GTCCCTGTTCAGGCG |
| 166 | Lys_TTT_chr1: 204475655- | GCCCGGATAGCTCAGTCGGTAGAGCATCA |
| | 204475727 (+) | GACTTTTAATCTGAGGGTCCAGGGTTCAA |
| | | GTCCCTGTTCGGGCG |
| 167 | Lys_TTT_chr6: 27559593- | GCCTGGATAGCTCAGTCGGTAGAGCATCA |
| | 27559665 (−) | GACTTTTAATCTGAGGGTCCAGGGTTCAA |
| | | GTCCCTGTTCAGGCG |
| 168 | Lys_TTT_chr11: 59323902- | GCCCGGATAGCTCAGTCGGTAGAGCATCA |
| | 59323974 (+) | GACTTTTAATCTGAGGGTCCGGGGTTCAA |
| | | GTCCCTGTTCGGGCG |
| 169 | Lys_TTT_chr6: 27302769- | GCCTGGGTAGCTCAGTCGGTAGAGCATCA |
| | 27302841 (−) | GACTTTTAATCTGAGGGTCCAGGGTTCAA |
| | | GTCCCTGTCCAGGCG |
| 170 | Lys_TTT_chr6: 28715521- | GCCTGGATAGCTCAGTTGGTAGAACATCA |
| | 28715593 (+) | GACTTTTAATCTGACGGTGCAGGGTTCAA |
| | | GTCCCTGTTCAGGCG |
| 171 | Met_CAT_chr8: 124169470- | GCCTCGTTAGCGCAGTAGGTAGCGCGTCA |
| | 124169542 (−) | GTCTCATAATCTGAAGGTCGTGAGTTCGA |
| | | TCCTCACACGGGGCA |
| 172 | Met_CAT_chr16: 71460396- | GCCCTCTTAGCGCAGTGGGCAGCGCGTCA |
| | 71460468 (+) | GTCTCATAATCTGAAGGTCCTGAGTTCGA |
| | | GCCTCAGAGAGGGCA |
| 173 | Met_CAT_chr6: 28912352- | GCCTCCTTAGCGCAGTAGGCAGCGCGTCA |
| | 28912424 (+) | GTCTCATAATCTGAAGGTCCTGAGTTCGA |
| | | ACCTCAGAGGGGGCA |
| 174 | Met_CAT_chr6: 26735574- | GCCCTCTTAGCGCAGCGGGCAGCGCGTCA |
| | 26735646 (−) | GTCTCATAATCTGAAGGTCCTGAGTTCGA |
| | | GCCTCAGAGAGGGCA |
| 175 | Met_CAT_chr6: 26701712- | GCCCTCTTAGCGCAGCTGGCAGCGCGTCA |
| | 26701784 (+) | GTCTCATAATCTGAAGGTCCTGAGTTCAA |
| | | GCCTCAGAGAGGGCA |
| 176 | Met_CAT_chr16: 87417628- | GCCTCGTTAGCGCAGTAGGCAGCGCGTCA |
| | 87417700 (−) | GTCTCATAATCTGAAGGTCGTGAGTTCGA |
| | | GCCTCACACGGGGCA |
| 177 | Met_CAT_chr6: 58168492- | GCCCTCTTAGTGCAGCTGGCAGCGCGTCA |
| | 58168564 (−) | GTTTCATAATCTGAAAGTCCTGAGTTCAA |
| | | GCCTCAGAGAGGGCA |
| 178 | Phe_GAA_chr6: 28758499- | GCCGAAATAGCTCAGTTGGGAGAGCGTTA |
| | 28758571 (−) | GACTGAAGATCTAAAGGTCCCTGGTTCGA |
| | | TCCCGGGTTTCGGCA |
| 179 | Phe_GAA_chr11: 59333853- | GCCGAAATAGCTCAGTTGGGAGAGCGTTA |
| | 59333925 (−) | GACTGAAGATCTAAAGGTCCCTGGTTCAA |
| | | TCCCGGGTTTCGGCA |
| 180 | Phe_GAA_chr6: 28775610- | GCCGAGATAGCTCAGTTGGGAGAGCGTTA |
| | 28775682 (−) | GACTGAAGATCTAAAGGTCCCTGGTTCAA |
| | | TCCCGGGTTTCGGCA |
| 181 | Phe_GAA_chr6: 28791093- | GCCGAAATAGCTCAGTTGGGAGAGCGTTA |
| | 28791166 (−) | GACCGAAGATCTTAAAGGTCCCTGGTTCA |
| | | ATCCCGGGTTTCGGCA |
| 182 | Phe_GAA_chr6: 28731374- | GCTGAAATAGCTCAGTTGGGAGAGCGTTA |
| | 28731447 (−) | GACTGAAGATCTTAAAGTTCCCTGGTTCA |
| | | ACCCTGGGTTTCAGCC |
| 183 | Pro_AGG_chr16: 3241989- | GGCTCGTTGGTCTAGGGGTATGATTCTCG |
| | 3242060 (+) | CTTAGGATGCGAGAGGTCCCGGGTTCAAA |
| | | TCCCGGACGAGCCC |
| 184 | Pro_AGG_chr1: 167684725- | GGCTCGTTGGTCTAGGGGTATGATTCTCG |
| | 167684796 (−) | CTTAGGGTGCGAGAGGTCCCGGGTTCAAA |
| | | TCCCGGACGAGCCC |
| 185 | Pro_CGG_chr1: 167683962- | GGCTCGTTGGTCTAGGGGTATGATTCTCG |
| | 167684033 (+) | CTTCGGGTGCGAGAGGTCCCGGGTTCAAA |
| | | TCCCGGACGAGCCC |
| 186 | Pro_CGG_chr6: 27059521- | GGCTCGTTGGTCTAGGGGTATGATTCTCG |
| | 27059592 (+) | CTTCGGGTGTGAGAGGTCCCGGGTTCAAA |
| | | TCCCGGACGAGCCC |
| 187 | Pro_TGG_chr14: 21101165- | GGCTCGTTGGTCTAGTGGTATGATTCTCG |
| | 21101236 (+) | CTTTGGGTGCGAGAGGTCCCGGGTTCAAA |
| | | TCCCGGACGAGCCC |
| 188 | Pro_TGG_chr11: 75946869- | GGCTCGTTGGTCTAGGGGTATGATTCTCG |
| | 75946940 (−) | GTTTGGGTCCGAGAGGTCCCGGGTTCAAA |
| | | TCCCGGACGAGCCC |
| 189 | Pro_TGG_chr5: 180615854- | GGCTCGTTGGTCTAGGGGTATGATTCTCG |
| | 180615925 (−) | CTTTGGGTGCGAGAGGTCCCGGGTTCAAA |
| | | TCCCGGACGAGCCC |
| 190 | SeC_TCA_chr19: 45981859- | GCCCGGATGATCCTCAGTGGTCTGGGGTG |
| | 45981945 (−) | CAGGCTTCAAACCTGTAGCTGTCTAGCGA |
| | | CAGAGTGGTTCAATTCCACCTTTCGGGCG |
| 191 | SeC_TCA_chr22: 44546537- | GCTCGGATGATCCTCAGTGGTCTGGGGTG |
| | 44546620 (+) | CAGGCTTCAAACCTGTAGCTGTCTAGTGA |
| | | CAGAGTGGTTCAATTCCACCTTTGTA |
| 192 | Ser_AGA_chr6: 27509554- | GTAGTCGTGGCCGAGTGGTTAAGGCGATG |
| | 27509635 (−) | GACTAGAAATCCATTGGGGTTTCCCCGCG |
| | | CAGGTTCGAATCCTGCCGACTACG |
| 193 | Ser_AGA_chr6: 26327817- | GTAGTCGTGGCCGAGTGGTTAAGGCGATG |
| | 26327898 (+) | GACTAGAAATCCATTGGGGTCTCCCCGCG |
| | | CAGGTTCGAATCCTGCCGACTACG |
| 194 | Ser_AGA_chr6: 27499987- | GTAGTCGTGGCCGAGTGGTTAAGGCGATG |
| | 27500068 (+) | GACTAGAAATCCATTGGGGTTTCCCCACG |
| | | CAGGTTCGAATCCTGCCGACTACG |
| 195 | Ser_AGA_chr6: 27521192- | GTAGTCGTGGCCGAGTGGTTAAGGTGATG |
| | 27521273 (−) | GACTAGAAACCCATTGGGGTCTCCCCGCG |
| | | CAGGTTCGAATCCTGCCGACTACG |
| 196 | Ser_CGA_chr17: 8042199- | GCTGTGATGGCCGAGTGGTTAAGGCGTTG |
| | 8042280 (−) | GACTCGAAATCCAATGGGGTCTCCCCGCG |
| | | CAGGTTCGAATCCTGCTCACAGCG |
| 197 | Ser_CGA_chr6: 27177628- | GCTGTGATGGCCGAGTGGTTAAGGCGTTG |
| | 27177709 (+) | GACTCGAAATCCAATGGGGTCTCCCCGCG |
| | | CAGGTTCAAATCCTGCTCACAGCG |
| 198 | Ser_CGA_chr6: 27640229- | GCTGTGATGGCCGAGTGGTTAAGGTGTTG |
| | 27640310 (−) | GACTCGAAATCCAATGGGGGTTCCCCGCG |
| | | CAGGTTCAAATCCTGCTCACAGCG |
| 199 | Ser_CGA_chr12: 56584148- | GTCACGGTGGCCGAGTGGTTAAGGCGTTG |
| | 56584229 (+) | GACTCGAAATCCAATGGGGTTTCCCCGCA |
| | | CAGGTTCGAATCCTGTTCGTGACG |
| 200 | Ser_GCT_chr6: 27065085- | GACGAGGTGGCCGAGTGGTTAAGGCGAT |
| | 27065166 (+) | GGACTGCTAATCCATTGTGCTCTGCACGC |
| | | GTGGGTTCGAATCCCACCCTCGTCG |
| 201 | Ser_GCT_chr6: 27265775- | GACGAGGTGGCCGAGTGGTTAAGGCGAT |
| | 27265856 (+) | GGACTGCTAATCCATTGTGCTCTGCACGC |
| | | GTGGGTTCGAATCCCACCTTCGTCG |
| 202 | Ser_GCT_chr11: 66115591- | GACGAGGTGGCCGAGTGGTTAAGGCGAT |
| | 66115672 (+) | GGACTGCTAATCCATTGTGCTTTGCACGC |
| | | GTGGGTTCGAATCCCATCCTCGTCG |
| 203 | Ser_GCT_chr6: 28565117- | GACGAGGTGGCCGAGTGGTTAAGGCGAT |
| | 28565198 (−) | GGACTGCTAATCCATTGTGCTCTGCACGC |
| | | GTGGGTTCGAATCCCATCCTCGTCG |
| 204 | Ser_GCT_chr6: 28180815- | GACGAGGTGGCCGAGTGGTTAAGGCGAT |
| | 28180896 (+) | GGACTGCTAATCCATTGTGCTCTGCACAC |
| | | GTGGGTTCGAATCCCATCCTCGTCG |
| 205 | Scr_GCT_chr6: 26305718- | GGAGAGGCCTGGCCGAGTGGTTAAGGCG |
| | 26305801 (−) | ATGGACTGCTAATCCATTGTGCTCTGCAC |
| | | GCGTGGGTTCGAATCCCATCCTCGTCG |
| 206 | Ser_TGA_chr10: 69524261- | GCAGCGATGGCCGAGTGGTTAAGGCGTTG |
| | 69524342 (+) | GACTTGAAATCCAATGGGGTCTCCCCGCG |
| | | CAGGTTCGAACCCTGCTCGCTGCG |
| 207 | Ser_TGA_chr6: 27513468- | GTAGTCGTGGCCGAGTGGTTAAGGCGATG |
| | 27513549 (+) | GACTTGAAATCCATTGGGGTTTCCCCGCG |
| | | CAGGTTCGAATCCTGCCGACTACG |
| 208 | Ser_TGA_chr6: 26312824- | GTAGTCGTGGCCGAGTGGTTAAGGCGATG |
| | 26312905 (−) | GACTTGAAATCCATTGGGGTCTCCCCGCG |
| | | CAGGTTCGAATCCTGCCGACTACG |
| 209 | Ser_TGA_chr6: 27473607- | GTAGTCGTGGCCGAGTGGTTAAGGCGATG |
| | 27473688 (−) | GACTTGAAATCCATTGGGGTTTCCCCGCG |
| | | CAGGTTCGAATCCTGTCGGCTACG |
| 210 | Thr_AGT_chr17: 8090478- | GGCGCCGTGGCTTAGTTGGTTAAAGCGCC |
| | 8090551 (+) | TGTCTAGTAAACAGGAGATCCTGGGTTCG |
| | | AATCCCAGCGGTGCCT |
| 211 | Thr_AGT_chr6: 26533145- | GGCTCCGTGGCTTAGCTGGTTAAAGCGCC |
| | 26533218 (−) | TGTCTAGTAAACAGGAGATCCTGGGTTCG |
| | | AATCCCAGCGGGGCCT |
| 212 | Thr_AGT_chr6: 28693795- | GGCTCCGTAGCTTAGTTGGTTAAAGCGCC |
| | 28693868 (+) | TGTCTAGTAAACAGGAGATCCTGGGTTCG |
| | | ACTCCCAGCGGGGCCT |
| 213 | Thr_AGT_chr6: 27694473- | GGCTTCGTGGCTTAGCTGGTTAAAGCGCC |
| | 27694546 (+) | TGTCTAGTAAACAGGAGATCCTGGGTTCG |
| | | AATCCCAGCGAGGCCT |
| 214 | Thr_AGT_chr17: 8042770- | GGCGCCGTGGCTTAGCTGGTTAAAGCGCC |
| | 8042843 (−) | TGTCTAGTAAACAGGAGATCCTGGGTTCG |
| | | AATCCCAGCGGTGCCT |
| 215 | Thr_AGT_chr6: 27130050- | GGCCCTGTGGCTTAGCTGGTCAAAGCGCC |
| | 27130123 (+) | TGTCTAGTAAACAGGAGATCCTGGGTTCG |
| | | AATCCCAGCGGGGCCT |
| 216 | Thr_CGT_chr6: 28456770- | GGCTCTATGGCTTAGTTGGTTAAAGCGCC |
| | 28456843 (−) | TGTCTCGTAAACAGGAGATCCTGGGTTCG |
| | | ACTCCCAGTGGGGCCT |
| 217 | Thr_CGT_chr16: 14379750- | GGCGCGGTGGCCAAGTGGTAAGGCGTCG |
| | 14379821 (+) | GTCTCGTAAACCGAAGATCACGGGTTCGA |
| | | ACCCCGTCCGTGCCT |
| 218 | Thr_CGT_chr6: 28615984- | GGCTCTGTGGCTTAGTTGGCTAAAGCGCC |
| | 28616057 (−) | TGTCTCGTAAACAGGAGATCCTGGGTTCG |
| | | AATCCCAGCGGGGCCT |
| 219 | Thr_CGT_chr17: 29877093- | GGCGCGGTGGCCAAGTGGTAAGGCGTCG |
| | 29877164 (+) | GTCTCGTAAACCGAAGATCGCGGGTTCGA |
| | | ACCCCGTCCGTGCCT |
| 220 | Thr_CGT_chr6: 27586135- | GGCCCTGTAGCTCAGCGGTTGGAGCGCTG |
| | 27586208 (+) | GTCTCGTAAACCTAGGGGTCGTGAGTTCA |
| | | AATCTCACCAGGGCCT |
| 221 | Thr_TGT_chr6: 28442329- | GGCTCTATGGCTTAGTTGGTTAAAGCGCC |
| | 28442402 (−) | TGTCTTGTAAACAGGAGATCCTGGGTTCG |
| | | AATCCCAGTAGAGCCT |
| 222 | Thr_TGT_chr1: 222638347- | GGCTCCATAGCTCAGTGGTTAGAGCACTG |
| | 222638419 (+) | GTCTTGTAAACCAGGGGTCGCGAGTTCGA |
| | | TCCTCGCTGGGGCCT |
| 223 | Thr_TGT_chr14: 21081949- | GGCTCCATAGCTCAGGGGTTAGAGCGCTG |
| | 21082021 (−) | GTCTTGTAAACCAGGGGTCGCGAGTTCAA |
| | | TTCTCGCTGGGGCCT |
| 224 | Thr_TGT_chr14: 21099319- | GGCTCCATAGCTCAGGGGTTAGAGCACTG |
| | 21099391 (−) | GTCTTGTAAACCAGGGGTCGCGAGTTCAA |
| | | ATCTCGCTGGGGCCT |
| 225 | Thr_TGT_chr14: 21149849- | GGCCCTATAGCTCAGGGGTTAGAGCACTG |
| | 21149921 (+) | GTCTTGTAAACCAGGGGTCGCGAGTTCAA |
| | | ATCTCGCTGGGGCCT |
| 226 | Thr_TGT_chr5: 180618687- | GGCTCCATAGCTCAGGGGTTAGAGCACTG |
| | 180618758 (−) | GTCTTGTAAACCAGGGTCGCGAGTTCAAA |
| | | TCTCGCTGGGGCCT |
| 227 | Trp_CCA_chr17: 8124187- | GGCCTCGTGGCGCAACGGTAGCGCGTCTG |
| | 8124258 (−) | ACTCCAGATCAGAAGGTTGCGTGTTCAAA |
| | | TCACGTCGGGGTCA |
| 228 | Trp_CCA_chr17: 19411494- | GACCTCGTGGCGCAATGGTAGCGCGTCTG |
| | 19411565 (+) | ACTCCAGATCAGAAGGTTGCGTGTTCAAG |
| | | TCACGTCGGGGTCA |
| 229 | Trp_CCA_chr6: 26319330- | GACCTCGTGGCGCAACGGTAGCGCGTCTG |
| | 26319401 (−) | ACTCCAGATCAGAAGGTTGCGTGTTCAAA |
| | | TCACGTCGGGGTCA |
| 230 | Trp_CCA_chr12: 98898030- | GACCTCGTGGCGCAACGGTAGCGCGTCTG |
| | 98898101 (+) | ACTCCAGATCAGAAGGCTGCGTGTTCGAA |
| | | TCACGTCGGGGTCA |
| 231 | Trp_CCA_chr7: 99067307- | GACCTCGTGGCGCAACGGCAGCGCGTCTG |
| | 99067378 (+) | ACTCCAGATCAGAAGGTTGCGTGTTCAAA |
| | | TCACGTCGGGGTCA |
| 232 | Tyr_ATA_chr2: 219110549- | CCTTCAATAGTTCAGCTGGTAGAGCAGAG |
| | 219110641 (+) | GACTATAGCTACTTCCTCAGTAGGAGACG |
| | | TCCTTAGGTTGCTGGTTCGATTCCAGCTTG |
| | | AAGGA |
| 233 | Tyr_GTA_chr6: 26569086- | CCTTCGATAGCTCAGTTGGTAGAGCGGAG |
| | 26569176 (+) | GACTGTAGTTGGCTGTGTCCTTAGACATC |
| | | CTTAGGTCGCTGGTTCGAATCCGGCTCGA |
| | | AGGA |
| 234 | Tyr_GTA_chr2: 27273650- | CCTTCGATAGCTCAGTTGGTAGAGCGGAG |
| | 27273738 (+) | GACTGTAGTGGATAGGGCGTGGCAATCCT |
| | | TAGGTCGCTGGTTCGATTCCGGCTCGAAG |
| | | GA |
| 235 | Tyr_GTA_chr6: 26577332- | CCTTCGATAGCTCAGTTGGTAGAGCGGAG |
| | 26577420 (+) | GACTGTAGGCTCATTAAGCAAGGTATCCT |
| | | TAGGTCGCTGGTTCGAATCCGGCTCGGAG |
| | | GA |
| 236 | Tyr_GTA_chr14: 21125623- | CCTTCGATAGCTCAGCTGGTAGAGCGGAG |
| | 21125716 (−) | GACTGTAGATTGTATAGACATTTGCGGAC |
| | | ATCCTTAGGTCGCTGGTTCGATTCCAGCTC |
| | | GAAGGA |
| 237 | Tyr_GTA_chr8: 67025602- | CCTTCGATAGCTCAGCTGGTAGAGCGGAG |
| | 67025694 (+) | GACTGTAGCTACTTCCTCAGCAGGAGACA |
| | | TCCTTAGGTCGCTGGTTCGATTCCGGCTCG |
| | | AAGGA |
| 238 | Tyr_GTA_chr8: 67026223- | CCTTCGATAGCTCAGCTGGTAGAGCGGAG |
| | 67026311 (+) | GACTGTAGGCGCGCGCCCGTGGCCATCCT |
| | | TAGGTCGCTGGTTCGATTCCGGCTCGAAG |
| | | GA |
| 239 | Tyr_GTA_chr14: 21121258- | CCTTCGATAGCTCAGCTGGTAGAGCGGAG |
| | 21121351 (−) | GACTGTAGCCTGTAGAAACATTTGTGGAC |
| | | ATCCTTAGGTCGCTGGTTCGATTCCGGCTC |
| | | GAAGGA |
| 240 | Tyr_GTA_chr14: 21131351- | CCTTCGATAGCTCAGCTGGTAGAGCGGAG |
| | 21131444 (−) | GACTGTAGATTGTACAGACATTTGCGGAC |
| | | ATCCTTAGGTCGCTGGTTCGATTCCGGCTC |
| | | GAAGGA |
| 241 | Tyr_GTA_chr14: 21151432- | CCTTCGATAGCTCAGCTGGTAGAGCGGAG |
| | 21151520 (+) | GACTGTAGTACTTAATGTGTGGTCATCCTT |
| | | AGGTCGCTGGTTCGATTCCGGCTCGAAGG |
| | | A |
| 242 | Tyr_GTA_chr6: 26595102- | CCTTCGATAGCTCAGCTGGTAGAGCGGAG |
| | 26595190 (+) | GACTGTAGGGGTTTGAATGTGGTCATCCT |
| | | TAGGTCGCTGGTTCGAATCCGGCTCGGAG |
| | | GA |
| 243 | Tyr_GTA_chr14: 21128117- | CCTTCGATAGCTCAGCTGGTAGAGCGGAG |
| | 21128210 (−) | GACTGTAGACTGCGGAAACGTTTGTGGAC |
| | | ATCCTTAGGTCGCTGGTTCAATTCCGGCTC |
| | | GAAGGA |
| 244 | Tyr_GTA_chr6: 26575798- | CTTTCGATAGCTCAGTTGGTAGAGCGGAG |
| | 26575887 (+) | GACTGTAGGTTCATTAAACTAAGGCATCC |
| | | TTAGGTCGCTGGTTCGAATCCGGCTCGAA |
| | | GGA |
| 245 | Tyr_GTA_chr8: 66609532- | TCTTCAATAGCTCAGCTGGTAGAGCGGAG |
| | 66609619 (−) | GACTGTAGGTGCACGCCCGTGGCCATTCT |
| | | TAGGTGCTGGTTTGATTCCGACTTGGAGA |
| | | G |
| 246 | Val_AAC_chr3: 169490018- | GTTTCCGTAGTGTAGTGGTTATCACGTTCG |
| | 169490090 (+) | CCTAACACGCGAAAGGTCCCCGGTTCGAA |
| | | ACCGGGCGGAAACA |
| 247 | Val_AAC_chr5: 180615416- | GTTTCCGTAGTGTAGTGGTCATCACGTTC |
| | 180615488 (−) | GCCTAACACGCGAAAGGTCCCCGGTTCGA |
| | | AACCGGGCGGAAACA |
| 248 | Val_AAC_chr6: 27618707- | GTTTCCGTAGTGTAGTGGTTATCACGTTCG |
| | 27618779 (−) | CCTAACACGCGAAAGGTCCCTGGATCAAA |
| | | ACCAGGCGGAAACA |
| 249 | Val_AAC_chr6: 27648885- | GTTTCCGTAGTGTAGTGGTTATCACGTTCG |
| | 27648957 (−) | CCTAACACGCGAAAGGTCCGCGGTTCGAA |
| | | ACCGGGCGGAAACA |
| 250 | Val_AAC_chr6: 27203288- | GTTTCCGTAGTGTAGTGGTTATCACGTTTG |
| | 27203360 (+) | CCTAACACGCGAAAGGTCCCCGGTTCGAA |
| | | ACCGGGCAGAAACA |
| 251 | Val_AAC_chr6: 28703206- | GGGGGTGTAGCTCAGTGGTAGAGCGTATG |
| | 28703277 (−) | CTTAACATTCATGAGGCTCTGGGTTCGAT |
| | | CCCCAGCACTTCCA |
| 252 | Val_CAC_chr1: 161369490- | GTTTCCGTAGTGTAGTGGTTATCACGTTCG |
| | 161369562 (−) | CCTCACACGCGAAAGGTCCCCGGTTCGAA |
| | | ACCGGGCGGAAACA |
| 253 | Val_CAC_chr6: 27248049- | GCTTCTGTAGTGTAGTGGTTATCACGTTCG |
| | 27248121 (−) | CCTCACACGCGAAAGGTCCCCGGTTCGAA |
| | | ACCGGGCAGAAGCA |
| 254 | Val_CAC_chr19: 4724647- | GTTTCCGTAGTGTAGCGGTTATCACATTC |
| | 4724719 (−) | GCCTCACACGCGAAAGGTCCCCGGTTCGA |
| | | TCCCGGGCGGAAACA |
| 255 | Val_CAC_chr1: 149298555- | GTTTCCGTAGTGTAGTGGTTATCACGTTCG |
| | 149298627 (−) | CCTCACACGCGAAAGGTCCCCGGTTCGAA |
| | | ACTGGGCGGAAACA |
| 256 | Val_CAC_chr1: 149684088- | GTTTCCGTAGTGTAGTGGTTATCACGTTCG |
| | 149684161 (−) | CCTCACACGCGTAAAGGTCCCCGGTTCGA |
| | | AACCGGGCGGAAACA |
| 257 | Val_CAC_chr6: 27173867- | GTTTCCGTAGTGGAGTGGTTATCACGTTC |
| | 27173939 (−) | GCCTCACACGCGAAAGGTCCCCGGTTTGA |
| | | AACCAGGCGGAAACA |
| 258 | Val_TAC_chr11: 59318102- | GGTTCCATAGTGTAGTGGTTATCACGTCT |
| | 59318174 (−) | GCTTTACACGCAGAAGGTCCTGGGTTCGA |
| | | GCCCCAGTGGAACCA |
| 259 | Val_TAC_chr11: 59318460- | GGTTCCATAGTGTAGCGGTTATCACGTCT |
| | 59318532 (−) | GCTTTACACGCAGAAGGTCCTGGGTTCGA |
| | | GCCCCAGTGGAACCA |
| 260 | Val_TAC_chr10: 5895674- | GGTTCCATAGTGTAGTGGTTATCACATCT |
| | 5895746 (−) | GCTTTACACGCAGAAGGTCCTGGGTTCAA |
| | | GCCCCAGTGGAACCA |
| 261 | Val_TAC_chr6: 27258405- | GTTTCCGTGGTGTAGTGGTTATCACATTCG |
| | 27258477 (+) | CCTTACACGCGAAAGGTCCTCGGGTCGAA |
| | | ACCGAGCGGAAACA |
| 262 | iMet_CAT_chr1: 153643726- | AGCAGAGTGGCGCAGCGGAAGCGTGCTG |
| | 153643797 (+) | GGCCCATAACCCAGAGGTCGATGGATCGA |
| | | AACCATCCTCTGCTA |
| 263 | iMet_CAT_chr6: 27745664- | AGCAGAGTGGCGCAGCGGAAGCGTGCTG |
| | 27745735 (+) | GGCCCATAACCCAGAGGTCGATGGATCTA |
| | | AACCATCCTCTGCTA |
| 264 | Glu_TTC_chr1: 16861773- | TCCCTGGTGGTCTAGTGGCTAGGATTCGG |
| | 16861845 (−) | CGCTTTCACCGCCGCGGCCCGGGTTCGAT |
| | | TCCCGGTCAGGGAAT |
| 265 | Gly_CCC_chr1: 17004765- | GCGTTGGTGGTTTAGTGGTAGAATTCTCG |
| | 17004836 (−) | CCTCCCATGCGGGAGACCCGGGTTCAATT |
| | | CCCGGCCACTGCAC |
| 266 | Gly_CCC_chr1: 17053779- | GGCCTTGGTGGTGCAGTGGTAGAATTCTC |
| | 17053850 (+) | GCCTCCCACGTGGGAGACCCGGGTTCAAT |
| | | TCCCGGCCAATGCA |
| 267 | Glu_TTC_chr1: 17199077- | GTCCCTGGTGGTCTAGTGGCTAGGATTCG |
| | 17199149 (+) | GCGCTTTCACCGCCGCGGCCCGGGTTCGA |
| | | TTCCCGGCCAGGGAA |
| 268 | Asn_GTT_chr1: 17216171- | TGTCTCTGTGGCGCAATCGGTTAGCGCGT |
| | 17216245 (+) | TCGGCTGTTAACCGAAAGATTGGTGGTTC |
| | | GAGCCCACCCAGGGACG |
| 269 | Arg_TCT_chr1: 94313128- | TGGCTCCGTGGCGCAATGGATAGCGCATT |
| | 94313213 (+) | GGACTTCTAGAGGCTGAAGGCATTCAAAG |
| | | GTTCCGGGTTCGAGTCCCGGCGGAGTCG |
| 270 | Lys_CTT_chr1: 145395521- | GCCCGGCTAGCTCAGTCGGTAGAGCATGA |
| | 145395594 (−) | GACTCTTAATCTCAGGGTCGTGGGTTCGA |
| | | GCCCCACGTTGGGCGC |
| 271 | His_GTG_chr1: 145396880- | GCCGTGATCGTATAGTGGTTAGTACTCTG |
| | 145396952 (−) | CGTTGTGGCCGCAGCAACCTCGGTTCGAA |
| | | TCCGAGTCACGGCAG |
| 272 | Gly_TCC_chr1: 145397863- | GCGTTGGTGGTATAGTGGTGAGCATAGCT |
| | 145397935 (−) | GCCTTCCAAGCAGTTGACCCGGGTTCGAT |
| | | TCCCGGCCAACGCAG |
| 273 | Glu_CTC_chr1: 145399232- | TCCCTGGTGGTCTAGTGGTTAGGATTCGG |
| | 145399304 (−) | CGCTCTCACCGCCGCGGCCCGGGTTCGAT |
| | | TCCCGGTCAGGGAAA |
| 274 | Gln_CTG_chr1: 145963303- | AGGTTCCATGGTGTAATGGTGAGCACTCT |
| | 145963375 (+) | GGACTCTGAATCCAGCGATCCGAGTTCGA |
| | | GTCTCGGTGGAACCT |
| 275 | Asn_GTT_chr1: 148000804- | TGTCTCTGTGGCGTAGTCGGTTAGCGCGT |
| | 148000878 (+) | TCGGCTGTTAACCGAAAAGTTGGTGGTTC |
| | | GAGCCCACCCAGGAACG |
| 276 | Asn_GTT_chr1: 148248114- | TGTCTCTGTGGCGCAATCGGTTAGCGCGT |
| | 148248188 (+) | TCGGCTGTTAACCGAAAGGTTGGTGGTTC |
| | | GAGCCCACCCAGGGACG |
| 277 | Asn_GTT_chr1: 148598313- | GTCTCTGTGGCGCAATCGGTTAGCGCATT |
| | 148598387 (−) | CGGCTGTTAACCGAAAGGTTGGTGGTTCG |
| | | AGCCCACCCAGGGACGC |
| 278 | Asn_GTT_chr1: 149230569- | GTCTCTGTGGCGCAATGGGTTAGCGCGTT |
| | 149230643 (−) | CGGCTGTTAACCGAAAGGTTGGTGGTTCG |
| | | AGCCCATCCAGGGACGC |
| 279 | Val_CAC_chr1: 149294665- | GCACTGGTGGTTCAGTGGTAGAATTCTCG |
| | 149294736 (−) | CCTCACACGCGGGACACCCGGGTTCAATT |
| | | CCCGGTCAAGGCAA |
| 280 | Val_CAC_chr1: 149298554- | GTTTCCGTAGTGTAGTGGTTATCACGTTCG |
| | 149298627 (−) | CCTCACACGCGAAAGGTCCCCGGTTCGAA |
| | | ACTGGGCGGAAACAG |
| 281 | Gly_CCC_chr1: 149680209- | GCACTGGTGGTTCAGTGGTAGAATTCTCG |
| | 149680280 (−) | CCTCCCACGCGGGAGACCCGGGTTTAATT |
| | | CCCGGTCAAGATAA |
| 282 | Val_CAC_chr1: 149684087- | GTTTCCGTAGTGTAGTGGTTATCACGTTCG |
| | 149684161 (−) | CCTCACACGCGTAAAGGTCCCCGGTTCGA |
| | | AACCGGGCGGAAACAT |
| 283 | Met_CAT_chr1: 153643725- | TAGCAGAGTGGCGCAGCGGAAGCGTGCT |
| | 153643797 (+) | GGGCCCATAACCCAGAGGTCGATGGATCG |
| | | AAACCATCCTCTGCTA |
| 284 | Val_CAC_chr1: 161369489- | GTTTCCGTAGTGTAGTGGTTATCACGTTCG |
| | 161369562 (−) | CCTCACACGCGAAAGGTCCCCGGTTCGAA |
| | | ACCGGGCGGAAACAA |
| 285 | Asp_GTC_chr1: 161410614- | TCCTCGTTAGTATAGTGGTGAGTATCCCC |
| | 161410686 (−) | GCCTGTCACGCGGGAGACCGGGGTTCGAT |
| | | TCCCCGACGGGGAGG |
| 286 | Gly_GCC_chr1: 161413093- | TGCATGGGTGGTTCAGTGGTAGAATTCTC |
| | 161413164 (+) | GCCTGCCACGCGGGAGGCCCGGGTTCGAT |
| | | TCCCGGCCCATGCA |
| 287 | Glu_CTC_chr1: 161417017- | TCCCTGGTGGTCTAGTGGTTAGGATTCGG |
| | 161417089 (−) | CGCTCTCACCGCCGCGGCCCGGGTTCGAT |
| | | TCCCGGTCAGGGAAG |
| 288 | Asp_GTC_chr1: 161492934- | ATCCTTGTTACTATAGTGGTGAGTATCTCT |
| | 161493006 (+) | GCCTGTCATGCGTGAGAGAGGGGGTCGAT |
| | | TCCCCGACGGGGAG |
| 289 | Gly_GCC_chr1: 161493636- | GCATTGGTGGTTCAGTGGTAGAATTCTCG |
| | 161493707 (−) | CCTGCCACGCGGGAGGCCCGGGTTCGATT |
| | | CCCGGCCAATGCAC |
| 290 | Leu_CAG_chr1: 161500131- | GTCAGGATGGCCGAGCGGTCTAAGGCGCT |
| | 161500214 (−) | GCGTTCAGGTCGCAGTCTCCCCTGGAGGC |
| | | GTGGGTTCGAATCCCACTCCTGACAA |
| 291 | Gly_TCC_chr1: 161500902- | CGCGTTGGTGGTATAGTGGTGAGCATAGC |
| | 161500974 (+) | TGCCTTCCAAGCAGTTGACCCGGGTTCGA |
| | | TTCCCGGCCAACGCA |
| 292 | Asn_GTT_chr1: 161510030- | CGTCTCTGTGGCGCAATCGGTTAGCGCGT |
| | 161510104 (+) | TCGGCTGTTAACCGAAAGGTTGGTGGTTC |
| | | GATCCCACCCAGGGACG |
| 293 | Glu_TTC_chr1: 161582507- | CGCGTTGGTGGTGTAGTGGTGAGCACAGC |
| | 161582579 (+) | TGCCTTTCAAGCAGTTAACGCGGGTTCGA |
| | | TTCCCGGGTAACGAA |
| 294 | Pro_CGG_chr1: 167683961- | CGGCTCGTTGGTCTAGGGGTATGATTCTC |
| | 167684033 (+) | GCTTCGGGTGCGAGAGGTCCCGGGTTCAA |
| | | ATCCCGGACGAGCCC |
| 295 | Pro_AGG_chr1: 167684724- | GGCTCGTTGGTCTAGGGGTATGATTCTCG |
| | 167684796 (−) | CTTAGGGTGCGAGAGGTCCCGGGTTCAAA |
| | | TCCCGGACGAGCCCT |
| 296 | Lys_TTT_chr1: 204475654- | CGCCCGGATAGCTCAGTCGGTAGAGCATC |
| | 204475727 (+) | AGACTTTTAATCTGAGGGTCCAGGGTTCA |
| | | AGTCCCTGTTCGGGCG |
| 297 | Lys_TTT_chr1: 204476157- | GCCCGGATAGCTCAGTCGGTAGAGCATCA |
| | 204476230 (−) | GACTTTTAATCTGAGGGTCCAGGGTTCAA |
| | | GTCCCTGTTCGGGCGT |
| 298 | Leu_CAA_chr1: 249168053- | TGTCAGGATGGCCGAGTGGTCTAAGGCGC |
| | 249168159 (+) | CAGACTCAAGGTAAGCACCTTGCCTGCGG |
| | | GCTTTCTGGTCTCCGGATGGAGGCGTGGG |
| | | TTCGAATCCCACTTCTGACA |
| 299 | Glu_CTC_chr1: 249168446- | TTCCCTGGTGGTCTAGTGGTTAGGATTCG |
| | 249168518 (+) | GCGCTCTCACCGCCGCGGCCCGGGTTCGA |
| | | TTCCCGGTCAGGAAA |
| 300 | Tyr_GTA_chr2: 27273649- | GCCTTCGATAGCTCAGTTGGTAGAGCGGA |
| | 27273738 (+) | GGACTGTAGTGGATAGGGCGTGGCAATCC |
| | | TTAGGTCGCTGGTTCGATTCCGGCTCGAA |
| | | GGA |
| 301 | Ala_AGC_chr2: 27274081- | CGGGGGATTAGCTCAAATGGTAGAGCGCT |
| | 27274154 (+) | CGCTTAGCATGCGAGAGGTAGCGGGATCG |
| | | ATGCCCGCATCCTCCA |
| 302 | Ile_TAT_chr2: 43037675- | AGCTCCAGTGGCGCAATCGGTTAGCGCGC |
| | 43037768 (+) | GGTACTTATACAGCAGTACATGCAGAGCA |
| | | ATGCCGAGGTTGTGAGTTCGAGCCTCACC |
| | | TGGAGCA |
| 303 | Gly_CCC_chr2: 70476122- | GCGCCGCTGGTGTAGTGGTATCATGCAAG |
| | 70476193 (−) | ATTCCCATTCTTGCGACCCGGGTTCGATTC |
| | | CCGGGCGGCGCAT |
| 304 | Glu_TTC_chr2: 131094700- | TCCCATATGGTCTAGCGGTTAGGATTCCT |
| | 131094772 (−) | GGTTTTCACCCAGGTGGCCCGGGTTCGAC |
| | | TCCCGGTATGGGAAC |
| 305 | Ala_CGC_chr2: 157257280- | GGGGGATGTAGCTCAGTGGTAGAGCGCG |
| | 157257352 (+) | CGCTTCGCATGTGTGAGGTCCCGGGTTCA |
| | | ATCCCCGGCATCTCCA |
| 306 | Gly_GCC_chr2: 157257658- | GCATTGGTGGTTCAGTGGTAGAATTCTCG |
| | 157257729 (−) | CCTGCCACGCGGGAGGCCCGGGTTCGATT |
| | | CCCGGCCAATGCAA |
| 307 | Arg_ACG_chr3: 45730490- | GGGCCAGTGGCGCAATGGATAACGCGTCT |
| | 45730563 (−) | GACTACGGATCAGAAGATTCTAGGTTCGA |
| | | CTCCTGGCTGGCTCGC |
| 308 | Val_AAC_chr3: 169490017- | GGTTTCCGTAGTGTAGTGGTTATCACGTTC |
| | 169490090 (+) | GCCTAACACGCGAAAGGTCCCCGGTTCGA |
| | | AACCGGGCGGAAACA |
| 309 | Val_AAC_chr5: 180596609- | AGTTTCCGTAGTGTAGTGGTTATCACGTTC |
| | 180596682 (+) | GCCTAACACGCGAAAGGTCCCCGGTTCGA |
| | | AACCGGGCGGAAACA |
| 310 | Leu_AAG_chr5: 180614700- | AGGTAGCGTGGCCGAGCGGTCTAAGGCG |
| | 180614782 (+) | CTGGATTAAGGCTCCAGTCTCTTCGGGGG |
| | | CGTGGGTTCGAATCCCACCGCTGCCA |
| 311 | Val_AAC_chr5: 180615415- | GTTTCCGTAGTGTAGTGGTCATCACGTTC |
| | 180615488 (−) | GCCTAACACGCGAAAGGTCCCCGGTTCGA |
| | | AACCGGGCGGAAACAT |
| 312 | Pro_TGG_chr5: 180615853- | GGCTCGTTGGTCTAGGGGTATGATTCTCG |
| | 180615925 (−) | CTTTGGGTGCGAGAGGTCCCGGGTTCAAA |
| | | TCCCGGACGAGCCCA |
| 313 | Thr_TGT_chr5: 180618686- | GGCTCCATAGCTCAGGGGTTAGAGCACTG |
| | 180618758 (−) | GTCTTGTAAACCAGGGTCGCGAGTTCAAA |
| | | TCTCGCTGGGGCCTG |
| 314 | Ala_TGC_chr5: 180633867- | TGGGGATGTAGCTCAGTGGTAGAGCGCAT |
| | 180633939 (+) | GCTTTGCATGTATGAGGCCCCGGGTTCGA |
| | | TCCCCGGCATCTCCA |
| 315 | Lys_CTT_chr5: 180634754- | CGCCCGGCTAGCTCAGTCGGTAGAGCATG |
| | 180634827 (+) | AGACTCTTAATCTCAGGGTCGTGGGTTCG |
| | | AGCCCCACGTTGGGCG |
| 316 | Val_AAC_chr5: 180645269- | GTTTCCGTAGTGTAGTGGTTATCACGTTCG |
| | 180645342 (−) | CCTAACACGCGAAAGGTCCCCGGTTCGAA |
| | | ACCGGGCGGAAACAA |
| 317 | Lys_CTT_chr5: 180648978- | GCCCGGCTAGCTCAGTCGGTAGAGCATGA |
| | 180649051 (−) | GACTCTTAATCTCAGGGTCGTGGGTTCGA |
| | | GCCCCACGTTGGGCGT |
| 318 | Val_CAC_chr5: 180649394- | GTTTCCGTAGTGTAGTGGTTATCACGTTCG |
| | 180649467 (−) | CCTCACACGCGAAAGGTCCCCGGTTCGAA |
| | | ACCGGGCGGAAACAC |
| 319 | Met_CAT_chr6: 26286753- | CAGCAGAGTGGCGCAGCGGAAGCGTGCT |
| | 26286825 (+) | GGGCCCATAACCCAGAGGTCGATGGATCG |
| | | AAACCATCCTCTGCTA |
| 320 | Ser_GCT_chr6: 26305717- | GGAGAGGCCTGGCCGAGTGGTTAAGGCG |
| | 26305801 (−) | ATGGACTGCTAATCCATTGTGCTCTGCAC |
| | | GCGTGGGTTCGAATCCCATCCTCGTCGC |
| 321 | Gln_TTG_chr6: 26311423- | GGCCCCATGGTGTAATGGTTAGCACTCTG |
| | 26311495 (−) | GACTTTGAATCCAGCGATCCGAGTTCAAA |
| | | TCTCGGTGGGACCTG |
| 322 | Gln_TTG_chr6: 26311974- | GGCCCCATGGTGTAATGGTTAGCACTCTG |
| | 26312046 (−) | GACTTTGAATCCAGCGATCCGAGTTCAAA |
| | | TCTCGGTGGGACCTA |
| 323 | Ser_TGA_chr6: 26312823- | GTAGTCGTGGCCGAGTGGTTAAGGCGATG |
| | 26312905 (−) | GACTTGAAATCCATTGGGGTCTCCCCGCG |
| | | CAGGTTCGAATCCTGCCGACTACGG |
| 324 | Met_CAT_chr6: 26313351- | AGCAGAGTGGCGCAGCGGAAGCGTGCTG |
| | 26313423 (−) | GGCCCATAACCCAGAGGTCGATGGATCGA |
| | | AACCATCCTCTGCTAT |
| 325 | Arg_TCG_chr6: 26323045- | GGACCACGTGGCCTAATGGATAAGGCGTC |
| | 26323118 (+) | TGACTTCGGATCAGAAGATTGAGGGTTCG |
| | | AATCCCTCCGTGGTTA |
| 326 | Ser_AGA_chr6: 26327816- | TGTAGTCGTGGCCGAGTGGTTAAGGCGAT |
| | 26327898 (+) | GGACTAGAAATCCATTGGGGTCTCCCCGC |
| | | GCAGGTTCGAATCCTGCCGACTACG |
| 327 | Met_CAT_chr6: 26330528- | AGCAGAGTGGCGCAGCGGAAGCGTGCTG |
| | 26330600 (−) | GGCCCATAACCCAGAGGTCGATGGATCGA |
| | | AACCATCCTCTGCTAG |
| 328 | Leu_CAG_chr6: 26521435- | CGTCAGGATGGCCGAGCGGTCTAAGGCGC |
| | 26521518 (+) | TGCGTTCAGGTCGCAGTCTCCCCTGGAGG |
| | | CGTGGGTTCGAATCCCACTCCTGACA |
| 329 | Thr_AGT_chr6: 26533144- | GGCTCCGTGGCTTAGCTGGTTAAAGCGCC |
| | 26533218 (−) | TGTCTAGTAAACAGGAGATCCTGGGTTCG |
| | | AATCCCAGCGGGGCCTG |
| 330 | Arg_ACG_chr6: 26537725- | AGGGCCAGTGGCGCAATGGATAACGCGT |
| | 26537798 (+) | CTGACTACGGATCAGAAGATTCCAGGTTC |
| | | GACTCCTGGCTGGCTCG |
| 331 | Val_CAC_chr6: 26538281- | GGTTTCCGTAGTGTAGTGGTTATCACGTTC |
| | 26538354 (+) | GCCTCACACGCGAAAGGTCCCCGGTTCGA |
| | | AACCGGGCGGAAACA |
| 332 | Ala_CGC_chr6: 26553730- | AGGGGATGTAGCTCAGTGGTAGAGCGCAT |
| | 26553802 (+) | GCTTCGCATGTATGAGGTCCCGGGTTCGA |
| | | TCCCCGGCATCTCCA |
| 333 | Ile_AAT_chr6: 26554349- | TGGCCGGTTAGCTCAGTTGGTTAGAGCGT |
| | 26554423 (+) | GGTGCTAATAACGCCAAGGTCGCGGGTTC |
| | | GATCCCCGTACGGGCCA |
| 334 | Pro_AGG_chr6: 26555497- | CGGCTCGTTGGTCTAGGGGTATGATTCTC |
| | 26555569 (+) | GCTTAGGGTGCGAGAGGTCCCGGGTTCAA |
| | | ATCCCGGACGAGCCC |
| 335 | Lys_CTT_chr6: 26556773- | AGCCCGGCTAGCTCAGTCGGTAGAGCATG |
| | 26556846 (+) | AGACTCTTAATCTCAGGGTCGTGGGTTCG |
| | | AGCCCCACGTTGGGCG |
| 336 | Tyr_GTA_chr6: 26569085- | TCCTTCGATAGCTCAGTTGGTAGAGCGGA |
| | 26569176 (+) | GGACTGTAGTTGGCTGTGTCCTTAGACAT |
| | | CCTTAGGTCGCTGGTTCGAATCCGGCTCG |
| | | AAGGA |
| 337 | Ala_AGC_chr6: 26572091- | GGGGAATTAGCTCAAATGGTAGAGCGCTC |
| | 26572164 (−) | GCTTAGCATGCGAGAGGTAGCGGGATCG |
| | | ATGCCCGCATTCTCCAG |
| 338 | Met_CAT_chr6: 26766443- | CGCCCTCTTAGCGCAGCGGGCAGCGCGTC |
| | 26766516 (+) | AGTCTCATAATCTGAAGGTCCTGAGTTCG |
| | | AGCCTCAGAGAGGGCA |
| 339 | Ile_TAT_chr6: 26988124- | TGCTCCAGTGGCGCAATCGGTTAGCGCGC |
| | 26988218 (+) | GGTACTTATATGGCAGTATGTGTGCGAGT |
| | | GATGCCGAGGTTGTGAGTTCGAGCCTCAC |
| | | CTGGAGCA |
| 340 | His_GTG_chr6: 27125905- | TGCCGTGATCGTATAGTGGTTAGTACTCT |
| | 27125977 (+) | GCGTTGTGGCCGCAGCAACCTCGGTTCGA |
| | | ATCCGAGTCACGGCA |
| 341 | Ile_AAT_chr6: 27144993- | GGCCGGTTAGCTCAGTTGGTTAGAGCGTG |
| | 27145067 (−) | GTGCTAATAACGCCAAGGTCGCGGGTTCG |
| | | ATCCCCGTACGGGCCAC |
| 342 | Val_AAC_chr6: 27203287- | AGTTTCCGTAGTGTAGTGGTTATCACGTTT |
| | 27203360 (+) | GCCTAACACGCGAAAGGTCCCCGGTTCGA |
| | | AACCGGGCAGAAACA |
| 343 | Val_CAC_chr6: 27248048- | GCTTCTGTAGTGTAGTGGTTATCACGTTCG |
| | 27248121 (−) | CCTCACACGCGAAAGGTCCCCGGTTCGAA |
| | | ACCGGGCAGAAGCAA |
| 344 | Asp_GTC_chr6: 27447452- | TTCCTCGTTAGTATAGTGGTGAGTATCCCC |
| | 27447524 (+) | GCCTGTCACGCGGGAGACCGGGGTTCGAT |
| | | TCCCCGACGGGGAG |
| 345 | Ser_TGA_chr6: 27473606- | GTAGTCGTGGCCGAGTGGTTAAGGCGATG |
| | 27473688 (−) | GACTTGAAATCCATTGGGGTTTCCCCGCG |
| | | CAGGTTCGAATCCTGTCGGCTACGG |
| 346 | Gln_CTG_chr6: 27487307- | AGGTTCCATGGTGTAATGGTTAGCACTCT |
| | 27487379 (+) | GGACTCTGAATCCAGCGATCCGAGTTCAA |
| | | ATCTCGGTGGAACCT |
| 347 | Asp_GTC_chr6: 27551235- | TCCTCGTTAGTATAGTGGTGAGTGTCCCC |
| | 27551307 (−) | GTCTGTCACGCGGGAGACCGGGGTTCGAT |
| | | TCCCCGACGGGGAGA |
| 348 | Val_AAC_chr6: 27618706- | GTTTCCGTAGTGTAGTGGTTATCACGTTCG |
| | 27618779 (−) | CCTAACACGCGAAAGGTCCCTGGATCAAA |
| | | ACCAGGCGGAAACAA |
| 349 | Ile_AAT_chr6: 27655966- | CGGCCGGTTAGCTCAGTTGGTTAGAGCGT |
| | 27656040 (+) | GGTGCTAATAACGCCAAGGTCGCGGGTTC |
| | | GATCCCCGTACTGGCCA |
| 350 | Gln_CTG_chr6: 27759134- | GGCCCCATGGTGTAATGGTCAGCACTCTG |
| | 27759206 (−) | GACTCTGAATCCAGCGATCCGAGTTCAAA |
| | | TCTCGGTGGGACCCA |
| 351 | Gln_TTG_chr6: 27763639- | GGCCCCATGGTGTAATGGTTAGCACTCTG |
| | 27763711 (−) | GACTTTGAATCCAGCGATCCGAGTTCAAA |
| | | TCTCGGTGGGACCTT |
| 352 | Ala_AGC_chr6: 28574932- | TGGGGGTGTAGCTCAGTGGTAGAGCGCGT |
| | 28575004 (+) | GCTTAGCATGTACGAGGTCCCGGGTTCAA |
| | | TCCCCGGCACCTCCA |
| 353 | Ala_AGC_chr6: 28626013- | GGGGATGTAGCTCAGTGGTAGAGCGCATG |
| | 28626085 (−) | CTTAGCATGCATGAGGTCCCGGGTTCGAT |
| | | CCCCAGCATCTCCAG |
| 354 | Ala_CGC_chr6: 28697091- | AGGGGGTGTAGCTCAGTGGTAGAGCGCGT |
| | 28697163 (+) | GCTTCGCATGTACGAGGCCCCGGGTTCGA |
| | | CCCCCGGCTCCTCCA |
| 355 | Ala_AGC_chr6: 28806220- | GGGGGTGTAGCTCAGTGGTAGAGCGCGTG |
| | 28806292 (−) | CTTAGCATGCACGAGGCCCCGGGTTCAAT |
| | | CCCCGGCACCTCCAT |
| 356 | Ala_AGC_chr6: 28831461- | GGGGGTGTAGCTCAGTGGTAGAGCGCGTG |
| | 28831533 (−) | CTTAGCATGCACGAGGCCCCGGGTTCAAT |
| | | CCCCGGCACCTCCAG |
| 357 | Leu_CAA_chr6: 28863999- | GTCAGGATGGCCGAGTGGTCTAAGGCGCC |
| | 28864105 (−) | AGACTCAAGCTAAGCTTCCTCCGCGGTGG |
| | | GGATTCTGGTCTCCAATGGAGGCGTGGGT |
| | | TCGAATCCCACTTCTGACAC |
| 358 | Leu_CAA_chr6: 28908829- | TGTCAGGATGGCCGAGTGGTCTAAGGCGC |
| | 28908934 (+) | CAGACTCAAGCTTGGCTTCCTCGTGTTGA |
| | | GGATTCTGGTCTCCAATGGAGGCGTGGGT |
| | | TCGAATCCCACTTCTGACA |
| 359 | Gln_CTG_chr6: 28909377- | GGTTCCATGGTGTAATGGTTAGCACTCTG |
| | 28909449 (−) | GACTCTGAATCCAGCGATCCGAGTTCAAA |
| | | TCTCGGTGGAACCTT |
| 360 | Leu_AAG_chr6: 28911398- | GGTAGCGTGGCCGAGCGGTCTAAGGCGCT |
| | 28911480 (−) | GGATTAAGGCTCCAGTCTCTTCGGGGGCG |
| | | TGGGTTCGAATCCCACCGCTGCCAG |
| 361 | Met_CAT_chr6: 28912351- | TGCCTCCTTAGCGCAGTAGGCAGCGCGTC |
| | 28912424 (+) | AGTCTCATAATCTGAAGGTCCTGAGTTCG |
| | | AACCTCAGAGGGGGCA |
| 362 | Lys_TTT_chr6: 28918805- | AGCCCGGATAGCTCAGTCGGTAGAGCATC |
| | 28918878 (+) | AGACTTTTAATCTGAGGGTCCAGGGTTCA |
| | | AGTCCCTGTTCGGGCG |
| 363 | Met_CAT_chr6: 28921041- | GCCTCCTTAGCGCAGTAGGCAGCGCGTCA |
| | 28921114 (−) | GTCTCATAATCTGAAGGTCCTGAGTTCGA |
| | | ACCTCAGAGGGGGCAG |
| 364 | Glu_CTC_chr6: 28949975- | TTCCCTGGTGGTCTAGTGGTTAGGATTCG |
| | 28950047 (+) | GCGCTCTCACCGCCGCGGCCCGGGTTCGA |
| | | TTCCCGGTCAGGGAA |
| 365 | Leu_TAA_chr6: 144537683- | CACCAGGATGGCCGAGTGGTTAAGGCGTT |
| | 144537766 (+) | GGACTTAAGATCCAATGGACATATGTCCG |
| | | CGTGGGTTCGAACCCCACTCCTGGTA |
| 366 | Pro_AGG_chr7: 128423503- | TGGCTCGTTGGTCTAGGGGTATGATTCTC |
| | 128423575 (+) | GCTTAGGGTGCGAGAGGTCCCGGGTTCAA |
| | | ATCCCGGACGAGCCC |
| 367 | Arg_CCT_chr7: 139025445- | AGCCCCAGTGGCCTAATGGATAAGGCATT |
| | 139025518 (+) | GGCCTCCTAAGCCAGGGATTGTGGGTTCG |
| | | AGTCCCATCTGGGGTG |
| 368 | Cys_GCA_chr7: 149388271- | GGGGATATAGCTCAGGGGTAGAGCATTTG |
| | 149388343 (−) | ACTGCAGATCAAGAGGTCCCCGGTTCAAA |
| | | TCCGGGTGCCCCCCC |
| 369 | Tyr_GTA_chr8: 67025601- | CCCTTCGATAGCTCAGCTGGTAGAGCGGA |
| | 67025694 (+) | GGACTGTAGCTACTTCCTCAGCAGGAGAC |
| | | ATCCTTAGGTCGCTGGTTCGATTCCGGCTC |
| | | GAAGGA |
| 370 | Tyr_GTA_chr8: 67026222- | CCCTTCGATAGCTCAGCTGGTAGAGCGGA |
| | 67026311 (+) | GGACTGTAGGCGCGCGCCCGTGGCCATCC |
| | | TTAGGTCGCTGGTTCGATTCCGGCTCGAA |
| | | GGA |
| 371 | Ala_AGC_chr8: 67026423- | TGGGGGATTAGCTCAAATGGTAGAGCGCT |
| | 67026496 (+) | CGCTTAGCATGCGAGAGGTAGCGGGATCG |
| | | ATGCCCGCATCCTCCA |
| 372 | Ser_AGA_chr8: 96281884- | GTAGTCGTGGCCGAGTGGTTAAGGCGATG |
| | 96281966 (−) | GACTAGAAATCCATTGGGGTCTCCCCGCG |
| | | CAGGTTCGAATCCTGCCGACTACGG |
| 373 | Met_CAT_chr8: 124169469- | GCCTCGTTAGCGCAGTAGGTAGCGCGTCA |
| | 124169542 (−) | GTCTCATAATCTGAAGGTCGTGAGTTCGA |
| | | TCCTCACACGGGGCAC |
| 374 | Arg_TCT_chr9: 131102354- | GGCTCTGTGGCGCAATGGATAGCGCATTG |
| | 131102445 (−) | GACTTCTAGCTGAGCCTAGTGTGGTCATT |
| | | CAAAGGTTGTGGGTTCGAGTCCCACCAGA |
| | | GTCGA |
| 375 | Asn_GTT_chr10: 22518437- | GTCTCTGTGGCGCAATCGGTTAGCGCGTT |
| | 22518511 (−) | CGGCTGTTAACCGAAAGGTTGGTGGTTCG |
| | | AGCCCACCCAGGGACGC |
| 376 | Ser_TGA_chr10: 69524260- | GGCAGCGATGGCCGAGTGGTTAAGGCGTT |
| | 69524342 (+) | GGACTTGAAATCCAATGGGGTCTCCCCGC |
| | | GCAGGTTCGAACCCTGCTCGCTGCG |
| 377 | Val_TAC_chr11: 59318101- | GGTTCCATAGTGTAGTGGTTATCACGTCT |
| | 59318174 (−) | GCTTTACACGCAGAAGGTCCTGGGTTCGA |
| | | GCCCCAGTGGAACCAT |
| 378 | Val_TAC_chr11: 59318459- | GGTTCCATAGTGTAGCGGTTATCACGTCT |
| | 59318532 (−) | GCTTTACACGCAGAAGGTCCTGGGTTCGA |
| | | GCCCCAGTGGAACCAC |
| 379 | Arg_TCT_chr11: 59318766- | TGGCTCTGTGGCGCAATGGATAGCGCATT |
| | 59318852 (+) | GGACTTCTAGATAGTTAGAGAAATTCAAA |
| | | GGTTGTGGGTTCGAGTCCCACCAGAGTCG |
| 380 | Leu_TAA_chr11: 59319227- | TACCAGAATGGCCGAGTGGTTAAGGCGTT |
| | 59319310 (+) | GGACTTAAGATCCAATGGATTCATATCCG |
| | | CGTGGGTTCGAACCCCACTTCTGGTA |
| 381 | Lys_TTT_chr11: 59323901- | GGCCCGGATAGCTCAGTCGGTAGAGCATC |
| | 59323974 (+) | AGACTTTTAATCTGAGGGTCCGGGGTTCA |
| | | AGTCCCTGTTCGGGCG |
| 382 | Phe_GAA_chr11: 59324969- | GCCGAAATAGCTCAGTTGGGAGAGCGTTA |
| | 59325042 (−) | GACTGAAGATCTAAAGGTCCCTGGTTCGA |
| | | TCCCGGGTTTCGGCAG |
| 383 | Lys_TTT_chr11: 59327807- | GCCCGGATAGCTCAGTCGGTAGAGCATCA |
| | 59327880 (−) | GACTTTTAATCTGAGGGTCCAGGGTTCAA |
| | | GTCCCTGTTCGGGCGG |
| 384 | Phe_GAA_chr11: 59333852- | GCCGAAATAGCTCAGTTGGGAGAGCGTTA |
| | 59333925 (−) | GACTGAAGATCTAAAGGTCCCTGGTTCAA |
| | | TCCCGGGTTTCGGCAG |
| 385 | Ser_GCT_chr11: 66115590- | GGACGAGGTGGCCGAGTGGTTAAGGCGA |
| | 66115672 (+) | TGGACTGCTAATCCATTGTGCTTTGCACG |
| | | CGTGGGTTCGAATCCCATCCTCGTCG |
| 386 | Pro_TGG_chr11: 75946868- | GGCTCGTTGGTCTAGGGGTATGATTCTCG |
| | 75946940 (−) | GTTTGGGTCCGAGAGGTCCCGGGTTCAAA |
| | | TCCCGGACGAGCCCC |
| 387 | Ser_CGA_chr12: 56584147- | AGTCACGGTGGCCGAGTGGTTAAGGCGTT |
| | 56584229 (+) | GGACTCGAAATCCAATGGGGTTTCCCCGC |
| | | ACAGGTTCGAATCCTGTTCGTGACG |
| 388 | Asp_GTC_chr12: 98897280- | CTCCTCGTTAGTATAGTGGTTAGTATCCCC |
| | 98897352 (+) | GCCTGTCACGCGGGAGACCGGGGTTCAAT |
| | | TCCCCGACGGGGAG |
| 389 | Trp_CCA_chr12: 98898029- | GGACCTCGTGGCGCAACGGTAGCGCGTCT |
| | 98898101 (+) | GACTCCAGATCAGAAGGCTGCGTGTTCGA |
| | | ATCACGTCGGGGTCA |
| 390 | Ala_TGC_chr12: 125406300- | GGGGATGTAGCTCAGTGGTAGAGCGCATG |
| | 125406372 (−) | CTTTGCATGTATGAGGCCCCGGGTTCGAT |
| | | CCCCGGCATCTCCAT |
| 391 | Phe_GAA_chr12: 125412388- | GCCGAAATAGCTCAGTTGGGAGAGCGTTA |
| | 125412461 (−) | GACTGAAGATCTAAAGGTCCCTGGTTCGA |
| | | TCCCGGGTTTCGGCAC |
| 392 | Ala_TGC_chr12: 125424511- | AGGGGATGTAGCTCAGTGGTAGAGCGCAT |
| | 125424583 (+) | GCTTTGCACGTATGAGGCCCCGGGTTCAA |
| | | TCCCCGGCATCTCCA |
| 393 | Asn_GTT_chr13: 31248100- | GTCTCTGTGGCGCAATCGGTTAGCGCGTT |
| | 31248174 (−) | CGGCTGTTAACCGAAAGGTTGGTGGTTCG |
| | | AGCCCACCCAGGGACGG |
| 394 | Glu_TTC_chr13: 45492061- | TCCCACATGGTCTAGCGGTTAGGATTCCT |
| | 45492133 (−) | GGTTTTCACCCAGGCGGCCCGGGTTCGAC |
| | | TCCCGGTGTGGGAAC |
| 395 | Thr_TGT_chr14: 21081948- | GGCTCCATAGCTCAGGGGTTAGAGCGCTG |
| | 21082021 (−) | GTCTTGTAAACCAGGGGTCGCGAGTTCAA |
| | | TTCTCGCTGGGGCCTG |
| 396 | Leu_TAG_chr14: 21093528- | TGGTAGTGTGGCCGAGCGGTCTAAGGCGC |
| | 21093610 (+) | TGGATTTAGGCTCCAGTCTCTTCGGGGGC |
| | | GTGGGTTCGAATCCCACCACTGCCA |
| 397 | Thr_TGT_chr14: 21099318- | GGCTCCATAGCTCAGGGGTTAGAGCACTG |
| | 21099391 (−) | GTCTTGTAAACCAGGGGTCGCGAGTTCAA |
| | | ATCTCGCTGGGGCCTC |
| 398 | Pro_TGG_chr14: 21101164- | TGGCTCGTTGGTCTAGTGGTATGATTCTCG |
| | 21101236 (+) | CTTTGGGTGCGAGAGGTCCCGGGTTCAAA |
| | | TCCCGGACGAGCCC |
| 399 | Tyr_GTA_chr14: 21131350- | CCTTCGATAGCTCAGCTGGTAGAGCGGAG |
| | 21131444 (−) | GACTGTAGATTGTACAGACATTTGCGGAC |
| | | ATCCTTAGGTCGCTGGTTCGATTCCGGCTC |
| | | GAAGGAA |
| 400 | Thr_TGT_chr14: 21149848- | AGGCCCTATAGCTCAGGGGTTAGAGCACT |
| | 21149921 (+) | GGTCTTGTAAACCAGGGGTCGCGAGTTCA |
| | | AATCTCGCTGGGGCCT |
| 401 | Tyr_GTA_chr14: 21151431- | TCCTTCGATAGCTCAGCTGGTAGAGCGGA |
| | 21151520 (+) | GGACTGTAGTACTTAATGTGTGGTCATCC |
| | | TTAGGTCGCTGGTTCGATTCCGGCTCGAA |
| | | GGA |
| 402 | Pro_TGG_chr14: 21152174- | TGGCTCGTTGGTCTAGGGGTATGATTCTC |
| | 21152246 (+) | GCTTTGGGTGCGAGAGGTCCCGGGTTCAA |
| | | ATCCCGGACGAGCCC |
| 403 | Lys_CTT_chr14: 58706612- | GCCCGGCTAGCTCAGTCGGTAGAGCATGG |
| | 58706685 (−) | GACTCTTAATCCCAGGGTCGTGGGTTCGA |
| | | GCCCCACGTTGGGCGC |
| 404 | Ile_AAT_chr14: 102783428- | CGGCCGGTTAGCTCAGTTGGTTAGAGCGT |
| | 102783502 (+) | GGTGCTAATAACGCCAAGGTCGCGGGTTC |
| | | GATCCCCGTACGGGCCA |
| 405 | Glu_TTC_chr15: 26327380- | TCCCACATGGTCTAGCGGTTAGGATTCCT |
| | 26327452 (−) | GGTTTTCACCCAGGCGGCCCGGGTTCGAC |
| | | TCCCGGTGTGGGAAT |
| 406 | Ser_GCT_chr15: 40886022- | GACGAGGTGGCCGAGTGGTTAAGGCGAT |
| | 40886104 (−) | GGACTGCTAATCCATTGTGCTCTGCACGC |
| | | GTGGGTTCGAATCCCATCCTCGTCGA |
| 407 | His_GTG_chr15: 45490803- | GCCGTGATCGTATAGTGGTTAGTACTCTG |
| | 45490875 (−) | CGTTGTGGCCGCAGCAACCTCGGTTCGAA |
| | | TCCGAGTCACGGCAT |
| 408 | His_GTG_chr15: 45493348- | CGCCGTGATCGTATAGTGGTTAGTACTCT |
| | 45493420 (+) | GCGTTGTGGCCGCAGCAACCTCGGTTCGA |
| | | ATCCGAGTCACGGCA |
| 409 | Gln_CTG_chr15: 66161399- | GGTTCCATGGTGTAATGGTTAGCACTCTG |
| | 66161471 (−) | GACTCTGAATCCAGCGATCCGAGTTCAAA |
| | | TCTCGGTGGAACCTG |
| 410 | Lys_CTT_chr15: 79152903- | TGCCCGGCTAGCTCAGTCGGTAGAGCATG |
| | 79152976 (+) | GGACTCTTAATCCCAGGGTCGTGGGTTCG |
| | | AGCCCCACGTTGGGCG |
| 411 | Arg_TCG_chr15: 89878303- | GGGCCGCGTGGCCTAATGGATAAGGCGTC |
| | 89878376 (+) | TGACTTCGGATCAGAAGATTGCAGGTTCG |
| | | AGTCCTGCCGCGGTCG |
| 412 | Gly_CCC_chr16: 686735- | GCGCCGCTGGTGTAGTGGTATCATGCAAG |
| | 686806 (−) | ATTCCCATTCTTGCGACCCGGGTTCGATTC |
| | | CCGGGCGGCGCAC |
| 413 | Arg_CCG_chr16: 3200674- | GGGCCGCGTGGCCTAATGGATAAGGCGTC |
| | 3200747 (+) | TGATTCCGGATCAGAAGATTGAGGGTTCG |
| | | AGTCCCTTCGTGGTCG |
| 414 | Arg_CCT_chr16: 3202900- | CGCCCCGGTGGCCTAATGGATAAGGCATT |
| | 3202973 (+) | GGCCTCCTAAGCCAGGGATTGTGGGTTCG |
| | | AGTCCCACCCGGGGTA |
| 415 | Lys_CTT_chr16: 3207405- | GCCCGGCTAGCTCAGTCGGTAGAGCATGA |
| | 3207478 (−) | GACCCTTAATCTCAGGGTCGTGGGTTCGA |
| | | GCCCCACGTTGGGCGT |
| 416 | Thr_CGT_chr16: 14379749- | AGGCGCGGTGGCCAAGTGGTAAGGCGTC |
| | 14379821 (+) | GGTCTCGTAAACCGAAGATCACGGGTTCG |
| | | AACCCCGTCCGTGCCT |
| 417 | Leu_TAG_chr16: 22207031- | GGTAGCGTGGCCGAGTGGTCTAAGGCGCT |
| | 22207113 (−) | GGATTTAGGCTCCAGTCATTTCGATGGCG |
| | | TGGGTTCGAATCCCACCGCTGCCAC |
| 418 | Leu_AAG_chr16: 22308460- | GGGTAGCGTGGCCGAGCGGTCTAAGGCG |
| | 22308542 (+) | CTGGATTAAGGCTCCAGTCTCTTCGGGGG |
| | | CGTGGGTTCGAATCCCACCGCTGCCA |
| 419 | Leu_CAG_chr16: 57333862- | AGTCAGGATGGCCGAGCGGTCTAAGGCG |
| | 57333945 (+) | CTGCGTTCAGGTCGCAGTCTCCCCTGGAG |
| | | GCGTGGGTTCGAATCCCACTTCTGACA |
| 420 | Leu_CAG_chr16: 57334391- | GTCAGGATGGCCGAGCGGTCTAAGGCGCT |
| | 57334474 (−) | GCGTTCAGGTCGCAGTCTCCCCTGGAGGC |
| | | GTGGGTTCGAATCCCACTTCTGACAG |
| 421 | Met_CAT_chr16: 87417627- | GCCTCGTTAGCGCAGTAGGCAGCGCGTCA |
| | 87417700 (−) | GTCTCATAATCTGAAGGTCGTGAGTTCGA |
| | | GCCTCACACGGGGCAG |
| 422 | Leu_TAG_chr17: 8023631- | GGTAGCGTGGCCGAGCGGTCTAAGGCGCT |
| | 8023713 (−) | GGATTTAGGCTCCAGTCTCTTCGGAGGCG |
| | | TGGGTTCGAATCCCACCGCTGCCAG |
| 423 | Arg_TCT_chr17: 8024242- | TGGCTCTGTGGCGCAATGGATAGCGCATT |
| | 8024330 (+) | GGACTTCTAGTGACGAATAGAGCAATTCA |
| | | AAGGTTGTGGGTTCGAATCCCACCAGAGT |
| | | CG |
| 424 | Gly_GCC_chr17: 8029063- | CGCATTGGTGGTTCAGTGGTAGAATTCTC |
| | 8029134 (+) | GCCTGCCACGCGGGAGGCCCGGGTTCGAT |
| | | TCCCGGCCAATGCA |
| 425 | Ser_CGA_chr17: 8042198- | GCTGTGATGGCCGAGTGGTTAAGGCGTTG |
| | 8042280 (−) | GACTCGAAATCCAATGGGGTCTCCCCGCG |
| | | CAGGTTCGAATCCTGCTCACAGCGT |
| 426 | Thr_AGT_chr17: 8042769- | GGCGCCGTGGCTTAGCTGGTTAAAGCGCC |
| | 8042843 (−) | TGTCTAGTAAACAGGAGATCCTGGGTTCG |
| | | AATCCCAGCGGTGCCTG |
| 427 | Trp_CCA_chr17: 8089675- | CGACCTCGTGGCGCAACGGTAGCGCGTCT |
| | 8089747 (+) | GACTCCAGATCAGAAGGTTGCGTGTTCAA |
| | | ATCACGTCGGGGTCA |
| 428 | Ser_GCT_chr17: 8090183- | AGACGAGGTGGCCGAGTGGTTAAGGCGA |
| | 8090265 (+) | TGGACTGCTAATCCATTGTGCTCTGCACG |
| | | CGTGGGTTCGAATCCCATCCTCGTCG |
| 429 | Thr_AGT_chr17: 8090477- | CGGCGCCGTGGCTTAGTTGGTTAAAGCGC |
| | 8090551 (+) | CTGTCTAGTAAACAGGAGATCCTGGGTTC |
| | | GAATCCCAGCGGTGCCT |
| 430 | Trp_CCA_chr17: 8124186- | GGCCTCGTGGCGCAACGGTAGCGCGTCTG |
| | 8124258 (−) | ACTCCAGATCAGAAGGTTGCGTGTTCAAA |
| | | TCACGTCGGGGTCAA |
| 431 | Gly_TCC_chr17: 8124865- | AGCGTTGGTGGTATAGTGGTAAGCATAGC |
| | 8124937 (+) | TGCCTTCCAAGCAGTTGACCCGGGTTCGA |
| | | TTCCCGGCCAACGCA |
| 432 | Asp_GTC_chr17: 8125555- | TCCTCGTTAGTATAGTGGTGAGTATCCCC |
| | 8125627 (−) | GCCTGTCACGCGGGAGACCGGGGTTCGAT |
| | | TCCCCGACGGGGAGA |
| 433 | Pro_CGG_chr17: 8126150- | GGCTCGTTGGTCTAGGGGTATGATTCTCG |
| | 8126222 (−) | CTTCGGGTGCGAGAGGTCCCGGGTTCAAA |
| | | TCCCGGACGAGCCCT |
| 434 | Thr_AGT_chr17: 8129552- | GGCGCCGTGGCTTAGTTGGTTAAAGCGCC |
| | 8129626 (−) | TGTCTAGTAAACAGGAGATCCTGGGTTCG |
| | | AATCCCAGCGGTGCCTT |
| 435 | Ser_AGA_chr17: 8129927- | GTAGTCGTGGCCGAGTGGTTAAGGCGATG |
| | 8130009 (−) | GACTAGAAATCCATTGGGGTCTCCCCGCG |
| | | CAGGTTCGAATCCTGCCGACTACGT |
| 436 | Trp_CCA_chr17: 19411493- | TGACCTCGTGGCGCAATGGTAGCGCGTCT |
| | 19411565 (+) | GACTCCAGATCAGAAGGTTGCGTGTTCAA |
| | | GTCACGTCGGGGTCA |
| 437 | Thr_CGT_chr17: 29877092- | AGGCGCGGTGGCCAAGTGGTAAGGCGTC |
| | 29877164 (+) | GGTCTCGTAAACCGAAGATCGCGGGTTCG |
| | | AACCCCGTCCGTGCCT |
| 438 | Cys_GCA_chr17: 37023897- | AGGGGGTATAGCTCAGTGGTAGAGCATTT |
| | 37023969 (+) | GACTGCAGATCAAGAGGTCCCCGGTTCAA |
| | | ATCCGGGTGCCCCCT |
| 439 | Cys_GCA_chr17: 37025544- | GGGGGTATAGCTCAGTGGTAGAGCATTTG |
| | 37025616 (−) | ACTGCAGATCAAGAGGTCCCTGGTTCAAA |
| | | TCCGGGTGCCCCCTC |
| 440 | Cys_GCA_chr17: 37309986- | GGGGGTATAGCTCAGTGGTAGAGCATTTG |
| | 37310058 (−) | ACTGCAGATCAAGAGGTCCCCGGTTCAAA |
| | | TCCGGGTGCCCCCTC |
| 441 | Gln_TTG_chr17: 47269889- | AGGTCCCATGGTGTAATGGTTAGCACTCT |
| | 47269961 (+) | GGACTTTGAATCCAGCGATCCGAGTTCAA |
| | | ATCTCGGTGGGACCT |
| 442 | Arg_CCG_chr17: 66016012- | GACCCAGTGGCCTAATGGATAAGGCATCA |
| | 66016085 (−) | GCCTCCGGAGCTGGGGATTGTGGGTTCGA |
| | | GTCCCATCTGGGTCGC |
| 443 | Arg_CCT_chr17: 73030000- | AGCCCCAGTGGCCTAATGGATAAGGCACT |
| | 73030073 (+) | GGCCTCCTAAGCCAGGGATTGTGGGTTCG |
| | | AGTCCCACCTGGGGTA |
| 444 | Arg_CCT_chr17: 73030525- | GCCCCAGTGGCCTAATGGATAAGGCACTG |
| | 73030598 (−) | GCCTCCTAAGCCAGGGATTGTGGGTTCGA |
| | | GTCCCACCTGGGGTGT |
| 445 | Arg_TCG_chr17: 73031207- | AGACCGCGTGGCCTAATGGATAAGGCGTC |
| | 73031280 (+) | TGACTTCGGATCAGAAGATTGAGGGTTCG |
| | | AGTCCCTTCGTGGTCG |
| 446 | Asn_GTT_chr19: 1383561- | CGTCTCTGTGGCGCAATCGGTTAGCGCGT |
| | 1383635 (+) | TCGGCTGTTAACCGAAAGGTTGGTGGTTC |
| | | GAGCCCACCCAGGGACG |
| 447 | Gly_TCC_chr19: 4724081- | GGCGTTGGTGGTATAGTGGTTAGCATAGC |
| | 4724153 (+) | TGCCTTCCAAGCAGTTGACCCGGGTTCGA |
| | | TTCCCGGCCAACGCA |
| 448 | Val_CAC_chr19: 4724646- | GTTTCCGTAGTGTAGCGGTTATCACATTC |
| | 4724719 (−) | GCCTCACACGCGAAAGGTCCCCGGTTCGA |
| | | TCCCGGGCGGAAACAG |
| 449 | Thr_AGT_chr19: 33667962- | TGGCGCCGTGGCTTAGTTGGTTAAAGCGC |
| | 33668036 (+) | CTGTCTAGTAAACAGGAGATCCTGGGTTC |
| | | GAATCCCAGCGGTGCCT |
| 450 | Ile_TAT_chr19: 39902807- | GCTCCAGTGGCGCAATCGGTTAGCGCGCG |
| | 39902900 (−) | GTACTTATATGACAGTGCGAGCGGAGCAA |
| | | TGCCGAGGTTGTGAGTTCGATCCTCACCT |
| | | GGAGCAC |
| 451 | Gly_GCC_chr21: 18827106- | GCATGGGTGGTTCAGTGGTAGAATTCTCG |
| | 18827177 (−) | CCTGCCACGCGGGAGGCCCGGGTTCGATT |
| | | CCCGGCCCATGCAG |

### Asialoglycoprotein Receptor Binding Moieties

The present disclosure features a TREM comprising an asialoglycoprotein receptor (ASGPR) binding moiety. The ASGPR binding moiety may be bound to any nucleotide within the TREM, as well as to the 5′ or 3′ termini. In an embodiment, the ASGPR binding moiety is bound (e.g., directly bound) to a nucleotide, for example, to a sugar moiety, a nucleobase, and/or the internucleotide region. In an embodiment, the ASGPR binding moiety is conjugated to a sugar moiety and/or the internucleotide region within the TREM. In an embodiment, the ASGPR binding moiety is bound to the pos or 4′ positions on the sugar moiety (e.g., ribose moiety) within a nucleotide of the TREM. In an embodiment, the ASGPR binding moiety is bound to the phosphate linker between nucleotides within the TREM.

The ASGPR is a C-type lectin primarily expressed on the sinusoidal surface of hepatocytes, and comprises a major (48 kDa, ASGPR-1) and a minor (40 kDa, ASGPR-2) subunit. The ASGPR is involved in the binding, internalization, and subsequent clearance of glycoproteins containing an N-terminal galactose (Gal) or N-terminal N-acetylgalactosamine (GalNAc) residues from circulation, such as antibodies. ASGPRs have also been shown to be involved in the clearance of low density lipoprotein, fibronectin, and certain immune cells, and may be utilized by certain viruses for hepatocyte entry (see, e.g., Yang J., et al (2006) J Viral Hepat 13:158-165 and Guy, C S et al (2011) Nat Rev Immunol 8:874-887).

The ASGPR binding moiety as described herein may refer to structure comprising: (i) a ASGPR carbohydrate and (ii) an ASGPR linker (e.g., a linker connecting the carbohydrate to the TREM). The term “carbohydrate” as used herein refers to compound comprising one or more monosaccharide moieties comprising at least 3 carbon atoms (e.g., arranged in a linear, branched, or cyclic structure) and an oxygen, nitrogen, or sulfur atom, or a fragment or variant of a monosaccharide moiety comprising at least 3 carbon atoms (e.g., arranged in a linear, branched, or cyclic structure) and an oxygen, nitrogen, or sulfur atom. Each monosaccharide moiety or fragment or variant thereof may be a tetrose, pentose, hexose, or heptose. Each monosaccharide moiety or fragment or variant thereof may exist as an aldose, ketose, sugar alcohol, and, where appropriate, in the L or D form. Exemplary monosaccharide moieties may be amino sugars, N-acetylamino sugars, imino sugars, deoxysugars, or sugar acids. Carbohydrates may comprise individual monosaccharide moieties, or may further comprise a disaccharide, oligosaccharide (e.g., a trisaccharide, tetrasaccharide, pentasaccharide, hexasaccharide, heptasaccharide, octasaccharide), a polysaccharide, or combinations thereof. Exemplary carbohydrates include ribose, arabinose, lyxose, xylose, deoxyribose, ribulose, xylulose, glucose, galactose, mannose, gulose, idose, talose, allose, altrose, psicose, fructose, sorbose, tagatose, rhamnose, pneumose, quinovose, fucose, mannuheptulose, sedoheptulose, galactosamine, mannosamine, glucosamine, N-acetylglucosamine, N-acetylgalactosamine, N-acetylmannosamine, glucuronic acid, galacturonic acid, mannuronic acid, guluronic acid, iduronic acid, tagaturonic acid, frucuronic acid, galactosaminuronic acid, mannosaminuronic acid, glucosaminuronic acid, N-acctylglucosaminuronic acid, N-acetylgalactosaminuronic acid, N-acetylmannosaminuronic acid, maltose, lactose, sucrose, trehalose, gentiobiose, cellobiose, chitobiose, kojibiose, nigerose, sophorose, trehalulose, isomaltose, xylobiose, starch, cellulose, chitin, and dextran.

The carbohydrate may comprise one or more monosaccharide moieties linked by a glycosidic bond. In some embodiments, the glycosidic bond comprises a 1->2 glycosidic bond, a 1->3 glycosidic bond, a 1->4 glycosidic bond, or a 1->6 glycosidic bond. In some embodiments, each glycosidic bond may be present in the alpha or beta configuration. In an embodiment, the one or more monosaccharide moieties are linked directly by a glycosidic bond or are separated by a linker.

In some embodiments, the ASGPR binding moiety comprises a galactose (Gal), galactosamine (GalNH₂), or an N-acetylgalactosamine (GalNAc) moiety, for example, a Gal, GalNH₂, or GalNAc, or an analog thereof. In an embodiment, the ASGPR binding moiety comprises a GalNAc moiety (e.g., GalNAc). In an embodiment, the ASGPR binding moiety comprises a plurality of GalNAc moieties (e.g., GalNAcs), e.g., at least 2, 3, 4, 5, 6, 7, 8, 9, 10, 11, 12, 13, 14, 15, 16, 17, 18, 19, 20, or more GalNAc moieties (e.g., GalNAcs). In an embodiment, the ASGPR binding moiety comprises between 2 and 20 GalNAcs moieties (e.g., 2, 3, 4, 5, 6, 7, 8, 9, 10, 11, 12, 13, 14, 15, 16, 17, 18, 19, or 20 GalNAc moieties). In an embodiment, the ASGPR binding moiety comprises between 2 and 10 GalNAc moieties (e.g., 2, 3, 4, 5, 6, 7, 8, 9, or 10 GalNAc moieties). In an embodiment, the ASGPR binding moiety comprises between 2 and 5 GalNAc moieties (e.g., 2, 3, 4, or 5 GalNAc moieties). In an embodiment, the ASGPR binding moiety comprises 2 GalNAc moieties. In an embodiment, the ASGPR binding moiety comprises 3 GalNAc moieties. In an embodiment, the ASGPR binding moiety comprises 4 GalNAc moieties. In an embodiment, the ASGPR moieties comprises 5 GalNAc moieties.

In some embodiments, the GalNAc moiety comprises a structure of Formula (I):

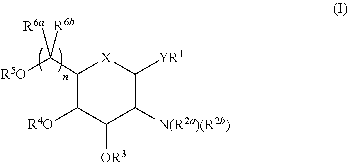

or a salt thereof, wherein each of X and Y is independently O, N(R⁷), or S; each of R¹, R³, R⁴, and R⁵ arc independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; or R³ and R⁴ are taken together with the oxygen atoms to which they are connected to form a heterocyclyl ring optionally substituted with one or more R⁸; R^{2a} is hydrogen or alkyl; R^{2b} is —C(O)alkyl (e.g., C(O)CH₃); each of R^{6a} and R^{6b} is hydrogen, alkyl, alkenyl, alkynyl, heteroalkyl, haloalkyl, halo, cyano, nitro, —OR^{A}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; RA is hydrogen, or alkyl, alkenyl, alkynyl, and n is an integer between 0 and 6, wherein the structure of Formula (I) may be connected to a linker or TREM at any position.

In some embodiments, X is O. In some embodiments, Y is O. In some embodiments, each of R¹, R³, R⁴, and R⁵ are independently hydrogen or alkyl (e.g., CH₃). In some embodiments, R^{2a} is hydrogen. In some embodiments, R^{2b} is C(O)CH₃. In some embodiments, each of R^{6a} and R^{6b} is hydrogen. In some embodiments, n is 0, 1, 2, or 3. In some embodiments, n is 1, 2, or 3. In some embodiments, n is 1. In some embodiments, the GalNAc moiety is connected to a linker or TREM at R^{2a}. In some embodiments, the GalNAc moiety is connected to a linker or TREM at R^{2b}. In some embodiments, the GalNAc moiety is connected to a linker or TREM at R³. In some embodiments, the GalNAc moiety is connected to a linker or TREM at R⁴. In some embodiments, the GalNAc moiety is connected to a linker or TREM at R⁵. In some embodiments, the GalNAc moiety is connected to a linker or TREM at R^{6a} or R^{6b}. In some embodiments, the GalNAc moiety is connected to a linker or TREM at a plurality of positions, e.g., at least two of R¹, R2a, R2b, R³, R⁴, R⁵, R^{6a}, and R^{6b}.

In some embodiments, the GalNAc moiety is comprises a structure of Formula (I-a)

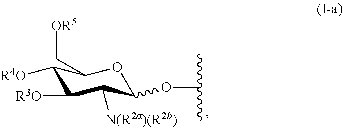

or a salt thereof, wherein R^{2a} is hydrogen or alkyl; R^{2b} is —C(O)alkyl (e.g., C(O)CH₃); each of R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; or R³ and R⁴ are taken together with the oxygen atoms to which they are connected to form a heterocyclyl ring optionally substituted with one or more R⁸; and R⁸ is hydrogen, halo, cyano, alkyl, alkenyl, alkynyl, heteroalkyl, haloalkyl, cycloalkyl, or heterocyclyl, wherein the “ [symbol] ” represents a bond in any configuration, and “ [symbol] ” represents an attachment point to a TREM, e.g., a linker, a nucleobase, internucleotide linkage, or terminus within the TREM sequence.

In some embodiments, each of R³, R⁴, and R⁵ are independently hydrogen or alkyl (e.g., CH₃). In some embodiments, R^{2a} is hydrogen. In some embodiments, R^{2b} is C(O)CH₃.

In some embodiments, the GalNAc moiety comprises a structure of Formula (II):

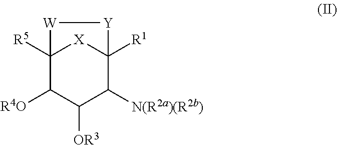

or a salt thereof, wherein X is O, N (R⁷), or S; each of W or Y is independently O or C (R^{10a})(R^{10b}), wherein one of W and Y is O; each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; or R³ and R⁴ are taken together with the oxygen atoms to which they are connected to form a heterocyclyl ring optionally substituted with one or more R⁸; R^{2a} is hydrogen or alkyl; R^{2b} is —C(O)alkyl (e.g., C(O)CH₃); each of R^{6a} and R^{6b} is hydrogen, alkyl, alkenyl, alkynyl, heteroalkyl, haloalkyl, halo, cyano, nitro, —OR^{A}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; each of R^{10a} and R^{10b} is independently hydrogen, heteroalkyl, haloalkyl, or halo; and R^{A} is hydrogen, or alkyl, alkenyl, alkynyl, wherein the structure of Formula (I) may be connected to a TREM, e.g., a linker, a nucleobase, internucleotide linkage, or terminus within the TREM sequence.

In some embodiments, the GalNAc moiety comprises a structure of Formula (II-a):

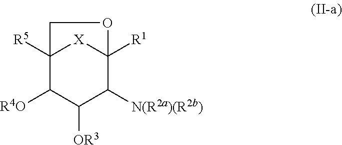

or a salt thereof, wherein X is O, N (R⁷), or S; each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; or R³ and R⁴ are taken together with the oxygen atoms to which they are connected to form a heterocyclyl ring optionally substituted with one or more R⁸; R^{2a} is hydrogen or alkyl; R^{2b} is —C(O)alkyl (e.g., C(O)CH₃); each of R^{6a} and R^{6b} is hydrogen, alkyl, alkenyl, alkynyl, heteroalkyl, haloalkyl, halo, cyano, nitro, —OR^{A}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; and RA is hydrogen, or alkyl, alkenyl, alkynyl, wherein the structure of Formula (I) may be connected to a TREM, e.g., a linker, a nucleobase, internucleotide linkage, or terminus within the TREM sequence.

In some embodiments, the GalNAc moiety comprises a structure of Formula (II-b):

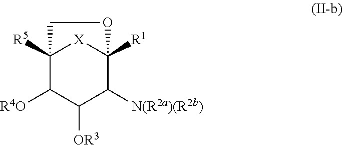

or a salt thereof, wherein X is O, N (R⁷), or S; each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; or R³ and R⁴ are taken together with the oxygen atoms to which they are connected to form a heterocyclyl ring optionally substituted with one or more R⁸; R^{2a} is hydrogen or alkyl; R^{2b} is —C(O)alkyl (e.g., C(O)CH₃); each of R^{6a} and R^{6b} is hydrogen, alkyl, alkenyl, alkynyl, heteroalkyl, haloalkyl, halo, cyano, nitro, —OR^{A}, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; and RA is hydrogen, or alkyl, alkenyl, alkynyl, wherein the structure of Formula (I) may be connected to a TREM, e.g., a linker, a nucleobase, internucleotide linkage, or terminus within the TREM sequence.

In some embodiments, the ASGPR binding moiety comprises a structure of Formula (III):

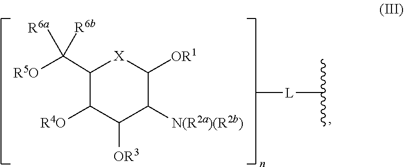

or a salt thereof, wherein each of R¹, R2a, R2b, R³, R⁴, R⁵, R^{6a}, and R^{6b} and subvariables thereof are as defined for Formula (I), L is a linker, and n is an integer between 1 and 100, wherein “ [symbol] ” represents an attachment point to a branching point, additional linker, or TREM, e.g., a linker, a nucleobase, internucleotide linkage, or terminus within the TREM sequence.

In some embodiments, X is O. In some embodiments, each of R¹, R³, R⁴, and R⁵ arc independently hydrogen or alkyl (e.g., CH₃). In some embodiments, R^{2a} is hydrogen. In some embodiments, R^{2b} is C(O)CH₃. In some embodiments, each of R^{6a} and R^{6b} is hydrogen. In some embodiments, n is an integer between 1 and 50. In some embodiments, n is an integer between 1 and 25. In some embodiments, n is an integer between 1 and 10. In some embodiments, n is an integer between 1 and 5. In some embodiments, n is 1, 2, 3, 4, or 5. In some embodiments, n is 1.

In an embodiment, L comprises an alkylene, alkenylene, alkynylene, heteroalkylene, or haloalkylene group. In an embodiment, L comprises an ester, amide, disulfide, ether, carbonate, aryl, heteroaryl, cycloalkyl, or heterocyclyl group. In an embodiment, L is cleavable or non-cleavable.

The term “linker” as used herein refers to an organic moiety that connects two or more parts of a compound, e.g., through a covalent bond. A linker may linear or branched. In some embodiments, a linker comprises a heteroatom, such as a nitrogen, sulfur, oxygen, phosphorus, silicon, or boron atom. In some embodiments, the linker comprises a cyclic group (e.g., an aryl, heteroaryl, cycloalkyl, or heterocyclyl group). In some embodiments, a linker comprises a functional group such as an amide, ketone, ester, ether, thioester, thioether, thiol, hydroxyl, amine, cyano, nitro, azide, triazole, pyrroline, p-nitrophenyl, alkene, or alkyne group. Any atom within a linker may be substituted or unsubstituted. In some embodiments, a linker comprises an arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, or alkynylhereroaryl group. In some embodiments, a linker comprises a polyethylene glycol group (e.g., PEG1, PEG2, PEG3, PEG4, PEG5, PEG6, PEG7, PEG8, PEG10, PEG12, PEG14, PEG16, PEG18, PEG20, PEG24, PEG28, PEG32, PEG100, PEG200, PEG250, PEG500, PEG600, PEG700, PEG750, PEG800, PEG900, PEG1000, PEG2000, or PEG3000). In some embodiments, L comprises a PEG1, PEG2, PEG3, PEG4, PEG5, or PEG6 group. In some embodiments, L comprises a plurality of PEG1, PEG2, PEG3, PEG4, PEG5, or PEG6 groups (e.g., 2, 3, 4, or 5 PEG1, PEG2, PEG3, PEG4, PEG5, or PEG6 groups). In some embodiments, L comprises a PEG2 group. In some embodiments, L comprises a plurality of PEG2 groups. In some embodiments, L comprises a PEG3 group. In some embodiments, L comprises a plurality of PEG3 groups. In some embodiments, L comprises a PEG4 group. In some embodiments, L comprises a plurality of PEG4 groups.

In some embodiments, the linker comprises between 1 and 1000 atoms (e.g., between 1 and 750 atoms, 1 and 500 atoms, 1 and 250 atoms, 1 and 100 atoms, 1 and 75 atoms, 1 and 50 atoms, 1 and 25 atoms, and 1 and 10 atoms). In some embodiments, the linker comprises between 1 and 100 atoms. In some embodiments, the linker comprises between 1 and 50 atoms. In some embodiments, the linker comprises between 1 and 25 atoms.

In some embodiments, the linker is linear and comprises between 1 and 1000 atoms (e.g., between 1 and 750 atoms, 1 and 500 atoms, 1 and 250 atoms, 1 and 100 atoms, 1 and 75 atoms, 1 and 50 atoms, 1 and 25 atoms, and 1 and 10 atoms). In some embodiments, the linker is linear and comprises between 1 and 100 atoms. In some embodiments, the linker is linear and comprises between 1 and 50 atoms. In some embodiments, the linker is linear and comprises between 1 and 25 atoms.

In some embodiments, the linker is branched, and each branch comprises between 1 and 1000 atoms (e.g., between 1 and 750 atoms, 1 and 500 atoms, 1 and 250 atoms, 1 and 100 atoms, 1 and 75 atoms, 1 and 50 atoms, 1 and 25 atoms, and 1 and 10 atoms). In some embodiments, the linker is branched, and each branch comprises between 1 and 100 atoms. In some embodiments, the linker is branched, and each branch comprises between 1 and 50 atoms. In some embodiments, the linker is branched, and each branch comprises between 1 and 25 atoms.

In some embodiments, the ASGPR binding moiety comprises a structure of Formula (III-a):

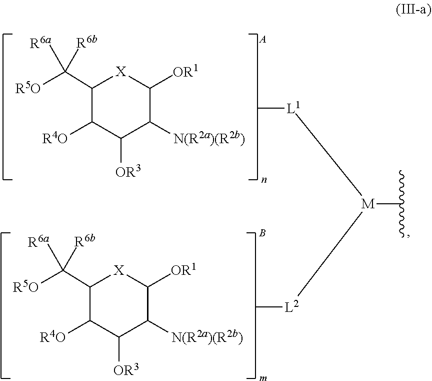

or a salt thereof, wherein each of R¹, R^{2a}, R^{2b}, R³, R⁴, R⁵, R^{6a}, and R^{6b} and subvariables thereof are as defined for Formula (I), each of L¹ and L² is independently a linker, each of m and n is independently an integer between 1 and 100, and M is a linker, wherein “ [symbol] ” represents an attachment point to a branching point, additional linker, or TREM, e.g., a linker, a nucleobase, internucleotide linkage, or terminus within the TREM sequence.

In some embodiments, X is O (e.g., X in each of A and B is O). In some embodiments, each of R¹, R³, R⁴, and R⁵ are independently hydrogen or alkyl (e.g., CH₃) (e.g., R¹, R³, R⁴, and R⁵ in each of A and B is independently hydrogen or alkyl). In some embodiments, R^{2a} is hydrogen (e.g., R^{2a} in each of A and B is hydrogen). In some embodiments, R^{2b} is C(O)CH₃ (e.g., R^{2b} in each of A and B is C(O)CH₃). In some embodiments, each of R^{6a} and R^{6b} is hydrogen (e.g., R^{6a} and R^{6b} in each of A and B is hydrogen). In some embodiments, each of m and n is independently an integer between 1 and 50. In some embodiments, each of m and n is independently an integer between 1 and 25. In some embodiments, each of m and n is independently an integer between 1 and 10. In some embodiments, each of m and n is independently an integer between 1 and 5. In some embodiments, each of m and n is independently 1, 2, 3, 4, or 5. In some embodiments, each of m and n is independently 1.

In an embodiment, each of L¹ and L² independently comprises an alkylene, alkenylene, alkynylene, heteroalkylene, or haloalkylene group. In an embodiment, each of L¹ and L² independently comprises an ester, amide, disulfide, ether, carbonate, aryl, heteroaryl, cycloalkyl, or heterocyclyl group. In an embodiment, each of L¹ and L² independently is cleavable or non-cleavable. In some embodiments, each of L¹ and L² independently comprises a polyethylene glycol group (e.g., PEG1, PEG2, PEG3, PEG4, PEG5, PEG6, PEG7, PEG8, PEG10, PEG12, PEG14, PEG16, PEG18, PEG20, PEG24, PEG28, PEG32, PEG100, PEG200, PEG250, PEG500, PEG600, PEG700, PEG750, PEG800, PEG900, PEG1000, PEG2000, or PEG3000). In some embodiments, each of L¹ and L² independently comprises a PEG1, PEG2, PEG3, PEG4, PEG5, or PEG6 group. In some embodiments, each of L¹ and L² independently comprises a plurality of PEG1, PEG2, PEG3, PEG4, PEG5, or PEG6 groups (e.g., 2, 3, 4, or 5 PEG1, PEG2, PEG3, PEG4, PEG5, or PEG6 groups). In some embodiments, each of L¹ and L² independently comprises a PEG2 group. In some embodiments, each of L¹ and L² independently comprises a plurality of PEG2 groups. In some embodiments, each of L¹ and L² independently comprises a PEG3 group. In some embodiments, each of L¹ and L² independently comprises a plurality of PEG3 groups. In some embodiments, each of L¹ and L² independently comprises a PEG4 group. In some embodiments, each of L¹ and L² independently comprises a plurality of PEG4 groups.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the ASGPR binding moiety comprises a structure of Formula (III-b):

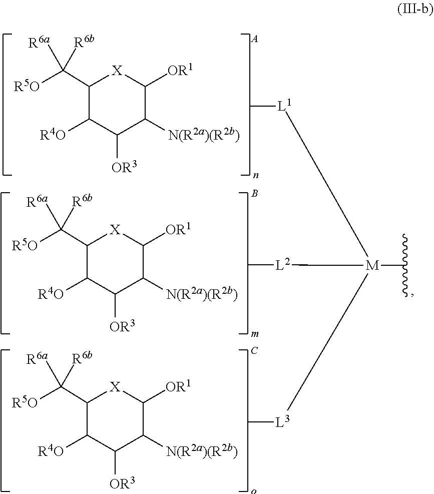

or a salt thereof, wherein each of R¹, R^{2a}, R^{2b}, R³, R⁴, R⁵, R^{6a}, and R^{6b} and subvariables thereof are as defined for Formula (I), each of L¹, L², and L³ is independently a linker, each of m, n, and o is independently an integer between 1 and 100, and M is a linker, wherein “ [symbol] ” represents an attachment point to a branching point, additional linker, or TREM, e.g., a linker, a nucleobase, internucleotide linkage, or terminus within the TREM sequence.

In some embodiments, X is O (e.g., X in each of A, B, and C is O). In some embodiments, each of R¹, R³, R⁴, and R⁵ are independently hydrogen or alkyl (e.g., CH₃) (e.g., R¹, R³, R⁴, and R⁵ in each of A, B, and C is independently hydrogen or alkyl). In some embodiments, R^{2a} is hydrogen (e.g., R^{2a} in each of A, B, and C is hydrogen). In some embodiments, R^{2b} is C(O)CH₃ (e.g., R^{2b} in each of A, B, and C is C(O)CH₃). In some embodiments, each of R^{6a} and R^{6b} is hydrogen (e.g., R^{6a} and R^{6b} in each of A, B, and C is hydrogen). In some embodiments, each of m, n, and o is independently an integer between 1 and 50. In some embodiments, each of m, n, and o is independently an integer between 1 and 25. In some embodiments, each of m, n, and o is independently an integer between 1 and 10. In some embodiments, each of m, n, and o is independently an integer between 1 and 5. In some embodiments, each of m, n, and o is independently 1, 2, 3, 4, or 5. In some embodiments, each of m, n, and o is independently 1.

In an embodiment, each of L¹, L², and L³ independently comprises an alkylene, alkenylene, alkynylene, heteroalkylene, or haloalkylene group. In an embodiment, each of L¹, L², and L³ independently comprises an ester, amide, disulfide, ether, carbonate, aryl, heteroaryl, cycloalkyl, or heterocyclyl group. In an embodiment, each of L¹, L², and L³ independently is cleavable or non-cleavable. In an embodiment, each of L¹ and L² independently is cleavable or non-cleavable. In some embodiments, each of L¹, L², and L³ independently comprises a polyethylene glycol group (e.g., PEG1, PEG2, PEG3, PEG4, PEG5, PEG6, PEG7, PEG8, PEG10, PEG12, PEG14, PEG16, PEG18, PEG20, PEG24, PEG28, PEG32, PEG100, PEG200, PEG250, PEG500, PEG600, PEG700, PEG750, PEG800, PEG900, PEG1000, PEG2000, or PEG3000). In some embodiments, each of L¹, L², and L³ independently comprises a PEG1, PEG2, PEG3, PEG4, PEG5, or PEG6 group. In some embodiments, each of L¹, L², and L³ independently comprises a plurality of PEG1, PEG2, PEG3, PEG4, PEG5, or PEG6 groups (e.g., 2, 3, 4, or 5 PEG1, PEG2, PEG3, PEG4, PEG5, or PEG6 groups). In some embodiments, each of L¹, L², and L³ independently comprises a PEG2 group. In some embodiments, each of L¹, L², and L³ independently comprises a plurality of PEG2 groups. In some embodiments, each of L¹, L², and L³ independently comprises a PEG3 group. In some embodiments, each of L¹, L², and L³ independently comprises a plurality of PEG3 groups. In some embodiments, each of L¹, L², and L³ independently comprises a PEG4 group. In some embodiments, each of L¹, L², and L³ independently comprises a plurality of PEG4 groups.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the ASGPR binding moiety comprises a structure of Formula (III-c):

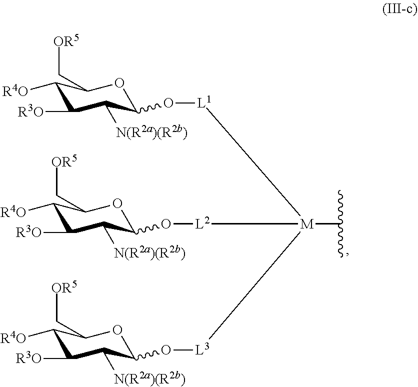

or a salt thereof, wherein each of R^{2a}, R^{2b} R³, R⁴, R⁵, and subvariables thereof are as defined for Formula (I), each of L¹, L², and L³ is independently a linker, and M is a linker, wherein “ [symbol] ” represents an attachment point to a branching point, additional linker, or TREM, e.g., a linker, a nucleobase, internucleotide linkage, or terminus within the TREM sequence.

In some embodiments, each of R³, R⁴, and R⁵ are independently hydrogen or alkyl (e.g., CH₃). In some embodiments, R^{2a} is hydrogen. In some embodiments, R^{2b} is C(O)CH₃.

In an embodiment, each of L¹, L², and L³ independently comprises an alkylene, alkenylene, alkynylene, heteroalkylene, or haloalkylene group. In an embodiment, each of L¹, L², and L³ independently comprises an ester, amide, disulfide, ether, carbonate, aryl, heteroaryl, cycloalkyl, or heterocyclyl group. In an embodiment, each of L¹, L², and L³ independently is cleavable or non-cleavable. In an embodiment, each of L¹ and L² independently is cleavable or non-cleavable. In some embodiments, each of L¹, L², and L³ independently comprises a polyethylene glycol group (e.g., PEG1, PEG2, PEG3, PEG4, PEG5, PEG6, PEG7, PEG8, PEG10, PEG12, PEG14, PEG16, PEG18, PEG20, PEG24, PEG28, PEG32, PEG100, PEG200, PEG250, PEG500, PEG600, PEG700, PEG750, PEG800, PEG900, PEG1000, PEG2000, or PEG3000). In some embodiments, each of L¹, L², and L³ independently comprises a PEG1, PEG2, PEG3, PEG4, PEG5, or PEG6 group. In some embodiments, each of L¹, L², and L³ independently comprises a plurality of PEG1, PEG2, PEG3, PEG4, PEG5, or PEG6 groups (e.g., 2, 3, 4, or 5 PEG1, PEG2, PEG3, PEG4, PEG5, or PEG6 groups). In some embodiments, each of L¹, L², and L³ independently comprises a PEG2 group. In some embodiments, each of L¹, L², and L³ independently comprises a plurality of PEG2 groups. In some embodiments, each of L¹, L², and L³ independently comprises a PEG3 group. In some embodiments, each of L¹, L², and L³ independently comprises a plurality of PEG3 groups. In some embodiments, each of L¹, L², and L³ independently comprises a PEG4 group. In some embodiments, each of L¹, L², and L³ independently comprises a plurality of PEG4 groups.

In some embodiments, M comprises an alkylene, alkenylene, alkynylene, heteroalkylene, or haloalkylene group. In an embodiment, M comprises an ester, amide, disulfide, ether, carbonate, aryl, heteroaryl, cycloalkyl, or heterocyclyl group. In an embodiment, M is cleavable or non-cleavable.

In some embodiments, the ASGPR binding moiety comprises a compound selected from:

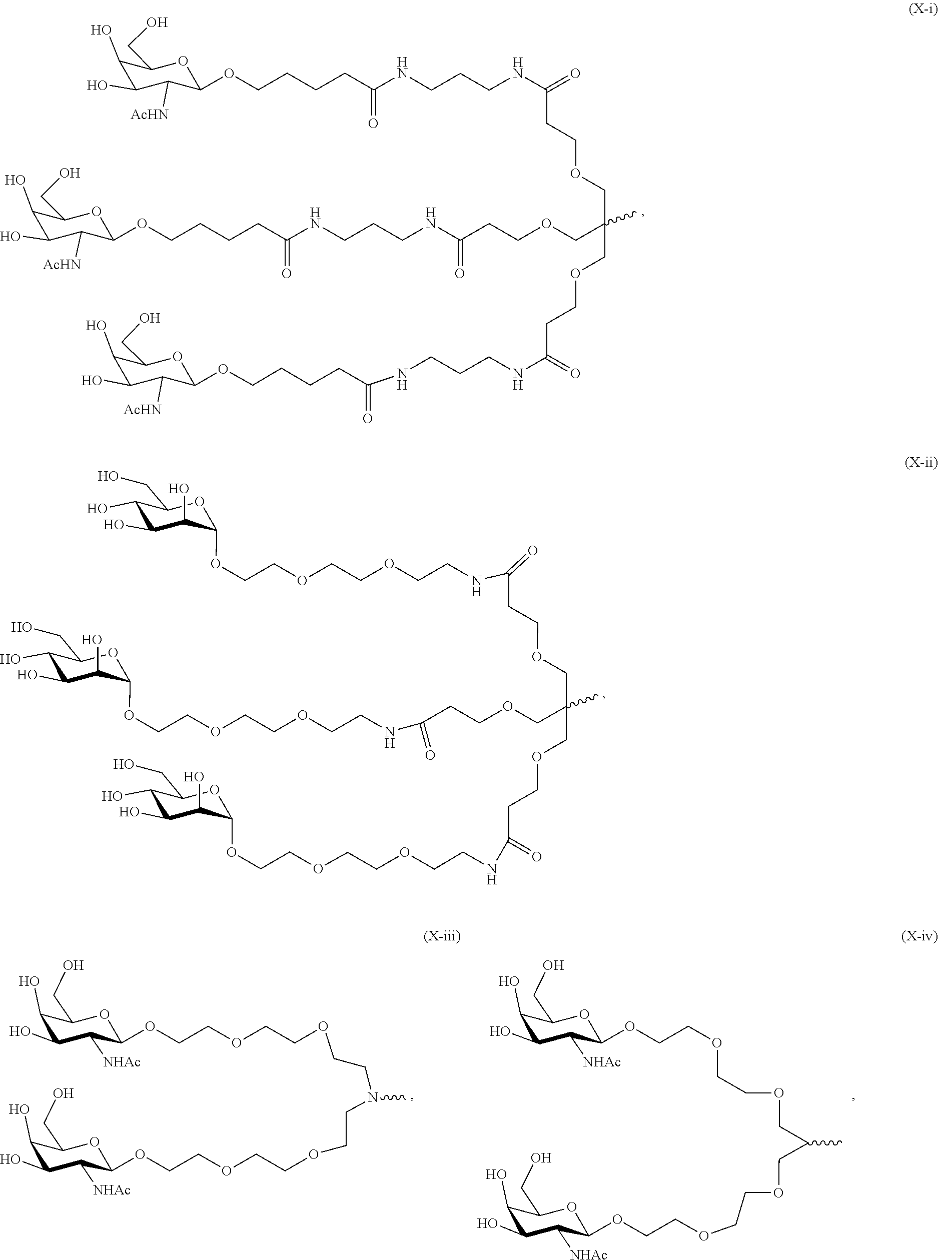

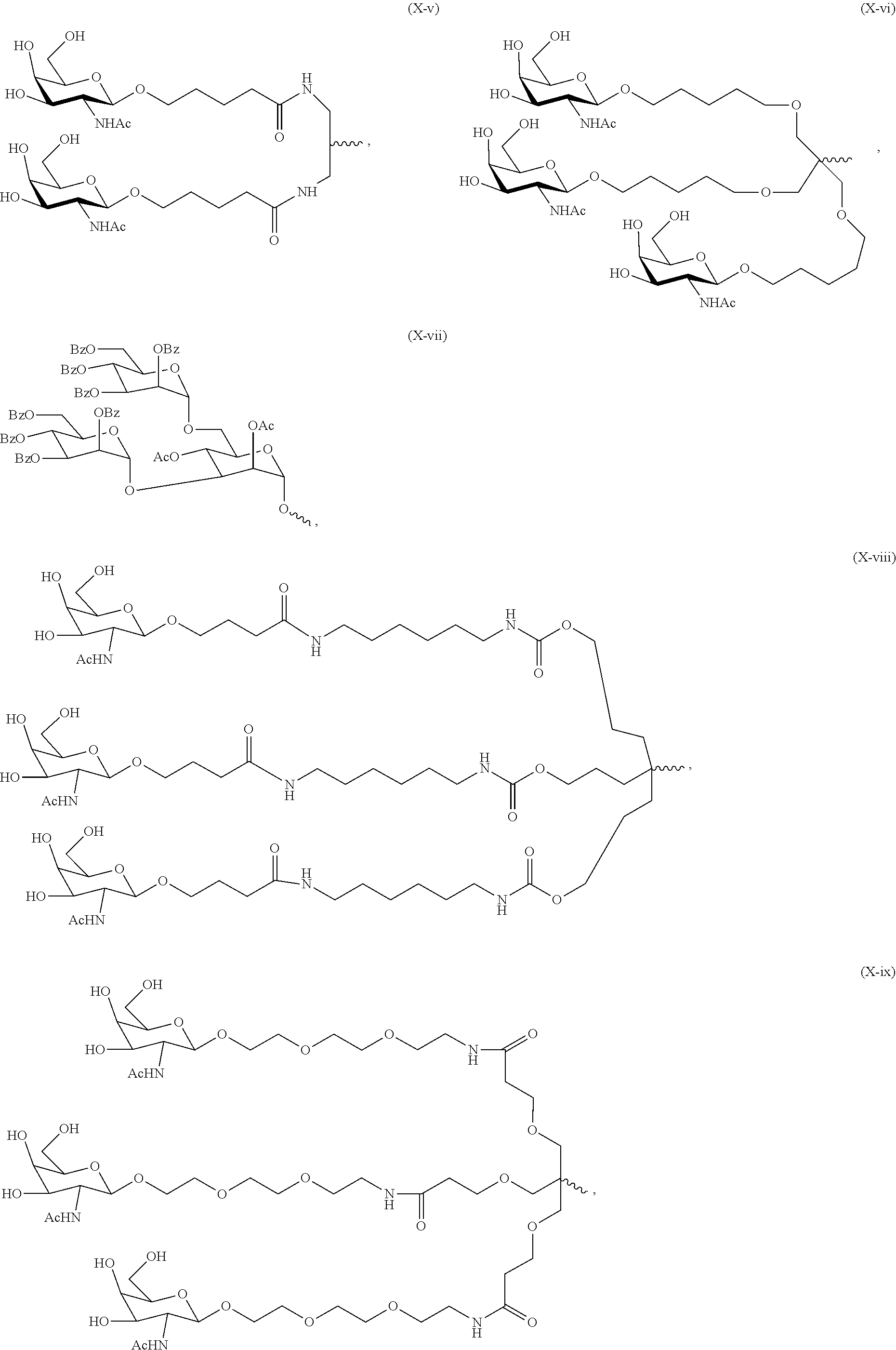

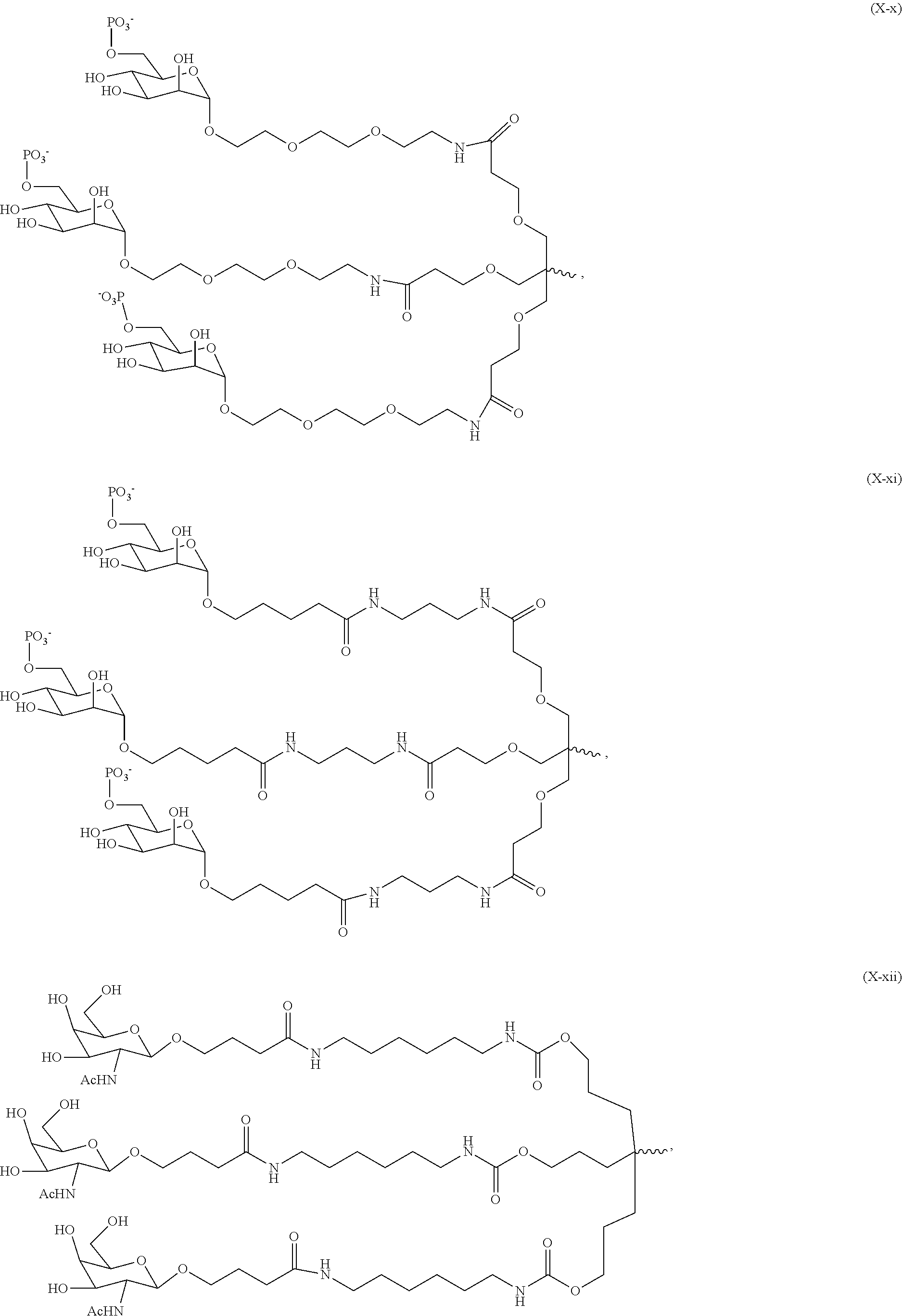

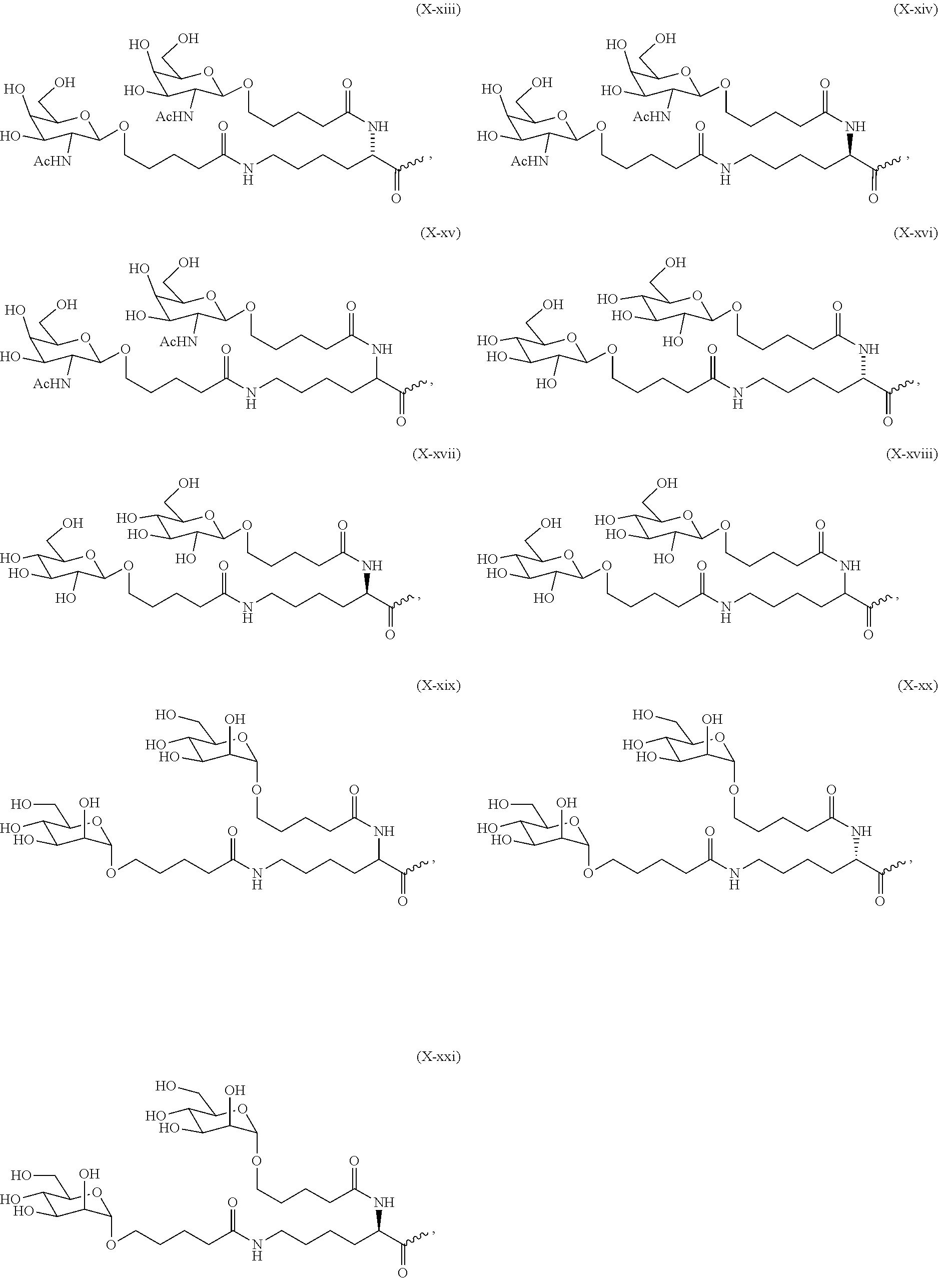

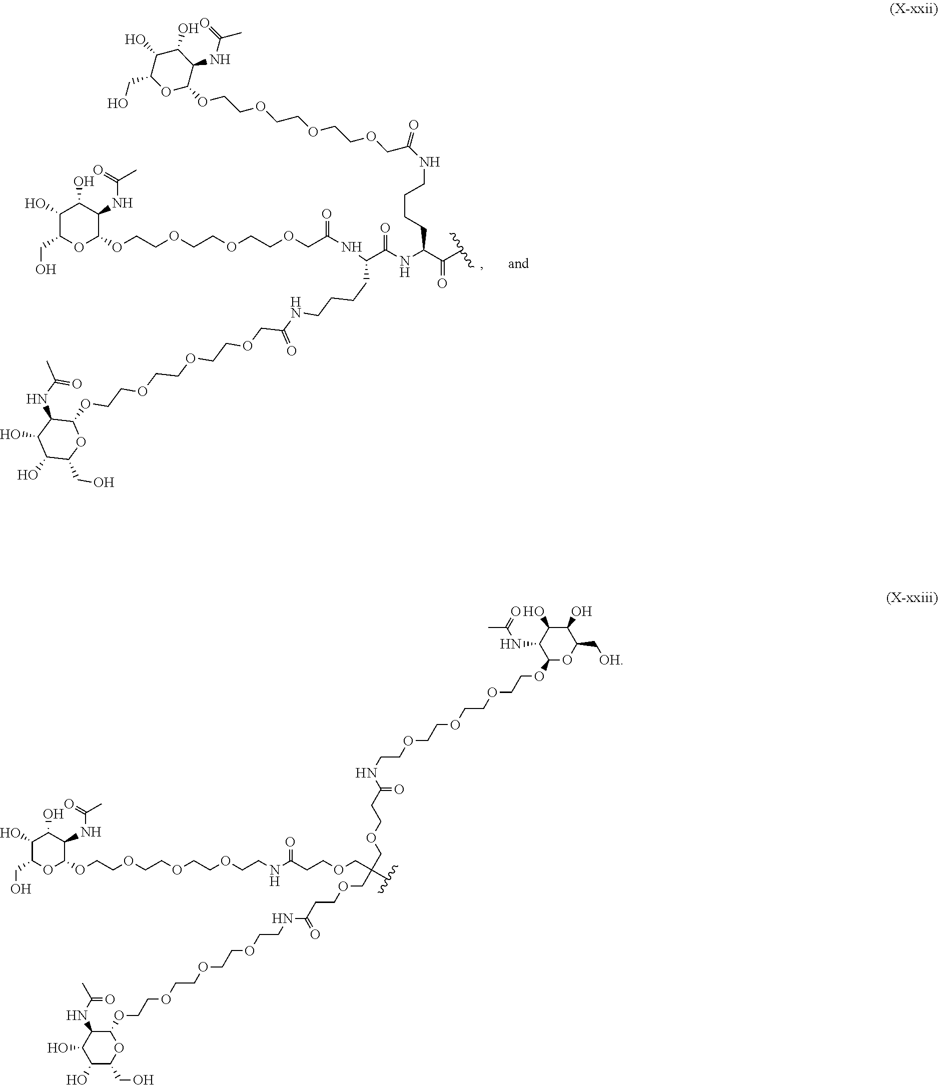 [SMILES: *C(=O)[C@H](CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O ; *C(COCCC(=O)NCCOCCOCCOCCO[C@@H]1O[C@H](CO)[C@H](O)[C@H](O)[C@H]1NC(C)=O)(COCCC(=O)NCCOCCOCCOCCO[C@@H]1O[C@H](CO)[C@H](O)[C@H](O)[C@H]1NC(C)=O)COCCC(=O)NCCOCCOCCOCCO[C@@H]1O[C@H](CO)[C@H](O)[C@H](O)[C@H]1NC(C)=O]

In some embodiments, the ASGPR binding moiety is a compound (X-i). In some embodiments, the ASGPR binding moiety is compound (X-ii). In some embodiments, the ASGPR binding moiety is compound (X-iii). In some embodiments, the ASGPR binding moiety is compound (X-iv). In some embodiments, the ASGPR binding moiety is compound (X-v). In some embodiments, the ASGPR binding moiety is compound (X-vi). In some embodiments, the ASGPR binding moiety is compound (X-vii). In some embodiments, the ASGPR binding moiety is compound (X-viii). In some embodiments, the ASGPR binding moiety is compound (X-ix). In some embodiments, the ASGPR binding moiety is compound (X-x). In some embodiments, the ASGPR binding moiety is compound (X-xi). In some embodiments, the ASGPR binding moiety is compound (X-xii). In some embodiments, the ASGPR binding moiety is compound (X-xiii). In some embodiments, the ASGPR binding moiety is compound (X-xiv). In some embodiments, the ASGPR binding moiety is compound (X-xv). In some embodiments, the ASGPR binding moiety is compound (X-xvi). In some embodiments, the ASGPR binding moiety is compound (X-xvii). In some embodiments, the ASGPR binding moiety is compound (X-xviii). In some embodiments, the ASGPR binding moiety is compound (X-xix). In some embodiments, the ASGPR binding moiety is compound (X-xx). In some embodiments, the ASGPR binding moiety is compound (X-xxi). In some embodiments, the ASGPR binding moiety is compound (X-xxii). In some embodiments, the ASGPR binding moiety is compound (X-xxiii). In some embodiments, the ASGPR binding moiety is a compound selected from compound (X-i), (X-xxii), and (X-xxii).

In some embodiments, the ASGPR binding moiety comprises a linker comprising a cyclic moiety, such as a pyrroline ring. In an embodiment, the ASGPR binding moiety comprises a structure of Formula (CII):

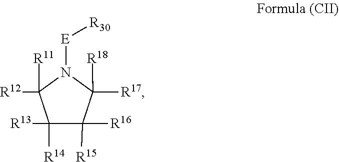

or a salt thereof, wherein E is absent or C(O), C(O)O, C(O)NH, C(S), C(S)NH, SO, SO₂, or SO₂NH; R¹¹, R¹², R¹³, R¹⁴, R¹⁵, R¹⁶, R¹⁷, and R¹⁸ are each independently for each occurrence H, —CH₂OR^{a}, or OR^{b}; R^{a} and R^{b} are each independently for each occurrence hydrogen, a hydroxyl protecting group, optionally substituted alkyl, optionally substituted aryl, optionally substituted cycloalkyl, optionally substituted aralkyl, optionally substituted alkenyl, optionally substituted heteroaryl, polyethyleneglycol (PEG), a phosphate, a diphosphate, a triphosphate, a phosphonate, a phosphonothioate, a phosphonodithioate, a phosphorothioate, a phosphorothiolate, a phosphorodithioate, a phosphorothiolothionate, a phosphodicster, a phosphotricster, an activated phosphate group, an activated phosphite group, a phosphoramidite, a solid support, —P(Z¹)(Z²)—O-nucleoside, —P(Z¹)(Z²)—O-oligonucleotide, —P(Z¹)(O-linker-R^{L})—O-nucleoside, or —P(Z¹)(O-linker-R^{L})—O-oligonucleotide; R³⁰ is independently for each occurrence-linker-R^{L} or R³¹; R^{L} is hydrogen or a ligand; R³¹ is —C(O)CH(N(R³²)₂)(CH₂)ₕN(R³²)₂; R³² is independently for each occurrence H, —R^{L}, -linker-R^{L} or R³¹; Z¹ is independently for each occurrence O or S; Z² is independently for each occurrence O, S, N (alkyl) or optionally substituted alkyl; and h is independently for each occurrence 1-20.

In some embodiments, the compound of Formula (CII) is selected from:

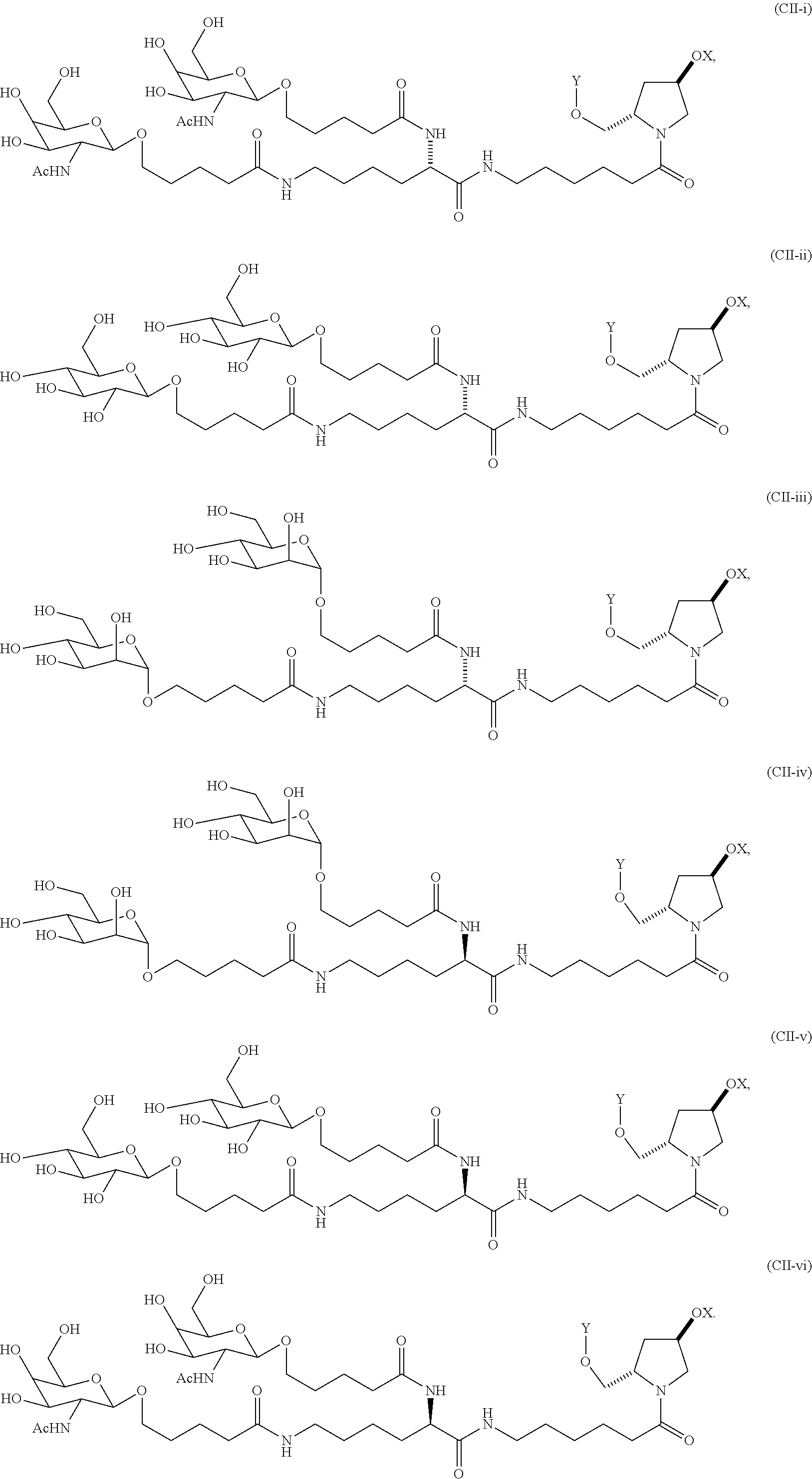

In some embodiments, the ASGPR binding moiety is a compound or substructure disclosed in U.S. Pat. No. 8,106,022, which is incorporated herein by reference in its entirety.

In some embodiments, the ASGPR binding moiety is a compound (CII-i). In some embodiments, the ASGPR binding moiety is a compound (CII-ii). In some embodiments, the ASGPR binding moiety is a compound (CII-iii). In some embodiments, the ASGPR binding moiety is a compound (CII-iv). In some embodiments, the ASGPR binding moiety is a compound (CII-v). In some embodiments, the ASGPR binding moiety is a compound (CII-vi).

In some embodiments, the ASGPR binding moiety is a compound of Formula (C-1), (C-2), (C-3) or (C4):

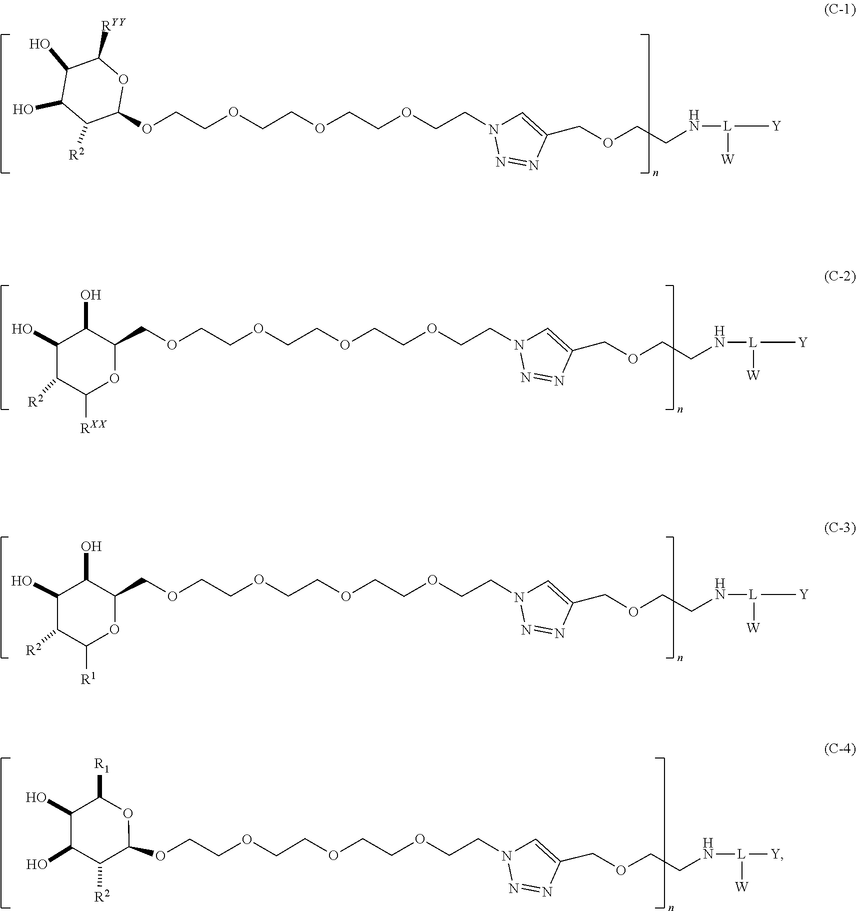

or a pharmaceutically acceptable salt thereof, wherein: n is 1, 2, or 3; W is absent or a peptide; L is -(T-Q-T-Q)ₘ-, wherein each T is independently absent or is (C₁-C₁₀) alkylene, (C₂-C₁₀) alkenylene, or (C₂-C₁₀) alkynylene, wherein one or more carbon groups of said T may each independently be replaced with a heteroatom group independently selected from —O—, —S—, and —N(R⁴)— wherein the heteroatom groups are separated by at least 2 carbon atoms, and wherein alkylene, alkenylene, and alkynylene may each be independently substituted with one or more halo atoms; each Q is independently absent or is C(O), C(O)—NR⁴, NR⁴—C(O), O—C(O)—NR⁴, NR⁴—C(O)—O, —CH₂—, a heteroaryl, or a heteroatom group selected from O, S, S—S, S(O), S(O)₂, and NR⁴, wherein at least two carbon atoms separate the heteroatom groups O, S, S—S, S(O), S(O)₂ and NR⁴ from any other heteroatom group; each R⁴ is independently —H, —(C₁-C₂₀)alkyl, or (C₃-C₈)cycloalkyl wherein one to six —CH₂— groups of the alkyl or cycloalkyl separated by at least two carbon atoms may be replaced with —O—, —S—, or —N(R⁴)—, and —CH₃— of the alkyl may each be independently replaced with a heteroatom group selected from —N(R⁴)₂, —OR⁴, and —S(R⁴) wherein the heteroatom groups are separated by at least 2 carbon atoms; and wherein the alkyl and cycloalkyl may be substituted with halo atoms; and m is independently 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40.

In some embodiments, the ASGPR binding moiety is a compound (C-1). In some embodiments, the ASGPR binding moiety is a compound (C-2). In some embodiments, the ASGPR binding moiety is a compound (C-3). In some embodiments, the ASGPR binding moiety is a compound (C-4).

In some embodiments, the compound of Formula (C-1), (C-2), (C-3) or (C4) comprises:

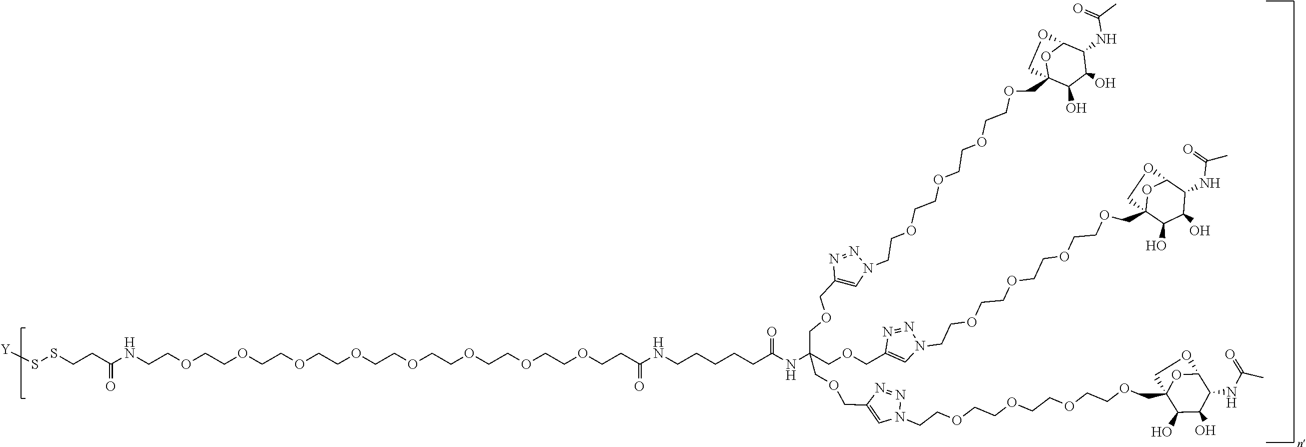

wherein n′ is 1 or 2 or a pharmaceutically acceptable salt thereof.

In some embodiments, the ASGPR binding moiety is a compound of Formula (E):

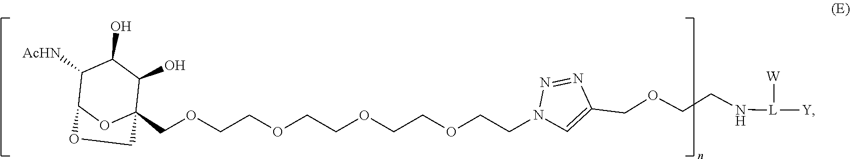

or a pharmaceutically acceptable salt thereof, wherein: n is i, 2 or 3; W is absent or is a peptide; L is -(T-Q-T-Q)ₘ-, wherein each T is independently absent or is (C₁-C₁₀) alkylene, (C₂-C₁₀) alkenylene, or (C2-C10) alkynylene, wherein one or more carbon groups of said T may each independently be replaced with a heteroatom group independently selected from —O—, —S—, and —N(R⁴)— wherein the heteroatom groups are separated by at least 2 carbon atoms, wherein said alkylene, alkenylene, alkynylene, may each independently be substituted by one or more halo atoms; each Q is independently absent or is C(O), C(0)-R⁴, R⁴—C(O), O—C(O)—R⁴, R⁴—C(O)—O, —CH₂—, a heteroaryl, or a heteroatom group selected from O, S, S—S, S(O), S(0)₂, and NR⁴, wherein at least two carbon atoms separate the heteroatom groups O, S, S—S, S(O), S(0)₂ and NR⁴ from any other heteroatom group; each R⁴ is independently —H, —(C₁-C2₀)alkyl, —(C₁-C₂₀)alkenyl, —(C₂-C₂₀) alkynyl, or (C₃-C₆) cycloalkyl wherein one to six —CH₂— groups of the alkyl or cycloalkyl separated by at least two carbon atoms may be replaced with —O—, —S—, or —N(R⁴)—, and —CH₃ of the alkyl may be replaced with a heteroatom group selected from —N(R⁴)₂, —OR⁴, and —S(R⁴) wherein the heteroatom groups are separated by at least 2 carbon atoms; and wherein the alkyl, alkenyl, alkynyl, and cycloalkyl may be substituted with halo atoms; each m is independently 0, 1, 2, 3, 4, 5, 6, 7, 8, 9, 10,11, 12,13, 14, 15, 16, 17, 18, 19, 20, 21, 22, 23, 24, 25, 26, 27, 28, 29, 30, 31, 32, 33, 34, 35, 36, 37, 38, 39, or 40.

In some embodiments, the compound of Formula (E) is selected from:

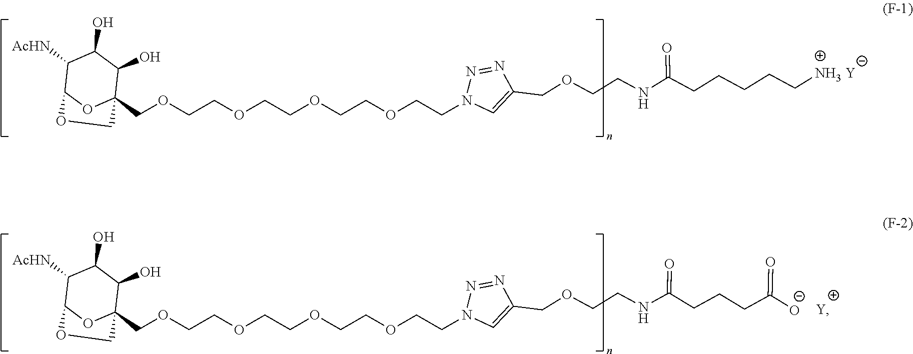

or a pharmaceutically acceptable salt thereof, and Y is as defined in Formula (E).

In some embodiments. n is 1. In some embodiments, n is 2. In some embodiments, n is 3.

In some embodiments of a compound of Formula (E), the compound is:

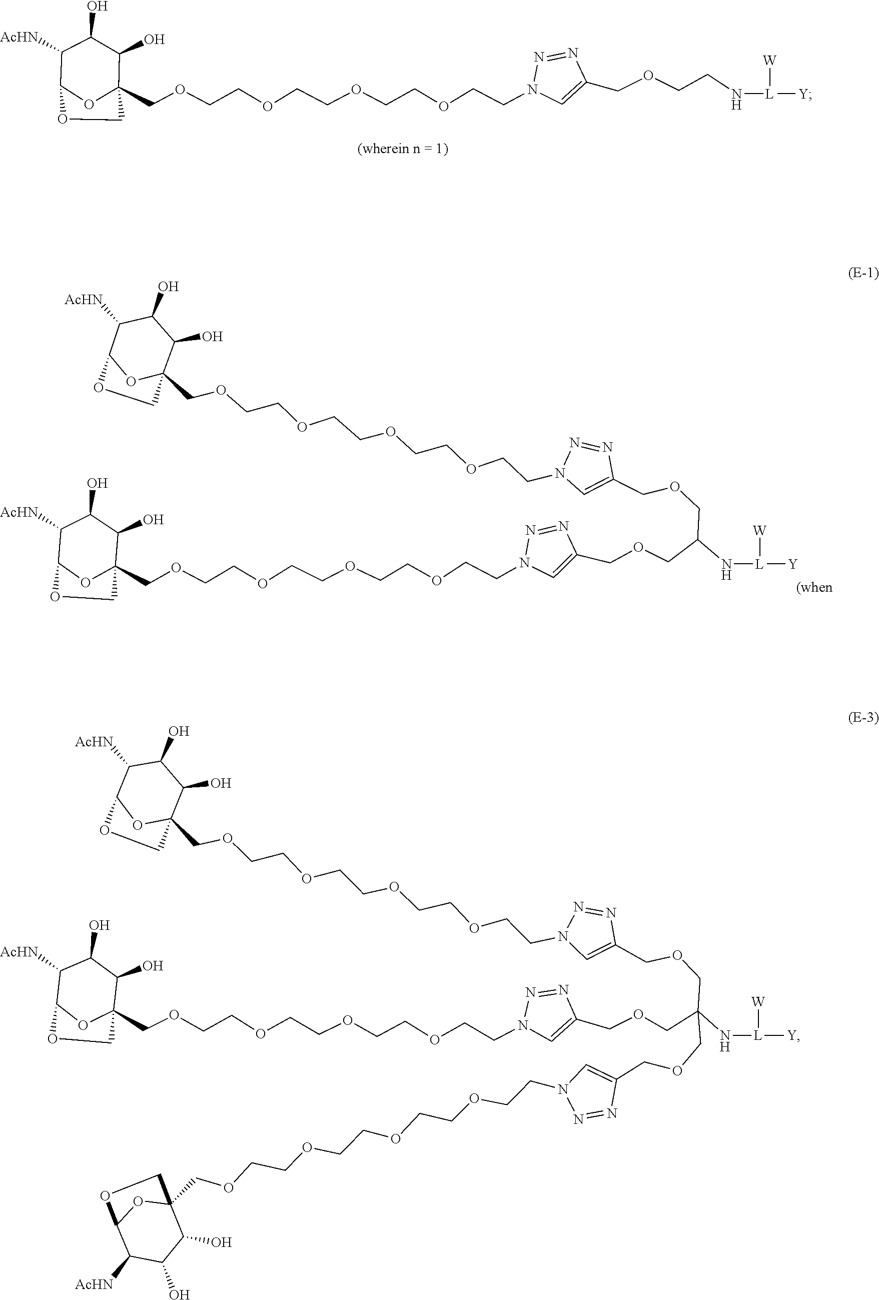

or a pharmaceutically acceptable salt thereof.

In some embodiments, the ASGPR binding moiety is a compound or substructure disclosed in WO2017/083368, which is incorporated herein by reference in its entirety.

In other embodiments, the ASGPR binding moiety is selected from:

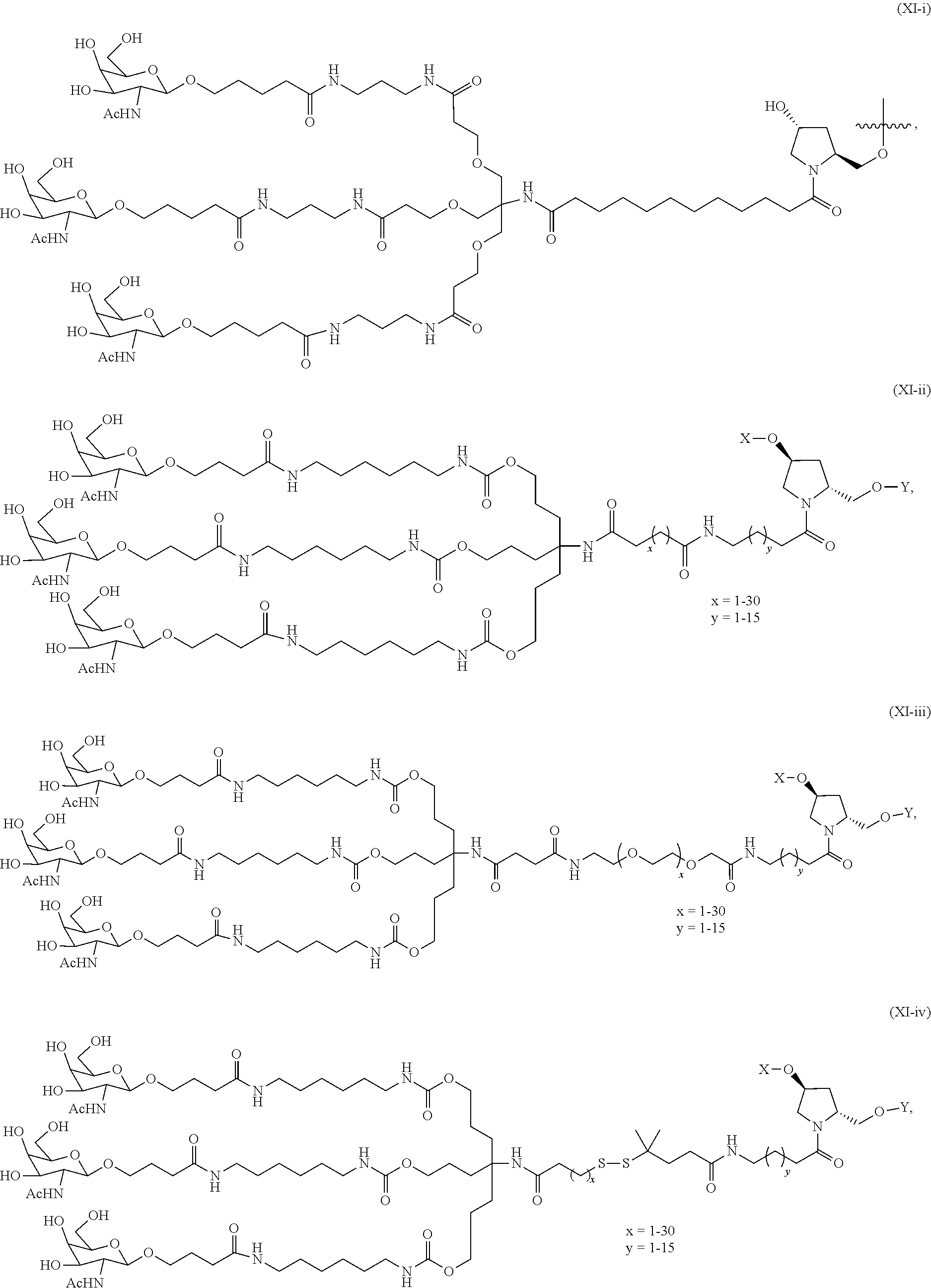

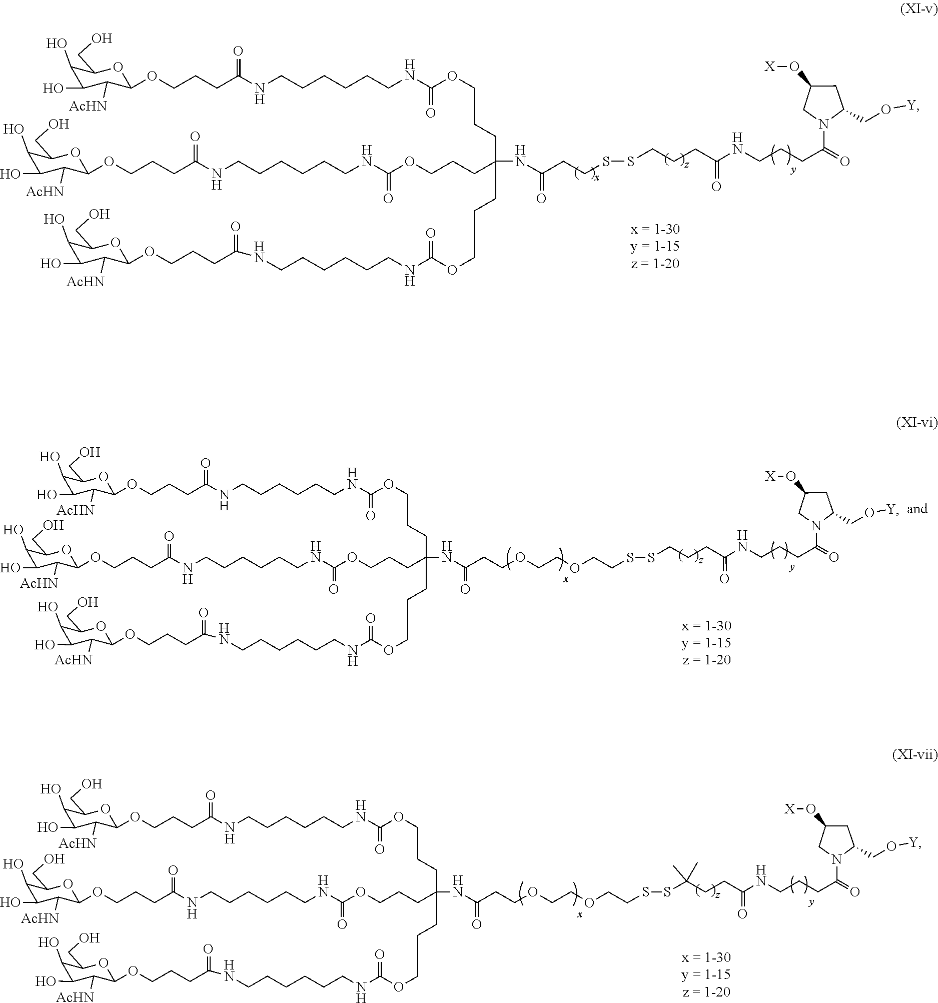

wherein one of X or Y is a branching point, a linker, or a TREM, e.g., a linker, a nucleobase, internucleotide linkage, or terminus within the TREM sequence, and the other of X and Y is hydrogen.

In an embodiment, the ASGPR binding moiety comprises a structure of Formula (XII-a):

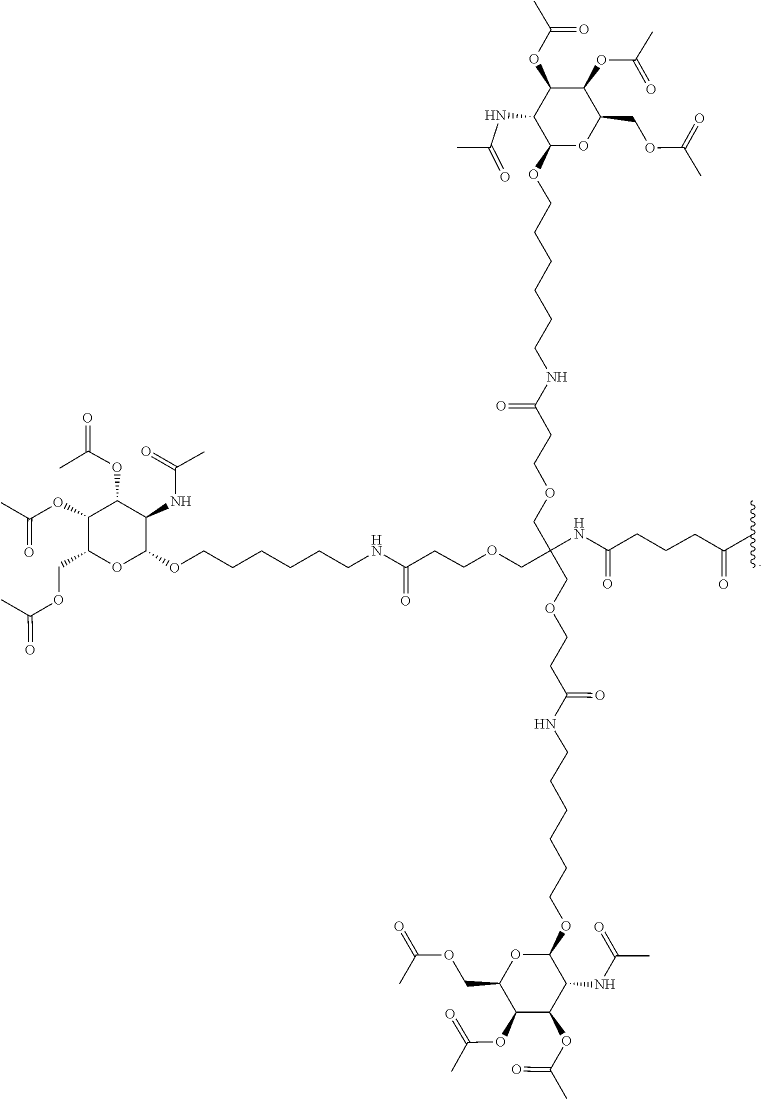 [SMILES: *C(=O)CCCC(=O)NC(COCCC(=O)NCCCCCCO[C@@H]1O[C@H](COC(C)=O)[C@H](OC(C)=O)[C@H](OC(C)=O)[C@H]1NC(C)=O)(COCCC(=O)NCCCCCCO[C@@H]1O[C@H](COC(C)=O)[C@H](OC(C)=O)[C@H](OC(C)=O)[C@H]1NC(C)=O)COCCC(=O)NCCCCCCO[C@@H]1O[C@H](COC(C)=O)[C@H](OC(C)=O)[C@H](OC(C)=O)[C@H]1NC(C)=O]

In an embodiment, the ASGPR binding moiety is a compound or substructure disclosed in Nucleic Acids (2016) 5:e317 or WO2015/042447, each of which is incorporated herein by reference in its entirety.

In some embodiments, the ASGPR binding moiety comprises a structure of Formula (V-a):

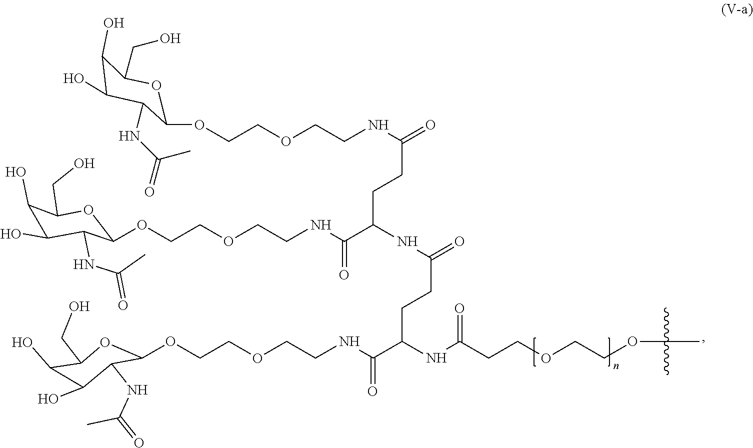

wherein n is an integer from 1 to 20. In some embodiments, the compound of Formula (V-a) is selected from:

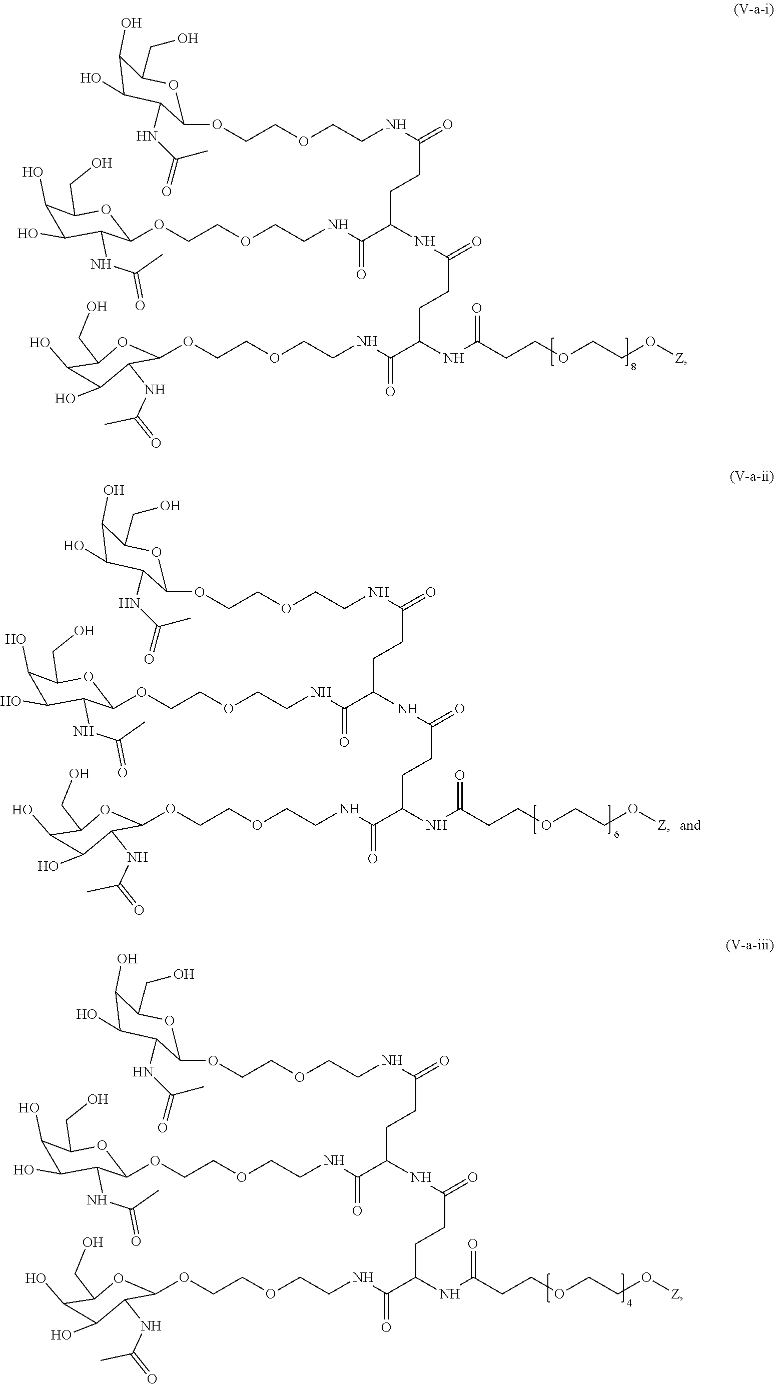

wherein Z is an oligomeric compound, e.g., a linker or a nucleobase within the ASt of a TREM.

In another embodiment, the ASGPR binding moiety comprises a structure of Formula (V-b):

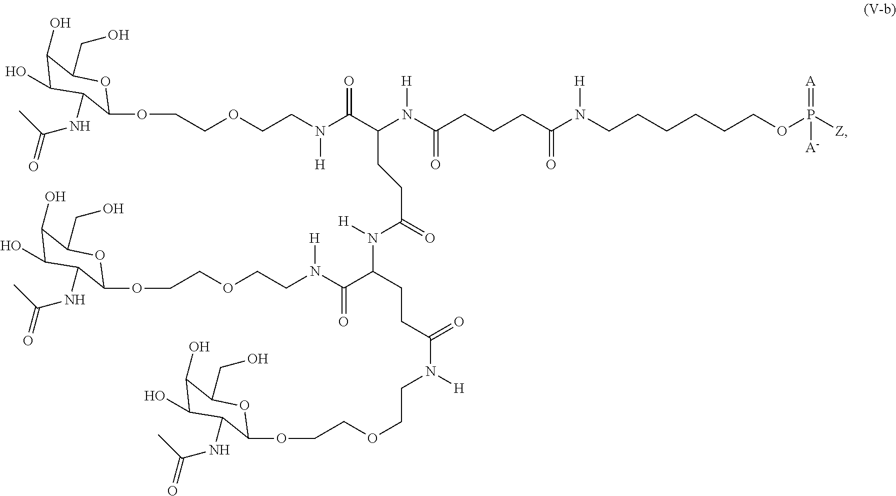

wherein A is O or S, A′ is O, S, or NH, and Z is an oligomeric compound, e.g., a linker or a TREM, e.g., a linker, a nucleobase, internucleotide linkage, or terminus within the TREM sequence.

In some embodiments, the ASGPR binding moiety comprises

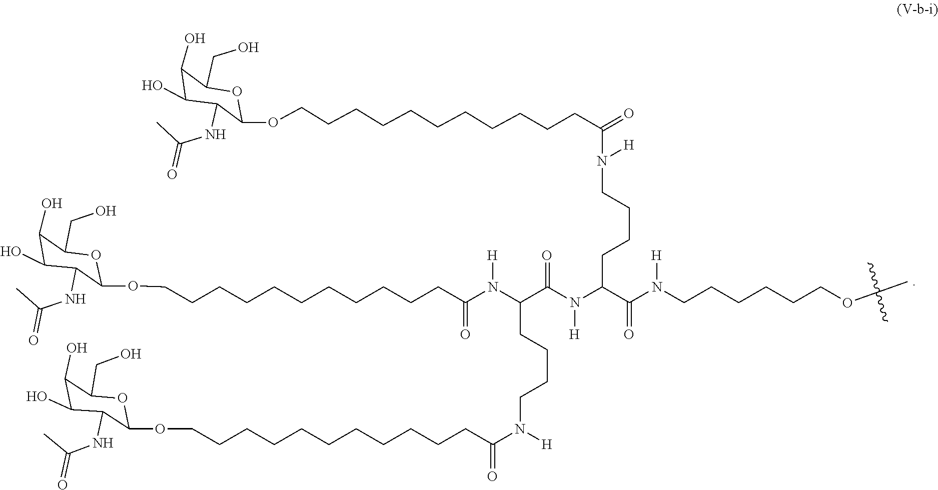 [SMILES: *OCCCCCCNC(=O)C(CCCCNC(=O)CCCCCCCCCCCOC1OC(CO)C(O)C(O)C1NC(C)=O)NC(=O)C(CCCCNC(=O)CCCCCCCCCCCOC1OC(CO)C(O)C(O)C1NC(C)=O)NC(=O)CCCCCCCCCCCOC1OC(CO)C(O)C(O)C1NC(C)=O]

In some embodiments, the ASGPR binding moiety is selected from:

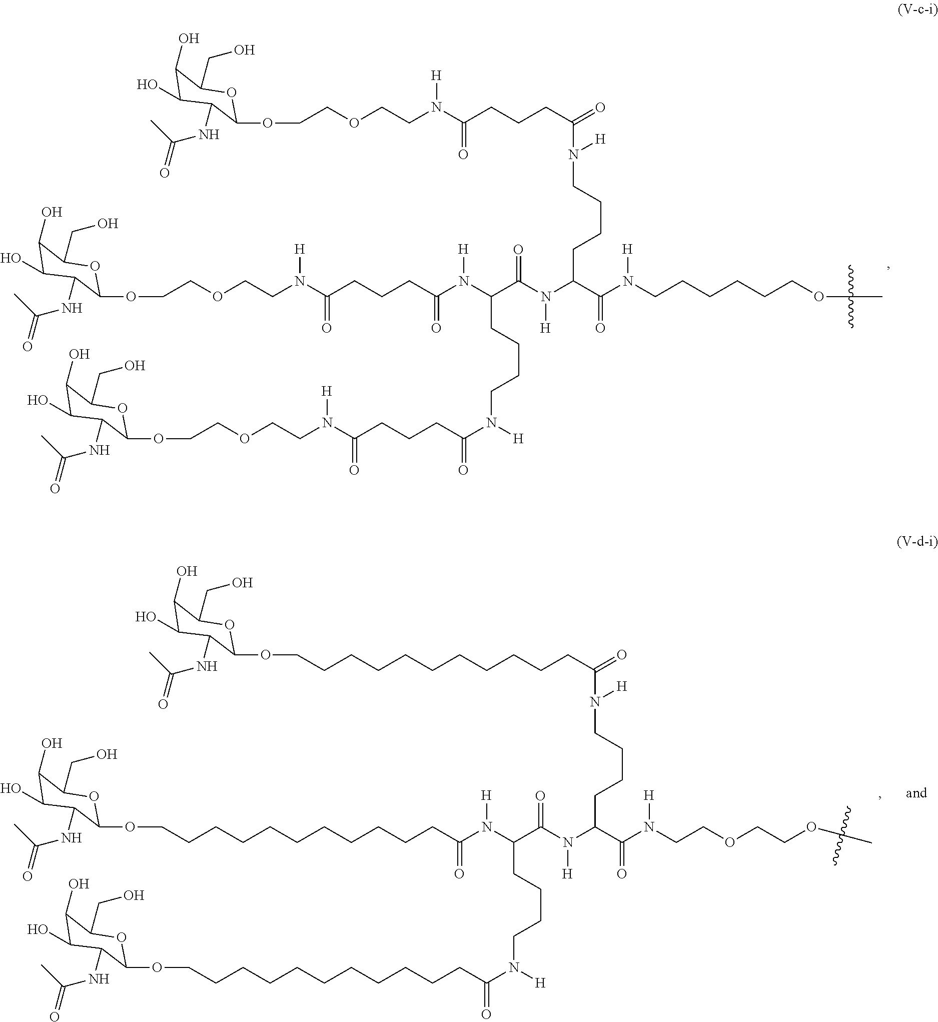 [SMILES: *OCCCCCCNC(=O)C(CCCCNC(=O)CCCC(=O)NCCOCCOC1OC(CO)C(O)C(O)C1NC(C)=O)NC(=O)C(CCCCNC(=O)CCCC(=O)NCCOCCOC1OC(CO)C(O)C(O)C1NC(C)=O)NC(=O)CCCC(=O)NCCOCCOC1OC(CO)C(O)C(O)C1NC(C)=O ; *OCCOCCNC(=O)C(CCCCNC(=O)CCCCCCCCCCCOC1OC(CO)C(O)C(O)C1NC(C)=O)NC(=O)C(CCCCNC(=O)CCCCCCCCCCCOC1OC(CO)C(O)C(O)C1NC(C)=O)NC(=O)CCCCCCCCCCCOC1OC(CO)C(O)C(O)C1NC(C)=O]

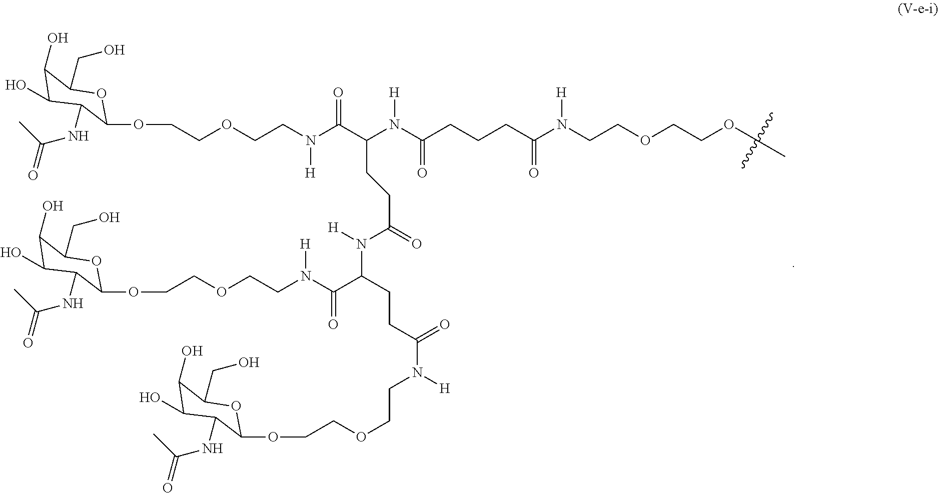 [SMILES: *OCCOCCNC(=O)CCCC(=O)NC(CCC(=O)NC(CCC(=O)NCCOCCOC1OC(CO)C(O)C(O)C1NC(C)=O)C(=O)NCCOCCOC1OC(CO)C(O)C(O)C1NC(C)=O)C(=O)NCCOCCOC1OC(CO)C(O)C(O)C1NC(C)=O]

In an embodiment, the ASGPR binding moiety is a compound or substructure disclosed in WO 2017/156012, which is incorporated herein by reference in its entirety.

In some embodiments, a hydroxyl group within an ASGPR binding moiety is protected, for example, with an acetyl or acetonide moiety. In some embodiments, a hydroxyl group within an ASGPR binding moiety is protected with an acetyl group. In some embodiments, a hydroxyl group within an ASGPR binding moiety is protected with acetonide group. For example, 1, 2, 3, 4, 5, 6, 7, 8, 9, 10, 11, 12, or more hydroxyl groups within an ASGPR binding moiety may be protected, e.g., with an acetyl group or an acetonide group. In some embodiments, all of the hydroxyl groups with in an ASGPR binding moiety are protected.

In some embodiments, the ASGPR binding moiety is bound to the 2′ or 4′ positions on the sugar moiety (e.g., ribose moiety) within a nucleotide of the TREM. In an embodiment, the ASGPR moiety is bound to a carbon atom at the 2′ or 4′ position. In an embodiment, the ASGPR moiety is bound to an oxygen atom at the 2′ or 4′ position. In an embodiment, the ASGPR is bound through a linker to the 2′ or 4′ position on the sugar moiety. Methods for installing an ASGPR moiety at the 4′-ribose position may carried out based on protocols described in, e.g., Liczner et al. (2021) Beilstein J. Org Chem 17:908-931, which is incorporated herein by reference in its entirety.

Exemplary TREMs comprising an ASGPR binding moiety may have a binding affinity for an ASGPR of between 0.01 nM to 100 mM. In some embodiments, a TREM comprising an ASGPR binding moiety has a binding affinity of less than 10 mM, e.g., 7.5 mM, 5 mM, 2.5 mM, 1 mM, 0.75 mM, 0.5 mM, 0.25 mM, 0.1 mM, 75 nM, 50 nM, 25 nM, 10 nM, 5 nM, or less.

Exemplary TREMs comprising an ASGPR binding moiety may be internalized into a cell, e.g., a hepatocyte. In some embodiments, a TREM comprising an ASGPR binding moiety has an increased uptake into a cell compared with a TREM that does not comprise an ASGPR binding moiety. For example, a TREM comprising an ASGPR binding moiety may be internalized into a cell more than 1.1, 1.2, 1.3, 1.4, 1.5, 1.75, 2, 3, 4, 5, 6, 7, 8, 9, 10, 15, 20, 25, 30, 35, 40, 45, 50, 75, 100 times or more than a TREM that does not comprise an ASGPR binding moiety.

Additional exemplary ASGPR moieties are described in further detail in U.S. Pat. Nos. 8,828,956; 9,867,882; 10,450,568; 10,808,246; U.S. Patent Publication Nos. 2015/0246133; 2015/0203843; and 2012/0095200; and PCT Publication Nos. WO 2013/166155, 2012/030683, and 2013/166121, each of which are incorporated herein by reference in its entirety.

### ASGPR Linkers

The ASGPR binding moiety comprises at least one linker that connects the carbohydrate to the TREM. In some embodiments, the TREM is connected to one or more carbohydrates (e.g., GalNAc moieties, e.g., of Formula (I)), through a linker as described herein. The linker may be monovalent or multivalent, e.g., bivalent, trivalent, tetravalent, or pentavalent. In some embodiments, the linker comprises a structure selected from:

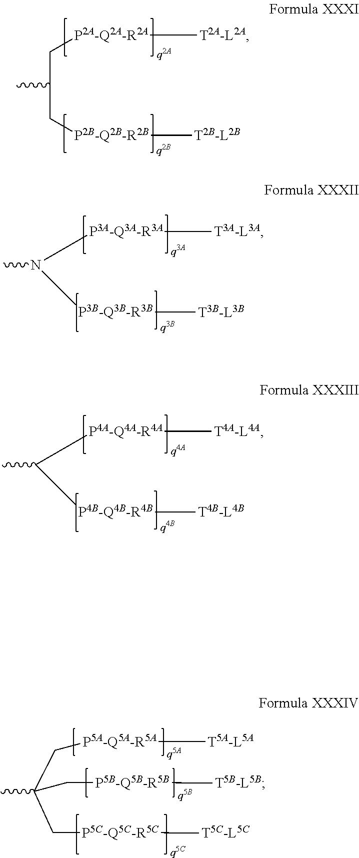

wherein q2A, q2B, q3A, q3B, q4A, q4B, q5A, q5B and q5C represent independently for each occurrence 0-20 and wherein the repeating unit can be the same or different; P^{2A}, P^{2B}, p^{3A}, p^{3B}, P^{4A}, p^{4B}, p^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O; Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O); R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—O,

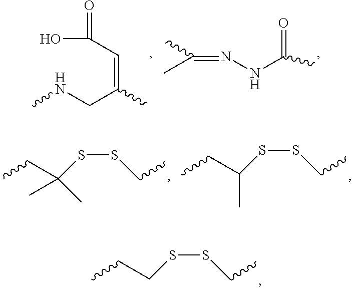

or heterocyclyl; L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain.

In some embodiments, the linker comprises:

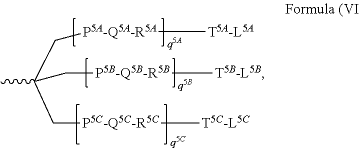

wherein L^{5A}, L^{5B} and L^{5C} represent a monosaccharide, such as GalNAc derivative, e.g., as described herein.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases. A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis. Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular TREM moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or scrum conditions. In one, candidate compounds arc cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

In another embodiment, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, —O—P(S)(H)—S—. A preferred embodiment is —O—P(O)(OH)—O—. These candidates can be evaluated using methods analogous to those described above.

In another embodiment, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

In another embodiment, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula-NHCHRAC(O)NHCHRBC(O)— (SEQ ID NO: 13), where RA and RB are the R groups of the two adjacent amino acids. These candidates can be evaluated using methods analogous to those described above.

The ASGPR binding moiety may be bound to a sugar at any nucleotide position within the acceptor stem domain (ASt Domain1 and/or ASt Domain2). In an embodiment, the ASGPR binding moiety is bound to any carbon atom within a sugar in the acceptor stem domain (ASt Domain1 and/or ASt Domain2). In an embodiment, the ASGPR binding moiety is bound to any nitrogen atom within a sugar in the acceptor stem domain (ASt Domain1 and/or ASt Domain2). In an embodiment, the ASGPR binding moiety is bound to any oxygen atom within a sugar in the acceptor stem domain (ASt Domain1 and/or ASt Domain2). In an embodiment, the ASGPR binding moiety is bound to any sulfur atom within a sugar in the acceptor stem domain (ASt Domain1 and/or ASt Domain2). In an embodiment, the ASGPR binding moiety is bound to any phosphorus atom within a sugar in the acceptor stem domain (ASt Domain1 and/or ASt Domain2).

The ASGPR binding moiety may be bound to the phosphate backbone at any nucleotide position within the acceptor stem domain (ASt Domain1 and/or ASt Domain2). In an embodiment, the ASGPR binding moiety is bound to an oxygen atom within the phosphate backbone within the acceptor stem domain (ASt Domain1 and/or ASt Domain2). In an embodiment, ASGPR binding moiety is bound to a phosphorus atom in phosphate backbone within the acceptor stem domain (ASt Domain1 and/or ASt Domain2). In an embodiment, it is bound to a nitrogen atom in the phosphate backbone within the acceptor stem domain (ASt Domain1 and/or ASt Domain2).

In an embodiment, the ASGPR binding moiety is bound to a sugar at TREM position 1 (G). In an embodiment, the ASGPR binding moiety is bound to a sugar at TREM position 2 (G). In an embodiment, the ASGPR binding moiety is bound to a sugar at TREM position 3 (C). In an embodiment, the ASGPR binding moiety is bound to a sugar at TREM position 4 (U). In an embodiment, the ASGPR binding moiety is bound to a sugar at TREM position 5 (C). In an embodiment, the ASGPR binding moiety is bound to a sugar at TREM position 6 (C). In an embodiment, the ASGPR binding moiety is bound to a sugar at TREM position 7 (G). In an embodiment, the ASGPR binding moiety is bound to a sugar at TREM position 8 (U). In an embodiment, the ASGPR binding moiety is bound to a sugar at TREM position 9 (G).

In an embodiment, the ASGPR binding moiety is bound to the phosphate backbone at TREM position 1 (G). In an embodiment, the ASGPR binding moiety is bound to the phosphate backbone at TREM position 2 (G). In an embodiment, the ASGPR binding moiety is bound to the phosphate backbone at TREM position 3 (C). In an embodiment, the ASGPR binding moiety is bound to the phosphate backbone at TREM position 4 (U). In an embodiment, the ASGPR binding moiety is bound to the phosphate backbone at TREM position 5 (C). In an embodiment, the ASGPR binding moiety is bound to the phosphate backbone at TREM position 6 (C). In an embodiment, the ASGPR binding moiety is bound to the phosphate backbone at TREM position 7 (G). In an embodiment, the ASGPR binding moiety is bound to the phosphate backbone at TREM position 8 (U). In an embodiment, the ASGPR binding moiety is bound to the phosphate backbone at TREM position 9 (G).

In an embodiment, the ASGPR binding moiety is bound to a sugar moiety (e.g., ribose moiety) at TREM position 76 (A). In an embodiment, the ASGPR binding moiety is bound to a sugar moiety (e.g., ribose moiety) at TREM position 75 (C). In an embodiment, the ASGPR binding moiety is bound to a sugar moiety (e.g., ribose moiety) at TREM position 74 (C). In an embodiment, the ASGPR binding moiety is bound to a sugar moiety (e.g., ribose moiety) at TREM position 73 (G). In an embodiment, the ASGPR binding moiety is bound to a sugar moiety (e.g., ribose moiety) at TREM position 72 (C). In an embodiment, the ASGPR binding moiety is bound to a sugar moiety (e.g., ribose moiety) at TREM position 71 (U). In an embodiment, the ASGPR binding moiety is bound to a sugar moiety (e.g., ribose moiety) at TREM position 70 (G). In an embodiment, the ASGPR binding moiety is bound to a sugar moiety (e.g., ribose moiety) at TREM position 69 (A). In an embodiment, the ASGPR binding moiety is bound to a sugar moiety (e.g., ribose moiety) at TREM position 68 (G). In an embodiment, the ASGPR binding moiety is bound to a sugar moiety (e.g., ribose moiety) at TREM position 67 (G). In an embodiment, the ASGPR binding moiety is bound to a sugar moiety (e.g., ribose moiety) at TREM position 66 (C). In an embodiment, the ASGPR binding moiety is bound to a sugar moiety (e.g., ribose moiety) at TREM position 65 (G).

In an embodiment, the ASGPR binding moiety is bound to the phosphate backbone at TREM position 76 (A). In an embodiment, the ASGPR binding moiety is bound to the phosphate backbone at TREM position 75 (C). In an embodiment, the ASGPR binding moiety is bound to the phosphate backbone at TREM position 74 (C). In an embodiment, the ASGPR binding moiety is bound to the phosphate backbone at TREM position 73 (G). In an embodiment, the ASGPR binding moiety is bound to the phosphate backbone at TREM position 72 (C). In an embodiment, the ASGPR binding moiety is bound to the phosphate backbone at TREM position 71 (U). In an embodiment, the ASGPR binding moiety is bound to the phosphate backbone at TREM position 70 (G). In an embodiment, the ASGPR binding moiety is bound to the phosphate backbone at TREM position 69 (A). In an embodiment, the ASGPR binding moiety is bound to the phosphate backbone at TREM position 68 (G). In an embodiment, the ASGPR binding moiety is bound to the phosphate backbone at TREM position 67 (G). In an embodiment, the ASGPR binding moiety is bound to the phosphate backbone at TREM position 66 (C). In an embodiment, the ASGPR binding moiety is bound to the phosphate backbone at TREM position 65 (G).

The ASGPR binding moiety may be bound to any nucleotide position within a domain (ASt Domain1, DH Domain, ACH Domain, VL Domain, TH Domain, and/or ASt Domain2) of a TREM. In an embodiment, the ASGPR moiety is bound to a nucleobase, terminus, or internucleotide linkage within a TREM. In an embodiment, the ASGPR binding moiety is bound to a nucleobase within a TREM. In an embodiment, the ASGPR binding moiety is bound to any adenine nucleobase within a domain (ASt Domain1, DH Domain, ACH Domain, VL Domain, TH Domain, and/or ASt Domain2) of the TREM. In an embodiment, ASGPR binding moiety is bound to any cytosine nucleobase within a domain (ASt Domain1, DH Domain, ACH Domain, VL Domain, TH Domain, and/or ASt Domain2) of the TREM. In an embodiment, it is bound to any guanosine nucleobase within a domain (ASt Domain1, DH Domain, ACH Domain, VL Domain, TH Domain, and/or ASt Domain2) of the TREM. In an embodiment, it is bound to any uracil nucleobase within a domain (ASt Domain1, DH Domain, ACH Domain, VL Domain, TH Domain, and/or ASt Domain2) of the TREM. In an embodiment, it is bound to any thymine nucleobase within a domain (ASt Domain1, DH Domain, ACH Domain, VL Domain, TH Domain, and/or ASt Domain2) of the TREM.

In an embodiment, the ASGPR binding moiety is present within a TREM at TREM position 1 (e.g., present within a nucleobase at TREM position 1). In an embodiment, the ASGPR binding moiety is present within a TREM at TREM position 2 (e.g., present within a nucleobase at TREM position 2). In an embodiment, the ASGPR binding moiety is present within a TREM at TREM position 3 (e.g., present within a nucleobase at TREM position 3). In an embodiment, the ASGPR binding moiety is present within a TREM at TREM position 4 (e.g., present within a nucleobase at TREM position 4). In an embodiment, the ASGPR binding moiety is present within a TREM at TREM position 5 (e.g., present within a nucleobase at TREM position 5). In an embodiment, the ASGPR binding moiety is present within a TREM at TREM position 6 (e.g., present within a nucleobase at TREM position 6). In an embodiment, the ASGPR binding moiety is present within a TREM at TREM position 7 (e.g., present within a nucleobase at TREM position 7). In an embodiment, the ASGPR binding moiety is present within a TREM at TREM position 8 (e.g., present within a nucleobase at TREM position 8). In an embodiment, the ASGPR binding moiety is present within a TREM at TREM position 9 (e.g., present within a nucleobase at TREM position 9).

In an embodiment, the ASGPR binding moiety is present within a TREM at TREM position 10 (e.g., present within a nucleobase at TREM position 10). In an embodiment, the ASGPR binding moiety is present within a TREM at TREM position 11 (e.g., present within a nucleobase at TREM position 11). In an embodiment, the ASGPR binding moiety is present within a TREM at TREM position 12 (e.g., present within a nucleobase at TREM position 12). In an embodiment, the ASGPR binding moiety is present within a TREM at TREM position 13 (e.g., present within a nucleobase at TREM position 13). In an embodiment, the ASGPR binding moiety is present within a TREM at TREM position 14 (e.g., present within a nucleobase at TREM position 14). In an embodiment, the ASGPR binding moiety is present within a TREM at TREM position 15 (e.g., present within a nucleobase at TREM position 15). In an embodiment, the ASGPR binding moiety is present within a TREM at TREM position 16 (e.g., present within a nucleobase at TREM position 16). In an embodiment, the ASGPR binding moiety is not present within a TREM at TREM position 16. In an embodiment, the ASGPR binding moiety is present within a TREM at TREM position 17 (e.g., present within a nucleobase at TREM position 17). In an embodiment, the ASGPR binding moiety is present within a TREM at TREM position 18 (e.g., present within a nucleobase at TREM position 18). In an embodiment, the ASGPR binding moiety is present within a TREM at TREM position 19 (e.g., present within a nucleobase at TREM position 19). In an embodiment, the ASGPR binding moiety is present within a TREM at TREM position 20 (e.g., present within a nucleobase at TREM position 20). In an embodiment, the ASGPR binding moiety is not present within a TREM at TREM position 20. In an embodiment, the ASGPR binding moiety is present within a TREM at TREM position 21 (e.g., present within a nucleobase at TREM position 21). In an embodiment, the ASGPR binding moiety is present within a TREM at TREM position 22 (e.g., present within a nucleobase at TREM position 22). In an embodiment, the ASGPR binding moiety is present within a TREM at TREM position 23 (e.g., present within a nucleobase at TREM position 23). In an embodiment, the ASGPR binding moiety is present within a TREM at TREM position 24 (e.g., present within a nucleobase at TREM position 24). In an embodiment, the ASGPR binding moiety is present within a TREM at TREM position 25 (e.g., present within a nucleobase at TREM position 25). In an embodiment, the ASGPR binding moiety is present within a TREM at TREM position 26 (e.g., present within a nucleobase at TREM position 26).

In an embodiment, the ASGPR binding moiety is present within a TREM at TREM position 27 (e.g., present within a nucleobase at TREM position 27). In an embodiment, the ASGPR binding moiety is present within a TREM at TREM position 28 (e.g., present within a nucleobase at TREM position 28). In an embodiment, the ASGPR binding moiety is present within a TREM at TREM position 29 (e.g., present within a nucleobase at TREM position 29). In an embodiment, the ASGPR binding moiety is present within a TREM at TREM position 30 (e.g., present within a nucleobase at TREM position 30). In an embodiment, the ASGPR binding moiety is present within a TREM at TREM position 31 (e.g., present within a nucleobase at TREM position 31). In an embodiment, the ASGPR binding moiety is present within a TREM at TREM position 32 (e.g., present within a nucleobase at TREM position 32). In an embodiment, the ASGPR binding moiety is present within a TREM at TREM position 33 (e.g., present within a nucleobase at TREM position 33). In an embodiment, the ASGPR binding moiety is present within a TREM at TREM position 34 (e.g., present within a nucleobase at TREM position 34). In an embodiment, the ASGPR binding moiety is present within a TREM at TREM position 35 (e.g., present within a nucleobase at TREM position 35). In an embodiment, the ASGPR binding moiety is present within a TREM at TREM position 36 (e.g., present within a nucleobase at TREM position 36). In an embodiment, the ASGPR binding moiety is present within a TREM at TREM position 37 (e.g., present within a nucleobase at TREM position 37). In an embodiment, the ASGPR binding moiety is present within a TREM at TREM position 38 (e.g., present within a nucleobase at TREM position 38). In an embodiment, the ASGPR binding moiety is present within a TREM at TREM position 39 (e.g., present within a nucleobase at TREM position 39). In an embodiment, the ASGPR binding moiety is present within a TREM at TREM position 40 (e.g., present within a nucleobase at TREM position 40). In an embodiment, the ASGPR binding moiety is present within a TREM at TREM position 41 (e.g., present within a nucleobase at TREM position 41). In an embodiment, the ASGPR binding moiety is present within a TREM at TREM position 42 (e.g., present within a nucleobase at TREM position 42). In an embodiment, the ASGPR binding moiety is present within a TREM at TREM position 43 (e.g., present within a nucleobase at TREM position 43).

In an embodiment, the ASGPR binding moiety is present within a TREM at TREM position 44 (e.g., present within a nucleobase at TREM position 44). In an embodiment, the ASGPR binding moiety is present within a TREM at TREM position 45 (e.g., present within a nucleobase at TREM position 45). In an embodiment, the ASGPR binding moiety is present within a TREM at TREM position 46 (e.g., present within a nucleobase at TREM position 46). In an embodiment, the ASGPR binding moiety is present within a TREM at TREM position 47 (e.g., present within a nucleobase at TREM position 47). In an embodiment, the ASGPR binding moiety is not present within a TREM at TREM position 47. In an embodiment, the ASGPR binding moiety is present within a TREM at TREM position 48 (e.g., present within a nucleobase at TREM position 48). In an embodiment, the ASGPR binding moiety is present within a TREM at TREM position 49 (e.g., present within a nucleobase at TREM position 49).

In an embodiment, the ASGPR binding moiety is present within a TREM at TREM position 50 (e.g., present within a nucleobase at TREM position 50). In an embodiment, the ASGPR binding moiety is present within a TREM at TREM position 51 (e.g., present within a nucleobase at TREM position 51). In an embodiment, the ASGPR binding moiety is present within a TREM at TREM position 52 (e.g., present within a nucleobase at TREM position 52). In an embodiment, the ASGPR binding moiety is present within a TREM at TREM position 53 (e.g., present within a nucleobase at TREM position 53). In an embodiment, the ASGPR binding moiety is present within a TREM at TREM position 54 (e.g., present within a nucleobase at TREM position 54). In an embodiment, the ASGPR binding moiety is present within a TREM at TREM position 55 (e.g., present within a nucleobase at TREM position 55). In an embodiment, the ASGPR binding moiety is present within a TREM at TREM position 56 (e.g., present within a nucleobase at TREM position 56). In an embodiment, the ASGPR binding moiety is present within a TREM at TREM position 57 (e.g., present within a nucleobase at TREM position 57). In an embodiment, the ASGPR binding moiety is present within a TREM at TREM position 58 (e.g., present within a nucleobase at TREM position 58). In an embodiment, the ASGPR binding moiety is present within a TREM at TREM position 59 (e.g., present within a nucleobase at TREM position 59). In an embodiment, the ASGPR binding moiety is present within a TREM at TREM position 60 (e.g., present within a nucleobase at TREM position 60). In an embodiment, the ASGPR binding moiety is present within a TREM at TREM position 61 (e.g., present within a nucleobase at TREM position 61). In an embodiment, the ASGPR binding moiety is present within a TREM at TREM position 62 (e.g., present within a nucleobase at TREM position 62). In an embodiment, the ASGPR binding moiety is present within a TREM at TREM position 63 (e.g., present within a nucleobase at TREM position 63). In an embodiment, the ASGPR binding moiety is present within a TREM at TREM position 64 (e.g., present within a nucleobase at TREM position 64).

In an embodiment, the ASGPR binding moiety is present within a TREM at TREM position 65 (e.g., present within a nucleobase at TREM position 65). In an embodiment, the ASGPR binding moiety is present within a TREM at TREM position 66 (e.g., present within a nucleobase at TREM position 66). In an embodiment, the ASGPR binding moiety is present within a TREM at TREM position 67 (e.g., present within a nucleobase at TREM position 67). In an embodiment, the ASGPR binding moiety is present within a TREM at TREM position 68 (e.g., present within a nucleobase at TREM position 68). In an embodiment, the ASGPR binding moiety is present within a TREM at TREM position 69 (e.g., present within a nucleobase at TREM position 69). In an embodiment, the ASGPR binding moiety is present within a TREM at TREM position 70 (e.g., present within a nucleobase at TREM position 70). In an embodiment, the ASGPR binding moiety is present within a TREM at TREM position 71 (e.g., present within a nucleobase at TREM position 71). In an embodiment, the ASGPR binding moiety is present within a TREM at TREM position 72 (e.g., present within a nucleobase at TREM position 72). In an embodiment, the ASGPR binding moiety is present within a TREM at TREM position 73 (e.g., present within a nucleobase at TREM position 73). In an embodiment, the ASGPR binding moiety is present within a TREM at TREM position 74 (e.g., present within a nucleobase at TREM position 74). In an embodiment, the ASGPR binding moiety is present within a TREM at TREM position 75 (e.g., present within a nucleobase at TREM position 75). In an embodiment, the ASGPR binding moiety is present within a TREM at TREM position 76 (e.g., present within a nucleobase at TREM position 76).

In an embodiment, the ASGPR binding moiety is bound to a nucleobase at TREM position 1 (G). In an embodiment, the ASGPR binding moiety is bound to a nucleobase at TREM position 2 (G). In an embodiment, the ASGPR binding moiety is bound to a nucleobase at TREM position 3 (C). In an embodiment, the ASGPR binding moiety is bound to a nucleobase at TREM position 4 (U). In an embodiment, the ASGPR binding moiety is bound to a nucleobase at TREM position 5 (C). In an embodiment, the ASGPR binding moiety is bound to a nucleobase at TREM position 6 (C). In an embodiment, the ASGPR binding moiety is bound to a nucleobase at TREM position 7 (G). In an embodiment, the ASGPR binding moiety is bound to a nucleobase at TREM position 8 (U). In an embodiment, the ASGPR binding moiety is bound to a nucleobase at TREM position 9 (G).

In an embodiment, the ASGPR binding moiety is bound to a nucleobase at TREM position 10 (G). In an embodiment, the ASGPR binding moiety is bound to a nucleobase at TREM position 11 (C). In an embodiment, the ASGPR binding moiety is bound to a nucleobase at TREM position 12 (G). In an embodiment, the ASGPR binding moiety is bound to a nucleobase at TREM position 13 (C). In an embodiment, the ASGPR binding moiety is bound to a nucleobase at TREM position 14 (A). In an embodiment, the ASGPR binding moiety is bound to a nucleobase at TREM position 15 (A). In an embodiment, the ASGPR binding moiety is bound to a nucleobase at TREM position 16 (U). In an embodiment, the ASGPR binding moiety is bound to a nucleobase at TREM position 17 (G). In an embodiment, the ASGPR binding moiety is bound to a nucleobase at TREM position 18 (G). In an embodiment, the ASGPR binding moiety is bound to a nucleobase at TREM position 19 (A). In an embodiment, the ASGPR binding moiety is bound to a nucleobase at TREM position 20 (U). In an embodiment, the ASGPR binding moiety is bound to a nucleobase at TREM position 21 (A). In an embodiment, the ASGPR binding moiety is bound to a nucleobase at TREM position 22 (G). In an embodiment, the ASGPR binding moiety is bound to a nucleobase at TREM position 23 (C). In an embodiment, the ASGPR binding moiety is bound to a nucleobase at TREM position 24 (G). In an embodiment, the ASGPR binding moiety is bound to a nucleobase at TREM position 25 (C). In an embodiment, the ASGPR binding moiety is bound to a nucleobase at TREM position 26 (A).

In an embodiment, the ASGPR binding moiety is bound to a nucleobase at TREM position 27 (U). In an embodiment, the ASGPR binding moiety is bound to a nucleobase at TREM position 28 (U). In an embodiment, the ASGPR binding moiety is bound to a nucleobase at TREM position 29 (G). In an embodiment, the ASGPR binding moiety is bound to a nucleobase at TREM position 30 (G). In an embodiment, the ASGPR binding moiety is bound to a nucleobase at TREM position 31 (A). In an embodiment, the ASGPR binding moiety is bound to a nucleobase at TREM position 32 (C). In an embodiment, the ASGPR binding moiety is bound to a nucleobase at TREM position 33 (U). In an embodiment, the ASGPR binding moiety is bound to a nucleobase at TREM position 34 (U). In an embodiment, the ASGPR binding moiety is bound to a nucleobase at TREM position 35 (C). In an embodiment, the ASGPR binding moiety is bound to a nucleobase at TREM position 36 (A). In an embodiment, the ASGPR binding moiety is bound to a nucleobase at TREM position 37 (A). In an embodiment, the ASGPR binding moiety is bound to a nucleobase at TREM position 38 (A). In an embodiment, the ASGPR binding moiety is bound to a nucleobase at TREM position 39 (U). In an embodiment, the ASGPR binding moiety is bound to a nucleobase at TREM position 40 (U). In an embodiment, the ASGPR binding moiety is bound to a nucleobase at TREM position 41 (C). In an embodiment, the ASGPR binding moiety is bound to a nucleobase at TREM position 42 (A). In an embodiment, the ASGPR binding moiety is bound to a nucleobase at TREM position 43 (A).

In an embodiment, the ASGPR binding moiety is bound to a nucleobase at TREM position 44 (A). In an embodiment, the ASGPR binding moiety is bound to a nucleobase at TREM position 45 (G). In an embodiment, the ASGPR binding moiety is bound to a nucleobase at TREM position 46 (G). In an embodiment, the ASGPR binding moiety is bound to a nucleobase at TREM position 47 (U). In an embodiment, the ASGPR binding moiety is bound to a nucleobase at TREM position 48 (U). In an embodiment, the ASGPR binding moiety is bound to a nucleobase at TREM position 49 (C)

In an embodiment, the ASGPR binding moiety is bound to a nucleobase at TREM position 50 (C). In an embodiment, the ASGPR binding moiety is bound to a nucleobase at TREM position 51 (G). In an embodiment, the ASGPR binding moiety is bound to a nucleobase at TREM position 52 (G). In an embodiment, the ASGPR binding moiety is bound to a nucleobase at TREM position 53 (G). In an embodiment, the ASGPR binding moiety is bound to a nucleobase at TREM position 54 (U). In an embodiment, the ASGPR binding moiety is bound to a nucleobase at TREM position 55 (U). In an embodiment, the ASGPR binding moiety is bound to a nucleobase at TREM position 56 (C). In an embodiment, the ASGPR binding moiety is bound to a nucleobase at TREM position 57 (G). In an embodiment, the ASGPR binding moiety is bound to a nucleobase at TREM position 58 (A). In an embodiment, the ASGPR binding moiety is bound to a nucleobase at TREM position 59 (G). In an embodiment, the ASGPR binding moiety is bound to a nucleobase at TREM position 60 (U). In an embodiment, the ASGPR binding moiety is bound to a nucleobase at TREM position 61 (C). In an embodiment, the ASGPR binding moiety is bound to a nucleobase at TREM position 62 (C). In an embodiment, the ASGPR binding moiety is bound to a nucleobase at TREM position 63 (C). In an embodiment, the ASGPR binding moiety is bound to a nucleobase at TREM position 64 (G).

In an embodiment, the ASGPR binding moiety is bound to a nucleobase at TREM position 76 (A). In an embodiment, the ASGPR binding moiety is bound to a nucleobase at TREM position 75 (C). In an embodiment, the ASGPR binding moiety is bound to a nucleobase at TREM position 74 (C). In an embodiment, the ASGPR binding moiety is bound to a nucleobase at TREM position 73 (G). In an embodiment, the ASGPR binding moiety is bound to a nucleobase at TREM position 72 (C). In an embodiment, the ASGPR binding moiety is bound to a nucleobase at TREM position 71 (U). In an embodiment, the ASGPR binding moiety is bound to a nucleobase at TREM position 70 (G). In an embodiment, the ASGPR binding moiety is bound to a nucleobase at TREM position 69 (A). In an embodiment, the ASGPR binding moiety is bound to a nucleobase at TREM position 68 (G). In an embodiment, the ASGPR binding moiety is bound to a nucleobase at TREM position 67 (G). In an embodiment, the ASGPR binding moiety is bound to a nucleobase at TREM position 66 (C). In an embodiment, the ASGPR binding moiety is bound to a nucleobase at TREM position 65 (G).

In an embodiment, the TREM comprising an ASGPR binding moiety comprises a ribonucleic acid (RNA) sequence encoded by a deoxyribonucleic acid (DNA) sequence disclosed in Table 1, e.g., any one of SEQ ID NOs: 1-451 disclosed in Table 1. In an embodiment the TREM comprising an ASGPR binding moiety comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, the TREM comprising an ASGPR binding moiety comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, the TREM comprising an ASGPR binding moiety comprises at least 5, 10, 15, 20, 25, or 30 consecutive nucleotides of an RNA sequence encoded by a DNA sequence disclosed in Table 1, e.g., at least 5, 10, 15, 20, 25, or 30 consecutive nucleotides of an RNA sequence encoded by any one of SEQ ID NOs: 1-451 disclosed in Table 1. In an embodiment, the TREM comprising an ASGPR binding moiety comprises at least 5, 10, 15, 20, 25, or 30 consecutive nucleotides of an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, the TREM comprising an ASGPR binding moiety comprises at least 5, 10, 15, 20, 25, or 30 consecutive nucleotides of an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 1, e.g., any one of SEQ ID NOs: 1-451 disclosed in Table 1.

In an embodiment, the TREM comprising an ASGPR binding moiety comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence provided in Table 1, e.g., any one of SEQ ID NOs: 1-451 disclosed in Table 1. In an embodiment, the TREM comprising an ASGPR binding moiety comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence at least 80%, 85%, 90%, 95%, 96%, 97%, 98%, or 99% identical to an RNA sequence encoded by a DNA sequence provided in Table 1, e.g., any one of SEQ ID NOs: 1-451 disclosed in Table 1. In an embodiment, the TREM comprising an ASGPR binding moiety comprises at least 5%, 10%, 15%, 20%, 25%, 30%, 35%, 40%, 50%, 55%, 60%, 65%, 70%, 75%, 80%, 85%, 90%, 95%, 96%, 97%, 98% or 99% of an RNA sequence encoded by a DNA sequence at least 80%, 85%, 90%, 95%, 96%, 97%, 98%, or 99% identical to a DNA sequence provided in Table 1, e.g., any one of SEQ ID NOs: 1-451 disclosed in Table 1.

In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence disclosed in Table 1, e.g., any one of SEQ ID NOs: 1-451 disclosed in Table 1. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 1, e.g., any one of SEQ ID NOs: 1-451 disclosed in Table 1. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 1, e.g., any one of SEQ ID NOs: 1-451 disclosed in Table 1.

In an embodiment, the TREM comprising an ASGPR binding moiety comprises a ribonucleic acid (RNA) sequence encoded by a deoxyribonucleic acid (DNA) sequence disclosed in Table 4, e.g., any one of SEQ ID NOs: 452-561 disclosed in Table 4. In an embodiment the TREM comprising an ASGPR binding moiety comprises an RNA sequence at least 60%, 65%, 70%, 75%, 80%, 82%, 85%, 87%, 88%, 90%, 92%, 95%, 96%, 97%, 98%, or 99% identical to an RNA sequence encoded by a DNA sequence provided in Table 4, e.g., any one of SEQ ID NOs: 452-561 disclosed in Table 4. In an embodiment, the TREM comprising an ASGPR binding moiety comprises an RNA sequence encoded by a DNA sequence at least 60%, 65%, 70%, 75%, 80%, 82%, 85%, 87%, 88%, 90%, 92%, 95%, 96%, 97%, 98%, or 99% identical to a DNA sequence provided in Table 4, e.g., any one of SEQ ID NOs: 452-561 disclosed in Table 4.

In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence provided in Table 4, e.g., any one of SEQ ID NOs: 452-561 disclosed in Table 4. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence which is at least 80%, 85%, 90%, 95%, 96%, 97%, 98%, 99% or 100% identical to an RNA sequence encoded by a DNA sequence provided in Table 4, e.g., any one of SEQ ID NOS: 452-561 disclosed in Table 4. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of an RNA sequence encoded by a DNA sequence with at least 80%, 82%, 85%, 87%, 88%, 90%, 92%, 95%, 96%, 97%, 98%, 99% or 100% identity to a DNA sequence provided in Table 4, e.g., any one of SEQ ID NOs: 452-561 disclosed in Table 4.

In an embodiment, the TREM comprising an ASGPR binding moiety comprises a sequence provided in Table 12, e.g., any one of SEQ ID NOs: 622-1116. In an embodiment, the TREM comprising an ASGPR binding moiety comprises SEQ ID NO. 622. In an embodiment, the TREM comprising an ASGPR binding moiety comprises SEQ ID NO. 623. In an embodiment, the TREM comprising an ASGPR binding moiety comprises SEQ ID NO. 624. In an embodiment, the TREM comprising an ASGPR binding moiety comprises SEQ ID NO. 625. In an embodiment, the TREM comprising an ASGPR binding moiety comprises SEQ ID NO. 626. In an embodiment, the TREM comprising an ASGPR binding moiety comprises SEQ ID NO. 627. In an embodiment, the TREM comprising an ASGPR binding moiety comprises SEQ ID NO. 628. In an embodiment, the TREM comprising an ASGPR binding moiety comprises SEQ ID NO. 629. In an embodiment, the TREM comprising an ASGPR binding moiety comprises SEQ ID NO. 630. In an embodiment, the TREM comprising an ASGPR binding moiety comprises SEQ ID NO. 631. In an embodiment, the TREM comprising an ASGPR binding moiety comprises SEQ ID NO. 632. In an embodiment, the TREM comprising an ASGPR binding moiety comprises SEQ ID NO. 633. In an embodiment, the TREM comprising an ASGPR binding moiety comprises SEQ ID NO. 634. In an embodiment, the TREM comprising an ASGPR binding moiety comprises SEQ ID NO. 635. In an embodiment, the TREM comprising an ASGPR binding moiety comprises SEQ ID NO. 636. In an embodiment, the TREM comprising an ASGPR binding moiety comprises SEQ ID NO. 637. In an embodiment, the TREM comprising an ASGPR binding moiety comprises SEQ ID NO. 638. In an embodiment, the TREM comprising an ASGPR binding moiety comprises SEQ ID NO. 639. In an embodiment, the TREM comprising an ASGPR binding moiety comprises SEQ ID NO. 640. In an embodiment, the TREM comprising an ASGPR binding moiety comprises SEQ ID NO. 641. In an embodiment, the TREM comprising an ASGPR binding moiety comprises SEQ ID NO. 642. In an embodiment, the TREM comprising an ASGPR binding moiety comprises SEQ ID NO. 643. In an embodiment, the TREM comprising an ASGPR binding moiety comprises SEQ ID NO. 644. In an embodiment, the TREM comprising an ASGPR binding moiety comprises SEQ ID NO. 645. In an embodiment, the TREM comprising an ASGPR binding moiety comprises SEQ ID NO. 646. In an embodiment, the TREM comprising an ASGPR binding moiety comprises SEQ ID NO. 647. In an embodiment, the TREM comprising an ASGPR binding moiety comprises SEQ ID NO. 648. In an embodiment, the TREM comprising an ASGPR binding moiety comprises SEQ ID NO. 649. In an embodiment, the TREM comprising an ASGPR binding moiety comprises SEQ ID NO. 650. In an embodiment, the TREM comprising an ASGPR binding moiety comprises SEQ ID NO. 651. In an embodiment, the TREM comprising an ASGPR binding moiety comprises SEQ ID NO. 652. In an embodiment, the TREM comprising an ASGPR binding moiety comprises SEQ ID NO. 653. In an embodiment, the TREM comprising an ASGPR binding moiety comprises SEQ ID NO. 654. In an embodiment, the TREM comprising an ASGPR binding moiety comprises a TREM selected from any one of SEQ ID NO. 622-1116. In an embodiment, the TREM comprising an ASGPR binding moiety comprises a TREM selected from any one of SEQ ID NO. 655-786. In an embodiment, the TREM comprising an ASGPR binding moiety comprises a TREM selected from any one of SEQ ID NO. 787-896. In an embodiment, the TREM comprising an ASGPR binding moiety comprises a TREM selected from any one of SEQ ID NO. 897-1006. In an embodiment, the TREM comprising an ASGPR binding moiety comprises a TREM selected from any one of SEQ ID NO. 1007-1116.

In an embodiment, the TREM comprising an ASGPR binding moiety comprises a sequence that is at least 60%, 65%, 70%, 75%, 80%, 82%, 85%, 87%, 88%, 90%, 92%, 95%, 96%, 97%, 98%, or 99% identical to a sequence of a TREM provided in Table 12, e.g., any one of SEQ ID NOs. 622-1116 provided in Table 12. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of a TREM provided in Table 12, e.g., any one of SEQ ID NOs. 622-1116 disclosed in Table 12. In an embodiment, the TREM comprising an ASGPR binding moiety comprises at least 5 ribonucleotides (nt), 10 nt, 15 nt, 20 nt, 25 nt, 30 nt, 35 nt, 40 nt, 45 nt, 50 nt, 55 nt or 60 nt (but less than the full length) of a TREM which is at least 80%, 85%, 90%, 95%, 96%, 97%, 98%, 99% or 100% identical to TREM provided in Table 12, e.g., any one of SEQ ID NOs. 622-1116 disclosed in Table 12.

In an embodiment, the TREM comprising an ASGPR binding moiety comprises a sequence that differs no more than 1 ribonucleotide (nt), 2 nt, 3 nt, 4 nt, 5 nt, 6 nt, 7 nt, 8 nt, 9 nt, 10 nt, 12 nt, 14 nt, 16 nt, 18, nt, or 20 nt from a TREM provided in Table 12, e.g., any one of SEQ ID NOs. 622-1116 provided in Table 12.

In an embodiment, the TREM comprising an ASGPR binding moiety is at least 60%, 65%, 70%, 75%, 80%, 82%, 85%, 87%, 88%, 90%, 92%, 95%, 96%, 97%, 98%, or 99% identical to SEQ ID NO. 622. In an embodiment, the TREM comprising an ASGPR binding moiety is at least 60%, 65%, 70%, 75%, 80%, 82%, 85%, 87%, 88%, 90%, 92%, 95%, 96%, 97%, 98%, or 99% identical to SEQ ID NO. 650. In an embodiment, the TREM comprising an ASGPR binding moiety is at least 60%, 65%, 70%, 75%, 80%, 82%, 85%, 87%, 88%, 90%, 92%, 95%, 96%, 97%, 98%, or 99% identical to SEQ ID NO. 653.

In an embodiment, the TREM comprising an ASGPR binding moiety comprises a sequence that differs comprises by least 1 ribonucleotide (nt), 2 nt, 3 nt, 4 nt, 5 nt, 6 nt, 7 nt, 8 nt, 9 nt, 10 nt, 12 nt, 14 nt, 16 nt, 18 nt, 20 nt, 25 nt, 30 nt, 40 nt, 45 nt, 50 nt, 55 nt, or more from SEQ ID NO. 622. In an embodiment, the TREM comprising an ASGPR binding moiety comprises a sequence that differs no more than 1 ribonucleotide (nt), 2 nt, 3 nt, 4 nt, 5 nt, 6 nt, 7 nt, 8 nt, 9 nt, 10 nt, 12 nt, 14 nt, 16 nt, 18, nt, or 20 nt from SEQ ID NO. 622. In an embodiment, the TREM comprising an ASGPR binding moiety comprises a sequence that differs comprises by least 1 ribonucleotide (nt), 2 nt, 3 nt, 4 nt, 5 nt, 6 nt, 7 nt, 8 nt, 9 nt, 10 nt, 12 nt, 14 nt, 16 nt, 18 nt, 20 nt, 25 nt, 30 nt, 40 nt, 45 nt, 50 nt, 55 nt, or more from SEQ ID NO. 650. In an embodiment, the TREM comprising an ASGPR binding moiety comprises a sequence that differs no more than 1 ribonucleotide (nt), 2 nt, 3 nt, 4 nt, 5 nt, 6 nt, 7 nt, 8 nt, 9 nt, 10 nt, 12 nt, 14 nt, 16 nt, 18, nt, or 20 nt from SEQ ID NO. 650. In an embodiment, the TREM comprising an ASGPR binding moiety comprises a sequence that differs comprises by least 1 ribonucleotide (nt), 2 nt, 3 nt, 4 nt, 5 nt, 6 nt, 7 nt, 8 nt, 9 nt, 10 nt, 12 nt, 14 nt, 16 nt, 18 nt, 20 nt, 25 nt, 30 nt, 40 nt, 45 nt, 50 nt, 55 nt, or more from SEQ ID NO. 653. In an embodiment, the TREM comprising an ASGPR binding moiety comprises a sequence that differs no more than 1 ribonucleotide (nt), 2 nt, 3 nt, 4 nt, 5 nt, 6 nt, 7 nt, 8 nt, 9 nt, 10 nt, 12 nt, 14 nt, 16 nt, 18, nt, or 20 nt from SEQ ID NO. 653.

### Non-Naturally Occurring Modifications

A TREM, a TREM core fragment or a TREM fragment described herein may comprise a non-naturally occurring modification, e.g., a modification described in Table 5. A non-naturally occurring modification can be made according to methods known in the art. In an embodiment, a non-naturally occurring modification is a modification that a cell, e.g., a human cell, does not make on an endogenous tRNA. In an embodiment, a non-naturally occurring modification is a modification that a cell, e.g., a human cell, can make on an endogenous tRNA, but wherein such modification is in a location in which it does not occur on a native tRNA. In an embodiment, the non-naturally occurring modification is in a domain, linker or arm which does not have such modification in nature. In an embodiment, the non-naturally occurring modification is at a position within a domain, linker or arm, which does not have such modification in nature. In an embodiment, the non-naturally occurring modification is on a nucleotide which does not have such modification in nature. In an embodiment, the non-naturally occurring modification is on a nucleotide at a position within a domain, linker or arm, which does not have such modification in nature.

In an embodiment, a TREM, a TREM core fragment or a TREM fragment described herein comprises a modification provided in Table 5, or a combination thereof. The modifications provided in Table 5 are non-naturally occurring or occur naturally in RNAs, and are used herein on a synthetic TREM, a TREM core fragment or a TREM fragment at a position that does not occur in nature.

TABLE 5
Exemplary modifications

Chemical Modification

| | |
|---|---|
| (S)-constrained ethyl (cEt) | 5-(methoxycarbonyl-methyl)uracil |
| (+)1-(2-Hydroxypropyl)pseudouridine | 5-(methyl)2(thio)uracil |
| (2R)-1-(2-Hydroxypropyl)pseudouridine | 5-(methyl)2,4(dithio)uracil |
| (2S)-1-(2-Hydroxypropyl)pseudouridine | 5-(methyl)4(thio)uracil |
| (3-(3-amino-3-carboxypropyl)uridine | 5-(methyl)-2-(thio)pseudouracil |
| (E)-5-(2-Bromo-vinyl)ara-uridine | 5-(methyl)-2-(thio)uracil |
| (E)-5-(2-Bromo-vinyl)cytidine | 5-(methyl)-2,4(dithio)pseudouracil |
| (E)-5-(2-Bromo-vinyl)uridine | 5-(methyl)-2,4-(dithio)uracil |
| (E)-vinylphosphonate | 5-(methyl)-4(thio)pseudouracil |
| (R)5′-C-methyl | 5-(methyl)isocarbostyrilyl |
| (R)5′-C-methyl with phosphate | 5-(methyl)pseudouracil |
| (S)5′-C-methyl | 5-(methylaminomethyl)-2(thio)uracil |
| (S)5′-C-methyl with phosphate | 5-(methylaminomethyl)-2,4(dithio)uracil |
| (Z)-5-(2-Bromo-vinyl)ara-uridine | 5-(methylaminomethyl)-4-(thio)uracil |
| (Z)-5-(2-Bromo-vinyl)uridine | 5-(propynyl)uracil |
| 1(4-Nitro-phenyl)pseudouridine | 5-(propynyl)cytosine |
| 1-(aminocarbonylethylenyl)-2(thio)- | 5-(trifluoromethyl)cytosine |
| pseudouracil | |
| 1-(aminocarbonylethylenyl)-2,4- | 5-(trifluoromethyl)uracil |
| (dithio)pseudouracil | |
| 1-(2,2,2-Trifluoroethyl)-pseudouridine | 5,2′-O-dimethylcytidine |
| 1-(2,2,3,3,3-Pentafluoropropyl)pseudouridine | 5,2′-O-dimethyluridine |
| 1-(2,2-Diethoxyethyl)pseudouridine | 5,6-dihydro-uridine |
| 1-(2,4,6-Trimethylbenzyl)pseudouridine | 5-Aminoallyl-cytosine |
| 1-(2,4,6-Trimethyl-benzyl)pseudo-uridine | 5-aminoallyl-uridine |
| 1-(2,4,6-Trimethyl-phenyl)pseudo-uridine | 5-aminomethyl-2-thiouridine |
| 1-(2-Amino-2-carboxyethyl)pseudo-uridine | 5-aza-2-thio-zebularine |
| 1-(2-Amino-ethyl)pseudouridine | 5-aza-cytidine |
| 1-(2-Hydroxyethyl)pseudouridine | 5-aza-uridine |
| 1-(2-Methoxyethyl)pseudouridine | 5-aza-zebularine |
| 1-(3,4-Bis- | 5-bromo-cytidine |
| trifluoromethoxvbenzyl)pseudouridine | |
| 1-(3,4-Dimethoxybenzyl)pseudouridine | 5-bromo-uridine |
| 1-(3-Amino-3-carboxypropyl)pseudo-uridine | 5-carbamoylmethyl-2′-O-methyluridine |
| 1-(3-Amino-propyl)pseudouridine | 5-carbamoylmethyluridine |
| 1-(3-Cyclopropyl-prop-2-ynyl)pseudouridine | 5-carboxyhydroxymethyluridine |
| TP | |
| 1-(4-Amino-4-carboxybutyl)pseudouridine | 5-carboxyhydroxymethyluridine methyl ester |
| 1-(4-Amino-benzyl)pseudouridine | 5-carboxymethylaminomethyl-2-thiouridine |
| 1-(4-Amino-butyl)pseudouridine | 5-carboxymethylaminomethyl-2′-O- |
| | methyluridine |
| 1-(4-Amino-phenyl)pseudouridine | 5-carboxymethylaminomethyl-2-thiouridine |
| 1-(4-Azidobenzyl)pseudouridine | 5-carboxymethylaminomethyluridine |
| 1-(4-Bromobenzyl)pseudouridine | 5-carboxymethyluridine |
| 1-(4-Chlorobenzyl)pscudouridinc | 5-Cyanocytidinc |
| 1-(4-Fluorobenzyl)pseudouridine | 5-Cyanouridine |
| 1-(4-Iodobenzyl)pseudouridine | 5-Dimethylaminouridine |
| 1-(4-Methanesulfonylbenzyl)pseudouridine | 5-Ethynylara-cytidine |
| 1-(4-Methoxybenzyl)pseudouridine | 5-Ethynylcytidine |
| 1-(4-Methoxy-phenyl)pseudouridine | 5-formyl-2′-O-methylcytidine |
| 1-(4-Methylbenzyl)pseudouridine | 5-formylcytidine |
| 1-(4-Nitrobenzyl)pseudouridine | 5′-Homo-adenosine |
| 1-(4-Thiomethoxybenzyl)pseudouridine | 5′-Homo-cytidine |
| 1-(4-Trifluoromethoxybenzyl)pseudouridine | 5′-Homo-guanosine |
| 1-(4-Trifluoromethylbenzyl)pseudouridine | 5′-Homo-uridine |
| 1-(5-Amino-pentyl)pseudouridine | 5-hydroxymethylcytidine |
| 1-(6-Amino-hexyl)pseudouridine | 5-hydroxyuridine |
| 1-(aminoalkylamino-carbonylethylenyl)- | 5-iodo-2′-fluoro-deoxyuridine |
| 2(thio)-pseudouracil | |
| 1-(aminoalkylaminocarbonylethylenyl)-2,4- | 5-iodo-cytidine |
| (dithio)pseudouracil | |
| 1-(aminoalkylaminocarbonylethylenyl)- | 5-iodo-uridine |
| pseudouracil | |
| 1-(aminoalkylaminocarbonylethylenyl)-4- | 5-methoxycarbonylmethyl-2-thiouridine |
| (thio)pseudouracil | |
| 1-(aminocarbonylethylenyl)-4- | 5-methoxycarbonylmethyl-2′-O- |
| (thio)pseudouracil | methyluridine |
| 1-(aminocarbonylethylenyl)-pseudouracil | 5-methoxycarbonylmethyluridine |
| 1-(aza)-2-(thio)-3-(aza)-phenoxazin-1-yl | 5-Methoxycytidine |
| 1,2′-O-dimethyladenosine | 5-methoxyuridine |
| 1,2′-O-dimethylguanosine | 5-methyl-2-thiouridine |
| 1,2′-O-dimethylinosine | 5-methylaminomethyl-2-selenouridine |
| 1,3-(diaza)-2-(oxo)-phenthiazin-1-yl | 5-methylaminomethyl-2-thiouridine |
| 1,3-(diaza)-2-(oxo)-phenoxazin-1-yl | 5-methylaminomethyluridine |
| 1,3,5-(triaza)-2,6-(dioxa)-naphthalene | 5-methylcytidine |
| 1,6-Dimethyl-pseudouridine | 5-Methyldihydrouridine |
| 1-{3-[2-(2-Aminoethoxy)-ethoxy]- | 5-methyluridine |
| propionvl}pseudouridine | |
| 1-Acetylpseudouridine | 5-methyl-zebularine |
| 1-Allylpseudouridine | 5-nitroindole |
| 1-Aminomethyl-pseudo-uridine | 5-Oxyacetic acid-Uridine |
| 1-Benzoylpseudouridine | 5-Oxyacetic acid-methyl ester-Uridin Nl- |
| | methyl-pseudouridine |
| 1-Benzyloxymethylpseudouridine | 5-Phenylethynyluridine |
| 1-Benzyl-pseudo-uridine | 5′-phosphorothioate |
| 1-Biotinylpseudouridine | 5-propynyl cytosine |
| 1-Butyl-pseudo-uridine | 5-propynyl uracil |
| 1-carboxymethyl-pseudouridine | 5-taurinomethyl-2-thiouridine |
| 1-Cyanomethylpseudouridine | 5-taurinomethyluridine |
| 1-Cyclobutylmethyl-pseudo-uridine | 5-Trideuteromethyl-6-deuterouridine |
| 1-Cyclobutyl-pseudo-uridine | 5-Trifluoromethyl-Cytidine |
| 1-Cycloheptylmethyl-pseudo-uridine | 5-Trifluoromethyl-Uridine |
| 1-Cycloheptyl-pseudo-uridine | 5-uracil |
| 1-Cyclohexylmethyl-pseudo-uridine | 5-Vinylarauridine |
| 1-Cyclohexyl-pseudo-uridine | 6(azo)uracil |
| 1-Cyclooctylmethyl-pseudo-uridine | 6-(2,2,2-Trifluoroethyl)-pseudo-uridine |
| 1-Cyclooctyl-pseudo-uridine | 6-(4-Morpholino)-pseudo-uridine |
| 1-Cyclopentylmethyl-pseudo-uridine | 6-(4-Thiomorpholino)-pseudo-uridine |
| 1-Cyclopentyl-pseudo-uridine | 6-(alkyl)guanine |
| 1-Cyclopropylmethyl-pseudo-uridine | 6-(alkyl)adenine |
| 1-Cyclopropyl-pseudo-uridine | 6-(aza)pyrimidine |
| 1-deazaadenosine | 6-(azo)cytosine |
| 1-Ethyl-pseudo-uridine | 6-(azo)thymine |
| 1-Hexyl-pseudo-uridine | 6-(azo)uracil |
| 1-Homoallylpseudouridine | 6-(methyl)-7-(aza)indolyl |
| 1-Hydroxymethylpseudouridine | 6-(methyl)adenine |
| 1-iso-propyl-pseudo-uridine | 6-(methyl)guanine |
| 1-Me-2-thio-pseudo-uridine | 6-(Substituted-Phenyl)-pseudo-uridine |
| 1-Me-4-thio-pseudo-uridine | 6-Amino-pseudo-uridine |
| 1-Me-alpha-thio-pseudo-uridine | 6-aza-cytidine |
| 1-Me-guanosine | 6-aza-uridine |
| 1-Methanesulfonylmethylpseudouridine | 6-Azido-pseudo-uridine |
| 1-Methoxymethylpseudouridine | 6-Bromo-pseudo-uridine |
| 1-Methyl-6-amino-pseudo-uridine | 6-Butyl-pseudo-uridine |
| 1-Methyl-6-bromo-pseudo-uridine | 6-Chloro-pseudo-uridine |
| 1-Methyl-6-cyano-pseudo-uridine | 6-chloro-purine |
| 1-Methyl-6-hydroxyamino-pseudo-uridine | 6-Cyano-pseudo-uridine |
| 1-Methyl-6-trifluoromethoxy-pseudo-uridine | 6-Dimethylamino-pseudo-uridine |
| 1-methyladenosine | 6-Ethoxy-pseudo-uridine |
| 1-methylguanosine | 6-Ethylcarboxylate-pseudo-uridine |
| 1-methylinosine | 6-Ethyl-pseudo-uridine |
| 1-methylpseduouridine | 6-Fluoro-pseudo-uridine |
| 1-methyl-pseudoisocytidine | 6-Formyl-pseudo-uridine |
| 1-methyl-pseudouridine | 6-Hydroxyamino-pseudo-uridine |
| 1-Methyl-pseudo-UTP | 6-Hydroxy-pseudo-uridine |
| 1-Morpholinomethylpseudouridine | 6-Iodo-pseudo-uridine |
| 1-Pentyl-pseudo-uridine | 6-iso-Propyl-pseudo-uridine |
| 1-Phenyl-pseudo-uridine | 6-methoxy-guanosine |
| 1-Pivaloylpseudouridine | 6-Methoxy-pseudo-uridine |
| 1-Propargylpseudouridine | 6-Methylamino-pseudo-uridine |
| 1-Propyl-pseudo-uridine | 6-methyl-guanosine |
| 1-propynyl-pseudouridine | 6-Methyl-pseudo-uridine |
| 1-propynyl-uridine | 6-Phenyl-pseudo-uridine |
| 1-p-tolyl-pseudo-uridine | 6-phenyl-pyrrolo-pyrimidin-2-on-3-yl |
| 1-substituted 2-(thio)-pseudouracil | 6-Propyl-pseudo-uridine |
| 1-substituted 2,4-(dithio)pseudouracil | 6-tert-Butyl-pseudo-uridine |
| 1-substituted 4-(thio)pseudouracil | 6-thio-7-deaza-8-aza-guanosine |
| 1-substituted pseudouracil | 6-thio-7-deaza-guanosine |
| 1-taurinomethyl-pseudouridine | 6-thio-7-methyl-guanosine |
| 1-tert-Butyl-pseudo-uridine | 6-thio-guanosine |
| 1-Thiomethoxymethylpseudouridine | 6-Trifluoromethoxy-pseudo-uridine |
| 1-Thiomorpholinomethylpseudouridine | 6-Trifluoromethyl-pseudo-uridine |
| 1-Trifluoroacetylpseudouridine | 7-(alkyl)guanine |
| 1-Trifluoromethylpseudouridine | 7-(aminoalkylhydroxy)-1-(aza)-2-(thio)-3- |
| | (aza)-phenthiazin-1-yl |
| 1-Vinylpseudouridine | 7-(aminoalkylhydroxy)-1-(aza)-2-(thio)-3- |
| | (aza)-phenoxazin-1-yl |
| 2-(amino)purine | 7-(aminoalkylhydroxy)-1,3-(diaza)-2-(oxo)- |
| | phenthiazin-1-yl |
| 2-(thio)pseudouracil | 7-(aminoalkylhydroxy)-1,3-(diaza)-2-(oxo)- |
| | phenoxazin-1-yl |
| 2′-alpha-Ethynylcytidinc | 7-(aza)indolyl |
| 2′-alpha-Ethynylguanosine | 7-(deaza)adenine |
| 2′-alpha-Ethynyluridine | 7-(deaza)guanine |
| 2′-alpha-Trifluoromethyladenosine | 7-(guanidiniumalkylhydroxy)-1-(aza)-2- |
| | (thio)-3-(aza)-phenoxazinl-yl |
| 2′-alpha-Trifluoromethylguanosine | 7-(guanidiniumalkylhydroxy)-1-(aza)-2- |
| | (thio)-3-(aza)-phenthiazin-1-yl |
| 2′-alpha-Trifluoromethyluridine | 7-(guanidiniumalkylhydroxy)-1-(aza)-2- |
| | (thio)-3-(aza)-phenoxazin-1-yl |
| 2′-Amino-2′-deoxycytosine | 7-(guanidiniumalkyl-hydroxy)-1,3-(diaza)-2- |
| | (oxo)-phenthiazin-1-yl |
| 2′-amino-2′-deoxyribose | 7-(guanidiniumalkylhydroxy)-1,3-(diaza)-2- |
| | (oxo)-phenoxazin-1-yl |
| 2′-alpha-Trifluoromethylcytidine | 7-(methyl)guanine |
| 2′-Azido-2′-deoxycytosine | 7-(propynyl)isocarbostyrilyl |
| 2′-azido-2′-deoxyribose | 7-(propynyl)isocarbostyrilyl |
| 2′-Azido-deoxyuridine | 7-propynyl(aza)indolyl |
| 2′-beta-Ethynyladenosine | 7-aminomethyl-7-deazaguanosine |
| 2′-beta-Ethynylguanosine | 7-cyano-7-deazaguanosine |
| 2′-beta-Ethynyluridine | 7-deaza-2-aminopurine |
| 2′-beta-Trifluoromethyluridine | 7-deaza-2,6-diaminopurine |
| 2′-beta-Ethynylcytidine | 7-deaza-2-amino-purine |
| 2′-bromo-deoxyuridine | 7-deaza-8-aza-2,6-diaminopurine |
| 2′-deoxyuridine | 7-deaza-8-aza-2-aminopurine |
| 2′-Deoxy-2′,2′-difluoroadenosine | 7-deaza-8-aza-adenine |
| 2′-Deoxy-2′,2′-difluorocytidine | 7-deaza-8-aza-adenosine |
| 2′-Deoxy-2′,2′-difluoroguanosine | 7-deaza-8-aza-guanosine |
| 2′-Deoxy-2′,2′-difluorouridine | 7-deaza-adenosine |
| 2′-Deoxy-2′-alpha-aminocytidine | 7-deaza-guanosine |
| 2′-Deoxy-2′-alpha-aminouridine TP | 7-deaza-inosinyl |
| 2′-Deoxy-2′-alpha-azidocytidine | 7-methyl-8-oxo-guanosine |
| 2′-Deoxy-2′-alpha-azidouridine TP | 7-methyladenine |
| 2′-Deoxy-2′-alpha-mercaptoadenosine | 7-methylguanosine |
| 2′-Deoxy-2′-alpha-mercaptocytidine | 7-methylinosine |
| 2′-Deoxy-2′-alpha-mercaptoguanosine | 7-substituted 1-(aza)-2-(thio)-3-(aza)- |
| | phenoxazin-1-yl |
| 2′-Deoxy-2′-alpha-thiomethoxyadenosine | 7-substituted 1,3-(diaza)-2-(oxo)-phenoxazin- |
| | 1-yl |
| 2′-Deoxy-2′-alpha-thiomethoxycytidine | 8-(alkenyl)adenine |
| 2′-Deoxy-2′-alpha-thiomethoxyguanosine | 8-(alkenyl)guanine |
| 2′-Deoxy-2′-alpha-thiomethoxyuridine | 8-(alkyl)adenine |
| 2′-Deoxy-2′-alpha-mercaptouridine | 8-(alkyl)guanine |
| 2′-Deoxy-2′-beta-aminoadenosine | 8-(alkynyl)adenine |
| 2′-Deoxy-2′-beta-aminoguanosine | 8-(alkynyl)guanine |
| 2′-Deoxy-2′-beta-aminouridine | 8-(amino)adenine |
| 2′-Deoxy-2′-beta-azidoadenosine | 8-(amino)guanine |
| 2′-Deoxy-2′-beta-azidocytidine | 8-(halo)adenine |
| 2′-Deoxy-2′-beta-azidoguanosine | 8-(halo)guanine |
| 2′-Deoxy-2′-beta-azidouridine | 8-(hydroxyl)adenine |
| 2′-Deoxy-2′-beta-aminocytidine | 8-(hydroxyl)guanine |
| 2′-Deoxy-2′-beta-bromoadenosine | 8-(thioalkyl)adenine |
| 2′-Deoxy-2′-beta-bromocytidine | 8-(thioalkyl)guanine |
| 2′-Deoxy-2′-beta-bromoguanosine | 8-(thiol)adenine |
| 2′-Deoxy-2′-beta-bromouridine | 8-(thiol)guanine |
| 2′-Deoxy-2′-beta-chloroadenosine | 8-Aza-adenosine |
| 2′-Deoxy-2′-beta-chlorocytidine | 8-azido-adenosine |
| 2′-Deoxy-2′-beta-chloroguanosine | 8-bromo-adenosine |
| 2′-Deoxy-2′-beta-chlorouridine | 8-bromo-guanosine |
| 2′-Deoxy-2′-beta-fluoroadenosine | 8-oxo-guanosine |
| 2′-Deoxy-2′-beta-fluorocytidine | 8-Trifluoromethyladenosine |
| 2′-Deoxy-2′-beta-fluoroguanosine | 9-(methyl)-imidizopyridinyl |
| 2′-Deoxy-2′-beta-fluorouridine | 9-Deazaadenosine |
| 2′-Deoxy-2′-beta-iodoadenosine | 9-Deazaguanosine |
| 2′-Deoxy-2′-beta-iodocytidine | alkene containing backbones |
| 2′-Deoxy-2′-beta-iodoguanosine | alkyl phosphonates |
| 2′-Deoxy-2′-beta-iodouridine | allyamino-thymidine |
| 2′-Deoxy-2′-beta-mercaptoadenosine | allyamino-uracil |
| 2′-Deoxy-2′-beta-mercaptocytidine | alpha-thio-cytidine |
| 2′-Deoxy-2′-beta-mercaptoguanosine | alpha-thio-guanosine |
| 2′-Deoxy-2′-beta-mercaptouridine | alpha-thio-pseudo-uridine |
| 2′-Deoxy-2′-beta-thiomethoxyadenosine | alpha-thio-uridine |
| 2′-Deoxy-2′-beta-thiomethoxycytidine TP | altriol |
| 2′-Deoxy-2′-beta-thiomethoxyuridine | aminoalkylphosphoramidates |
| 2′-deoxyuridine | aminoalkylphosphotriesters |
| 2′-F-5-Methyl-2′-deoxyuridine | aminoindolyl |
| 2′-Fluoro | anthracenyl |
| 2′-fluoro-modified bases | archaeosine |
| 2′-fluorouridine | aza cytosine |
| 2′-methyl,2′-amino,2′-azido,2′-fluoro- | aza thymidine |
| adenine | |
| 2′-methyl,2′-amino,2′-azido,2′-fluroo- | aza uracil |
| cytidine | |
| 2′-OH-ara-adenosinc | aza adeninc |
| 2′-OH-ara-cytidine | azaguanine |
| 2′-OH-ara-guanosine | bis-ortho-(aminoalkylhydroxy)-6-phenyl- |
| | pyrrolo-nvrimidin-2-on-3-yl |
| 2′-OH-ara-uridine | bis-ortho-substituted-6-phenyl-pyrrolo- |
| | pyrimidin-2-on-3-yl |
| 2′-OMe-2-Aminoadenosine | boranophosphates |
| 2′-OMc-5-Mc-uridinc | —CH2—O—N(CH3)—CH2— |
| 2′-OMe-pseudouridine | —CH2—N(CH3)—N(CH3)—CH2— |
| 2′-O-Methyl-5-(1-propynyl)cytidine | —CH2—NH—CH2— |
| 2′-O-Methyl-5-(1-propynyl)uridine | chiral phosphonates |
| 2′-O-methyladenosine | chiral phosphorothioates |
| 2′-O-methylation | Constrained nucleic acid (CNA) |
| 2′-O-methylcytidine | deaza cytosine |
| 2′-O-mcthylguanosinc | dcaza guaninc |
| 2′-O-methylinosine | deaza thymidine |
| 2′-O-methyl-ribose | deaza uracil |
| 2′-O-methyluridine | deazaadenine |
| 2′-O-ribosyladenosine(phosphate) | deoxy-thymidine |
| 2-(alkyl)guanine | difluorotolyl |
| 2-(alkyl)adenine | dihydropseudouridine |
| 2-(amino)adenine | dihydrouridine |
| 2-(aminoalkyl)adenine | DNA |
| 2-(aminopropyl)adenine | epoxyqueuosine |
| 2-(halo)adenine | Fluoro hexitol nucleic acid (FHNA) |
| 2-(methylthio)N6(isopentenyl)adenine | formacetyl and thioformacetyl backbones |
| 2-(propyl)adenine | Formycin A |
| 2-(propyl)guanine | Formycin B |
| 2-(thio)cytosine | galactosyl-queuosine |
| 2-(thio)uracil | GNA (glycol nucleic acid) |
| 2,2′-anhydro-cytidine | hydroxywybutosine |
| 2,2′-anhydro-uridine | hypoxanthine |
| 2,4-(dithio)pseudouracil | imidizopyridinyl |
| 2,4,5-(trimethyl)phenyl | inosinyl |
| 2,6-(diamino)purine | isocarbostyrily1 |
| 2,6-diaminopurine | isoguanosine |
| 2′-alpha-ethynyladenosine | isopentenyladenosine |
| 2′-Amino-2′-deoxy-guanosine | isowyosme |
| 2′-Amino-2′-deoxy-uridine | 1-Alkyl-6-homoallyl-pseudo-uridine |
| 2-amino-6-Chloro-purine | 1-Methyl-6-(2,2,2-Trifluoroethyl)pseudo- |
| | uridine |
| 2-aminoadenine | 1-Methyl-6-(4-thiomorpholino)-pseudo- |
| | uridine |
| 2-Aminoadenosine | 1-Methyl-6-azido-pseudo-uridine |
| 2-aminopurine | 1-Methyl-6-chloro-pseudo-uridine |
| 2-Amino-riboside | 1-Methyl-6-dimethylamino-pseudo-uridine |
| 2-aza-inosinyl | 1-Methyl-6-ethoxy-pseudo-uridine |
| 2′-azido-2′-deoxyadenosine | 1-Methyl-6-ethylcarboxylate-pseudo-uridine |
| 2′-Azido-2′-deoxy-guanosine | 1-Methyl-6-fluoro-pseudo-uridine |
| 2′-Azido-2′-deoxy-uridine | 1-Methyl-6-hydroxy-pseudo-uridine |
| 2-Azidoadenosine | 1-Methyl-6-iodo-pseudo-uridine |
| 2′-beta-Trifluoromethyladenosine | 1-Methyl-6-methylamino-pseudo-uridine |
| 2′-beta-Trifluoromethylguanosine | 1-[3-(2-{2-[2-(2-Aminoethoxy)-ethoxy]- |
| | ethoxy}-ethoxy)-propionyllpseudouridine |
| 2-Bromoadcnosinc | 1-Alkyl-6-(1-propynyl)-pscudo-uridinc |
| 2′-beta-Trifluoromethylcytidine | 1-Alkyl-6-(2-propynyl)-pseudo-uridine |
| 2-Chloroadenosine | 1-Alkyl-6-allyl-pseudo-uridine |
| 2′-Deoxy-2′-alpha-aminoadenosine | 1-Alkyl-6-ethynyl-pseudo-uridine |
| 2′-Deoxy-2′-alpha-aminoguanosine | 1-Alkyl-6-vinyl-pseudo-uridine |
| 2′-Deoxy-2′-alpha-azidoadenosine | 1-Biotinyl-PEG2-pseudouridine |
| 2′-Deoxy-2′-alpha-azidoguanosine | 1-methyl-1-deaza-pseudoisocytidine |
| 2′-Deoxy-2′-beta-thiomethoxyguanosine | 1-methyl-1-deaza-pseudouridine |
| 2′-Fluor-N4-Bz-cytidine | 1-Methyl-3-(3-amino-3- |
| | carboxyproovl)pseudo-Uridine |
| 2′-fluoro-2′-deoxyribose | 1-Methyl-3-(3-amino-3-carboxypropyl) |
| | pseudouridine |
| 2-Fluoroadenosinc | 1-methyl-3-(3-amino-5- |
| | carboxypropyl)pseudouridine |
| 2′-Fluoro-N2-isobutyl-guanosine | 1-Methyl-6-(4-morpholino)-pseudo-uridine |
| 2′-Fluoro-N4-Acetyl-cytidine | 1-Methyl-6-(substituted phenyl)pseudo- |
| | uridine |
| 2′-Fluoro-N6-Bz-deoxyadenosine | 1-Methyl-6-butyl-pseudo-uridine |
| 2-Iodoadenosine | 1-Methyl-6-ethyl-pseudo-uridine |
| 2-Mercaptoadenosine | 1-Methyl-6-formyl-pseudo-uridine |
| 2-methoxy-4-thio-pseudouridine | 1-Methyl-6-iso-propyl-pseudo-uridine |
| 2-methoxy-4-thio-uridine | 1-Methyl-6-methoxy-pseudo-uridine |
| 2-methoxy-5-methyl-cytidine | 1-Methyl-6-phenyl-pseudo-uridine |
| 2-methoxy-adenine | 1-Methyl-6-propyl-pseudo-uridine |
| 2-methoxy-cytidine | 1-Methyl-6-tert-butyl-pseudo-uridine |
| 2-methoxyuridine | 1-methyl-6-thio-guanosine |
| 2′-methyl,2′-amino,2′-azido,2′-fluro- | 1-Methyl-6-trifluoromethyl-pseudo-uridine |
| guanosine | |
| 2′-methyl,2′-amino,2′-azido,2′fluro-uridine | Locked nucleic acid (LNA) |
| 2-methyladenosine | 1-taurinomethyl-1-methyl-uridine |
| 2-methylpseudouridine | 1-taurinomethyl-4-thio-uridine |
| 2-methylthioadenine | lysidine |
| 2-methylthio-N6 isopentenyladenosine | mannosyl-queuosine |
| 2-methylthio-N6-(cis- | Methyl phosphonate |
| hydroxyisopentenyl)adenosine | |
| 2-methylthio-N6-hydroxynorvalyl | methylene (methylimino) |
| carbamoyladenosine | |
| 2-methylthio-N6-isopentenyladenosine | methylene formacetyl and thioformacetyl |
| | backbones |
| 2-methylthio-N6-methyladenosine | methyleneimino and methylenehydrazino |
| | backbones |
| 2-methylthio-N6-threonyl | methylphosphonates |
| carbamoyladenosine | |
| 2′-O-methoxyethyl(MOE) | methylwyosine |
| 2′-O-methoxyethylribose(MOE) | morpholino linkages |
| 2′-O-methyl | mosme |
| 2′-O-methyladenosine | N(methyl)guanine |
| 2′-O-methylcytidine | —N(CH3)—CH2—CH2— |
| 2′-O-methylguanosine | N-(methyl)guanine |
| 2′-O-methylinosine | N2,7,2′-O-trimethylguanosine |
| 2′O-methyl-N2-isobutyl-guanosine | N2,2′-O-dimethylguanosine |
| 2′-O-Methyl-N4-Acetyl-cytidine | N2,7-dimethylguanosine |
| 2′-O-methyl-N4-Bz-cytidine | N2,N2,2′-O-trimethylguanosine |
| 2′-O-methyl-N6-Bz-deoxyadenosine | N2,N2,7-trimethylguanosine |
| 2′-O-methylpseudouridine | N2,N2-dimethyl-6-thio-guanosine |
| 2′-O-methyluridine | N2,N2-dimethylguanosine |
| 2′-O-ribosyladenosine(phosphate) | N2-isobutyl-guanosine |
| 2′-O-ribosylguanosine(phosphate) | N2-methyl-6-thio-guanosine |
| 2-oxo-7-aminopyridopyrimidin-3-yl | N2-methylguanosine |
| 2-oxo-pyridopyrimidine-3-yl | N2-substituted purines |
| 2-pyridinone | N3(methyl)uracil |
| 2-thio-1-methyl-1-deaza-pseudouridine | N4(acetyl)cytosine |
| 2-thio-1-methyl-pseudouridine | N4,2′-O-dimethylcytidine |
| 2-thio-2′-O-methyluridine | N4,N4-Dimethyl-2′-OMe-Cytidine |
| 2-thio-5-aza-uridine | N4-acetyl-2′-O-methylcytidine |
| 2-thio-5-methyl-cytidine | N4-acetylcytidine |
| 2-thiocytidine | N4-Amino-cytidine |
| 2-thio-dihydropseudouridine | N4-Benzoyl-cytidine |
| 2-thio-dihydrouridine | N4-methylcytidine |
| 2-thio-pseudouridine | N6-(19-Amino-pentaoxanonadecyl)adenosine |
| 2-thiouridine | N6-(cis-hydroxyisopentenyl)adenosine |
| 2-thio-zebularine | N6-(isopentyl)adenine |
| 2-Trifluoromethyladenosine | N6-(methyl)adenine |
| 3-(deaza)-5-(aza)cytosine | N6,N6(dimethyl)adenine |
| 3-(methyl)cytosine | N6,2′-O-dimethyladenosine |
| 3-nitropyrrole | N6,N6,2′-O-trimethyladenosine |
| 3-(3-amino-3-carboxypropyl)uracil | N6,N6-dimethyladenosine |
| 3-(3-amino-3-carboxypropyl)uridine | N6-acetyladenosine |
| 3-(alkyl)cytosine | N6-cis-hydroxy-isopentenyl-adenosine |
| 3-(methyl)-7-(propynyl)isocarbostyrilyl | N6-glycinylcarbamoyladenosine |
| 3-(methyl)cytidine | N6-hydroxynorvalylcarbamoyladenosine |
| 3-(methyl)isocarbostyrilyl | N6-isopentenyladenosine |
| 3,2′-0-dimethyluridine | N6-methyl-2-amino-purine |
| 3′-alkylene phosphonates | N6-methyladenosine |
| 3-alkyl-pseudouridine | N6-methyl-N6-threonylcarbamoyladenosine |
| 3′-aminophosphoramidate | N6-substituted purines |
| 3-deaza-3-bromoadenosine | N6-threonylcarbamoyladenosine |
| 3-deaza-3-chloroadenosine | N-alkylated derivative |
| 3-deaza-3-fluoroadenosine | napthalenyl |
| 3-deaza-3-iodoadenosine | nitrobenzimidazolyl |
| 3-deazaadenosine | nitroimidazolyl |
| 3′-ethynylcytidine | nitroindazolyl |
| 3-methylcytidine | nitropyrazolyl |
| 3-methyl-pseudouridine | N1-methyl-adenosine |
| 3-methyluridine | N1-methyl-guanosine |
| 4′-azidoadenosine | nubularine |
| 4′-azidouridine | 06-substituted purines |
| 4′-ethynyladenosine | O-alkylated derivative |
| 4′-ethynylcytidine | oligonucleosides with heteroatom |
| | internucleoside linkage |
| 4′-ethynylguanosine | ortho-(aminoalkylhydroxy)-6-phenyl-pyrrolo- |
| | pyrimidin-2-on-3-yl |
| 4′-ethynyluridine | ortho-substituted-6-phenyl-pyrrolo-pyrimidin- |
| | 2-on-3-yl |
| 4-(fluoro)-6-(methyl)benzimidazole | Oxoformycin TP |
| 4-(methyl)benzimidazole | para-(aminoalkylhydroxy)-6-phenyl-pyrrolo- |
| | pyrimidin-2-on-3-yl |
| 4-(methyl)indolyl | para-substituted-6-phenyl-pyrrolo-pyrimidin- |
| | 2-on-3-yl |
| 4-(thio)pseudouracil | pentacenyl |
| 4-(thio)uracil | peroxywybutosine |
| 4,2′-O-dimethylcytidine | phenanthracenyl |
| 4,6-(dimethyl)indolyl | phenyl |
| 4′-azidocytidine | phosphinates |
| 4′-azidoguanosine | phosphonoacetates |
| 4′-carbocyclic adenosine | phosphoramidates |
| 4′-carbocyclic cytidine | Phosphorodiamidate Morpholino Oligomer |
| | (PMO) |
| 4′-carbocyclic guanosine | phosphorodithioates |
| 4′-carbocyclic uridine | Phosphorothioate |
| 4-demethylwyosine | phosphorothioate internucleoside linkages |
| 4-methoxy-1-methyl-pscudoisocytidinc | phosphorothioates |
| 4-methoxy-2-thio-pseudouridine | phosphotriesters |
| 4-methoxy-pseudoisocytidine | PNA |
| 4-methoxy-pseudouridine | propynyl-7-(aza)indolyl |
| 4-methylcytidine | pseudoisocytidine |
| 4-thio-1-methyl-1-deaza-pseudoisocytidine | Pseudo-iso-cytidine |
| 4-thio-1-methyl-pseudoisocytidine | pseudouracil |
| 4-thio-1-methyl-pseudouridine | pseudouridine |
| 4-thio-pseudoisocytidine | Pseudouridine 1-(4-methylbenzenesulfonic |
| | acid) |
| 4-thio-pseudouridine | Pseudouridine 1-(4-methylbenzoic acid) TP |
| 4-thiouracil | Pseudouridine 1-methylphosphonic acid |
| 4-thiouridine | Pseudouridine 1-[3-(2-ethoxy)]propionic acid |
| 5(halo)cytosine | Pseudouridine 1-[3-{2-(2-[2-(2-ethoxy)- |
| | ethoxy]-ethoxy)-ethoxy}]propionic acid |
| 5(methyl)4(thio)uracil | Pseudouridine 1-[3-{2-(2-[2-{2(2-ethoxy)- |
| | ethoxy}-ethoxy]-ethoxy)-ethoxy}]propionic |
| | acid |
| 5(methyl)cytosine | Pseudouridine 1-[3-{2-(2-[2-ethoxy]-ethoxy)- |
| | ethoxy}]propionic acid |
| 5(methylaminomethyl)-2(thio)uracil | Pseudouridine 1-[3-{2-(2-ethoxy)-ethoxv}] |
| | propionic acid |
| 5(methylaminomethyl)-2,4(dithio)uracil | Pscudouridinc TP 1-methylphosphonic acid |
| | diethyl ester |
| 5(methylaminomethyl)-4(thio)uracil | Pseudo-uridine-1-2-ethanoic acid |
| 5(propynyl)cytosine | Pseudo-uridine-N1-5-pentanoic acid |
| 5(propynyl)uracil | Pseudo-uridine-N1-3-propionic acid |
| 5(trifluoromethyl)cytosine | Pseudo-uridine-N1-4-butanoic acid |
| 5(trifluoromethyl)uracil | Pseudo-uridine-N1-6-hexanoic acid |
| 5 nitroindole | Pseudo-uridine-N1-methy1-p-benzoic acid |
| 5 substituted pyrimidines | Pseudo-uridine-N1-p-benzoic acid |
| 5-(1,3-diazole-1-alkyl)uracil | Pseudo-uridine-N1-7-heptanoic acid |
| 5-(1-Propynyl)ara-cytidine | pyrenyl |
| 5-(1-Propynyl)ara-uridine | pyridin-4-one ribonucleoside |
| 5-(2-aminopropyl)uracil | pyridopyrimidin-3-yl |
| 5-(2-carbomethoxyvinyl)uridine | pyridopyrimidin-3-yl,2-oxo-7-amino- |
| | pyridopyrimidin-3-yl |
| 5-(2-Chloro-phenyl)-2-thiocytidine | pyrrolo-cytidine |
| 5-(2-Furanyl)uridine | pyrrolo-pseudoisocytidine |
| 5-(4-Amino-phenyl)-2-thiocytidine | pyrrolo-pyrimidin-2-on-3-yl |
| 5-(alky1)-2-(thio)pseudouracil | pyrrolopyrimidinyl |
| 5-(alky1)-4(thio)pseudouracil | pyrrolopyrizinyl |
| 5-(alkyl)-2,4(dithio)pseudouracil | Pyrrolosine |
| 5-(alkyl)cytosine | siloxane backbones |
| 5-(alkyl)pseudouracil | stilbenzyl |
| 5-(alkyl)uracil | substituted 1,2,4-triazoles |
| 5-(alkynyl)cytosine | sulfamate backbones |
| 5-(alkynyl)uracil | sulfide sulfoxide and sulfone backbones |
| 5-(allylamino)uracil | sulfonate and sulfonamide backbones |
| 5-(aminoalkyl)uracil | tetracenyl |
| 5-(carboxyhydroxymethyl)uridine | thio-adenosine |
| 5-(carboxyhydroxymethyl)uridine methyl | thionoalkylphosphonates |
| ester | |
| 5-(cyanoalkyl)uracil | thionoalkylphosphotriesters |
| 5-(dialkylaminoalkyl)uracil | thionophosphoramidates |
| 5-(dimethylaminoalkyl)uracil | Tricyclo-DNA (tcDNA) |
| 5-(guanidiniumalkyl)uracil | tubercidine |
| 5-(halo)cytosine | undermodified hydroxywybutosine |
| 5-(halo)uracil | uridine 5-oxyacetic acid |
| 5-(iso-Pentenylaminomethyl)-2-thiouridine | uridine 5-oxyacetic acid methyl ester |
| 5-(iso-Pentenylaminomethyl)-2′-O- | wybutosine |
| methyluridine | |
| 5-(iso-Pentenylaminomethyl)uridine | wyosme |
| 5-(1,3-diazole-1-alkyl)uracil | xanthine |
| 5-(methoxy)uracil | Xanthosine |
| 5-(methoxycarbonylmethyl)-2-(thio)uracil | zebularine |

A TREM may comprise a non-naturally occurring modification in each of the ASt Domain1, the DH Domain, the ACH Domain, the VL Domain, the TH Domain, and the ASt Domain2. In some embodiments, the TREM comprises 0-10, 10-20, 20-30, 30-40, 40-50, 50-60, 60-70, 70-80, or 80-90 non-naturally occurring modifications. In some embodiments, the TREM comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 non-naturally occurring modifications. In some embodiments, the TREM comprises a non-naturally occurring modification in each of the ASt Domain1, the DH Domain, the TH Domain, and the ASt Domain2. In some embodiments, the TREM comprises a non-naturally occurring modification in each of the ASt Domain1, the ACH Domain, the TH Domain, and the ASt Domain2. In some embodiments, the TREM comprises a non-naturally occurring modification in each of the ASt Domain1 and the ASt Domain2. In some embodiments, the TREM comprises a non-naturally occurring modification in each of the ASt Domain1, the VL Domain, the TH Domain, and the ASt Domain2. In some embodiments, the TREM comprises a non-naturally occurring modification in each of the ASt Domain1, the DH Domain, and the ASt Domain2. In some embodiments, the TREM comprises a non-naturally occurring modification in each of the ASt Domain1, the TH Domain, and the ASt Domain2. In some embodiments, the TREM comprises a non-naturally occurring modification in each of the ASt Domain1, the DH Domain, the ACH Domain, the TH Domain, and the ASt Domain2. In some embodiments, the TREM comprises a non-naturally occurring modification in each of the ASt Domain1, the ACH Domain, and the ASt Domain2. In some embodiments, the TREM comprises a non-naturally occurring modification in each of the ASt Domain1, the ACH Domain, the VL Domain, the TH Domain, and the ASt Domain2. In some embodiments, the TREM comprises a non-naturally occurring modification in the ASt Domain1. In some embodiments, the TREM comprises a non-naturally occurring modification in the DH Domain. In some embodiments, the TREM comprises a non-naturally occurring modification in the ACH Domain. In some embodiments, the TREM comprises a non-naturally occurring modification in the VL Domain. In some embodiments, the TREM comprises a non-naturally occurring modification in the TH Domain. In some embodiments, the TREM comprises a non-naturally occurring modification in the ASt Domain2.

In some embodiments, the TREM comprises a nucleotide sugar modification in each of the ASt Domain1, the DH Domain, the ACH Domain, the VL Domain, the TH Domain, and the ASt Domain2. In some embodiments, the nucleotide sugar modification comprises a 2′-O-methyl modification. In some embodiments, the nucleotide sugar modification comprises a 2′-fluoro modification. In some embodiments, the TREM comprises 0-5, 5-10, 10-20, or 20-30 2′-O-methyl modifications. In some embodiments, the TREM comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 2′-O-methyl modifications. In some embodiments, the TREM comprises 0-5, 5-10, 10-20, or 20-30 2′-fluoro modifications. In some embodiments, the TREM comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 2′-fluoro modifications. In some embodiments, the TREM comprises a 2′-O-methyl modification in each of the ASt Domain1, the DH Domain, the ACH Domain, the VL Domain, the TH Domain, and the ASt Domain2. In some embodiments, the TREM comprises a 2′-O-methyl modification in each of the ASt Domain1, the TH Domain, and the ASt Domain2. In some embodiments, the TREM comprises a 2′-O-methyl modification in each of the ASt Domain1 and the ASt Domain2. In some embodiments, the TREM comprises a 2′-O-methyl modification in each of the ASt Domain1, the DH Domain, and the ASt Domain2. In some embodiments, the TREM comprises a 2′-O-methyl modification in each of the ASt Domain1, the DH Domain, the ACH Domain, and the ASt Domain2. In some embodiments, the TREM comprises a 2′-O-methyl modification in each of the ASt Domain1, the ACH Domain, and the ASt Domain2. In some embodiments, the TREM comprises a 2′-O-methyl modification in each of the ASt Domain1, the DH Domain, the TH Domain, and the ASt Domain2. In some embodiments, the TREM comprises a 2′-O-methyl modification in the ASt Domain1. In some embodiments, the TREM comprises a 2′-fluoro modification in each of the ASt Domain1, the DH Domain, the ACH Domain, the VL Domain, the TH Domain, and the ASt Domain2. In some embodiments, the TREM comprises a 2′-fluoro modification in each of the DH Domain and the TH Domain. In some embodiments, the TREM comprises a 2′-fluoro modification in each of the VL Domain, the TH Domain, and the ASt Domain2. In some embodiments, the TREM comprises a 2′-fluoro modification in each of the DH Domain, the ACH Domain, the TH Domain, and the ASt Domain2. In some embodiments, the TREM comprises a 2′-fluoro modification in each of the DH Domain, the ACH Domain, the VL Domain, and the TH Domain. In some embodiments, the TREM comprises a 2′-fluoro modification in each of the ASt Domain1, the DH Domain, the ACH Domain, and the ASt Domain2. In some embodiments, the TREM comprises a 2′-fluoro modification in each of the DH Domain, the TH Domain, and the ASt Domain2. In some embodiments, the TREM comprises a 2′-fluoro modification in each of the DH Domain, the ACH Domain, the VL Domain, and the ASt Domain2. In some embodiments, the TREM comprises a 2′-fluoro modification in each of the ASt Domain1, the ACH Domain, the VL Domain, the TH Domain, and the ASt Domain2. In some embodiments, the TREM comprises a 2′-fluoro modification in the ASt Domain1. In some embodiments, the TREM comprises a 2′-fluoro modification in the DH Domain. In some embodiments, the TREM comprises a 2′-fluoro modification in the ACH Domain. In some embodiments, the TREM comprises a 2′-fluoro modification in the TH Domain. In some embodiments, the TREM comprises a 2′-fluoro modification in the ASt Domain2.

In some embodiments, the TREM comprises an internucleotide modification in each of the ASt Domain1, the DH Domain, the ACH Domain, the VL Domain, the TH Domain, and the ASt Domain2. In some embodiments, the internucleotide modification comprises a phosphorothioate linkage. In some embodiments, the TREM comprises 0-5, 5-10, 10-20, or 20-30 phosphorothioate linkages. In some embodiments, the TREM comprises 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 phosphorothioate linkages. In some embodiments, the TREM comprises a phosphorothioate linkage in each of the ASt Domain1, the ACH Domain, the TH Domain, and the ASt Domain2. In some embodiments, the TREM comprises a phosphorothioate linkage in each of the ASt Domain1 and the ASt Domain2. In some embodiments, the TREM comprises a phosphorothioate linkage in each of the ASt Domain1, the TH Domain, and the ASt Domain2. In some embodiments, the TREM comprises a phosphorothioate linkage in each of the ASt Domain1, the DH Domain, the TH Domain, and the ASt Domain2. In some embodiments, the TREM comprises a phosphorothioate linkage in each of the ASt Domain1, the ACH Domain, the VL Domain, and the ASt Domain2. In some embodiments, the TREM comprises a phosphorothioate linkage in each of the ASt Domain1, the DH Domain, the ACH Domain, TH Domain, and the ASt Domain2. In some embodiments, the TREM comprises a phosphorothioate linkage in each of the ASt Domain1, the DH Domain, the VL Domain, and the TH Domain. In some embodiments, the TREM comprises a phosphorothioate linkage in each of the ASt Domain1, the DH Domain, and the ASt Domain2. In some embodiments, the TREM comprises a phosphorothioate linkage in the ACH Domain.

A TREM may comprise a non-naturally occurring modification (e.g., a nucleotide sugar modification or an internucleotide modification) in each of the ASt Domain1, the DH Domain, the ACH Domain, the VL Domain, the TH Domain, or the ASt Domain2. In some embodiments, the TREM comprises the non-naturally occurring modification pattern of Pattern No: 1 in Table 6:1-m, 18-m, 19-m, 50-m, 52-m, 73-m. In some embodiments, the TREM comprises the non-naturally occurring modification pattern of Pattern No: 2 in Table 6:1-m*, 2-m*, 43-*, 55-*, 74-*, 75-m. In some embodiments, the TREM comprises the non-naturally occurring modification pattern of Pattern No: 3 in Table 6:1-m*, 2-m*, 74-*, 75-m. In some embodiments, the TREM comprises the non-naturally occurring modification pattern of Pattern No: 4 in Table 6:1-m*, 2-m*, 27-*, 51-*, 59-f, 74-*, 75-m. In some embodiments, the TREM comprises the non-naturally occurring modification pattern of Pattern No: 5 in Table 6:1-m*, 2-m*, 13-f, 14-f, 54-f, 59-m, 73-*, 75-*, 76-m. In some embodiments, the TREM comprises the non-naturally occurring modification pattern of Pattern No: 6 in Table 6:1-m*, 2-m*, 45-f, 57-f, 68-f, 74-*, 75-m*, 76-m. In some embodiments, the TREM comprises the non-naturally occurring modification pattern of Pattern No: 7 in Table 6:1-m*, 2-m, 74-*, 75-m. In some embodiments, the TREM comprises the non-naturally occurring modification pattern of Pattern No: 8 in Table 6:1-m*, 2-m*, 27-*, 51-*, 59-f, 73-*, 74-*, 75-m. In some embodiments, the TREM comprises the non-naturally occurring modification pattern of Pattern No: 9 in Table 6:1-m*, 2-m, 25-f, 74-*, 75-m. In some embodiments, the TREM comprises the non-naturally occurring modification pattern of Pattern No: 10 in Table 6:1-m*, 2-m*, 13-f, 14-f, 54-f, 59-m, 75-*, 76-m. In some embodiments, the TREM comprises the non-naturally occurring modification pattern of Pattern No: 11 in Table 6:1-m*, 2-m, 73-*, 74-*, 75-m. In some embodiments, the TREM comprises the non-naturally occurring modification pattern of Pattern No: 12 in Table 6:1-m*, 2-m*, 52-m, 63-*, 74-*, 75-m*, 76-m. In some embodiments, the TREM comprises the non-naturally occurring modification pattern of Pattern No: 13 in Table 6:1-m*, 2-m, 13-*, 14-m, 55-*, 74-*, 75-m. In some embodiments, the TREM comprises the non-naturally occurring modification pattern of Pattern No: 14 in Table 6:1-m*, 2-m*, 12-*, 13-f*, 23-*, 24-f, 43-f, 52-*, 59-m*, 61-f, 68-f, 70-f, 74-*, 75-m. In some embodiments, the TREM comprises the non-naturally occurring modification pattern of Pattern No: 15 in Table 6:1-m*, 2-m*, 14-f, 16-f, 22-f, 24-f, 40-*, 43-f, 44-f, 45-f*, 52-f, 61-f, 74-*. In some embodiments, the TREM comprises the non-naturally occurring modification pattern of Pattern No: 16 in Table 6:1-m, 40-f, 41-m*, 42-f, 43-m*, 73-m. In some embodiments, the TREM comprises the non-naturally occurring modification pattern of Pattern No: 17 in Table 6:1-m*, 2-m, 74-*, 75-m. In some embodiments, the TREM comprises the non-naturally occurring modification pattern of Pattern No: 18 in Table 6:1-m*, 2-m. In some embodiments, the TREM comprises the non-naturally occurring modification pattern of Pattern No: 19 in Table 6:1-m*, 2-m*, 75-*, 76-m. In some embodiments, the TREM comprises the non-naturally occurring modification pattern of Pattern No: 20 in Table 6:1-m*, 2-m*, 52-*, 74-*, 75-m. In some embodiments, the TREM comprises the non-naturally occurring modification pattern of Pattern No: 21 in Table 6:1-m*, 2-m*, 13-f, 14-f, 54-f, 59-m, 75-*, 76-m. In some embodiments, the TREM comprises the non-naturally occurring modification pattern of Pattern No: 22 in Table 6:1-m*, 2-m, 46-f, 59-m, 75-*, 76-m. In some embodiments, the TREM comprises the non-naturally occurring modification pattern of Pattern No: 23 in Table 6:1-m*, 2-m*, 14-f, 16-f, 22-f, 24-f, 40-*, 43-f, 44-f, 45-f*, 52-f, 61-f, 74-*. In some embodiments, the TREM comprises the non-naturally occurring modification pattern of Pattern No: 24 in Table 6:1-m*, 2-m*, 12-*, 13-f*, 23-*, 24-f, 43-f, 52-*, 59-m*, 61-f, 68-f, 70-f, 74-*, 75-m. In some embodiments, the TREM comprises the non-naturally occurring modification pattern of Pattern No: 25 in Table 6:1-m*, 2-f, 20-*, 20a-f, 23-*, 24-f, 28-f, 38-*, 39-f, 52-*, 61-f, 66-*, 74-*, 75-m. In some embodiments, the TREM comprises the non-naturally occurring modification pattern of Pattern No: 26 in Table 6:1-m*, 2-m, 13-*, 14-f, 22-f, 23-f, 27-f*, 40-m*, 41-f, 44-f, 56-*, 74-*, 75-*. In some embodiments, the TREM comprises the non-naturally occurring modification pattern of Pattern No: 27 in Table 6:1-m*, 2-m, 12-m, 13-*, 22-f, 23-f, 27-*, 28-f, 40-*, 41-f, 46-f, 56-m, 64-*, 65-*, 68-f, 74-*, 75-m. In some embodiments, the TREM comprises the non-naturally occurring modification pattern of Pattern No: 28 in Table 6:1-m*, 2-f*, 19-f, 20-f, 20a-f, 23-*, 24-f*, 25-*, 27-f, 31-f, 34-f, 44-f, 49-f*, 64-f*, 68-f, 72-f, 76-m. In some embodiments, the TREM comprises the non-naturally occurring modification pattern of Pattern No: 29 in Table 6:1-m*, 13-f*, 20-*, 20a-f, 28-f, 40-f, 42-f, 53-f*, 68-f, 75-*, 76-f. In some embodiments, the TREM comprises the non-naturally occurring modification pattern of Pattern No: 30 in Table 6:1-m*, 4-f*, 28-m, 43-f, 46-f, 56-f, 61-f, 62-f, 74-f *. In some embodiments, the TREM comprises the non-naturally occurring modification pattern of Pattern No: 31 in Table 6: 1-f*, 2-m*, 12-m, 13-*, 16-f, 22-f, 23-f, 28-f, 29-m, 44-f, 54-f*, 66-*, 67-*, 68-f, 74-*, 75-m, 76-f. In some embodiments, the TREM comprises the non-naturally occurring modification pattern of Pattern No: 32 in Table 6:1-m*, 2-m*, 12-*, 20-f, 22-f, 23-f, 31-m, 51-*, 52-f, 64-*, 67-f, 74-*, 75-m. In some embodiments, the TREM comprises the non-naturally occurring modification pattern of Pattern No: 33 in Table 6:74-f. In some embodiments, the TREM comprises the non-naturally occurring modification pattern of Pattern No: 34 in Table 6:75-f. In some embodiments, the TREM comprises the non-naturally occurring modification pattern of Pattern No: 35 in Table 6:1-m, 2-m, 3-m, 4-m, 13-m, 18-m, 19-m, 50-m, 52-m, 66-m, 73-m. In some embodiments, the TREM comprises the non-naturally occurring modification pattern of Pattern No: 36 in Table 6:76-f. In some embodiments, the TREM comprises the non-naturally occurring modification pattern of Pattern No: 37 in Table 6:1-m, 2-m, 3-m, 4-m, 13-m, 18-m, 19-m, 50-m, 52-m, 73-m. In some embodiments, the TREM comprises the non-naturally occurring modification pattern of Pattern No: 38 in Table 6:1-m*, 2-m*, 30-m*, 43-m, 46-m*, 52-f*, 54-*, 73-m. In some embodiments, the TREM comprises the non-naturally occurring modification pattern of Pattern No: 39 in Table 6:1-m*, 2-*, 25-f*, 30-*, V24-m*, 49-*, 53-m, 71-m, 74-*. In some embodiments, the TREM comprises the non-naturally occurring modification pattern of Pattern No: 40 in Table 6:1-m*, 2-m, 4-m, 15-m, 18-*, 27-f, 49-m*, 53-m. In some embodiments, the TREM comprises the non-naturally occurring modification pattern of Pattern No: 41 in Table 6:1-m*, 2-*, 19-*, 24-f, 37-*, V23-m, 73-m*. In some embodiments, the TREM comprises the non-naturally occurring modification pattern of Pattern No: 42 in Table 6:1-m*, 2-m*, 8-f, 14-m*, 24-*, 30-f*, 52-*, 54-m.

TABLE 6
Exemplary non-naturally occurring modification patterns of TREMs

| Pattern | |
|---|---|
| No. | Modification Pattern |
| 1 | 1-m, 18-m, 19-m, 50-m, 52-m, 73-m |
| 2 | 1-m*, 2-m*, 43-*, 55-*, 74-*, 75-m |
| 3 | 1-m*, 2-m*, 74-*, 75-m |
| 4 | 1-m*, 2-m*, 27-*, 51-*, 59-f, 74-*, 75-m |
| 5 | 1-m*, 2-m*, 13-f, 14-f, 54-f, 59-m, 73-*, 75-*, 76-m |
| 6 | 1-m*, 2-m*, 45-f, 57-f, 68-f, 74-*, 75-m*, 76-m |
| 7 | 1-m*, 2-m, 74-*, 75-m |
| 8 | 1-m*, 2-m*, 27-*, 51-*, 59-f, 73-*, 74-*, 75-m |
| 9 | 1-m*, 2-m, 25-f, 74-*, 75-m |
| 10 | 1-m*, 2-m*, 13-f, 14-f, 54-f, 59-m, 75-*, 76-m |
| 11 | 1-m*, 2-m, 73-*, 74-*, 75-m |
| 12 | 1-m*, 2-m*, 52-m, 63-*, 74-*, 75-m*, 76-m |
| 13 | 1-m*, 2-m, 13-*, 14-m, 55-*, 74-*, 75-m |
| 14 | 1-m*, 2-m*, 12-*, 13-f*, 23-*, 24-f, 43-f, 52-*, 59-m*, 61-f, 68-f, 70-f, 74-*, 75-m |
| 15 | 1-m*, 2-m*, 14-f, 16-f, 22-f, 24-f, 40-*, 43-f, 44-f, 45-f*, 52-f, 61-f, 74-* |
| 16 | 1-m, 40-f, 41-m*, 42-f, 43-m*, 73-m |
| 17 | 1-m*, 2-m, 74-*, 75-m |
| 18 | 1-m*, 2-m |
| 19 | 1-m*, 2-m*, 75-*, 76-m |
| 20 | 1-m*, 2-m*, 52-*, 74-*, 75-m |
| 21 | 1-m*, 2-m*, 13-1, 14-1, 54-1, 59-m, 75-*, 76-m |
| 22 | 1-m*, 2-m, 46-f, 59-m, 75-*, 76-m |
| 23 | 1-m*, 2-m*, 14-f, 16-f, 22-f, 24-f, 40-*, 43-f, 44-f, 45-f*, 52-f, 61-f, 74-* |
| 24 | 1-m*, 2-m*, 12-*, 13-f*, 23-*, 24-f, 43-f, 52-*, 59-m*, 61-f, 68-f, 70-f, 74-*, 75-m |
| 25 | 1-m*, 2-f, 20-*, 20a-f, 23-*, 24-f, 28-f, 38-*, 39-f, 52-*, 61-f, 66-*, 74-*, 75-m |
| 26 | 1-m*, 2-m, 13-*, 14-f, 22-f, 23-f, 27-f*, 40-m*, 41-f, 44-f, 56-*, 74-*, 75-* |
| 27 | 1-m*, 2-m, 12-m, 13-*, 22-f, 23-f, 27-*, 28-f, 40-*, 41-f, 46-f, 56-m, 64-*, 65-*, 68- |
| | f, 74-*, 75-m |
| 28 | 1-m*, 2-f*, 19-f, 20-f, 20a-f, 23-*, 24-f*, 25-*, 27-f, 31-f, 34-f, 44-f, 49-f*, 64-f*, |
| | 68-f, 72-f, 76-m |
| 29 | 1-m*, 13-f*, 20-*, 20a-f, 28-f, 40-f, 42-f, 53-f*, 68-f, 75-*, 76-f |
| 30 | 1-m*, 4-f*, 28-m, 43-f, 46-f, 56-f, 61-f, 62-f, 74-f* |
| 31 | 1-f*, 2-m*, 12-m, 13-*, 16-1, 22-1, 23-1, 28-1, 29-m, 44-1, 54-f*, 66-*, 67-*, 68-f, |
| | 74-*, 75-m, 76-f |
| 32 | 1-m*, 2-m*, 12-*, 20-f, 22-f, 23-f, 31-m, 51-*, 52-f, 64-*, 67-f, 74-*, 75-m |
| 33 | 74-f |
| 34 | 75-f |
| 35 | 1-m, 2-m, 3-m, 4-m, 13-m, 18-m, 19-m, 50-m, 52-m, 66-m, 73-m |
| 36 | 76-f |
| 37 | 1-m, 2-m, 3-m, 4-m, 13-m, 18-m, 19-m, 50-m, 52-m, 73-m |
| 38 | 1-m*, 2-m*, 30-m*, 43-m, 46-m*, 52-f*, 54-*, 73-m |
| 39 | 1-m*, 2-*, 25-f*, 30-*, V24-m*, 49-*, 53-m, 71-m, 74-* |
| 40 | 1-m*, 2-m, 4-m, 15-m, 18-*, 27-f, 49-m*, 53-m |
| 41 | 1-m*, 2-*, 19-*, 24-f, 37-*, V23-m, 73-m* |
| 42 | 1-m*, 2-m*, 8-f, 14-m*, 24-*, 30-f*, 52-*, 54-m |

A TREM may not comprise a non-naturally occurring modification (e.g., a nucleotide sugar modification or an internucleotide modification) in each of the ASt Domain1, the DH Domain, the ACH Domain, the VL Domain, the TH Domain, or the ASt Domain2. In some embodiments, the TREM does not comprise a non-naturally occurring modification in the ASt Domain1. In some embodiments, the TREM does not comprise a non-naturally occurring modification in the DH Domain. In some embodiments, the TREM does not comprise a non-naturally occurring modification in the ACH Domain. In some embodiments, the TREM does not comprise a non-naturally occurring modification in the VL Domain. In some embodiments, the TREM does not comprise a non-naturally occurring modification in the TH Domain. In some embodiments, the TREM does not comprise a non-naturally occurring modification in the ASt Domain2. In some embodiments, the TREM has 70%, 75%, 80%, 85%, 90%, 95%, or 99% identity to SEQ ID NO: 622. In some embodiments, the TREM has 70%, 75%, 80%, 85%, 90%, 95%, or 99% identity to SEQ ID NO: 650. In some embodiments, the TREM has 70%, 75%, 80%, 85%, 90%, 95%, or 99% identity to SEQ ID NO: 653.

### TREM, TREM Core Fragment and TREM Fragment Fusions

In an embodiment, a TREM, a TREM core fragment or a TREM fragment disclosed herein comprises an additional moiety, e.g., a fusion moiety. In an embodiment, the fusion moiety can be used for purification, to alter folding of the TREM, TREM core fragment or TREM fragment, or as a targeting moiety. In an embodiment, the fusion moiety can comprise a tag, a linker, can be cleavable or can include a binding site for an enzyme. In an embodiment, the fusion moiety can be disposed at the N terminal of the TREM or at the C terminal of the TREM,

TREM core fragment or TREM fragment. In an embodiment, the fusion moiety can be encoded by the same or different nucleic acid molecule that encodes the TREM, TREM core fragment or TREM fragment.

### TREM Consensus Sequence

In an embodiment, a TREM disclosed herein comprises a consensus sequence provided herein.

In an embodiment, a TREM disclosed herein comprises a consensus sequence of Formula I _{ZZZ}, wherein _{ZZZ} indicates any of the twenty amino acids and Formula I corresponds to all species.

In an embodiment, a TREM disclosed herein comprises a consensus sequence of Formula II _{ZZZ}, wherein _{ZZZ} indicates any of the twenty amino acids and Formula II corresponds to mammals.

In an embodiment, a TREM disclosed herein comprises a consensus sequence of Formula III _{ZZZ}, wherein _{ZZZ} indicates any of the twenty amino acids and Formula III corresponds to humans.

In an embodiment, _{ZZZ} indicates any of the twenty amino acids: alanine, arginine, asparagine, aspartate, cysteine, glutamine, glutamate, glycine, histidine, isoleucine, methionine, leucine, lysine, phenylalanine, proline, serine, threonine, tryptophan, tyrosine, or valine.

In an embodiment, a TREM disclosed herein comprises a property selected from the following:
a) under physiological conditions residue R₀ forms a linker region, e.g., a Linker 1 region;
b) under physiological conditions residues R₁-R₂-R₃-R₄-R₅-R₆-R₇ and residues R₆₅-R₆₆-R₆₇-R₆₈-R₆₉-R₇₀-R₇₁ form a stem region, e.g., an AStD stem region;
c) under physiological conditions residues R₈-R₉ forms a linker region, e.g., a Linker 2 region;
d) under physiological conditions residues-R₁₀-R₁₁-R₁₂-R₁₃-R₁₄ R₁₅-R₁₆-R₁₇-R₁₈-R₁₉-R₂₀-R₂₁-R₂₂-R₂₃-R₂₄-R₂₅-R₂₆-R₂₇-R₂₈ form a stem-loop region, e.g., a D arm Region;
e) under physiological conditions residue-R₂₉ forms a linker region, e.g., a Linker 3 Region;
f) under physiological conditions residues-R₃₀-R₃₁-R₃₂-R₃₃-R₃₄-R₃₅-R₃₆-R₃₇-R₃₈-R₃₉-R₄₀-R₄₁-R₄₂-R₄₃-R₄₄-R₄₅-R₄₆ form a stem-loop region, e.g., an AC arm region;
g) under physiological conditions residue-[R₄₇]ₓ comprises a variable region, e.g., as described herein;
h) under physiological conditions residues-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄ form a stem-loop region, e.g., a T arm Region; or
i) under physiological conditions residue R₇₂ forms a linker region, e.g., a Linker 4 region.

### Alanine TREM Consensus Sequence

In an embodiment, a TREM disclosed herein comprises the sequence of Formula I_{ALA} (SEQ ID NO: 562),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for Ala is:
R₀=absent;
R₁₄, R₅₇=are independently A or absent;
R₂₆=A, C, G or absent;
R₅, R₆, R₁₅, R₁₆, R₂₁, R₃₀, R₃₁, R₃₂, R₃₄, R₃₇, R₄₁, R₄₂, R₄₃, R₄₄, R₄₅, R₄₈, R₄₉, R₅₀, R₅₈, R₅₉, R₆₃, R₆₄, R₆₆, R₆₇=are independently N or absent;
R₁₁, R₃₅, R₆₅=are independently A, C, U or absent;
R₁, R₉, R₂₀, R₃₈, R₄₀, R₅₁, R₅₂, R₅₆=are independently A, G or absent;
R₇, R₂₂, R₂₅, R₂₇, R₂₉, R₄₆, R₅₃, R₇₂=are independently A, G, U or absent;
R₂₄, R₆₉=are independently A, U or absent;
R₇₀, R₇₁=are independently C or absent;
R₃, R₄=are independently C, G or absent;
R₁₂, R₃₃, R₃₆, R₆₂, R₆₈=are independently C, G, U or absent;
R₁₃, R₁₇, R₂₈, R₃₉, R₅₅, R₆₀, R₆₁=are independently C, U or absent;
R₁₀, R₁₉, R₂₃=are independently G or absent;
R₂=G, U or absent;
R₈, R₁₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II_{ALA} (SEQ ID NO: 563),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ala is:
R₀, R₁₈=are absent;
R₁₄, R₂₄, R₅₇=are independently A or absent;
R₁₅, R₂₆, R₆₄=are independently A, C, G or absent;
R₁₆, R₃₁, R₅₀, R₅₉=are independently N or absent;
R₁₁, R₃₂, R₃₇, R₄₁, R₄₃, R₄₅, R₄₉, R₆₅, R₆₆=are independently A, C, U or absent;
R₁, R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃, R₇₂=are independently A, G, U or absent;
R₆, R₃₅, R₆₉=are independently A, U or absent;
R₅₅, R₆₀, R₇₀, R₇₁=are independently C or absent;
R₃=C, G or absent;
R₁₂, R₃₆, R₄₈=are independently C, G, U or absent;
R₁₃, R₁₇, R₂₈, R₃₀, R₃₄, R₃₉, R₅₈, R₆₁, R₆₂, R₆₇, R₆₈=are independently C, U or absent;
R₄, R₁₀, R₁₉, R₂₀, R₂₃, R₅₂=are independently G or absent;
R₂, R₈, R₃₃=are independently G, U or absent;
R₂₁, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III_{ALA} (SEQ ID NO: 564),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for Ala is:
R₀, R₁₈=are absent;
R₁₄, R₂₄, R₅₇, R₇₂=are independently A or absent;
R₁₅, R₂₆, R₆₄=are independently A, C, G or absent;
R₁₆, R₃₁, R₅₀=are independently N or absent;
R₁₁, R₃₂, R₃₇, R₄₁, R₄₃, R₄₅, R₄₉, R₆₅, R₆₆=are independently A, C, U or absent;
R₅, R₉, R₂₅, R₂₇, R₃₈, R₄₀, R₄₆, R₅₁, R₅₆=are independently A, G or absent;
R₇, R₂₂, R₂₉, R₄₂, R₄₄, R₅₃, R₆₃=are independently A, G, U or absent;
R₆, R₃₅=are independently A, U or absent;
R₅₅, R₆₀, R₆₁, R₇₀, R₇₁=are independently C or absent;
R₁₂, R₄₈, R₅₉=are independently C, G, U or absent;
R₁₃, R₁₇, R₂₈, R₃₀, R₃₄, R₃₉, R₅₈, R₆₂, R₆₇, R₆₈=are independently C, U or absent;
R₁, R₂, R₃, R₄, R₁₀, R₁₉, R₂₀, R₂₃, R₅₂=are independently G or absent;
R₃₃, R₃₆=are independently G, U or absent;
R₈, R₂₁, R₅₄, R₆₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Arginine TREM Consensus Sequence

In an embodiment, a TREM disclosed herein comprises the sequence of Formula I ARG (SEQ ID NO: 565),

R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂

wherein R is a ribonucleotide residue and the consensus for Arg is:
R₅₇=A or absent;
R₉, R₂₇=are independently A, C, G or absent;
R₁, R₂, R₃, R₄, R₅, R₆, R₇, R₁₁, R₁₂, R₁₆, R₂₁, R₂₂, R₂₃, R₂₅, R₂₆, R₂₉, R₃₀, R₃₁, R₃₂, R₃₃, R₃₄, R₃₇, R₄₂, R₄₄, R₄₅, R₄₆, R₄₈, R₄₉, R₅₀, R₅₁, R₅₈, R₆₂, R₆₃, R₆₄, R₆₅, R₆₆, R₆₇, R₆₈, R₆₉, R₇₀, R₇₁=are independently N or absent;
R₁₃, R₁₇, R₄₁=are independently A, C, U or absent;
R₁₉, R₂₀, R₂₄, R₄₀, R₅₆=are independently A, G or absent;
R₁₄, R₁₅, R₇₂=are independently A, G, U or absent;
R₁₈=A, U or absent;
R₃₈=C or absent;
R₃₅, R₄₃, R₆₁=are independently C, G, U or absent;
R₂₈, R₅₅, R₅₉, R₆₀=are independently C, U or absent;
R₀, R₁₀, R₅₂=are independently G or absent;
R₈, R₃₉=are independently G, U or absent;
R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula II ARG (SEQ ID NO: 566),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
wherein R is a ribonucleotide residue and the consensus for Arg is:
R₁₈=absent;
R₂₄, R₅₇=are independently A or absent;
R₄₁=A, C or absent;
R₃, R₇, R₃₄, R₅₀=are independently A, C, G or absent;
R₂, R₅, R₆, R₁₂, R₂₆, R₃₂, R₃₇, R₄₄, R₅₈, R₆₆, R₆₇, R₆₈, R₇₀=are independently N or absent;
R₄₉, R₇₁=are independently A, C, U or absent;
R₁, R₁₅, R₁₉, R₂₅, R₂₇, R₄₀, R₄₅, R₄₆, R₅₆, R₇₂=are independently A, G or absent;
R₁₄, R₂₉, R₆₃=are independently A, G, U or absent;
R₁₆, R₂₁=are independently A, U or absent;
R₃₈, R₆₁=are independently C or absent;
R₃₃, R₄₈=are independently C, G or absent;
R₄, R₉, R₁₁, R₄₃, R₆₂, R₆₄, R₆₉=are independently C, G, U or absent;
R₁₃, R₂₂, R₂₈, R₃₀, R₃₁, R₃₅, R₅₅, R₆₀, R₆₅=are independently C, U or absent;
R₀, R₁₀, R₂₀, R₂₃, R₅₁, R₅₂=are independently G or absent;
R₈, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ARG (SEQ ID NO: 567),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Arg is:
R₁₈=is absent;
R₁₅, R₂₁, R₂₄, R₄₁, R₅₇=are independently A or absent;
R₃₄, R₄₄=are independently A, C or absent;
R₃, R₅, R₅₈=are independently A, C, G or absent;
R₂, R₆, R₆₆, R₇₀=are independently N or absent;
R₃₇, R₄₉=are independently A, C, U or absent;
R₁, R₂₅, R₂₉, R₄₀, R₄₅, R₄₆, R₅₀=are independently A, G or absent;
R₁₄, R₆₃, R₆₈=are independently A, G, U or absent;
R₁₆=A, U or absent;
R₃₈, R₆₁=are independently C or absent;
R₇, R₁₁, R₁₂, R₂₆, R₄₈=are independently C, G or absent;
R₆₄, R₆₇, R₆₉=are independently C, G, U or absent;
R₄, R₁₃, R₂₂, R₂₈, R₃₀, R₃₁, R₃₅, R₄₃, R₅₅, R₆₀, R₆₂, R₆₅, R₇₁=are independently C, U or absent;
R₀, R₁₀, R₁₉, R₂₀, R₂₃, R₂₇, R₃₃, R₅₁, R₅₂, R₅₆, R₇₂=are independently G or absent;
R₈, R₉, R₃₂, R₃₉, R₄₂=are independently G, U or absent;
R₁₇, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Asparagine TREM Consensus Sequence

In an embodiment, a TREM disclosed herein comprises the sequence of Formula I ASN (SEQ ID NO: 568),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Asn is:
R₀, R₁₈=are absent;
R₄₁=A or absent;
R₁₄, R₄₈, R₅₆=are independently A, C, G or absent;
R₂, R₄, R₅, R₆, R₁₂, R₁₇, R₂₆, R₂₉, R₃₀, R₃₁, R₄₄, R₄₅, R₄₆, R₄₉, R₅₀, R₅₈, R₆₂, R₆₃, R₆₅, R₆₆, R₆₇, R₆₈, R₇₀, R₇₁=are independently N or absent;
R₁₁, R₁₃, R₂₂, R₄₂, R₅₅, R₅₉=are independently A, C, U or absent;
R₉, R₁₅, R₂₄, R₂₇, R₃₄, R₃₇, R₅₁, R₇₂=are independently A, G or absent;
R₁, R₇, R₂₅, R₆₉=are independently A, G, U or absent;
R₄₀, R₅₇=are independently A, U or absent;
R₆₀=C or absent;
R₃₃=C, G or absent;
R₂₁, R₃₂, R₄₃, R₆₄=are independently C, G, U or absent;
R₃, R₁₆, R₂₈, R₃₅, R₃₆, R₆₁=are independently C, U or absent;
R₁₀, R₁₉, R₂₀, R₅₂=are independently G or absent;
R₅₄=G, U or absent;
R₈, R₂₃, R₃₈, R₃₉, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ASN (SEQ ID NO: 569),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Asn is:
R₀, R₁₈=are absent
R₂₄, R₄₁, R₄₆, R₆₂=are independently A or absent;
R₅₉=A, C or absent;
R₁₄, R₅₆, R₆₆=are independently A, C, G or absent;
R₁₇, R₂₉=are independently N or absent;
R₁₁, R₂₆, R₄₂, R₅₅=are independently A, C, U or absent;
R₁, R₉, R₁₂, R₁₅, R₂₅, R₃₄, R₃₇, R₄₈, R₅₁, R₆₇, R₆₈, R₆₉, R₇₀, R₇₂=are independently A, G or absent;
R₄₄, R₄₅, R₅₈=are independently A, G, U or absent;
R₄₀, R₅₇=are independently A, U or absent;
R₅, R₂₈, R₆₀=are independently C or absent;
R₃₃, R₆₅=are independently C, G or absent;
R₂₁, R₄₃, R₇₁=are independently C, G, U or absent;
R₃, R₆, R₁₃, R₂₂, R₃₂, R₃₅, R₃₆, R₆₁, R₆₃, R₆₄=are independently C, U or absent;
R₇, R₁₀, R₁₉, R₂₀, R₂₇, R₄₉, R₅₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASN (SEQ ID NO: 570),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂ wherein R is a ribonucleotide residue and the consensus for Asn is:
R₀, R₁₈=are absent
R₂₄, R₄₀, R₄₁, R₄₆, R₆₂=are independently A or absent;
R₅₉=A, C or absent;
R₁₄, R₅₆, R₆₆=are independently A, C, G or absent;
R₁₁, R₂₆, R₄₂, R₅₅=are independently A, C, U or absent;
R₁, R₉, R₁₂, R₁₅, R₃₄, R₃₇, R₄₈, R₅₁, R₆₇, R₆₈, R₆₉, R₇₀=are independently A, G or absent;
R₄₄, R₄₅, R₅₈=are independently A, G, U or absent;
R₅₇=A, U or absent;
R₅, R₂₈, R₆₀=are independently C or absent;
R₃₃, R₆₅=are independently C, G or absent;
R₁₇, R₂₁, R₂₉=are independently C, G, U or absent;
R₃, R₆, R₁₃, R₂₂, R₃₂, R₃₅, R₃₆, R₄₃, R₆₁, R₆₃, R₆₄, R₇₁=are independently C, U or absent;
R₇, R₁₀, R₁₉, R₂₀, R₂₅, R₂₇, R₄₉, R₅₂, R₇₂=are independently G or absent;
R₅₄=G, U or absent;
R₂, R₄, R₈, R₁₆, R₂₃, R₃₀, R₃₁, R₃₈, R₃₉, R₅₀, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Aspartate TREM Consensus Sequence

In an embodiment, a TREM disclosed herein comprises the sequence of Formula I ASP (SEQ ID NO: 571),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Asp is:
R₀=absent
R₂₄, R₇₁=are independently A, C or absent;
R₃₃, R₄₆=are independently A, C, G or absent;
R₂, R₃, R₄, R₅, R₆, R₁₂, R₁₆, R₂₂, R₂₆, R₂₉, R₃₁, R₃₂, R₄₄, R₄₈, R₄₉, R₅₈, R₆₃, R₆₄, R₆₆, R₆₇, R₆₈, R₆₉=are independently N or absent;
R₁₃, R₂₁, R₃₄, R₄₁, R₅₇, R₆₅=are independently A, C, U or absent;
R₀, R₁₀, R₁₄, R₁₅, R₂₀, R₂₇, R₃₇, R₄₀, R₅₁, R₅₆, R₇₂=are independently A, G or absent;
R₇, R₂₅, R₄₂=are independently A, G, U or absent;
R₃₉=C or absent;
R₅₀, R₆₂=are independently C, G or absent;
R₃₀, R₄₃, R₄₅, R₅₅, R₇₀=are independently C, G, U or absent;
R₈, R₁₁, R₁₇, R₁₈, R₂₈, R₃₅, R₅₃, R₅₉, R₆₀, R₆₁=are independently C, U or absent;
R₁₉, R₅₂=are independently G or absent;
R₁=G, U or absent;
R₂₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues arc N; or no more than 20 residues arc absent.

In an embodiment, a TREM disclosed herein comprises the sequence of Formula II ASP (SEQ ID NO: 572),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Asp is:
R₀, R₁₇, R₁₈, R₂₃=are independently absent;
R₀, R₄₀=are independently A or absent;
R₂₄, R₇₁=are independently A, C or absent;
R₆₇, R₆₈=are independently A, C, G or absent;
R₂, R₆, R₆₆=are independently N or absent;
R₆₇, R₆₃=are independently A, C, U or absent;
R₁₀, R₁₄, R₂₇, R₃₃, R₃₇, R₄₄, R₄₆, R₅₁, R₅₆, R₆₄, R₇₂=are independently A, G or absent;
R₇, R₁₂, R₂₆, R₆₅=are independently A, U or absent;
R₃₉, R₆₁, R₆₂=are independently C or absent;
R₃, R₃₁, R₄₅, R₇₀=are independently C, G or absent;
R₄, R₅, R₂₉, R₄₃, R₅₅=are independently C, G, U or absent;
R₈, R₁₁, R₁₃, R₃₀, R₃₂, R₃₄, R₃₅, R₄₁, R₄₈, R₅₃, R₅₉, R₆₀=are independently C, U or absent;
R₁₅, R₁₉, R₂₀, R₂₅, R₄₂, R₅₀, R₅₂=are independently G or absent;
R₁, R₂₂, R₄₉, R₅₈, R₆₉=are independently G, U or absent;
R₁₆, R₂₁, R₂₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III ASP (SEQ ID NO: 573),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Asp is:
R₀, R₁₇, R₁₈, R₂₃=are absent
R₉, R₁₂, R₄₀, R₆₅, R₇₁=are independently A or absent;
R₂, R₂₄, R₅₇=are independently A, C or absent;
R₆, R₁₄, R₂₇, R₄₆, R₅₁, R₅₆, R₆₄, R₆₇, R₆₈=are independently A, G or absent;
R₃, R₃₁, R₃₅, R₃₉, R₆₁, R₆₂=are independently C or absent;
R₆₆=C, G or absent;
R₅, R₈, R₂₉, R₃₀, R₃₂, R₃₄, R₄₁, R₄₃, R₄₈, R₅₅, R₅₉, R₆₀, R₆₃=are independently C, U or absent;
R₁₀, R₁₅, R₁₉, R₂₀, R₂₅, R₃₃, R₃₇, R₄₂, R₄₄, R₄₅, R₄₉, R₅₀, R₅₂, R₆₉, R₇₀, R₇₂=are independently G or absent;
R₂₂, R₅₈=are independently G, U or absent;
R₁, R₄, R₇, R₁₁, R₁₃, R₁₆, R₂₁, R₂₆, R₂₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Cysteine TREM Consensus Sequence

In an embodiment, a TREM disclosed herein comprises the sequence of Formula I _{CYS} (SEQ ID NO: 574),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Cys is:
R₀=absent
R₁₄, R₃₉, R₅₇=are independently A or absent;
R₄₁=A, C or absent;
R₁₀, R₁₅, R₂₇, R₃₃, R₆₂=are independently A, C, G or absent;
R₃, R₄, R₅, R₆, R₁₂, R₁₃, R₁₆, R₂₄, R₂₆, R₂₉, R₃₀, R₃₁, R₃₂, R₃₄, R₄₂, R₄₄, R₄₅, R₄₆, R₄₈, R₄₉,R 58, R₆₃, R₆₄, R₆₆, R₆₇, R₆₈, R₆₉, R₇₀=are independently N or absent;
R₆₅=A, C, U or absent;
R₀, R₂₅, R₃₇, R₄₀, R₅₂, R₅₆=are independently A, G or absent;
R₇, R₂₀, R₅₁=are independently A, G, U or absent;
R₁₈, R₃₈, R₅₅=are independently C or absent;
R₂=C, G or absent;
R₂₁, R₂₈, R₄₃, R₅₀=are independently C, G, U or absent;
R₁₁, R₂₂, R₂₃, R₃₅, R₃₆, R₅₉, R₆₀, R₆₁, R₇₁, R₇₂=are independently C, U or absent;
R₁, R₁₉=are independently G or absent;
R₁₇=G, U or absent;
R₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II CYS (SEQ ID NO: 575),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂ wherein R is a ribonucleotide residue and the consensus for Cys is:
R₀, R₁₈, R₂₃=are absent;
R₁₄, R₂₄, R₂₆, R₂₉, R₃₉, R₄₁, R₄₅, R₅₇=are independently A or absent;
R₄₄=A, C or absent;
R₂₇, R₆₂=are independently A, C, G or absent;
R₁₆=A, C, G, U or absent;
R₃₀, R₇₀=are independently A, C, U or absent;
R₅, R₇, R₉, R₂₅, R₃₄, R₃₇, R₄₀, R₄₆, R₅₂, R₅₆, R₅₈, R₆₆=are independently A, G or absent;
R₂₀, R₅₁=are independently A, G, U or absent;
R₃₅, R₃₈, R₄₃, R₅₅, R₆₉=are independently C or absent;
R₂, R₄, R₁₅=are independently C, G or absent;
R₁₃=C, G, U or absent;
R₆, R₁₁, R₂₈, R₃₆, R₄₈, R₄₉, R₅₀, R₆₀, R₆₁, R₆₇, R₆₈, R₇₁, R₇₂=are independently C, U or absent;
R₁, R₃, R₁₀, R₁₉, R₃₃, R₆₃=are independently G or absent;
R₈, R₁₇, R₂₁, R₆₄=are independently G, U or absent;
R₁₂, R₂₂, R₃₁, R₃₂, R₄₂, R₅₃, R₅₄, R₆₅=are independently U or absent;
R₅₉=U, or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III CYS (SEQ ID NO: 576),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Cys is:
R₀, R₁₈, R₂₃=are absent
R₁₄, R₂₄, R₂₆, R₂₉, R₃₄, R₃₉, R₄₁, R₄₅, R₅₇, R₅₈=are independently A or absent;
R₄₄, R₇₀=are independently A, C or absent;
R₆₂=A, C, G or absent;
R₁₆=N or absent;
R₅, R₇, R₉, R₂₀, R₄₀, R₄₆, R₅₁, R₅₂, R₅₆, R₆₆=are independently A, G or absent;
R₂₈, R₃₅, R₃₈, R₄₃, R₅₅, R₆₇, R₆₉=are independently C or absent;
R₄, R₁₅=are independently C, G or absent;
R₆, R₁₁, R₁₃, R₃₀, R₄₈, R₄₉, R₅₀, R₆₀, R₆₁, R₆₈, R₇₁, R₇₂=are independently C, U or absent;
R₁, R₂, R₃, R₁₀, R₁₉, R₂₅, R₂₇, R₃₃, R₃₇, R₆₃=are independently G or absent;
R₈, R₂₁, R₆₄=are independently G, U or absent;
R₁₂, R₁₇, R₂₂, R₃₁, R₃₂, R₃₆, R₄₂, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glutamine TREM Consensus Sequence

In an embodiment, a TREM disclosed herein comprises the sequence of Formula I GLN (SEQ ID NO: 577),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gln is:
R₀, R₁₈=are absent;
R₁₄, R₂₄, R₅₇=are independently A or absent;
R₀, R₂₆, R₂₇, R₃₃, R₅₆=are independently A, C, G or absent;
R₂, R₄, R₅, R₆, R₁₂, R₁₃, R₁₆, R₂₁, R₂₂, R₂₅, R₂₉, R₃₀, R₃₁, R₃₂, R₃₄, R₄₁, R₄₂, R₄₄, R₄₅, R₄₆, R₄₈, R₄₉, R₅₀, R₅₈,R 62, R₆₃, R₆₆, R₆₇, R₆₈, R₆₉, R₇₀=are independently N or absent;
R₁₇, R₂₃, R₄₃, R₆₅, R₇₁=are independently A, C, U or absent;
R₁₅, R₄₀, R₅₁, R₅₂=are independently A, G or absent;
R₁, R₇, R₇₂=are independently A, G, U or absent;
R₃, R₁₁, R₃₇, R₆₀, R₆₄=are independently C, G, U or absent;
R₂₈, R₃₅, R₅₅, R₅₉, R₆₁=are independently C, U or absent;
R₁₀, R₁₉, R₂₀=are independently G or absent;
R₃₉=G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLN (SEQ ID NO: 578),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gln is:
R₀, R₁₈, R₂₃=are absent
R₁₄, R₂₄, R₅₇=are independently A or absent;
R₁₇, R₇₁=are independently A, C or absent;
R₂₅, R₂₆, R₃₃, R₄₄, R₄₆, R₅₆, R₆₉=are independently A, C, G or absent;
R₄, R₅, R₁₂, R₂₂, R₂₉, R₃₀, R₄₈, R₄₉, R₆₃, R₆₇, R₆₈=are independently N or absent;
R₃₁, R₄₃, R₆₂, R₆₅, R₇₀=are independently A, C, U or absent;
R₁₅, R₂₇, R₃₄, R₄₀, R₄₁, R₅₁, R₅₂=are independently A, G or absent;
R₂, R₇, R₂₁, R₄₅, R₅₀, R₅₈, R₆₆, R₇₂=are independently A, G, U or absent;
R₃, R₁₃, R₃₂, R₃₇, R₄₂, R₆₀, R₆₄=are independently C, G, U or absent;
R₆, R₁, R₂₈, R₃₅, R₅₅, R₅₉, R₆₁=are independently C, U or absent;
R₀, R₁₀, R₁₉, R₂₀=are independently G or absent;
R₁, R₁₆, R₃₉=are independently G, U or absent;
R₈, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLN (SEQ ID NO: 579),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gln is:
R₀, R₁₈, R₂₃=are absent
R₁₄, R₂₄, R₄₁, R₅₇=are independently A or absent;
R₁₇, R₇₁=are independently A, C or absent;
R₅, R₂₅, R₂₆, R₄₆, R₅₆, R₆₉=are independently A, C, G or absent;
R₄, R₂₂, R₂₉, R₃₀, R₄₈, R₄₉, R₆₃, R₆₈=are independently N or absent;
R₄₃, R₆₂, R₆₅, R₇₀=are independently A, C, U or absent;
R₁₅, R₂₇, R₃₃, R₃₄, R₄₀, R₅₁, R₅₂=are independently A, G or absent;
R₂, R₇, R₁₂, R₄₅, R₅₀, R₅₈, R₆₆=are independently A, G, U or absent;
R₃₁=A, U or absent;
R₃₂, R₄₄, R₆₀=are independently C, G or absent;
R₃, R₁₃, R₃₇, R₄₂, R₆₄, R₆₇=are independently C, G, U or absent;
R₆, R₁, R₂₈, R₃₅, R₅₅, R₅₉, R₆₁=are independently C, U or absent;
R₉, R₁₀, R₁₉, R₂₀=are independently G or absent;
R₁, R₂₁, R₃₉, R₇₂=are independently G, U or absent;
R₈, R₁₆, R₃₆, R₃₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

### Glutamate TREM Consensus Sequence

In an embodiment, a TREM disclosed herein comprises the sequence of Formula I GLU (SEQ ID NO: 580),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Glu is:
R₀=absent;
R₃₄, R₄₃, R₆₈, R₆₉=are independently A, C, G or absent;
R₁, R₂, R₅, R₆, R₉, R₁₂, R₁₆, R₂₀, R₂₁, R₂₆, R₂₇, R₂₉, R₃₀, R₃₁, R₃₂, R₃₃, R₄₁, R₄₄, R₄₅, R₄₆, R₄₈, R₅₀, R₅₁, R₅₈, R₆₃, R₆₄, R₆₅, R₆₆, R₇₀, R₇₁=are independently N or absent;
R₁₃, R₁₇, R₂₃, R₆₁=are independently A, C, U or absent;
R₁₀, R₁₄, R₂₄, R₄₀, R₅₂, R₅₆=are independently A, G or absent;
R₇, R₁₅, R₂₅, R₆₇, R₇₂=are independently A, G, U or absent;
R₁₁, R₅₇=are independently A, U or absent;
R₃₉=C, G or absent;
R₃, R₄, R₂₂, R₄₂, R₄₉, R₅₅, R₆₂=are independently C, G, U or absent;
R₁₈, R₂₈, R₃₅, R₃₇, R₅₃, R₅₉, R₆₀=are independently C, U or absent;
R₁₉=G or absent;
R₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II GLU (SEQ ID NO: 581),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Glu is:
R₀, R₁₈, R₂₃=are absent
R₁₇, R₄₀=are independently A or absent;
R₂₆, R₂₇, R₃₄, R₄₃, R₆₈, R₆₉, R₇₁=are independently A, C, G or absent;
R₁, R₂, R₅, R₁₂, R₂₁, R₃₁, R₃₃, R₄₁, R₄₅, R₄₈, R₅₁, R₅₈, R₆₆, R₇₀=are independently N or absent;
R₄₄, R₆₁=are independently A, C, U or absent;
R₀, R₁₄, R₂₄, R₂₅, R₅₂, R₅₆, R₆₃=are independently A, G or absent;
R₇, R₁₅, R₄₆, R₅₀, R₆₇, R₇₂=are independently A, G, U or absent;
R₂₉, R₅₇=are independently A, U or absent;
R₆₀=C or absent;
R₃₉=C, G or absent;
R₃, R₆, R₂₀, R₃₀, R₃₂, R₄₂, R₅₅, R₆₂, R₆₅=are independently C, G, U or absent;
R₄, R₈, R₁₆, R₂₈, R₃₅, R₃₇, R₄₉, R₅₃, R₅₉=are independently C, U or absent;
R₁₀, R₁₉=are independently G or absent;
R₂₂, R₆₄=are independently G, U or absent;
R₁, R₁₃, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLU (SEQ ID NO: 582),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Glu is:
R₀, R₁₇, R₁₈, R₂₃=are absent
R₁₄, R₂₇, R₄₀, R₇₁=are independently A or absent;
R₄₄=A, C or absent;
R₄₃=A, C, G or absent;
R₁, R₃₁, R₃₃, R₄₅, R₅₁, R₆₆=are independently N or absent;
R₂₁, R₄₁=are independently A, C, U or absent;
R₇, R₂₄, R₂₅, R₅₀, R₅₂, R₅₆, R₆₃, R₆₈, R₇₀=are independently A, G or absent;
R₅, R₄₆=are independently A, G, U or absent;
R₂₉, R₅₇, R₆₇, R₇₂=are independently A, U or absent;
R₂, R₃₉, R₆₀=are independently C or absent;
R₃, R₁₂, R₂₀, R₂₆, R₃₄, R₆₉=are independently C, G or absent;
R₆, R₃₀, R₄₂, R₄₈, R₆₅=are independently C, G, U or absent;
R₄, R₁₆, R₂₈, R₃₅, R₃₇, R₄₉, R₅₃, R₅₅, R₅₈, R₆₁, R₆₂=are independently C, U or absent;
R₉, R₁₀, R₁₉, R₆₄=are independently G or absent;
R₁₅, R₂₂, R₃₂=are independently G, U or absent;
R₈, R₁, R₁₃, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Glycine TREM Consensus Sequence

In an embodiment, a TREM disclosed herein comprises the sequence of Formula I GLY (SEQ ID NO: 583),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gly is:
R₀=absent;
R₂₄=A or absent;
R₃, R₉, R₄₀, R₅₀, R₅₁=are independently A, C, G or absent;
R₄, R₅, R₆, R₇, R₁₂, R₁₆, R₂₁, R₂₂, R₂₆, R₂₉, R₃₀, R₃₁, R₃₂, R₃₃, R₃₄, R₄₁, R₄₂, R₄₃, R₄₄, R₄₅, R₄₆, R₄₈, R₄₉, R₅₈, R₆₃, R₆₄, R₆₅, R₆₆, R₆₇, R₆₈=are independently N or absent;
R₅₉=A, C, U or absent;
R₁, R₁₀, R₁₄, R₁₅, R₂₇, R₅₆=are independently A, G or absent;
R₂₀, R₂₅=are independently A, G, U or absent;
R₅₇, R₇₂=are independently A, U or absent;
R₃₈, R₃₉, R₆₀=are independently C or absent;
R₅₂=C, G or absent;
R₂, R₁₉, R₃₇, R₅₄, R₅₅, R₆₁, R₆₂, R₆₉, R₇₀=are independently C, G, U or absent;
R₁₁, R₁₃, R₁₇, R₂₈, R₃₅, R₃₆, R₇₁=are independently C, U or absent;
R₈, R₁₈, R₂₃, R₅₃=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues arc N; or no more than 20 residues arc absent.

In an embodiment, a TREM disclosed herein comprises the sequence of Formula II GLY (SEQ ID NO: 584),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₆₈=are independently A, C, G or absent;
R₅, R₃₀, R₄₁, R₄₂, R₄₄, R₄₉, R₆₇=are independently A, C, G, U or absent;
R₃₁, R₃₂, R₃₄=are independently A, C, U or absent;
R₀, R₁₀, R₁₄, R₁₅, R₃₃, R₅₀, R₅₆=are independently A, G or absent;
R₁₂, R₁₆, R₂₂, R₂₅, R₂₉, R₄₆=are independently A, G, U or absent;
R₅₇=A, U or absent;
R₁₇, R₃₈, R₃₉, R₆₀, R₆₁, R₇₁=are independently C or absent;
R₆, R₅₂, R₆₄, R₆₆=are independently C, G or absent;
R₂, R₄, R₃₇, R₄₈, R₅₅, R₆₅=are independently C, G, U or absent;
R₁₃, R₃₅, R₄₃, R₆₂, R₆₉=are independently C, U or absent;
R₁, R₁₉, R₂₀, R₅₁, R₇₀=are independently G or absent;
R₂₁, R₄₅, R₆₃=are independently G, U or absent;
R₈, R₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III GLY (SEQ ID NO: 585),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Gly is:
R₀, R₁₈, R₂₃=are absent
R₂₄, R₂₇, R₄₀, R₇₂=are independently A or absent;
R₂₆=A, C or absent;
R₃, R₇, R₄₉, R₆₈=are independently A, C, G or absent;
R₅, R₃₀, R₄₁, R₄₄, R₆₇=are independently N or absent;
R₃₁, R₃₂, R₃₄=are independently A, C, U or absent;
R₀, R₁₀, R₁₄, R₁₅, R₃₃, R₅₀, R₅₆=are independently A, G or absent;
R₁₂, R₂₅, R₂₉, R₄₂, R₄₆=are independently A, G, U or absent;
R₁₆, R₅₇=are independently A, U or absent;
R₁₇, R₃₈, R₃₉, R₆₀, R₆₁, R₇₁=are independently C or absent;
R₆, R₅₂, R₆₄, R₆₆=are independently C, G or absent;
R₃₇, R₄₈, R₆₅=are independently C, G, U or absent;
R₂, R₄, R₁₃, R₃₅, R₄₃, R₅₅, R₆₂, R₆₉=are independently C, U or absent;
R₁, R₁₉, R₂₀, R₅₁, R₇₀=are independently G or absent;
R₂₁, R₂₂, R₄₅, R₆₃=are independently G, U or absent;
R₈, R₁₁, R₂₈, R₃₆, R₅₃, R₅₄, R₅₈, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Histidine TREM Consensus Sequence

In an embodiment, a TREM disclosed herein comprises the sequence of Formula I HIS (SEQ ID NO: 586),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for His is:
R₂₃=absent;
R₁₄, R₂₄, R₅₇=are independently A or absent;
R₇₂=A, C or absent;
R₉, R₂₇, R₄₃, R₄₈, R₆₉=are independently A, C, G or absent;
R₃, R₄, R₅, R₆, R₁₂, R₂₅, R₂₆, R₂₉, R₃₀, R₃₁, R₃₄, R₄₂, R₄₅, R₄₆, R₄₉, R₅₀, R₅₈, R₆₂, R₆₃, R₆₆, R₆₇, R₆₈=are independently N or absent;
R₁₃, R₂₁, R₄₁, R₄₄, R₆₅=are independently A, C, U or absent;
R₄₀, R₅₁, R₅₆, R₇₀=are independently A, G or absent;
R₇, R₃₂=are independently A, G, U or absent;
R₅₅, R₆₀=are independently C or absent;
R₁₁, R₁₆, R₃₃, R₆₄=are independently C, G, U or absent;
R₂, R₁₇, R₂₂, R₂₈, R₃₅, R₅₃, R₅₉, R₆₁, R₇₁=are independently C, U or absent;
R₁, R₁₀, R₁₅, R₁₉, R₂₀, R₃₇, R₃₉, R₅₂=are independently G or absent;
R₀=G, U or absent;
R₈, R₁₈, R₃₆, R₃₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II HIS (SEQ ID NO: 587),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for His is:
R₀, R₁₇, R₁₈, R₂₃=are absent;
R₇, R₁₂, R₁₄, R₂₄, R₂₇, R₄₅, R₅₇, R₅₈, R₆₃, R₆₇, R₇₂=are independently A or absent;
R₃=A, C, U or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₂₅=C, G or absent;
R₉=C, G, U or absent;
R₈, R₁₃, R₂₆, R₃₃, R₃₅, R₅₀, R₅₃, R₆₁, R₆₈=are independently C, U or absent;
R₁, R₆, R₁₀, R₁₅, R₁₉, R₂₀, R₃₂, R₃₄, R₃₇, R₃₉, R₄₀, R₄₆, R₅₁, R₅₂, R₆₂, R₆₄, R₆₉=are independently G or absent;
R₁₆=G, U or absent;
R₅, R₁₁, R₂₁, R₂₂, R₂₉, R₃₁, R₃₆, R₃₈, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III HIS (SEQ ID NO: 588),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for His is:
R₀, R₁₇, R₁₈, R₂₃=are absent
R₇, R₁₂, R₁₄, R₂₄, R₂₇, R₄₅, R₅₇, R₅₈, R₆₃, R₆₇, R₇₂=are independently A or absent;
R₃=A, C or absent;
R₄, R₄₃, R₅₆, R₇₀=are independently A, G or absent;
R₄₉=A, U or absent;
R₂, R₂₈, R₃₀, R₄₁, R₄₂, R₄₄, R₄₈, R₅₅, R₆₀, R₆₆, R₇₁=are independently C or absent;
R₈, R₀, R₂₆, R₃₃, R₃₅, R₅₀, R₆₁, R₆₈=are independently C, U or absent;
R₁, R₆, R₁₀, R₁₅, R₁₉, R₂₀, R₂₅, R₃₂, R₃₄, R₃₇, R₃₉, R₄₀, R₄₆, R₅₁, R₅₂, R₆₂, R₆₄, R₆₉=are independently G or absent;
R₅, R₁₁, R₁₃, R₁₆, R₂₁, R₂₂, R₂₉, R₃₁, R₃₆, R₃₈, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Isoleucine TREM Consensus Sequence

In an embodiment, a TREM disclosed herein comprises the sequence of Formula I _{ILE} (SEQ ID NO: 589),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ile is:
R₂₃=absent;
R₃₈, R₄₁, R₅₇, R₇₂=are independently A or absent;
R₁, R₂₆=are independently A, C, G or absent;
R₀, R₃, R₄, R₆, R₁₆, R₃₁, R₃₂, R₃₄, R₃₇, R₄₂, R₄₃, R₄₄, R₄₅, R₄₆, R₄₈, R₄₉, R₅₀, R₅₈, R₅₉, R₆₂, R₆₃, R₆₄, R₆₆, R₆₇, R₆₈, R₆₉=are independently N or absent;
R₂₂, R₆₁, R₆₅=are independently A, C, U or absent;
R₉, R₁₄, R₁₅, R₂₄, R₂₇, R₄₀=are independently A, G or absent;
R₇, R₂₅, R₂₉, R₅₁, R₅₆=are independently A, G, U or absent;
R₁₈, R₅₄=are independently A, U or absent;
R₆₀=C or absent;
R₂, R₅₂, R₇₀=are independently C, G or absent;
R₅, R₁₂, R₂₁, R₃₀, R₃₃, R₇₁=are independently C, G, U or absent;
R₁₁, R₁₃, R₁₇, R₂₈, R₃₅, R₅₃, R₅₅=are independently C, U or absent;
R₁₀, R₁₉, R₂₀=are independently G or absent;
R₈, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II ILE (SEQ ID NO: 590),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ile is:
R₀, R₁₈, R₂₃=are absent
R₂₄, R₃₈, R₄₀, R₄₁, R₅₇, R₇₂=are independently A or absent;
R₂₆, R₆₅=are independently A, C or absent;
R₅₈, R₅₉, R₆₇=are independently N or absent;
R₂₂=A, C, U or absent;
R₆, R₉, R₁₄, R₁₅, R₂₉, R₃₄, R₄₃, R₄₆, R₄₈, R₅₀, R₅₁, R₆₃, R₆₉=are independently A, G or absent;
R₃₇, R₅₆=are independently A, G, U or absent;
R₅₄=A, U or absent;
R₂₈, R₃₅, R₆₀, R₆₂, R₇₁=are independently C or absent;
R₂, R₅₂, R₇₀=are independently C, G or absent;
R₅=C, G, U or absent;
R₃, R₄, R₁₁, R₁₃, R₁₇, R₂₁, R₃₀, R₄₂, R₄₄, R₄₅, R₄₉, R₅₃, R₅₅, R₆₁, R₆₄, R₆₆=are independently C, U or absent;
R₁, R₁₀, R₁₉, R₂₀, R₂₅, R₂₇, R₃₁, R₆₈=are independently G or absent;
R₇, R₁₂, R₃₂=are independently G, U or absent;
R₈, R₁₆, R₃₃, R₃₆, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{ILE} (SEQ ID NO: 591),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ile is:
R₀, R₁₈, R₂₃=are absent
R₁₄, R₂₄, R₃₈, R₄₀, R₄₁, R₅₇, R₇₂=are independently A or absent;
R₂₆, R₆₅=are independently A, C or absent;
R₂₂, R₅₉=are independently A, C, U or absent;
R₀, R₉, R₁₅, R₃₄, R₄₃, R₄₆, R₅₁, R₅₆, R₆₃, R₆₉=are independently A, G or absent;
R₃₇=A, G, U or absent;
R₁₃, R₂₈, R₃₅, R₄₄, R₅₅, R₆₀, R₆₂, R₇₁=are independently C or absent;
R₂, R₅, R₇₀=are independently C, G or absent;
R₅₈, R₆₇=are independently C, G, U or absent;
R₃, R₄, R₁₁, R₁₇, R₂₁, R₃₀, R₄₂, R₄₅, R₄₉, R₅₃, R₆₁, R₆₄, R₆₆=are independently C, U or absent;
R₁, R₁₀, R₁₉, R₂₀, R₂₅, R₂₇, R₂₉, R₃₁, R₃₂, R₄₈, R₅₀, R₅₂, R₆₈=are independently G or absent;
R₇, R₁₂=are independently G, U or absent;
R₈, R₁₆, R₃₃, R₃₆, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Methionine TREM Consensus Sequence

In an embodiment, a TREM disclosed herein comprises the sequence of Formula I _{MET} (SEQ ID NO: 592),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Met is:
R₀, R₂₃=are absent;
R₁₄, R₃₈, R₄₀, R₅₇=are independently A or absent;
R₆₀=A, C or absent;
R₃₃, R₄₈, R₇₀=are independently A, C, G or absent;
R₁, R₃, R₄, R₅, R₆, R₁₁, R₁₂, R₁₆, R₁₇, R₂₁, R₂₂, R₂₆, R₂₇, R₂₉, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₁=are independently N or absent;
R₁₈, R₃₅, R₄₁, R₅₉, R₆₅=are independently A, C, U or absent;
R₉, R₁₅, R₅₁=are independently A, G or absent;
R₇, R₂₄, R₂₅, R₃₄, R₅₃, R₅₆=are independently A, G, U or absent;
R₇₂=A, U or absent;
R₃₇=C or absent;
R₁₀, R₅₅=are independently C, G or absent;
R₂, R₁₃, R₂₈, R₄₃, R₆₄=are independently C, G, U or absent;
R₃₆, R₆₁=are independently C, U or absent;
R₁₉, R₂₀, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II MET (SEQ ID NO: 593),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₀, R₆₂, R₆₅=are independently A, C or absent;
R₆, R₄₅, R₆₇=are independently A, C, G or absent;
R₄=N or absent;
R₂₁, R₄₂=are independently A, C, U or absent;
R₁, R₉, R₂₇, R₂₉, R₃₂, R₄₆, R₅₁=are independently A, G or absent;
R₁₇, R₄₉, R₅₃, R₅₆, R₅₈=are independently A, G, U or absent;
R₆₃=A, U or absent;
R₃, R₁₃, R₃₇=are independently C or absent;
R₄₈, R₅₅, R₆₄, R₇₀=are independently C, G or absent;
R₂, R₅, R₆₆, R₆₈=are independently C, G, U or absent;
R₁₁, R₁₆, R₂₆, R₂₈, R₃₀, R₃₁, R₃₅, R₃₆, R₄₃, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues arc N; or no more than 20 residues arc absent.

In an embodiment, a TREM disclosed herein comprises the sequence of Formula III MET (SEQ ID NO: 594),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Met is:
R₀, R₁₈, R₂₂, R₂₃=are absent
R₁₄, R₂₄, R₃₈, R₄₀, R₄₁, R₅₇, R₇₂=are independently A or absent;
R₅₉, R₆₂, R₆₅=are independently A, C or absent;
R₆, R₆₇=are independently A, C, G or absent;
R₄, R₂₁=are independently A, C, U or absent;
R₁, R₉, R₂₇, R₂₉, R₃₂, R₄₅, R₄₆, R₅₁=are independently A, G or absent;
R₁₇, R₅₆, R₅₈=are independently A, G, U or absent;
R₄₉, R₅₃, R₆₃=are independently A, U or absent;
R₃, R₁₃, R₂₆, R₃₇, R₄₃, R₆₀=are independently C or absent;
R₂, R₄₈, R₅₅, R₆₄, R₇₀=are independently C, G or absent;
R₅, R₆₆=are independently C, G, U or absent;
R₁₁, R₁₆, R₂₈, R₃₀, R₃₁, R₃₅, R₃₆, R₄₂, R₄₄, R₆₁, R₇₁=are independently C, U or absent;
R₁₀, R₁₂, R₁₅, R₁₉, R₂₀, R₂₅, R₃₃, R₅₂, R₆₉=are independently G or absent;
R₇, R₃₄, R₅₀, R₆₈=are independently G, U or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Leucine TREM Consensus Sequence

In an embodiment, a TREM disclosed herein comprises the sequence of Formula I _{LEU} (SEQ ID NO: 595),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Leu is:
R₀=absent;
R₃₈, R₅₇=are independently A or absent;
R₆₀=A, C or absent;
R₁, R₁₃, R₂₇, R₄₈, R₅₁, R₅₆=are independently A, C, G or absent;
R₂, R₃, R₄, R₅, R₆, R₇, R₀, R₁₀, R₁₁, R₁₂, R₁₆, R₂₃, R₂₆, R₂₈, R₂₉, R₃₀, R₃₁, R₃₂, R₃₃, R₃₄, R₃₇, R₄₁, R₄₂, R₄₃, R₄₄, R₄₅, R₄₆, R₄₉, R₅₀, R₅₈, R₆₂, R₆₃, R₆₅, R₆₆, R₆₇, R₆₈, R₆₉, R₇₀=are independently N or absent;
R₁₇, R₁₈, R₂₁, R₂₂, R₂₅, R₃₅, R₅₅=are independently A, C, U or absent;
R₁₄, R₁₅, R₃₉, R₇₂=are independently A, G or absent;
R₂₄, R₄₀=are independently A, G, U or absent;
R₅₂, R₆₁, R₆₄, R₇₁=are independently C, G, U or absent;
R₃₆, R₅₃, R₅₉=are independently C, U or absent;
R₁₉=G or absent;
R₂₀=G, U or absent;
R₈, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II LEU (SEQ ID NO: 596),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₆₉=are independently A, C, G or absent;
R₆, R₃₃, R₄₁, R₄₃, R₄₆, R₄₉, R₅₈, R₆₃, R₆₆, R₇₀=are independently N or absent;
R₁₁, R₁₂, R₁₇, R₂₁, R₂₂, R₂₈, R₃₁, R₃₇, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₂₆, R₃₀, R₅₂, R₆₂, R₆₄, R₆₅, R₆₇, R₆₈=are independently C, G, U or absent;
R₁₈, R₃₅, R₄₂, R₅₃, R₅₉, R₆₁, R₇₁=are independently C, U or absent;
R₁₉, R₅₁=are independently G or absent;
R₁₀, R₂₀=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III LEU (SEQ ID NO: 597),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Leu is:
R₀=absent
R₃₈, R₅₇, R₇₂=are independently A or absent;
R₆₀=A, C or absent;
R₄, R₅, R₄₈, R₅₀, R₅₆, R₅₈, R₆₉=are independently A, C, G or absent;
R₆, R₃₃, R₄₃, R₄₆, R₄₉, R₆₃, R₆₆, R₇₀=are independently N or absent;
R₁₁, R₁₂, R₁₇, R₂₁, R₂₂, R₂₈, R₃₁, R₃₇, R₄₁, R₄₄, R₅₅=are independently A, C, U or absent;
R₁, R₉, R₁₄, R₁₅, R₂₄, R₂₇, R₃₄, R₃₉=are independently A, G or absent;
R₇, R₂₉, R₃₂, R₄₀, R₄₅=are independently A, G, U or absent;
R₂₅=A, U or absent;
R₁₃=C, G or absent;
R₂, R₃, R₁₆, R₃₀, R₅₂, R₆₂, R₆₄, R₆₇, R₆₈=are independently C, G, U or absent;
R₁₈, R₃₅, R₄₂, R₅₃, R₅₉, R₆₁, R₆₅, R₇₁=are independently C, U or absent;
R₁₉, R₅₁=are independently G or absent;
R₁₀, R₂₀, R₂₆=are independently G, U or absent;
R₈, R₂₃, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Lysine TREM Consensus Sequence

In an embodiment, a TREM disclosed herein comprises the sequence of Formula I LYS (SEQ ID NO: 598),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Lys is:
R₀=absent
R₁₄=A or absent;
R₄₀, R₄₁=are independently A, C or absent;
R₃₄, R₄₃, R₅₁=are independently A, C, G or absent;
R₁, R₂, R₃, R₄, R₅, R₆, R₇, R₁₁, R₁₂, R₁₆, R₂₁, R₂₆, R₃₀, R₃₁, R₃₂, R₄₄, R₄₅, R₄₆, R₄₈, R₄₉, R₅₀, R₅₈, R₆₂, R₆₃, R₆₅, R₆₆, R₆₇, R₆₈, R₆₉, R₇₀=are independently N or absent;
R₁₃, R₁₇, R₅₉, R₇₁=are independently A, C, U or absent;
R₀, R₁₅, R₁₉, R₂₀, R₂₅, R₂₇, R₅₂, R₅₆=are independently A, G or absent;
R₂₄, R₂₉, R₇₂=are independently A, G, U or absent;
R₁₈, R₅₇=are independently A, U or absent;
R₁₀, R₃₃=are independently C, G or absent;
R₄₂, R₆₁, R₆₄=are independently C, G, U or absent;
R₂₈, R₃₅, R₃₆, R₃₇, R₅₃, R₅₅, R₆₀=are independently C, U or absent;
R₈, R₂₂, R₂₃, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II LYS (SEQ ID NO: 599),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Lys is:
R₀, R₁₈, R₂₃=are absent
R₁₄=A or absent;
R₄₀, R₄₁, R₄₃=are independently A, C or absent;
R₃, R₇=are independently A, C, G or absent;
R₁, R₆, R₁₁, R₃₁, R₄₅, R₄₈, R₄₉, R₆₃, R₆₅, R₆₆, R₆₈=are independently N or absent;
R₂, R₁₂, R₁₃, R₁₇, R₄₄, R₆₇, R₇₁=are independently A, C, U or absent;
R₉, R₁₅, R₁₉, R₂₀, R₂₅, R₂₇, R₃₄, R₅₀, R₅₂, R₅₆, R₇₀, R₇₂=are independently A, G or absent;
R₅, R₂₄, R₂₆, R₂₉, R₃₂, R₄₆, R₆₉=are independently A, G, U or absent;
R₅₇=A, U or absent;
R₁₀, R₆₁=are independently C, G or absent;
R₄, R₁₆, R₂₁, R₃₀, R₅₈, R₆₄=are independently C, G, U or absent;
R₂₈, R₃₅, R₃₆, R₃₇, R₄₂, R₅₃, R₅₅, R₅₉, R₆₀, R₆₂=are independently C, U or absent;
R₃₃, R₅₁=are independently G or absent;
R₈-G, U or absent;
R₂₂, R₃₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues arc N; or no more than 20 residues arc absent.

In an embodiment, a TREM disclosed herein comprises the sequence of Formula III LYS (SEQ ID NO: 600),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Lys is:
R₀, R₁₈, R₂₃=absent
R₀, R₁₄, R₃₄, R₄₁=are independently A or absent;
R₄₀=A, C or absent;
R₁, R₃, R₇, R₃₁=are independently A, C, G or absent;
R₄₈, R₆₅, R₆₈=are independently N or absent;
R₂, R₁₃, R₁₇, R₄₄, R₆₃, R₆₆=are independently A, C, U or absent;
R₅, R₁₅, R₁₉, R₂₀, R₂₅, R₂₇, R₂₉, R₅₀, R₅₂, R₅₆, R₇₀, R₇₂=are independently A, G or absent;
R₆, R₂₄, R₃₂, R₄₉=are independently A, G, U or absent;
R₁₂, R₂₆, R₄₆, R₅₇=are independently A, U or absent;
R₁₁, R₂₈, R₃₅, R₄₃=are independently C or absent;
R₁₀, R₄₅, R₆₁=are independently C, G or absent;
R₄, R₂₁, R₆₄=are independently C, G, U or absent;
R₃₇, R₅₃, R₅₅, R₅₉, R₆₀, R₆₂, R₆₇, R₇₁=are independently C, U or absent;
R₃₃, R₅₁=are independently G or absent;
R₈, R₃₀, R₅₈, R₆₉=are independently G, U or absent;
R₁₆, R₂₂, R₃₆, R₃₈, R₃₉, R₄₂, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Phenylalanine TREM Consensus Sequence

In an embodiment, a TREM disclosed herein comprises the sequence of Formula I PHE (SEQ ID NO: 601),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Phe is:
R₀, R₂₃=are absent
R₀, R₁₄, R₃₈, R₃₉, R₅₇, R₇₂=are independently A or absent;
R₇₁=A, C or absent;
R₄₁, R₇₀=are independently A, C, G or absent;
R₄, R₅, R₆, R₃₀, R₃₁, R₃₂, R₃₄, R₄₂, R₄₄, R₄₅, R₄₆, R₄₈, R₄₉, R₅₈, R₆₂, R₆₃, R₆₆, R₆₇, R₆₈, R₆₉=are independently N or absent;
R₁₆, R₆₁, R₆₅=are independently A, C, U or absent;
R₁₅, R₂₆, R₂₇, R₂₉, R₄₀, R₅₆=are independently A, G or absent;
R₇, R₅₁=are independently A, G, U or absent;
R₂₂, R₂₄=are independently A, U or absent;
R₅₅, R₆₀=are independently C or absent;
R₂, R₃, R₂₁, R₃₃, R₄₃, R₅₀, R₆₄=are independently C, G, U or absent;
R₁₁, R₁₂, R₁₃, R₁₇, R₂₈, R₃₅, R₃₆, R₅₉=are independently C, U or absent;
R₁₀, R₁₉, R₂₀, R₂₅, R₃₇, R₅₂=are independently G or absent;
R₁=G, U or absent;
R₈, R₁₈, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PHE (SEQ ID NO: 602),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Phe is:
R₀, R₁₈, R₂₃=absent
R₁₄, R₂₄, R₃₈, R₃₉, R₅₇, R₇₂=are independently A or absent;
R₄₆, R₇₁=are independently A, C or absent;
R₄, R₇₀=are independently A, C, G or absent;
R₄₅=A, C, U or absent;
R₆, R₇, R₁₅, R₂₆, R₂₇, R₃₂, R₃₄, R₄₀, R₄₁, R₅₆, R₆₉=are independently A, G or absent;
R₂₉=A, G, U or absent;
R₅, R₉, R₆₇=are independently A, U or absent;
R₃₅, R₄₉, R₅₅, R₆₀=are independently C or absent;
R₂₁, R₄₃, R₆₂=are independently C, G or absent;
R₂, R₃₃, R₆₈=are independently C, G, U or absent;
R₃, R₁₁, R₁₂, R₁₃, R₂₈, R₃₀, R₃₆, R₄₂, R₄₄, R₄₈, R₅₈, R₅₉, R₆₁, R₆₆=are independently C, U or absent;
R₁₀, R₁₉, R₂₀, R₂₅, R₃₇, R₅₁, R₅₂, R₆₃, R₆₄=are independently G or absent;
R₁, R₃₁, R₅₀=are independently G, U or absent;
R₈, R₁₆, R₁₇, R₂₂, R₅₃, R₅₄, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III PHE (SEQ ID NO: 603),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Phe is:
R₀, R₁₈, R₂₂, R₂₃=absent
R₅, R₇, R₁₄, R₂₄, R₂₆, R₃₂, R₃₄, R₃₈, R₃₉, R₄₁, R₅₇, R₇₂=are independently A or absent;
R₄₆=A, C or absent;
R₇₀=A, C, G or absent;
R₄, R₆, R₁₅, R₅₆, R₆₉=are independently A, G or absent;
R₀, R₄₅=are independently A, U or absent;
R₂, R₁₁, R₁₃, R₃₅, R₄₃, R₄₉, R₅₅, R₆₀, R₆₈, R₇₁=are independently C or absent;
R₃₃=C, G or absent;
R₃, R₂₈, R₃₆, R₄₈, R₅₈, R₅₉, R₆₁=are independently C, U or absent;
R₁, R₁₀, R₁₉, R₂₀, R₂₁, R₂₅, R₂₇, R₂₉, R₃₇, R₄₀, R₅₁, R₅₂, R₆₂, R₆₃, R₆₄=are independently G or absent;
R₈, R₁₂, R₁₆, R₁₇, R₃₀, R₃₁, R₄₂, R₄₄, R₅₀, R₅₃, R₅₄, R₆₅, R₆₆, R₆₇=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Proline TREM Consensus Sequence

In an embodiment, a TREM disclosed herein comprises the sequence of Formula I PRO (SEQ ID NO: 604),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Pro is:
R₀=absent
R₁₄, R₅₇=are independently A or absent;
R₇₀, R₇₂=are independently A, C or absent;
R₉, R₂₆, R₂₇=are independently A, C, G or absent;
R₄, R₅, R₆, R₁₆, R₂₁, R₂₉, R₃₀, R₃₁, R₃₂, R₃₃, R₃₄, R₃₇, R₄₁, R₄₂, R₄₃, R₄₄, R₄₅, R₄₆, R₄₈, R₄₉, R₅₀, R 58, R₆₁, R₆₂, R₆₃, R₆₄, R₆₆, R₆₇, R₆₈=are independently N or absent;
R₃₅, R₆₅=are independently A, C, U or absent;
R₂₄, R₄₀, R₅₆=are independently A, G or absent;
R₇, R₂₅, R₅₁=are independently A, G, U or absent;
R₅₅, R₆₀=are independently C or absent;
R₁, R₃, R₇₁=are independently C, G or absent;
R₁₁, R₁₂, R₂₀, R₆₉=are independently C, G, U or absent;
R₁₃, R₁₇, R₁₈, R₂₂, R₂₃, R₂₈, R₅₉=are independently C, U or absent;
R₁₀, R₁₅, R₁₉, R₃₈, R₃₉, R₅₂=are independently G or absent;
R₂=are independently G, U or absent;
R₈, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II PRO (SEQ ID NO: 605),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Pro is:
R₀, R₁₇, R₁₈, R₂₂, R₂₃=absent;
R₁₄, R₄₅, R₅₆, R₅₇, R₅₈, R₆₅, R₆₈=are independently A or absent;
R₆₁=A, C, G or absent;
R₄₃=N or absent;
R₃₇=A, C, U or absent;
R₂₄, R₂₇, R₃₃, R₄₀, R₄₄, R₆₃=are independently A, G or absent;
R₃, R₁₂, R₃₀, R₃₂, R₄₈, R₅₅, R₆₀, R₇₀, R₇₁, R₇₂=are independently C or absent;
R₅, R₃₄, R₄₂, R₆₆=are independently C, G or absent;
R₂₀=C, G, U or absent;
R₃₅, R₄₁, R₄₉, R₆₂=are independently C, U or absent;
R₁, R₂, R₆, R₉, R₁₀, R₁₅, R₁₉, R₂₆, R₃₈, R₃₉, R₄₆, R₅₀, R₅₁, R₅₂, R₆₄, R₆₇, R₆₉=are independently G or absent;
R₁₁, R₁₆=are independently G, U or absent;
R₄, R₇, R₈, R₁₃, R₂₁, R₂₅, R₂₈, R₂₉, R₃₁, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula III PRO (SEQ ID NO: 606),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Pro is:
R₀, R₁₇, R₁₈, R₂₂, R₂₃=absent
R₁₄, R₄₅, R₅₆, R₅₇, R₅₈, R₆₅, R₆₈=are independently A or absent;
R₃₇=A, C, U or absent;
R₂₄, R₂₇, R₄₀=are independently A, G or absent;
R₃, R₅, R₁₂, R₃₀, R₃₂, R₄₈, R₄₉, R₅₅, R₆₀, R₆₁, R₆₂, R₆₆, R₇₀, R₇₁, R₇₂=are independently C or absent;
R₃₄, R₄₂=are independently C, G or absent;
R₄₃=C, G, U or absent;
R₄₁=C, U or absent;
R₁, R₂, R₆, R₀, R₁₀, R₁₅, R₁₉, R₂₀, R₂₆, R₃₃, R₃₈, R₃₉, R₄₄, R₄₆, R₅₀, R₅₁, R₅₂, R₆₃, R₆₄, R₆₇, R₆₉=are independently G or absent;
R₁₆=G, U or absent;
R₄, R₇, R₈, R₁₁, R₁₃, R₂₁, R₂₅, R₂₈, R₂₉, R₃₁, R₃₅, R₃₆, R₅₃, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues arc N; or no more than 20 residues arc absent.

### Serine TREM Consensus Sequence

In an embodiment, a TREM disclosed herein comprises the sequence of Formula I _{SER} (SEQ ID NO: 607),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R 22-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ser is:
R₀=absent;
R₁₄, R₂₄, R₅₇=are independently A or absent;
R₄₁=A, C or absent;
R₂, R₃, R₄, R₅, R₆, R₇, R₀, R₁₀, R₁₁, R₁₂, R₁₃, R₁₆, R₂₁, R₂₅, R₂₆, R₂₇, R₂₈, R₃₀, R₃₁, R₃₂, R₃₃, R₃₄, R₃₇, R₄₂, R₄₃, R₄₄, R₄₅, R₄₆, R₄₈, R₄₉, R₅₀, R₆₂, R₆₃, R₆₄, R₆₅, R₆₆, R₆₇, R₆₈, R₆₉, R₇₀=are independently N or absent;
R₁₈=A, C, U or absent;
R₁₅, R₄₀, R₅₁, R₅₆=are independently A, G or absent;
R₁, R₂₉, R₅₈, R₇₂=are independently A, G, U or absent;
R₃₉=A, U or absent;
R₆₀=C or absent;
R₃₈=C, G or absent;
R₁₇, R₂₂, R₂₃, R₇₁=are independently C, G, U or absent;
R₈, R₃₅, R₃₆, R₅₅, R₅₉, R₆₁=are independently C, U or absent;
R₁₉, R₂₀=are independently G or absent;
R₂=G, U or absent;
R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II _{SER} (SEQ ID NO: 608),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ser is:
R₀, R₂₃=absent
R₁₄, R₂₄, R₄₁, R₅₇=are independently A or absent;
R₄₄=A, C or absent;
R₂₅, R₄₅, R₄₈=are independently A, C, G or absent;
R₂, R₃, R₄, R₅, R₃₇, R₅₀, R₆₂, R₆₆, R₆₇, R₆₉, R₇₀=are independently N or absent;
R₁₂, R₂₈, R₆₅=are independently A, C, U or absent;
R₉, R₁₅, R₂₉, R₃₄, R₄₀, R₅₆, R₆₃=are independently A, G or absent;
R₇, R₂₆, R₃₀, R₃₃, R₄₆, R₅₈, R₇₂=are independently A, G, U or absent;
R₃₉=A, U or absent;
R₁₁, R₃₅, R₆₀, R₆₁=are independently C or absent;
R₁₃, R₃₈=are independently C, G or absent;
R₆, R₁₇, R₃₁, R₄₃, R₆₄, R₆₈=are independently C, G, U or absent;
R₃₆, R₄₂, R₄₉, R₅₅, R₅₉, R₇₁=are independently C, U or absent;
R₁₀, R₁₉, R₂₀, R₂₇, R₅₁=are independently G or absent;
R₁, R₁₆, R₃₂, R₅₂=are independently G, U or absent;
R₈, R₁₈, R₂₁, R₂₂, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III _{SER} (SEQ ID NO: 609),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Ser is:
R₀, R₂₃=absent
R₁₄, R₂₄, R₄₁, R₅₇, R₅₈=are independently A or absent;
R₄₄=A, C or absent;
R₂₅, R₄₈=are independently A, C, G or absent;
R₂, R₃, R₅, R₃₇, R₆₆, R₆₇, R₆₉, R₇₀=are independently N or absent;
R₁₂, R₂₈, R₆₂=are independently A, C, U or absent;
R₇, R₉, R₁₅, R₂₉, R₃₃, R₃₄, R₄₀, R₄₅, R₅₆, R₆₃=are independently A, G or absent;
R₄, R₂₆, R₄₆,R 50=are independently A, G, U or absent;
R₃₀, R₃₉=are independently A, U or absent;
R₁₁, R₁₇, R₃₅, R₆₀, R₆₁=are independently C or absent;
R₁₃, R₃₈=are independently C, G or absent;
R₆, R₆₄=are independently C, G, U or absent;
R₃₁, R₄₂, R₄₃, R₄₉, R₅₅, R₅₉, R₆₅, R₆₈, R₇₁=are independently C, U or absent;
R₁₀, R₁₉, R₂₀, R₂₇, R₅₁, R₅₂=are independently G or absent;
R₁, R₁₆, R₃₂, R₇₂=are independently G, U or absent;
R₈, R₁₈, R₂₁, R₂₂, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Threonine TREM Consensus Sequence

In an embodiment, a TREM disclosed herein comprises the sequence of Formula I _{THR} (SEQ ID NO: 610),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Thr is:
R₀, R₂₃=absent
R₁₄, R₄₁, R₅₇=are independently A or absent;
R₅₆, R₇₀=are independently A, C, G or absent;
R₄, R₅, R₆, R₇, R₁₂, R₁₆, R₂₆, R₃₀, R₃₁, R₃₂, R₃₄, R₃₇, R₄₂, R₄₄, R₄₅, R₄₆, R₄₈, R₄₉, R₅₀, R₅₈, R₆₂, R₆₃, R₆₄, R₆₅, R₆₆, R₆₇, R₆₈, R₇₂=are independently N or absent;
R₁₃, R₁₇, R₂₁, R₃₅, R₆₁=are independently A, C, U or absent;
R₁, R₉, R₂₄, R₂₇, R₂₉, R₆₉=are independently A, G or absent;
R₁₅, R₂₅, R₅₁=are independently A, G, U or absent;
R₄₀, R₅₃=are independently A, U or absent;
R₃₃, R₄₃=are independently C, G or absent;
R₂, R₃, R₅₉=are independently C, G, U or absent;
R₁₁, R₁₈, R₂₂, R₂₈, R₃₆, R₅₄, R₅₅, R₆₀, R₇₁=are independently C, U or absent;
R₁₀, R₂₀, R₃₈, R₅₂=are independently G or absent;
R₁₉=G, U or absent;
R₈, R₃₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x-80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II THR (SEQ ID NO: 611),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Thr is:
R₀, R₁₈, R₂₃=absent
R₁₄, R₄₁, R₅₇=are independently A or absent;
R₀, R₄₂, R₄₄, R₄₈, R₅₆, R₇₀=are independently A, C, G or absent;
R₄, R₆, R₁₂, R₂₆, R₄₉, R₅₈, R₆₃, R₆₄, R₆₆, R₆₈=are independently N or absent;
R₁₃, R₂₁, R₃₁, R₃₇, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₇=are independently A, G, U or absent;
R₄₀, R₅₃=are independently A, U or absent;
R₃₅=C or absent;
R₃₃, R₄₃=are independently C, G or absent;
R₂, R₃, R₅, R₁₆, R₃₂, R₃₄, R₅₉, R₆₅, R₇₂=are independently C, G, U or absent;
R₁₁, R₁₇, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₈, R₃₉, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

In an embodiment, a TREM disclosed herein comprises the sequence of Formula III THR (SEQ ID NO: 612),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Thr is:
R₀, R₁₈, R₂₃=absent
R₁₄, R₄₀, R₄₁, R₅₇=are independently A or absent;
R₄₄=A, C or absent;
R₀, R₄₂, R₄₈, R₅₆=are independently A, C, G or absent;
R₄, R₆, R₁₂, R₂₆, R₅₈, R₆₄, R₆₆, R₆₈=are independently N or absent;
R₁₃, R₂₁, R₃₁, R₃₇, R₄₉, R₆₂=are independently A, C, U or absent;
R₁, R₁₅, R₂₄, R₂₇, R₂₉, R₄₆, R₅₁, R₆₉=are independently A, G or absent;
R₇, R₂₅, R₄₅, R₅₀, R₆₃, R₆₇=are independently A, G, U or absent;
R₅₃=A, U or absent;
R₃₅=C or absent;
R₂, R₃₃, R₄₃,R 70=are independently C, G or absent;
R₅, R₁₆, R₃₄, R₅₉, R₆₅=are independently C, G, U or absent;
R₃, R₁₁, R₂₂, R₂₈, R₃₀, R₃₆, R₅₅, R₆₀, R₆₁, R₇₁=are independently C, U or absent;
R₁₀, R₁₉, R₂₀, R₃₈, R₅₂=are independently G or absent;
R₃₂=G, U or absent;
R₈, R₁₇, R₃₉, R₅₄, R₇₂=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tryptophan TREM Consensus Sequence

In an embodiment, a TREM disclosed herein comprises the sequence of Formula I TRP (SEQ ID NO: 613),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Trp is:
R₀=absent;
R₂₄, R₃₉, R₄₁, R₅₇=are independently A or absent;
R₂, R₃, R₂₆, R₂₇, R₄₀, R₄₈=are independently A, C, G or absent;
R₄, R₅, R₆, R₂₉, R₃₀, R₃₁, R₃₂, R₃₄, R₄₂, R₄₄, R₄₅, R₄₆, R₄₉, R₅₁, R₅₈, R₆₃, R₆₆, R₆₇, R₆₈=are independently N or absent;
R₁₃, R₁₄, R₁₆, R₁₈, R₂₁, R₆₁, R₆₅, R₇₁=are independently A, C, U or absent;
R₁, R₉, R₁₀, R₁₅, R₃₃, R₅₀, R₅₆=are independently A, G or absent;
R₇, R₂₅, R₇₂=are independently A, G, U or absent;
R₃₇, R₃₈, R₅₅, R₆₀=are independently C or absent;
R₁₂, R₃₅, R₄₃, R₆₄, R₆₉, R₇₀=are independently C, G, U or absent;
R₁₁, R₁₇, R₂₂, R₂₈, R₅₉, R₆₂=are independently C, U or absent;
R₁₉, R₂₀, R₅₂=are independently G or absent;
R₈, R₂₃, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II TRP (SEQ ID NO: 614),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀,R 50, R 52, R₆₉=are independently G or absent;
R₅, R₈, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III TRP (SEQ ID NO: 615),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Trp is:
R₀, R₁₈, R₂₂, R₂₃=absent
R₁₄, R₂₄, R₃₉, R₄₁, R₅₇, R₇₂=are independently A or absent;
R₃, R₄, R₁₃, R₆₁, R₇₁=are independently A, C or absent;
R₆, R₄₄=are independently A, C, G or absent;
R₂₁=A, C, U or absent;
R₂, R₇, R₁₅, R₂₅, R₃₃, R₃₄, R₄₅, R₅₆, R₆₃=are independently A, G or absent;
R₅₈=A, G, U or absent;
R₄₆=A, U or absent;
R₃₇, R₃₈, R₅₅, R₆₀, R₆₂=are independently C or absent;
R₁₂, R₂₆, R₂₇, R₃₅, R₄₀, R₄₈, R₆₇=are independently C, G or absent;
R₃₂, R₄₃, R₆₈=are independently C, G, U or absent;
R₁₁, R₁₆, R₂₈, R₃₁, R₄₉, R₅₉, R₆₅, R₇₀=are independently C, U or absent;
R₁, R₉, R₁₀, R₁₉, R₂₀, R₅₀, R₅₂, R₆₉=are independently G or absent;
R₅, R₅, R₂₉, R₃₀, R₄₂, R₅₁, R₆₄, R₆₆=are independently G, U or absent;
R₁₇, R₃₆, R₅₃, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Tyrosine TREM Consensus Sequence

In an embodiment, a TREM disclosed herein comprises the sequence of Formula I TYR (SEQ ID NO: 616),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Tyr is:
R₀=absent
R₁₄, R₃₉, R₅₇=are independently A or absent;
R₄₁, R₄₈, R₅₁, R₇₁=are independently A, C, G or absent;
R₃, R₄, R₅, R₆, R₀, R₁₀, R₁₂, R₁₃, R₁₆, R₂₅, R₂₆, R₃₀, R₃₁, R₃₂, R₄₂, R₄₄, R₄₅, R₄₆, R₄₉, R₅₀, R₅₈, R₆₂, R₆₃, R₆₆, R₆₇, R₆₈, R₆₉, R₇₀=are independently N or absent;
R₂₂, R₆₅=are independently A, C, U or absent;
R₁₅, R₂₄, R₂₇, R₃₃, R₃₇, R₄₀, R₅₆=are independently A, G or absent;
R₇, R₂₉, R₃₄, R₇₂=are independently A, G, U or absent;
R₂₃, R₅₃=are independently A, U or absent;
R₃₅, R₆₀=are independently C or absent;
R₂₀=C, G or absent;
R₁, R₂, R₂₈, R₆₁, R₆₄=are independently C, G, U or absent;
R₁₁, R₁₇, R₂₁, R₄₃, R₅₅=are independently C, U or absent;
R₁₉, R₅₂=are independently G or absent;
R₈, R₁₈, R₃₆, R₃₈, R₅₄, R₅₉=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II TYR (SEQ ID NO: 617),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R 22-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇=are independently A or absent;
R₄₄, R₆₉=are independently A, C or absent;
R₇₁=A, C, G or absent;
R₆₈=N or absent;
R₅₈=A, C, U or absent;
R₃₃, R₃₇, R₄₁, R₅₆, R₆₂, R₆₃=are independently A, G or absent;
R₆, R₂₉, R₇₂=are independently A, G, U or absent;
R₃₁, R₄₅, R₅₃=are independently A, U or absent;
R₁₃, R₃₅, R₄₉, R₆₀=are independently C or absent;
R₂₀, R₄₈, R₆₄, R₆₇, R₇₀=are independently C, G or absent;
R₁, R₂, R₅, R₁₆, R₆₆=are independently C, G, U or absent;
R₁₁, R₂₁, R₂₈, R₄₃, R₅₅, R₆₁=are independently C, U or absent;
R₁₀, R₁₅, R₁₉, R₂₅, R₂₇, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III TYR (SEQ ID NO: 618),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Tyr is:
R₀, R₁₈, R₂₃=absent
R₇, R₉, R₁₄, R₂₄, R₂₆, R₃₄, R₃₉, R₅₇, R₇₂=are independently A or absent;
R₄₄, R₆₉=are independently A, C or absent;
R₇₁=A, C, G or absent;
R₃₇, R₄₁, R₅₆, R₆₂, R₆₃=are independently A, G or absent;
R₆, R₂₉, R₆₈=are independently A, G, U or absent;
R₃₁, R₄₅, R₅₈=are independently A, U or absent;
R₁₃, R₂₈, R₃₅, R₄₉, R₆₀, R₆₁=are independently C or absent;
R₅, R₄₈, R₆₄, R₆₇, R₇₀=are independently C, G or absent;
R₁, R₂=are independently C, G, U or absent;
R₁₁, R₁₆, R₂₁, R₄₃, R₅₅, R₆₆=are independently C, U or absent;
R₁₀, R₁₅, R₁₉, R₂₀, R₂₅, R₂₇, R₃₃, R₄₀, R₅₁, R₅₂=are independently G or absent;
R₃, R₄, R₃₀, R₃₂, R₄₂, R₄₆=are independently G, U or absent;
R₈, R₁₂, R₁₇, R₂₂, R₃₆, R₃₈, R₅₀, R₅₃, R₅₄, R₅₉, R₆₅=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, provided that the TREM has one or both of the following properties: no more than 15% of the residues are N; or no more than 20 residues are absent.

### Valine TREM Consensus Sequence

In an embodiment, a TREM disclosed herein comprises the sequence of Formula I VAL (SEQ ID NO: 619),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Val is:
R₀, R₂₃=absent;
R₂₄, R₃₈, R₅₇=are independently A or absent;
R₉, R₇₂=are independently A, C, G or absent;
R₂, R₄, R₅, R₆, R₇, R₁₂, R₁₅, R₁₆, R₂₁, R₂₅, R₂₆, R₂₉, R₃₁, R₃₂, R₃₃, R₃₄, R₃₇, R₄₁, R₄₂, R₄₃, R₄₄, R₄₅, R₄₆, R₄₈, R₄₉, R₅₀, R₅₈, R₆₁, R₆₂, R₆₃, R₆₄, R₆₅, R₆₆, R₆₇, R₆₈, R₆₉, R₇₀=are independently N or absent;
R₁₇, R₃₅, R₅₉=are independently A, C, U or absent;
R₁₀, R₁₄, R₂₇, R₄₀, R₅₂, R₅₆=are independently A, G or absent;
R₁, R₃, R₅₁, R₅₃=are independently A, G, U or absent;
R₃₉=C or absent;
R₁₃, R₃₀, R₅₅=are independently C, G, U or absent;
R₁₁, R₂₂, R₂₈, R₆₀, R₇₁=are independently C, U or absent;
R₁₉=G or absent;
R₂₀=G, U or absent;
R₈, R₁₈, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, 271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula II VAL (SEQ ID NO: 620),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Val is:
R₀, R₁₈, R₂₃=absent;
R₂₄, R₃₈, R₅₇=are independently A or absent;
R₆₄, R₇₀, R₇₂=are independently A, C, G or absent;
R₁₅, R₁₆, R₂₆, R₂₉, R₃₁, R₃₂, R₄₃, R₄₄, R₄₅, R₄₉, R₅₀, R₅₈, R₆₂, R₆₅=are independently N or absent;
R₆, R₁₇, R₃₄, R₃₇, R₄₁, R₅₉=are independently A, C, U or absent;
R₉, R₁₀, R₁₄, R₂₇, R₄₀, R₄₆, R₅₁, R₅₂, R₅₆=are independently A, G or absent;
R₇, R₁₂, R₂₅, R₃₃, R₅₃, R₆₃, R₆₆, R₆₈=are independently A, G, U or absent;
R₆₉=A, U or absent;
R₃₉=C or absent;
R₅, R₆₇=are independently C, G or absent;
R₂, R₄, R₁₃, R₄₈, R₅₅, R₆₁=are independently C, G, U or absent;
R₁₁, R₂₂, R₂₈, R₃₀, R₃₅, R₆₀, R₇₁=are independently C, U or absent;
R₁₉=G or absent;
R₁, R₃, R₂₀, R₄₂=are independently G, U or absent;
R₈, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

In an embodiment, a TREM disclosed herein comprises the sequence of Formula III VAL (SEQ ID NO: 621),
R₀-R₁-R₂-R₃-R₄-R₅-R₆-R₇-R₈-R₉-R₁₀-R₁₁-R₁₂-R₁₃-R₁₄-R₁₅-R₁₆-R₁₇-R₁₈-R₁₉-R₂₀-R₂₁-R₂₂-R₂₃-R₂₄-R₂₅-R₂₆-R₂₇-R₂₈-R₂₉-R₃₀-R₃₁-R₃₂-R₃₃-R₃₄-R₃₅-R₃₆-R₃₇-R₃₈-R₃₉-R₄₀-R₄₁-R₄₂-R₄₃- R₄₄-R₄₅-R₄₆-[R₄₇]ₓ-R₄₈-R₄₉-R₅₀-R₅₁-R₅₂-R₅₃-R₅₄-R₅₅-R₅₆-R₅₇-R₅₈-R₅₉-R₆₀-R₆₁-R₆₂-R₆₃-R₆₄-R₆₅- R₆₆-R₆₇-R₆₈-R₆₉-R₇₀-R₇₁-R₇₂
   wherein R is a ribonucleotide residue and the consensus for Val is:
R₀, R₁₈, R₂₃=absent
R₂₄, R₃₈, R₄₀, R₅₇, R₇₂=are independently A or absent;
R₂₉, R₆₄, R₇₀=are independently A, C, G or absent;
R₄₉, R₅₀, R₆₂=are independently N or absent;
R₁₆, R₂₆, R₃₁, R₃₂, R₃₇, R₄₁, R₄₃, R₅₉, R₆₅=are independently A, C, U or absent;
R₉, R₁₄, R₂₇, R₄₆, R₅₂, R₅₆, R₆₆=are independently A, G or absent;
R₇, R₁₂, R₂₅, R₃₃, R₄₄, R₄₅, R₅₃, R₅₈, R₆₃, R₆₈=are independently A, G, U or absent;
R₆₉=A, U or absent;
R₃₉=C or absent;
R₅, R₆₇=are independently C, G or absent;
R₂, R₄, R₁₃, R₁₅, R₄₈, R₅₅=are independently C, G, U or absent;
R₆, R₁₁, R₂₂, R₂₈, R₃₀, R₃₄, R₃₅, R₆₀, R₆₁, R₇₁=are independently C, U or absent;
R₁₀, R₁₉, R₅₁=are independently G or absent;
R₁, R₃, R₂₀, R₄₂=are independently G, U or absent;
R₈, R₁₇, R₂₁, R₃₆, R₅₄=are independently U or absent;
[R₄₇]ₓ=N or absent;
wherein, e.g., x=1-271 (e.g., x=1-250, x=1-225, x=1-200, x=1-175, x=1-150, x=1-125, x=1-100, x=1-75, x=1-50, x=1-40, x=1-30, x=1-29, x=1-28, x=1-27, x=1-26, x=1-25, x=1-24, x=1-23, x=1-22, x=1-21, x=1-20, x=1-19, x=1-18, x=1-17, x=1-16, x=1-15, x=1-14, x=1-13, x=1-12, x=1-11, x=1-10, x=10-271, x=20-271, x=30-271, x=40-271, x=50-271, x=60-271, x=70-271, x=80-271, x=100-271, x=125-271, x=150-271, x=175-271, x=200-271, x=225-271, x=1, x=2, x=3, x=4, x=5, x=6, x=7, x=8, x=9, x=10, x=11, x=12, x=13, x=14, x=15, x=16, x=17, x=18, x=19, x=20, x=21, x=22, x=23, x=24, x=25, x=26, x=27, x=28, x=29, x=30, x=40, x=50, x=60, x=70, x=80, x=90, x=100, x=110, x=125, x=150, x=175, x=200, x=225, x=250, or x=271), provided that the TREM has one or both of the following properties: no more than 15% of the residues are N; or no more than 20 residues are absent.

### Variable Region Consensus Sequence

In an embodiment, a TREM disclosed herein comprises a variable region at position R₄₇. In an embodiment, the variable region is 1-271 ribonucleotides in length (e.g. 1-250, 1-225, 1-200, 1-175, 1-150, 1-125, 1-100, 1-75, 1-50, 1-40, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 10-271, 20-271, 30-271, 40-271, 50-271, 60-271, 70-271, 80-271, 100-271, 125-271, 150-271, 175-271, 200-271, 225-271, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 110, 125, 150, 175, 200, 225, 250, or 271 ribonucleotides). In an embodiment, the variable region comprises any one, all or a combination of Adenine, Cytosine, Guanine or Uracil.

In an embodiment, the variable region comprises a ribonucleic acid (RNA) sequence encoded by a deoxyribonucleic acid (DNA) sequence disclosed in Table 4, e.g., any one of SEQ ID NOs: 452-561 disclosed in Table 4.

TABLE 4
Exemplary variable region sequences.

| | SEQ ID NO | SEQUENCE |
|---|---|---|
| 1 | 452 | AAAATATAAATATATTTC |
| 2 | 453 | AAGCT |
| 3 | 454 | AAGTT |
| 4 | 455 | AATTCTTCGGAATGT |
| 5 | 456 | AGA |
| 6 | 457 | AGTCC |
| 7 | 458 | CAACC |
| 8 | 459 | CAATC |
| 9 | 460 | CAGC |
| 10 | 461 | CAGGCGGGTTCTGCCCGCGC |
| 11 | 462 | CATACCTGCAAGGGTATC |
| 12 | 463 | CGACCGCAAGGTTGT |
| 13 | 464 | CGACCTTGCGGTCAT |
| 14 | 465 | CGATGCTAATCACATCGT |
| 15 | 466 | CGATGGTGACATCAT |
| 16 | 467 | CGATGGTTTACATCGT |
| 17 | 468 | CGCCGTAAGGTGT |
| 18 | 469 | CGCCTTAGGTGT |
| 19 | 470 | CGCCTTTCGACGCGT |
| 20 | 471 | CGCTTCACGGCGT |
| 21 | 472 | CGGCAGCAATGCTGT |
| 22 | 473 | CGGCTCCGCCTTC |
| 23 | 474 | CGGGTATCACAGGGTC |
| 24 | 475 | CGGTGCGCAAGCGCTGT |
| 25 | 476 | CGTACGGGTGACCGTACC |
| 26 | 477 | CGTCAAAGACTTC |
| 27 | 478 | CGTCGTAAGACTT |
| 28 | 479 | CGTTGAATAAACGT |
| 29 | 480 | CTGTC |
| 30 | 481 | GGCC |
| 31 | 482 | GGGGATT |
| 32 | 483 | GGTC |
| 33 | 484 | GGTTT |
| 34 | 485 | GTAG |
| 35 | 486 | TAACTAGATACTTTCAGAT |
| 36 | 487 | TACTCGTATGGGTGC |
| 37 | 488 | TACTTTGCGGTGT |
| 38 | 489 | TAGGCGAGTAACATCGTGC |
| 39 | 490 | TAGGCGTGAATAGCGCCTC |
| 40 | 491 | TAGGTCGCGAGAGCGGCGC |
| 41 | 492 | TAGGTCGCGTAAGCGGCGC |
| 42 | 493 | TAGGTGGTTATCCACGC |
| 43 | 494 | TAGTC |
| 44 | 495 | TAGTT |
| 45 | 496 | TATACGTGAAAGCGTATC |
| 46 | 497 | TATAGGGTCAAAAACTCTATC |
| 47 | 498 | TATGCAGAAATACCTGCATC |
| 48 | 499 | TCCCCATACGGGGGC |
| 49 | 500 | TCCCGAAGGGGTTC |
| 50 | 501 | TCTACGTATGTGGGC |
| 51 | 502 | TCTCATAGGAGTTC |
| 52 | 503 | TCTCCTCTGGAGGC |
| 53 | 504 | TCTTAGCAATAAGGT |
| 54 | 505 | TCTTGTAGGAGTTC |
| 55 | 506 | TGAACGTAAGTTCGC |
| 56 | 507 | TGAACTGCGAGGTTCC |
| 57 | 508 | TGAC |
| 58 | 509 | TGACCGAAAGGTCGT |
| 59 | 510 | TGACCGCAAGGTCGT |
| 60 | 511 | TGAGCTCTGCTCTC |
| 61 | 512 | TGAGGCCTCACGGCCTAC |
| 62 | 513 | TGAGGGCAACTTCGT |
| 63 | 514 | TGAGGGTCATACCTCC |
| 64 | 515 | TGAGGGTGCAAATCCTCC |
| 65 | 516 | TGCCGAAAGGCGT |
| 66 | 517 | TGCCGTAAGGCGT |
| 67 | 518 | TGCGGTCTCCGCGC |
| 68 | 519 | TGCTAGAGCAT |
| 69 | 520 | TGCTCGTATAGAGCTC |
| 70 | 521 | TGGACAATTGTCTGC |
| 71 | 522 | TGGACAGATGTCCGT |
| 72 | 523 | TGGACAGGTGTCCGC |
| 73 | 524 | TGGACGGTTGTCCGC |
| 74 | 525 | TGGACTTGTGGTC |
| 75 | 526 | TGGAGATTCTCTCCGC |
| 76 | 527 | TGGCATAGGCCTGC |
| 77 | 528 | TGGCTTATGTCTAC |
| 78 | 529 | TGGGAGTTAATCCCGT |
| 79 | 530 | TGGGATCTTCCCGC |
| 80 | 531 | TGGGCAGAAATGTCTC |
| 81 | 532 | TGGGCGTTCGCCCGC |
| 82 | 533 | TGGGCTTCGCCCGC |
| 83 | 534 | TGGGGGATAACCCCGT |
| 84 | 535 | TGGGGGTTTCCCCGT |
| 85 | 536 | TGGT |
| 86 | 537 | TGGTGGCAACACCGT |
| 87 | 538 | TGGTTTATAGCCGT |
| 88 | 539 | TGTACGGTAATACCGTACC |
| 89 | 540 | TGTCCGCAAGGACGT |
| 90 | 541 | TGTCCTAACGGACGT |
| 91 | 542 | TGTCCTATTAACGGACGT |
| 92 | 543 | TGTCCTTCACGGGCGT |
| 93 | 544 | TGTCTTAGGACGT |
| 94 | 545 | TGTGCGTTAACGCGTACC |
| 95 | 546 | TGTGTCGCAAGGCACC |
| 96 | 547 | TGTTCGTAAGGACTT |
| 97 | 548 | TTCACAGAAATGTGTC |
| 98 | 549 | TTCCCTCGTGGAGT |
| 99 | 550 | TTCCCTCTGGGAGC |
| 100 | 551 | TTCCCTTGTGGATC |
| 101 | 552 | TTCCTTCGGGAGC |
| 102 | 553 | TTCTAGCAATAGAGT |
| 103 | 554 | TTCTCCACTGGGGAGC |
| 104 | 555 | TTCTCGAGAGGGAGC |
| 105 | 556 | TTCTCGTATGAGAGC |
| 106 | 557 | TTTAAGGTTTTCCCTTAAC |
| 107 | 558 | TTTCATTGTGGAGT |
| 108 | 559 | TTTCGAAGGAATCC |
| 109 | 560 | TTTCTTCGGAAGC |
| 110 | 561 | TTTGGGGCAACTCAAC |

### Corresponding Nucleotide Positions

To determine if a selected nucleotide position in a candidate sequence corresponds to a selected position in a reference sequence (e.g., SEQ ID NO: 622, SEQ ID NO: 623, SEQ ID NO: 624), one or more of the following Evaluations is performed.

### Evaluation A:

1. The candidate sequence is aligned with each of the consensus sequences in Tables 9 and 10. The consensus sequence(s) having the most positions aligned (and which has at least 60% of the positions of the candidate sequence aligned) is selected.

The alignment is performed as is follows. The candidate sequence and an isodecoder consensus sequence from Tables 10A-10B are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and the consensus sequence by counting the number of matched positions in the alignment, dividing it by the larger of the number of non-N bases in the candidate sequence or the consensus sequence, and multiplying the result by 100. In cases where multiple alignments (of the candidate and a single consensus sequence) tie for the same score, the percent similarity is the largest percent similarity calculated from the tied alignments. This process is repeated for the candidate sequence with each of the remaining isodecoder consensus sequences in Tables 10A-10B, and the alignment resulting in the greatest percent similarity is selected. If this alignment has a percent similarity equal to or greater than 60%, it is considered a valid alignment and used to relate positions in the candidate sequence to those in the consensus sequence, otherwise the candidate sequence is considered to have not aligned to any of the isodecoder consensus sequences. If there is a tie at this point, all tied consensus sequences are taken forward to step 2 in the analysis.
2. Using the selected consensus sequence(s) from step 1, one determines the consensus sequence position number that aligns with the selected position (e.g., a modified position) in the candidate sequence. One then assigns the position number of the aligned position in the consensus sequence to the selected position in the candidate sequence, in other words, the selected position in the candidate sequence is numbered according to the numbering of the consensus sequence. If there were tied consensus sequences from step one, and they give different position numbers in this step 2, then all such position numbers are taken forward to step 5.
3. The reference sequence is aligned with the consensus sequence chosen in step 1. The alignment is performed as described in step 1.
4. From the alignment in step 3, one determines the consensus sequence position number that aligns with the selected position (e.g., a modified position) in the reference sequence. One then assigns the position number of the aligned position in the consensus sequence to the selected position in the reference sequence, in other words, the selected position in the reference sequence is numbered according to the numbering of the consensus sequence. If there is a tie at this point, all tied consensus sequences are taken forward to step 5 in the analysis.
5. If a value for a position number determined for the reference sequence in step 2 is the same as the value for the position number determined for the candidate sequence in step 4, the positions are defined as corresponding.

### Evaluation B:

The reference sequence (e.g., a TREM sequence described herein) and the candidate sequence are aligned with one another. The alignment is performed as follows.

The reference sequence and the candidate sequence are aligned based on a global pairwise alignment calculated with the Needleman-Wunsch algorithm when run with match scores from Table 11, a mismatch penalty of −1, a gap opening penalty of −1, and a gap extension penalty of −0.5, and no penalty for end gaps. The alignment with the highest overall alignment score is then used to determine the percent similarity between the candidate and reference sequence by counting the number of matched based in the alignment, dividing it by the larger of the number of non-N bases in the candidate or reference sequence, and multiplying the result by 100. In cases where multiple alignments tie for the same score, the percent similarity is the largest percent similarity calculated from the tied alignments. If this alignment has a percent similarity equal to or greater than 60%, it is considered a valid alignment and used to relate positions in the candidate sequence to those in the reference sequence, otherwise the candidate sequence is considered to have not aligned to the reference sequence.

If the selected nucleotide position in the reference sequence (e.g., a modified position) is paired with a selected nucleotide position (e.g., a modified position) in the candidate sequence, the positions are defined as corresponding.

### Evaluation C:

The candidate sequence is assigned a nucleotide position number according to the comprehensive tRNA numbering system (CtNS), also referred to as the tRNAviz method (e.g., as described in Lin et al., Nucleic Acids Research, 47:W1, pages W542-W547, 2 Jul. 2019), which serves as a global numbering system for tRNA molecules. The alignment is performed as follows.
1. The candidate sequence is assigned a nucleotide position according to the tRNAviz method. For a novel sequence not present in the tRNAviz database, the numbering for the closest sequence in the database is obtained. For example, if a TREM differs at any given nucleotide position from a sequence in the database, the numbering for the tRNA having the wildtype sequence at said given nucleotide position is used.
2. The reference sequence is assigned a nucleotide position according to the method described in 1.
3. If a value for a position number determined for the reference sequence in step 1 is the same as the value for the position number determined for the candidate sequence in step 2, the positions are defined as corresponding.

If the selected position in the reference sequence and the candidate sequence are found to be corresponding in at least one of Evaluations A, B, and C, the positions correspond. For example, if two positions are found to be corresponding under Evaluation A, but do not correspond under Evaluation B or Evaluation C, the positions are defined as corresponding. Similarly, if two positions are found to be corresponding under Evaluation B, but do not correspond under Evaluation A or Evaluation C, the positions are defined as corresponding. In addition, if two positions are found to be corresponding under Evaluation C, but do not correspond under Evaluation A or Evaluation B, the positions are defined as corresponding

The numbering given above is used for ease of presentation and does not imply a required sequence. If more than one Evaluation is performed, they can be performed in any order.

TABLE 10A
Consensus sequence computationally generated
for each isodecoder by aligning
members of the isodecoder family

| SEQ | | | |
|---|---|---|---|
| ID | Amino | Anti- | |
| NO. | Acid | codon | Consensus sequence |
| 1200 | Ala | AGC | GGGGAATTAGCTCAAGTGGTAGAGCGCTTG |
| | | | CTTAGCATGCAAGAGGTAGTGGGATCGATG |
| | | | CCCACATTCTCCA |
| 1201 | Ala | CGC | GGGGATGTAGCTCAGTGGTAGAGCGCATGC |
| | | | TTCGCATGTATGAGGTCCCGGGTTCGATCCC |
| | | | CGGCATCTCCA |
| 1202 | Ala | TGC | GGGGGTGTAGCTCAGTGGTAGAGCGCATGC |
| | | | TTTGCATGTATGAGGCCCCGGGTTCGATCCC |
| | | | CGGCACCTCCA |
| 1203 | Arg | ACG | GGGCCAGTGGCGCAATGGATAACGCGTCTG |
| | | | ACTACGGATCAGAAGATTCCAGGTTCGACTC |
| | | | CTGGCTGGCTCG |
| 1204 | Arg | CCG | GGCCGCGTGGCCTAATGGATAAGGCGTCTG |
| | | | ATTCCGGATCAGAAGATTGAGGGTTCGAGTC |
| | | | CCTTCGTGGTCG |
| 1205 | Arg | CCT | GCCCCAGTGGCCTAATGGATAAGGCACTGG |
| | | | CCTCCTAAGCCAGGGATTGTGGGTTCGAGTC |
| | | | CCACCTGGGGTA |
| 1206 | Arg | TCG | GACCGCGTGGCCTAATGGATAAGGCGTCTG |
| | | | ACTTCGGATCAGAAGATTGAGGGTTCGAGTC |
| | | | CCTCCGTGGTCG |
| 1207 | Arg | TCT | GGCTCTGTGGCGCAATGGATNAGCGCATTG |
| | | | GACTTCTAATTCAAAGGTTGCGGGTTCGAGT |
| | | | CCCNCCAGAGTCG |
| 1208 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACG |
| 1209 | Asp | GTC | TCCTCGTTAGTATAGTGGTGAGTATCCCCGC |
| | | | CTGTCACGCGGGAGACCGGGGTTCGATTCCC |
| | | | CGACGGGGAG |
| 1210 | Cys | GCA | GGGGGTATAGCTCAGNGGGTAGAGCATTTG |
| | | | ACTGCAGATCAAGAGGTCCCCGGTTCAAATC |
| | | | CGGGTGCCCCCT |
| 1211 | Gln | CTG | GGTTCCATGGTGTAATGGTNAGCACTCTGGA |
| | | | CTCTGAATCCAGCGATCCGAGTTCAAGTCTC |
| | | | GGTGGAACCT |
| 1212 | Gln | TTG | GGTCCCATGGTGTAATGGTTAGCACTCTGGA |
| | | | CTTTGAATCCAGCGATCCGAGTTCAAATCTC |
| | | | GGTGGGACCT |
| 1213 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAA |
| 1214 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAA |
| 1215 | Gly | CCC | GCATTGGTGGTTCAGTGGTAGAATTCTCGCC |
| | | | TCCCACGCNGGAGACCCGGGTTCGATTCCCG |
| | | | GCCAATGCA |
| 1216 | Gly | GCC | GCATTGGTGGTTCAGTGGTAGAATTCTCGCC |
| | | | TGCCACGCGGGAGGCCCGGGTTCGATTCCCG |
| | | | GCCAATGCA |
| 1217 | Gly | TCC | GCGTTGGTGGTATAGTGGTGAGCATAGCTGC |
| | | | CTTCCAAGCAGTTGACCCGGGTTCGATTCCC |
| | | | GGCCAACGCA |
| 1218 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCA |
| 1219 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCA |
| 1220 | Leu | AAG | GGTAGCGTGGCCGAGCGGTCTAAGGCGCTG |
| | | | GATTAAGGCTCCAGTCTCTTCGGGGGCGTGG |
| | | | GTTCGAATCCCACCGCTGCCA |
| 1221 | Leu | CAA | GTCAGGATGGCCGAGTGGTCNTAAGGCGCC |
| | | | AGACTCAAGTTCTGGTCTCCGNATGGAGGCG |
| | | | TGGGTTCGAATCCCACTTCTGACA |
| 1222 | Leu | CAG | GTCAGGATGGCCGAGCGGTCTAAGGCGCTG |
| | | | CGTTCAGGTCGCAGTCTCCCCTGGAGGCGTG |
| | | | GGTTCGAATCCCACTCCTGACA |
| 1223 | Lcu | TAA | ACCAGGATGGCCGAGTGGTTAAGGCGTTGG |
| | | | ACTTAAGATCCAATGGACAGATGTCCGCGTG |
| | | | GGTTCGAACCCCACTCCTGGTA |
| 1224 | Leu | TAG | GGTAGCGTGGCCGAGCGGTCTAAGGCGCTG |
| | | | GATTTAGGCTCCAGTCTCTTCGGNGGCGTGG |
| | | | GTTCGAATCCCACCGCTGCCA |
| 1225 | Lys | CTT | GCCCGGCTAGCTCAGTCGGTAGAGCATGAG |
| | | | ACTCTTAATCTCAGGGTCGTGGGTTCGAGCC |
| | | | CCACGTTGGGCGNNN |
| 1226 | Lys | TTT | GCCTGGATAGCTCAGTCGGTAGAGCATCAG |
| | | | ACTTTTAATCTGAGGGTCCAGGGTTCAAGTC |
| | | | CCTGTTCAGGCG |
| 1227 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCA |
| 1228 | Phe | GAA | GCCGAAATAGCTCAGTTGGGAGAGCGTTAG |
| | | | ACTGAAGATCNTAAAGGTCCCTGGTTCAATC |
| | | | CCGGGTTTCGGCA |
| 1229 | Pro | AGG | GGCTCGTTGGTCTAGGGGTATGATTCTCGCT |
| | | | TAGGATGCGAGAGGTCCCGGGTTCAAATCC |
| | | | CGGACGAGCCC |
| 1230 | Pro | CGG | GGCTCGTTGGTCTAGGGGTATGATTCTCGCT |
| | | | TCGGGTGCGAGAGGTCCCGGGTTCAAATCCC |
| | | | GGACGAGCCC |
| 1231 | Pro | TGG | GGCTCGTTGGTCTAGGGGTATGATTCTCGCT |
| | | | TTGGGTGCGAGAGGTCCCGGGTTCAAATCCC |
| | | | GGACGAGCCC |
| 1232 | Ser | AGA | GTAGTCGTGGCCGAGTGGTTAAGGCGATGG |
| | | | ACTAGAAATCCATTGGGGTTTCCCCGCGCAG |
| | | | GTTCGAATCCTGCCGACTACG |
| 1233 | Ser | CGA | GCTGTGATGGCCGAGTGGTTAAGGCGTTGG |
| | | | ACTCGAAATCCAATGGGGTCTCCCCGCGCAG |
| | | | GTTCGAATCCTGCTCACAGCG |
| 1234 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCG |
| 1235 | Ser | TGA | GTAGTCGTGGCCGAGTGGTTAAGGCGATGG |
| | | | ACTTGAAATCCATTGGGGTCTCCCCGCGCAG |
| | | | GTTCGAATCCTGCCGGCTACG |
| 1236 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCT |
| 1237 | Thr | CGT | GGCNCTGTGGCTNAGTNGGNTAAAGCGCCG |
| | | | GTCTCGTAAACCNGGAGATCNTGGGTTCGA |
| | | | ATCCCANCNGGGCCT |
| 1238 | Thr | TGT | GGCTCCATAGCTCAGNGGGTTAGAGCACTG |
| | | | GTCTTGTAAACCAGGGGTCGCGAGTTCAAAT |
| | | | CTCGCTGGGGCCT |
| 1239 | Trp | CCA | GACCTCGTGGCGCAACGGTAGCGCGTCTGA |
| | | | CTCCAGATCAGAAGGTTGCGTGTTCAAATCA |
| | | | CGTCGGGGTCA |
| 1240 | Tyr | GTA | CCTTCGATAGCTCAGCTGGTAGAGCGGAGG |
| | | | ACTGTAGATCCTTAGGTCGCTGGTTCGATTC |
| | | | CGGCTCGAAGGA |
| 1241 | Val | AAC | GTTTCCGTAGTGTAGTGGTTATCACGTTCGC |
| | | | CTAACACGCGAAAGGTCCCCGGTTCGAAAC |
| | | | CGGGCGGAAACA |
| 1242 | Val | CAC | GTTTCCGTAGTGTAGTGGTTATCACGTTCGC |
| | | | CTCACACGCGAAAGGTCCCCGGTTCGAAAC |
| | | | CGGGCGGAAACA |
| 1243 | Val | TAC | GGTTCCATAGTGTAGTGGTTATCACGTCTGC |
| | | | TTTACACGCAGAAGGTCCTGGGTTCGAGCCC |
| | | | CAGTGGAACCA |
| 1244 | iMet | CAT | AGCAGAGTGGCGCAGCGGAAGCGTGCTGGG |
| | | | CCCATAACCCAGAGGTCGATGGATCGAAAC |
| | | | CATCCTCTGCTA |

TABLE 10B
Consensus sequence computationally generated
for each isodecoder by aligning
members of the isodecoder family

| SEQ | Amino | Anti- | |
|---|---|---|---|
| ID NO | Acid | codon | Consensus sequence |
| 1245 | Ala | AGC | GGGGAATTAGCTCAAGTGGTAGAGCGCTTGC |
| | | | TTAGCATGCAAGAGGTAGTGGGATCGATGCC |
| | | | CACATTCTCCANNN |
| 1246 | Ala | CGC | GGGGATGTAGCTCAGTGGTAGAGCGCATGCT |
| | | | TCGCATGTATGAGGTCCCGGGTTCGATCCCC |
| | | | GGCATCTCCANNN |
| 1247 | Ala | TGC | GGGGGTGTAGCTCAGTGGTAGAGCGCATGCT |
| | | | TTGCATGTATGAGGCCCCGGGTTCGATCCCC |
| | | | GGCACCTCCANNN |
| 1248 | Arg | ACG | GGGCCAGTGGCGCAATGGATAACGCGTCTGA |
| | | | CTACGGATCAGAAGATTCCAGGTTCGACTCC |
| | | | TGGCTGGCTCGNNN |
| 1249 | Arg | CCG | GGCCGCGTGGCCTAATGGATAAGGCGTCTGA |
| | | | TTCCGGATCAGAAGATTGAGGGTTCGAGTCC |
| | | | CTTCGTGGTCGNNN |
| 1250 | Arg | CCT | GCCCCAGTGGCCTAATGGATAAGGCACTGGC |
| | | | CTCCTAAGCCAGGGATTGTGGGTTCGAGTCC |
| | | | CACCTGGGGTANNN |
| 1251 | Arg | TCG | GACCGCGTGGCCTAATGGATAAGGCGTCTGA |
| | | | CTTCGGATCAGAAGATTGAGGGTTCGAGTCC |
| | | | CTCCGTGGTCGNNN |
| 1252 | Arg | TCT | GGCTCTGTGGCGCAATGGATNAGCGCATTGG |
| | | | ACTTCTAATTCAAAGGTTGCGGGTTCGAGTC |
| | | | CCNCCAGAGTCGNNN |
| 1253 | Asn | GTT | GTCTCTGTGGCGCAATCGGTTAGCGCGTTCG |
| | | | GCTGTTAACCGNAAAGGTTGGTGGTTCGAGC |
| | | | CCACCCAGGGACGNNN |
| 1254 | Asp | GTC | TCCTCGTTAGTATAGTGGTGAGTATCCCCGC |
| | | | CTGTCACGCGGGAGACCGGGGTTCGATTCCC |
| | | | CGACGGGGAGNNN |
| 1255 | Cys | GCA | GGGGGTATAGCTCAGNGGGTAGAGCATTTGA |
| | | | CTGCAGATCAAGAGGTCCCCGGTTCAAATCC |
| | | | GGGTGCCCCCTNNN |
| 1256 | Gln | CTG | GGTTCCATGGTGTAATGGTNAGCACTCTGGA |
| | | | CTCTGAATCCAGCGATCCGAGTTCAAGTCTC |
| | | | GGTGGAACCTNNN |
| 1257 | Gln | TTG | GGTCCCATGGTGTAATGGTTAGCACTCTGGA |
| | | | CTTTGAATCCAGCGATCCGAGTTCAAATCTC |
| | | | GGTGGGACCTNNN |
| 1258 | Glu | CTC | TCCCTGGTGGTCTAGTGGTTAGGATTCGGCG |
| | | | CTCTCACCGCCGCGGCCCGGGTTCGATTCCC |
| | | | GGTCAGGGAANNN |
| 1259 | Glu | TTC | TCCCTGGTGGTCTAGTGGCTAGGATTCGGCG |
| | | | CTTTCACCGCNGCGGCCCGGGTTCGATTCCC |
| | | | GGTCAGGGAANNN |
| 1260 | Gly | CCC | GCATTGGTGGTTCAGTGGTAGAATTCTCGCC |
| | | | TCCCACGCNGGAGACCCGGGTTCGATTCCCG |
| | | | GCCAATGCANNN |
| 1261 | Gly | GCC | GCATTGGTGGTTCAGTGGTAGAATTCTCGCC |
| | | | TGCCACGCGGGAGGCCCGGGTTCGATTCCCG |
| | | | GCCAATGCANNN |
| 1262 | Gly | TCC | GCGTTGGTGGTATAGTGGTGAGCATAGCTGC |
| | | | CTTCCAAGCAGTTGACCCGGGTTCGATTCCC |
| | | | GGCCAACGCANNN |
| 1263 | Ile | AAT | GGCCGGTTAGCTCAGTTGGTTAGAGCGTGGT |
| | | | GCTAATAACGCCAAGGTCGCGGGTTCGATCC |
| | | | CCGTACGGGCCANNN |
| 1264 | Ile | TAT | GCTCCAGTGGCGCAATCGGTTAGCGCGCGGT |
| | | | ACTTATAATGCCGAGGTTGTGAGTTCGAGCC |
| | | | TCACCTGGAGCANNN |
| 1265 | Leu | AAG | GGTAGCGTGGCCGAGCGGTCTAAGGCGCTGG |
| | | | ATTAAGGCTCCAGTCTCTTCGGGGGCGTGGG |
| | | | TTCGAATCCCACCGCTGCCANNN |
| 1266 | Leu | CAA | GTCAGGATGGCCGAGTGGTCNTAAGGCGCCA |
| | | | GACTCAAGTTCTGGTCTCCGNATGGAGGCGT |
| | | | GGGTTCGAATCCCACTTCTGACANNN |
| 1267 | Leu | CAG | GTCAGGATGGCCGAGCGGTCTAAGGCGCTGC |
| | | | GTTCAGGTCGCAGTCTCCCCTGGAGGCGTGG |
| | | | GTTCGAATCCCACTCCTGACANNN |
| 1268 | Leu | TAA | ACCAGGATGGCCGAGTGGTTAAGGCGTTGGA |
| | | | CTTAAGATCCAATGGACAGATGTCCGCGTGG |
| | | | GTTCGAACCCCACTCCTGGTANNN |
| 1269 | Leu | TAG | GGTAGCGTGGCCGAGCGGTCTAAGGCGCTGG |
| | | | ATTTAGGCTCCAGTCTCTTCGGNGGCGTGGG |
| | | | TTCGAATCCCACCGCTGCCANNN |
| 1270 | Lys | CTT | GCCCGGCTAGCTCAGTCGGTAGAGCATGAGA |
| | | | CTCTTAATCTCAGGGTCGTGGGTTCGAGCCC |
| | | | CACGTTGGGCGNNNNNN |
| 1271 | Lys | TTT | GCCTGGATAGCTCAGTCGGTAGAGCATCAGA |
| | | | CTTTTAATCTGAGGGTCCAGGGTTCAAGTCC |
| | | | CTGTTCAGGCGNNN |
| 1272 | Met | CAT | GCCCTCTTAGCGCAGTNGGCAGCGCGTCAGT |
| | | | CTCATAATCTGAAGGTCCTGAGTTCGAGCCT |
| | | | CAGAGAGGGCANNN |
| 1273 | Phe | GAA | GCCGAAATAGCTCAGTTGGGAGAGCGTTAGA |
| | | | CTGAAGATCNTAAAGGTCCCTGGTTCAATCC |
| | | | CGGGTTTCGGCANNN |
| 1274 | Pro | AGG | GGCTCGTTGGTCTAGGGGTATGATTCTCGCT |
| | | | TAGGATGCGAGAGGTCCCGGGTTCAAATCCC |
| | | | GGACGAGCCCNNN |
| 1275 | Pro | CGG | GGCTCGTTGGTCTAGGGGTATGATTCTCGCT |
| | | | TCGGGTGCGAGAGGTCCCGGGTTCAAATCCC |
| | | | GGACGAGCCCNNN |
| 1276 | Pro | TGG | GGCTCGTTGGTCTAGGGGTATGATTCTCGCT |
| | | | TTGGGTGCGAGAGGTCCCGGGTTCAAATCCC |
| | | | GGACGAGCCCNNN |
| 1277 | Ser | AGA | GTAGTCGTGGCCGAGTGGTTAAGGCGATGGA |
| | | | CTAGAAATCCATTGGGGTTTCCCCGCGCAGG |
| | | | TTCGAATCCTGCCGACTACGNNN |
| 1278 | Ser | CGA | GCTGTGATGGCCGAGTGGTTAAGGCGTTGGA |
| | | | CTCGAAATCCAATGGGGTCTCCCCGCGCAGG |
| | | | TTCGAATCCTGCTCACAGCGNNN |
| 1279 | Ser | GCT | GACGAGGNNTGGCCGAGTGGTTAAGGCGAT |
| | | | GGACTGCTAATCCATTGTGCTCTGCACGCGT |
| | | | GGGTTCGAATCCCATCCTCGTCGNNN |
| 1280 | Ser | TGA | GTAGTCGTGGCCGAGTGGTTAAGGCGATGGA |
| | | | CTTGAAATCCATTGGGGTCTCCCCGCGCAGG |
| | | | TTCGAATCCTGCCGGCTACGNNN |
| 1281 | Thr | AGT | GGCTCCGTGGCTTAGCTGGTTAAAGCGCCTG |
| | | | TCTAGTAAACAGGAGATCCTGGGTTCGAATC |
| | | | CCAGCGGGGCCTNNN |
| 1282 | Thr | CGT | GGCNCTGTGGCTNAGTNGGNTAAAGCGCCGG |
| | | | TCTCGTAAACCNGGAGATCNTGGGTTCGAAT |
| | | | CCCANCNGGGCCTNNN |
| 1283 | Thr | TGT | GGCTCCATAGCTCAGNGGGTTAGAGCACTGG |
| | | | TCTTGTAAACCAGGGGTCGCGAGTTCAAATC |
| | | | TCGCTGGGGCCTNNN |
| 1284 | Trp | CCA | GACCTCGTGGCGCAACGGTAGCGCGTCTGAC |
| | | | TCCAGATCAGAAGGTTGCGTGTTCAAATCAC |
| | | | GTCGGGGTCANNN |
| 1285 | Tyr | GTA | CCTTCGATAGCTCAGCTGGTAGAGCGGAGGA |
| | | | CTGTAGATCCTTAGGTCGCTGGTTCGATTCC |
| | | | GGCTCGAAGGANNN |
| 1286 | Val | AAC | GTTTCCGTAGTGTAGTGGTTATCACGTTCGC |
| | | | CTAACACGCGAAAGGTCCCCGGTTCGAAACC |
| | | | GGGCGGAAACANNN |
| 1287 | Val | CAC | GTTTCCGTAGTGTAGTGGTTATCACGTTCGC |
| | | | CTCACACGCGAAAGGTCCCCGGTTCGAAACC |
| | | | GGGCGGAAACANNN |
| 1288 | Val | TAC | GGTTCCATAGTGTAGTGGTTATCACGTCTGC |
| | | | TTTACACGCAGAAGGTCCTGGGTTCGAGCCC |
| | | | CAGTGGAACCANNN |
| 1289 | iMet | CAT | AGCAGAGTGGCGCAGCGGAAGCGTGCTGGG |
| | | | CCCATAACCCAGAGGTCGATGGATCGAAACC |
| | | | ATCCTCTGCTANNN |

TABLE 11
Score values alignment

| | Candidate | Reference | Match |
|---|---|---|---|
| Row | nucleotide | nucleotide | score |
| 1 | A | A | 1 |
| 2 | T | T | 1 |
| 3 | U | T | 1 |
| 4 | C | C | 1 |
| 5 | G | G | 1 |
| 6 | A | N | 0 |
| 7 | T | N | 0 |
| 8 | C | N | 0 |
| 9 | G | N | 0 |
| 10 | N | A | 0 |
| 11 | N | T | 0 |
| 12 | N | C | 0 |
| 13 | N | G | 0 |
| 14 | N | N | 0 |

### Method of Making TREMs, TREM Core Fragments, and TREM Fragments

Methods for synthesizing oligonucleotides are known in the art and can be used to make a TREM, a TREM core fragment or a TREM fragment disclosed herein. For example, a TREM, TREM core fragment or TREM fragment can be synthesized using solid phase synthesis or liquid phase synthesis.

In an embodiment, a TREM, a TREM core fragment or a TREM fragment made according to a synthetic method disclosed herein has a different modification profile compared to a TREM expressed and isolated from a cell, or compared to a naturally occurring tRNA.

An exemplary method for making a synthetic TREM via 5′-Silyl-2′-Orthoester (2′-ACE) chemistry is provided in Example 3. The method provided in Example 3 can also be used to make a synthetic TREM core fragment or synthetic TREM fragment. Additional synthetic methods are disclosed in Hartsel S A et al., (2005) Oligonucleotide Synthesis, 033-050, the entire contents of which are hereby incorporated by reference.

### TREM Composition

In an embodiment, a TREM composition, e.g., a TREM pharmaceutical composition, comprises a pharmaceutically acceptable excipient. Exemplary excipients include those provided in the FDA Inactive Ingredient Database (https://www.accessdata.fda.gov/scripts/cder/iig/index.Cfm).

In an embodiment, a TREM composition, e.g., a TREM pharmaceutical composition, comprises at least 1, 2, 3, 4, 5, 6, 7, 8, 9, 10, 15, 20, 30, 40, 50, 60, 70, 80, 90, 100 or 150 grams of TREM, TREM core fragment or TREM fragment. In an embodiment, a TREM composition, e.g., a TREM pharmaceutical composition, comprises at least 1, 2, 3, 4, 5, 6, 7, 8, 9, 10, 15, 20, 30, 40, 50 or 100 milligrams of TREM, TREM core fragment or TREM fragment.

In an embodiment, a TREM composition, e.g., a TREM pharmaceutical composition, is at least 10, 20, 30, 40, 50, 60, 70, 80, 90, 95 or 99% dry weight TREMs, TREM core fragments or TREM fragments.

In an embodiment, a TREM composition comprises at least 1×10⁶ TREM molecules, at least 1×10⁷ TREM molecules, at least 1×10⁸ TREM molecules or at least 1×10⁹ TREM molecules.

In an embodiment, a TREM composition comprises at least 1×10⁶ TREM core fragment molecules, at least 1×10⁷ TREM core fragment molecules, at least 1×10⁸ TREM core fragment molecules or at least 1×10⁹ TREM core fragment molecules.

In an embodiment, a TREM composition comprises at least 1×10⁶ TREM fragment molecules, at least 1×10⁷ TREM fragment molecules, at least 1×10⁸ TREM fragment molecules or at least 1×10⁹ TREM fragment molecules.

In an embodiment, a TREM composition produced by any of the methods of making disclosed herein can be charged with an amino acid using an in vitro charging reaction as known in the art.

In an embodiment, a TREM composition comprise one or more species of TREMs, TREM core fragments, or TREM fragments. In an embodiment, a TREM composition comprises a single species of TREM, TREM core fragment, or TREM fragment. In an embodiment, a TREM composition comprises a first TREM, TREM core fragment, or TREM fragment species and a second TREM, TREM core fragment, or TREM fragment species. In an embodiment, the TREM composition comprises X TREM, TREM core fragment, or TREM fragment species, wherein X=2, 3, 4, 5, 6, 7, 8, 9, or 10.

In an embodiment, the TREM, TREM core fragment, or TREM fragment has at least 70, 75, 80, 85, 90, or 95, or has 100%, identity with a sequence encoded by a nucleic acid in Table 1.

In an embodiment, the TREM comprises a consensus sequence provided herein.

A TREM composition can be formulated as a liquid composition, as a lyophilized composition or as a frozen composition.

In some embodiments, a TREM composition can be formulated to be suitable for pharmaceutical use, e.g., a pharmaceutical TREM composition. In an embodiment, a pharmaceutical TREM composition is substantially free of materials and/or reagents used to separate and/or purify a TREM, TREM core fragment, or TREM fragment.

In some embodiments, a TREM composition can be formulated with water for injection. In some embodiments, a TREM composition formulated with water for injection is suitable for pharmaceutical use, e.g., comprises a pharmaceutical TREM composition.

### TREM Characterization

A TREM, TREM core fragment, or TREM fragment, or a TREM composition, e.g., a pharmaceutical TREM composition, produced by any of the methods disclosed herein can be assessed for a characteristic associated with the TREM, TREM core fragment, or TREM fragment or the TREM composition, such as purity, sterility, concentration, structure, or functional activity of the TREM, TREM core fragment, or TREM fragment. Any of the above-mentioned characteristics can be evaluated by providing a value for the characteristic, e.g., by evaluating or testing the TREM, TREM core fragment, or TREM fragment, or the TREM composition, or an intermediate in the production of the TREM composition. The value can also be compared with a standard or a reference value. Responsive to the evaluation, the TREM composition can be classified, e.g., as ready for release, meets production standard for human trials, complies with ISO standards, complies with cGMP standards, or complies with other pharmaceutical standards. Responsive to the evaluation, the TREM composition can be subjected to further processing, e.g., it can be divided into aliquots, e.g., into single or multi-dosage amounts, disposed in a container, e.g., an end-use vial, packaged, shipped, or put into commerce. In embodiments, in response to the evaluation, one or more of the characteristics can be modulated, processed or re-processed to optimize the TREM composition. For example, the TREM composition can be modulated, processed or re-processed to (i) increase the purity of the TREM composition; (ii) decrease the amount of fragments in the composition; (iii) decrease the amount of endotoxins in the composition; (iv) increase the in vitro translation activity of the composition; (v) increase the TREM concentration of the composition; or (vi) inactivate or remove any viral contaminants present in the composition, e.g., by reducing the pH of the composition or by filtration.

In an embodiment, the TREM, TREM core fragment, or TREM fragment (e.g., TREM composition or an intermediate in the production of the TREM composition) has a purity of at least 30%, 40%, 50%, 60%, 70%, 80%, 85%, 90%, 91%, 92%, 93%, 94%, 95%, 96%, 97%, 98%, or 99%, i.e., by mass.

In an embodiment, the TREM (e.g., TREM composition or an intermediate in the production of the TREM composition) has less than 0.1%, 0.5%, 1%, 2%, 3%, 4%, 5%, 6%, 7%, 8%, 9%, 10%, 15%, 20%, 25% TREM fragments relative to full length TREMs.

In an embodiment, the TREM, TREM core fragment, or TREM fragment (e.g., TREM composition or an intermediate in the production of the TREM composition) has low levels or absence of endotoxins, e.g., a negative result as measured by the Limulus amebocyte lysate (LAL) test.

In an embodiment, the TREM, TREM core fragment, or TREM fragment (e.g., TREM composition or an intermediate in the production of the TREM composition) has in-vitro translation activity, e.g., as measured by an assay described in Examples 12-13.

In an embodiment, the TREM, TREM core fragment, or TREM fragment (e.g., TREM composition or an intermediate in the production of the TREM composition) has a TREM concentration of at least 0.1 ng/mL, 0.5 ng/ml, 1 ng/mL, 5 ng/mL, 10 ng/ml, 50 ng/mL, 0.1 ug/mL, 0.5 ug/mL, 1 ug/mL, 2 ug/mL, 5 ug/mL, 10 ug/mL, 20 ug/mL, 30 ug/mL, 40 ug/mL, 50 ug/mL, 60 ug/mL, 70 ug/mL, 80 ug/mL, 100 ug/mL, 200 ug/mL, 300 ug/mL, 500 ug/mL, 1000 ug/mL, 5000 ug/mL, 10,000 ug/mL, or 100,000 ug/mL.

In an embodiment, the TREM, TREM core fragment, or TREM fragment (e.g., TREM composition or an intermediate in the production of the TREM composition) is sterile, e.g., the composition or preparation supports the growth of fewer than 100 viable microorganisms as tested under aseptic conditions, the composition or preparation meets the standard of USP <71>, and/or the composition or preparation meets the standard of USP <85>.

In an embodiment, the TREM, TREM core fragment, or TREM fragment (e.g., TREM composition or an intermediate in the production of the TREM composition) has an undetectable level of viral contaminants, e.g., no viral contaminants. In an embodiment, any viral contaminant, e.g., residual virus, present in the composition is inactivated or removed. In an embodiment, any viral contaminant, e.g., residual virus, is inactivated, e.g., by reducing the pH of the composition. In an embodiment, any viral contaminant, e.g., residual virus, is removed, e.g., by filtration or other methods known in the field.

### TREM Administration

Any TREM composition or pharmaceutical composition described herein can be administered to a cell, tissue or subject, e.g., by direct administration to a cell, tissue and/or an organ in vitro, ex-vivo or in vivo. In-vivo administration may be via, e.g., by local, systemic and/or parenteral routes, for example intravenous, subcutaneous, intraperitoneal, intrathecal, intramuscular, ocular, nasal, urogenital, intradermal, dermal, enteral, intravitreal, intracerebral, intrathecal, or epidural.

### Vectors and Carriers

In some embodiments the TREM, TREM core fragment, or TREM fragment or TREM composition described herein, is delivered to cells, e.g. mammalian cells or human cells, using a vector. The vector may be, e.g., a plasmid or a virus. In some embodiments, delivery is in vivo, in vitro, ex vivo, or in situ. In some embodiments, the virus is an adeno associated virus (AAV), a lentivirus, an adenovirus. In some embodiments, the system or components of the system are delivered to cells with a viral-like particle or a virosome. In some embodiments, the delivery uses more than one virus, viral-like particle or virosome.

### Carriers

A TREM, a TREM composition or a pharmaceutical TREM composition described herein may comprise, may be formulated with, or may be delivered in, a carrier.

### Viral Vectors

The carrier may be a viral vector (e.g., a viral vector comprising a sequence encoding a TREM, a TREM core fragment or a TREM fragment). The viral vector may be administered to a cell or to a subject (e.g., a human subject or animal model) to deliver a TREM, a TREM core fragment or a TREM fragment, a TREM composition or a pharmaceutical TREM composition.

A viral vector may be systemically or locally administered (e.g., injected). Viral genomes provide a rich source of vectors that can be used for the efficient delivery of exogenous genes into a mammalian cell. Viral genomes are known in the art as useful vectors for delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference. In some embodiments the system or components of the system are delivered to cells with a viral-like particle or a virosome.

### Cell and Vesicle-Based Carriers

A TREM, a TREM core fragment or a TREM fragment, a TREM composition or a pharmaceutical TREM composition described herein can be administered to a cell in a vesicle or other membrane-based carrier.

In embodiments, a TREM, a TREM core fragment or a TREM fragment, or TREM composition, or pharmaceutical TREM composition described herein is administered in or via a cell, vesicle or other membrane-based carrier. In one embodiment, the TREM, TREM core fragment, TREM fragment, or TREM composition or pharmaceutical TREM composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi: 10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the TREM, TREM core fragment, TREM fragment, or TREM composition or pharmaceutical TREM composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi: 10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the TREM, TREM core fragment, TREM fragment, or TREM composition or pharmaceutical TREM composition described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the TREM, TREM core fragment, TREM fragment, or TREM composition or pharmaceutical TREM composition described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di (tetradecanoyloxy) propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygcris ct al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

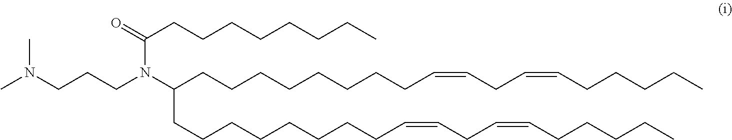 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a TREM composition described herein to the liver and/or hepatocyte cells.

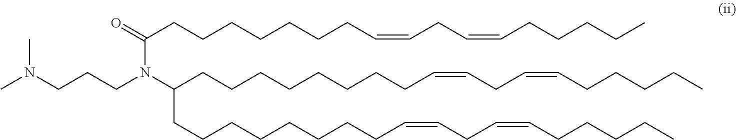 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a TREM composition described herein to the liver and/or hepatocyte cells.

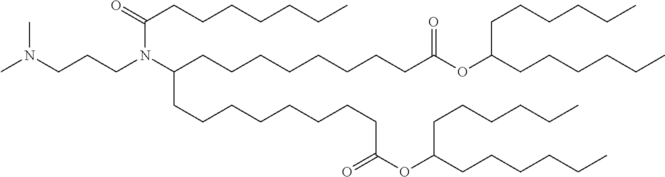 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a TREM composition described herein to the liver and/or hepatocyte cells.

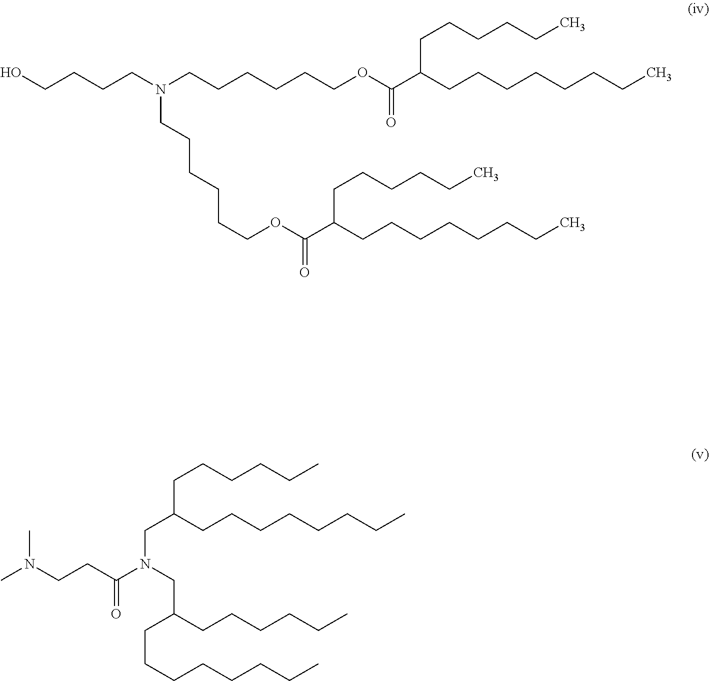

In some embodiments an LNP comprising Formula (v) is used to deliver a TREM composition described herein to the liver and/or hepatocyte cells.

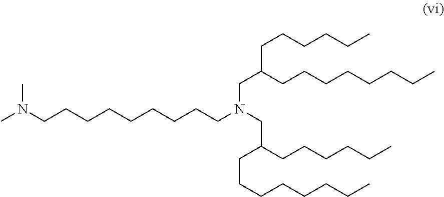 [SMILES: CCCCCCCCC(CCCCCC)CN(CCCCCCCCCN(C)C)CC(CCCCCC)CCCCCCCC]

In some embodiments an LNP comprising Formula (vi) is used to deliver a TREM composition described herein to the liver and/or hepatocyte cells.

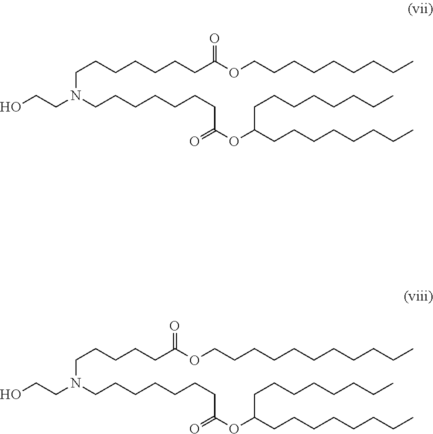 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver a TREM composition described herein to the liver and/or hepatocyte cells.

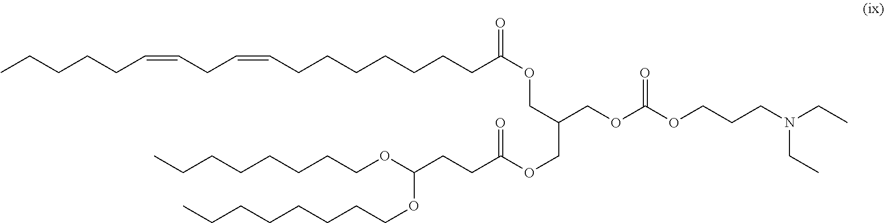 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver a TREM composition described herein to the liver and/or hepatocyte cells.

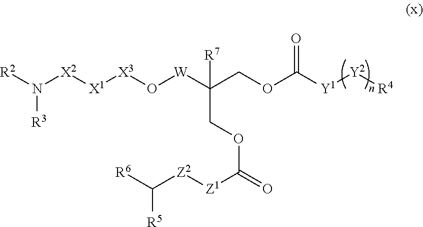

wherein X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

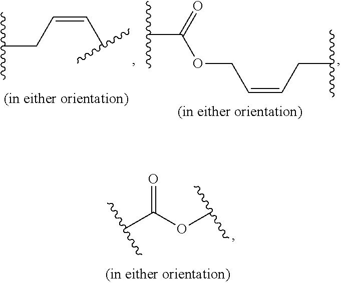

n is 0 to 3, R⁴ is Ci-15 alkyl, Z¹ is Ci-6 alkylene or a direct bond, Z² is

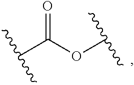 [SMILES: *OC(*)=O]

(in either orientation) or absent, provided that if Z¹ is a direct bond, Z² is absent; R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(=0), Y¹ is linear Ce alkylene, (Y²)n-R⁴ is:

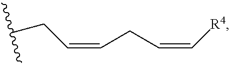

R⁴ is linear C5 alkyl, Z¹ is C2 alkylene, Z² is absent, W is methylene, and R⁷ is H, then R⁵ and R⁶ are not Cx alkoxy.

In some embodiments an LNP comprising Formula (xii) is used to deliver a TREM composition described herein to the liver and/or hepatocyte cells.

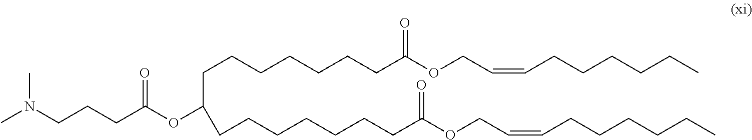 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a TREM composition described herein to the liver and/or hepatocyte cells.

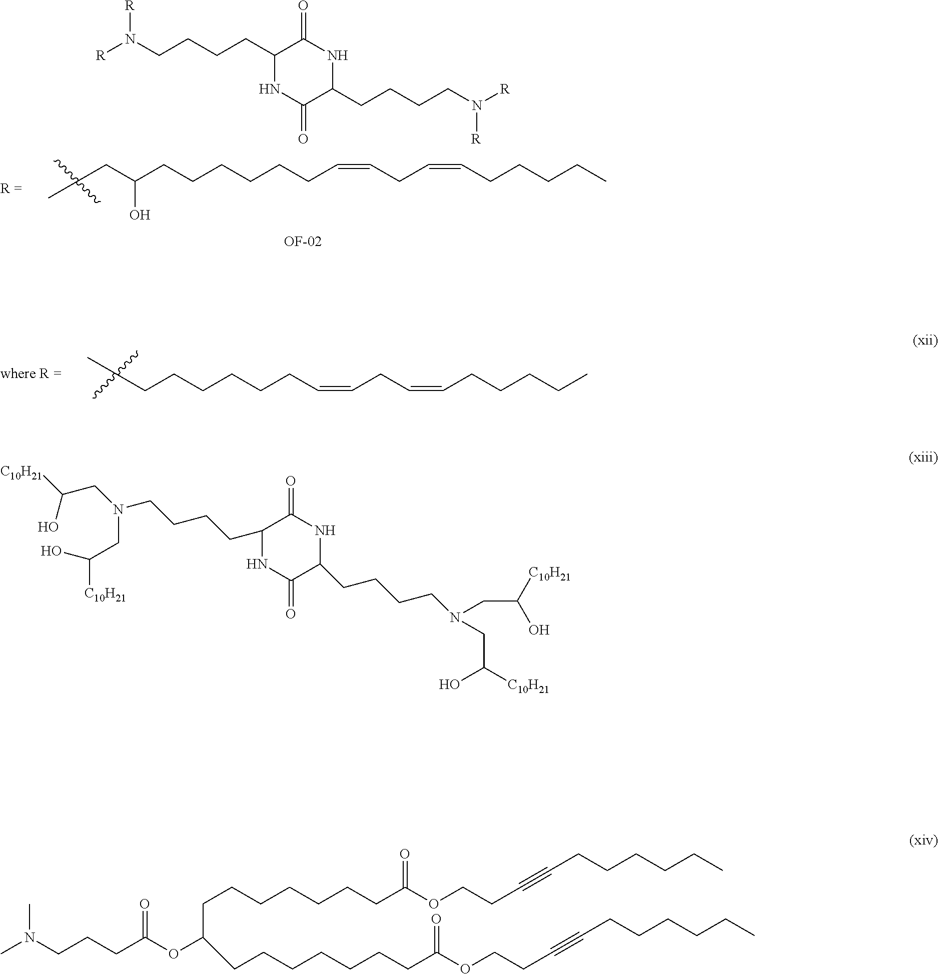

In some embodiments an LNP comprises a compound of Formula (xiii) and a compound of Formula (xiv).

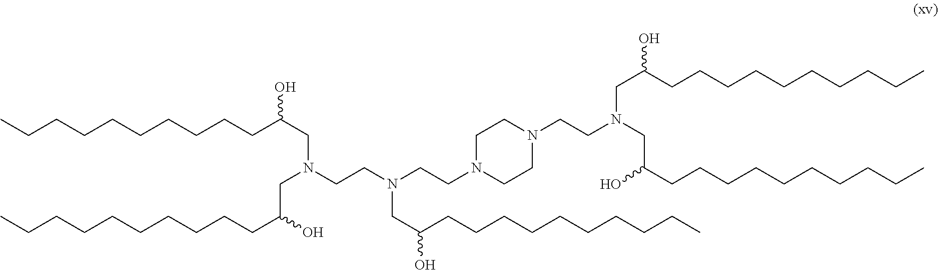 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments, an LNP comprising Formula (xv) is used to deliver a TREM composition described herein to the liver and/or hepatocyte cells.

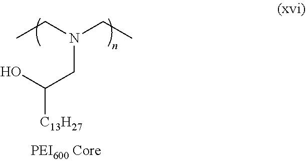

In some embodiments an LNP comprising a formulation of Formula (xvi) is used to deliver a TREM composition described herein to the lung endothelial cells.

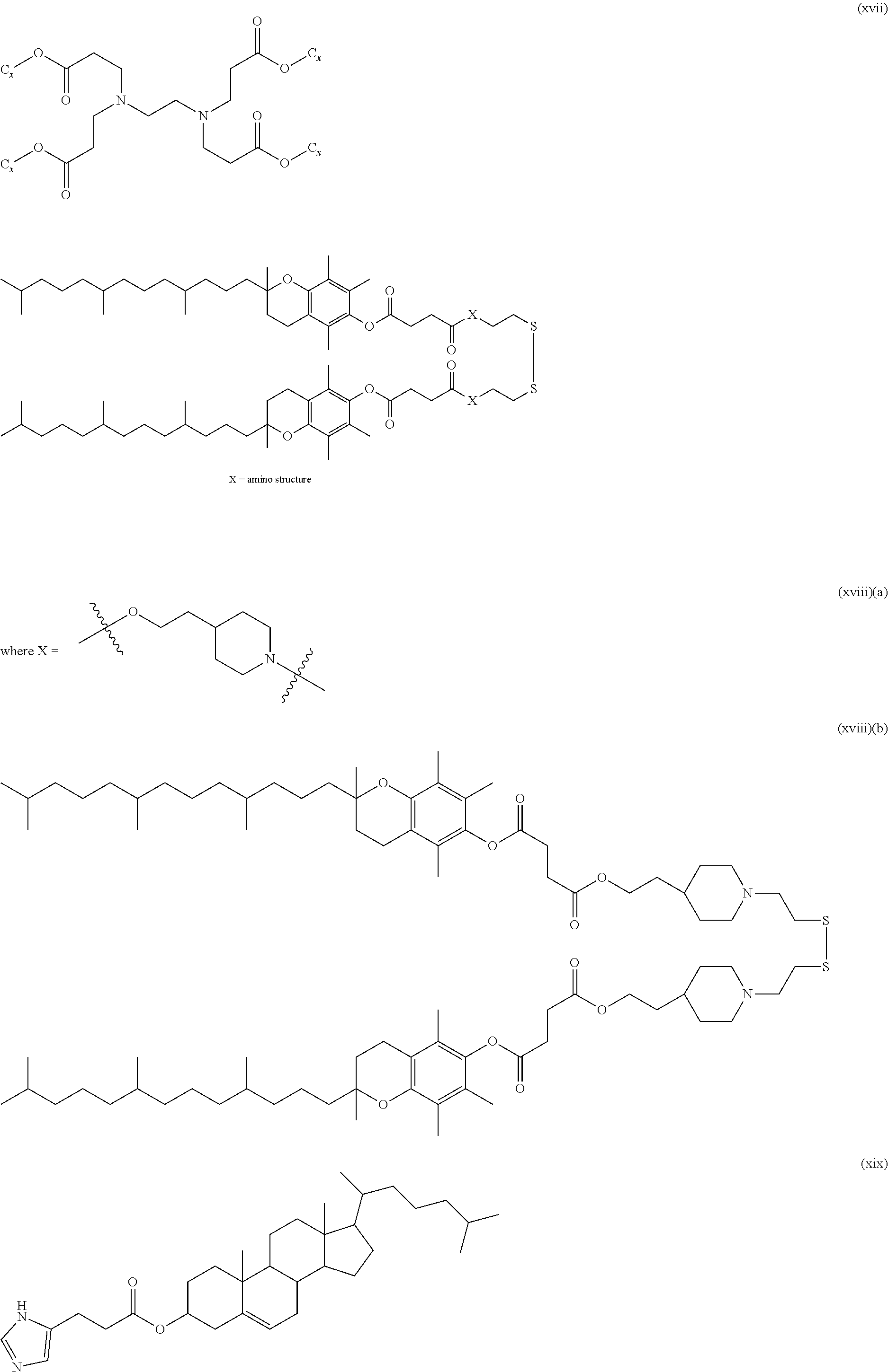

In some embodiments, a lipid compound used to form lipid nanoparticles for the delivery of compositions described herein, e.g., a TREM described herein is made by one of the following reactions:

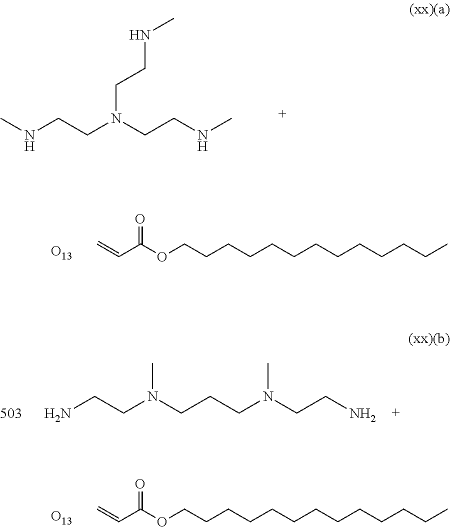

In some embodiments, a composition described herein (e.g., TREM composition) is provided in an LNP that comprises an ionizable lipid. In some embodiments, the ionizable lipid is heptadecan-9-yl 8-((2-hydroxyethyl) (6-oxo-6-(undecyloxy) hexyl) amino) octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis (octyloxy) butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl) propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a TREM described herein, encapsulated within or associated with the lipid nanoparticle. In some embodiments, the TREM is co-formulated with the cationic lipid. The TREM may be adsorbed to the surface of an LNP, e.g., an LNP comprising a cationic lipid. In some embodiments, the TREM may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a TREM.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 IZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyclin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di (tetradecanoyloxy) propyl-1-0-(w-methoxy (polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl] carbamoyl-[omega]-methyl-poly (ethylene glycol), PEG-DMB (3,4-Ditetradecoxylbenzyl-[omega]-methyl-poly (ethylene glycol) ether), and 1,2-dimyristoyl-sn-glycero-3-phosphoethanolamine-N-[methoxy (polyethylene glycol)-2000]. In some embodiments, the PEG-lipid comprises PEG-DMG, 1,2-dimyristoyl-sn-glycero-3-phosphoethanolamine-N-[methoxy (polyethylene glycol)-2000]. In some embodiments, the PEG-lipid comprises a structure selected from:

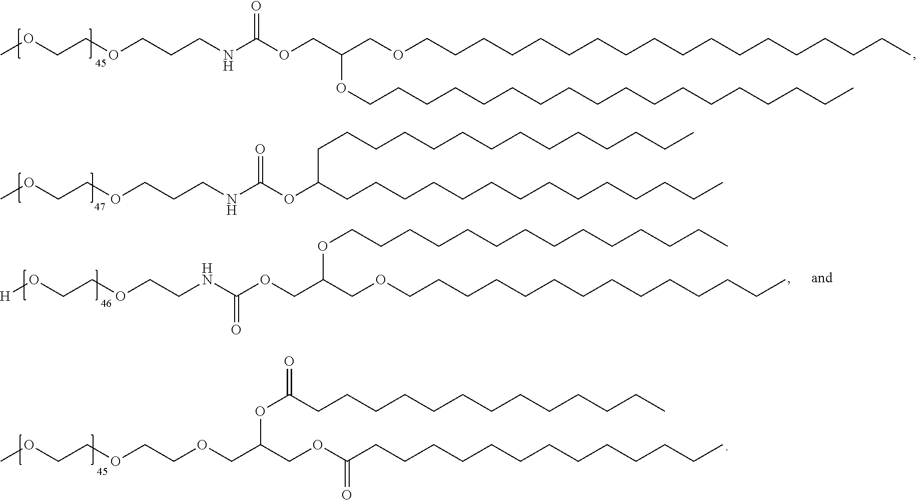

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1. 5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18 (7): 1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly (ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis (octyloxy) butanoyl)oxy)-2-((((3-(diethylamino) propoxy) carbonyl)oxy) methyl) propyl octadeca-9,12-dienoate, also called 3-((4,4-bis (octyloxy) butanoyl)oxy)-2-((((3-(diethylamino) propoxy) carbonyl)oxy) methyl) propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a TREM describes the amount of TREM that is encapsulated or otherwise associated with a LNP after preparation, relative to the initial amount provided. The encapsulation efficiency is desirably high (e.g., close to 100%). The encapsulation efficiency may be measured, for example, by comparing the amount of TREM in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free TREM in a solution. For the lipid nanoparticles described herein, the encapsulation efficiency of a TREM may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51 (34): 8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exosomes can also be used as drug delivery vehicles for the TREM, TREM core fragment, TREM fragment, or TREM compositions or pharmaceutical TREM composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a TREM, TREM core fragment, TREM fragment, or TREM composition, or pharmaceutical TREM composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111 (28): 10131-10136; U.S. Pat. No. 9,644,180; Huang et al. 2017. Nature Communications 8:423; Shi et al. 2014. Proc Natl Acad Sci USA. 111 (28): 10131-10136.

Fusosome compositions, e.g., as described in WO2018208728, can also be used as carriers to deliver the TREM, TREM core fragment, TREM fragment, or TREM composition, or pharmaceutical TREM composition described herein.

Virosomes and virus-like particles (VLPs) can also be used as carriers to deliver a TREM, TREM core fragment, TREM fragment, or TREM composition, or pharmaceutical TREM composition described herein to targeted cells.

Plant nanovesicles, e.g., as described in WO2011097480A1, WO2013070324A1, or WO2017004526A1 can also be used as carriers to deliver the TREM, TREM core fragment, TREM fragment, or TREM composition, or pharmaceutical TREM composition described herein.

Delivery without a Carrier

A TREM, a TREM core fragment or a TREM fragment, a TREM composition or a pharmaceutical TREM composition described herein can be administered to a cell without a carrier, e.g., via naked delivery of the TREM, a TREM core fragment or a TREM fragment, a TREM composition or a pharmaceutical TREM composition.

In some embodiments, naked delivery as used herein refers to delivery without a carrier. In some embodiments, delivery without a carrier, e.g., naked delivery, comprises delivery with a moiety, e.g., a targeting peptide.

In some embodiments, a TREM, a TREM core fragment or a TREM fragment, or TREM composition, or pharmaceutical TREM composition described herein is delivered to a cell without a carrier, e.g., via naked delivery. In some embodiments, the delivery without a carrier, e.g., naked delivery, comprises delivery with a moiety, e.g., a targeting peptide.

### Use of TREMs

A composition comprising a TREM comprising an ASGPR binding moiety (e.g., a pharmaceutical TREM composition described herein) can modulate a function in a cell, tissue or subject. In embodiments, a composition comprising a TREM comprising an ASGPR binding moiety (e.g., a pharmaceutical TREM composition) described herein is contacted with a cell or tissue, or administered to a subject in need thereof, in an amount and for a time sufficient to modulate (increase or decrease) one or more of the following parameters: adaptor function (e.g., cognate or non-cognate adaptor function), e.g., the rate, efficiency, robustness, and/or specificity of initiation or elongation of a polypeptide chain; ribosome binding and/or occupancy; regulatory function (e.g., gene silencing or signaling); cell fate; mRNA stability; protein stability; protein transduction; protein compartmentalization. A parameter may be modulated, e.g., by at least 5% (e.g., at least 10%, 15%, 20%, 25%, 30%, 40%, 50%, 60%, 70%, 80%, 90%, 100%, 150%, 200% or more) compared to a reference tissue, cell or subject (e.g., a healthy, wild-type or control cell, tissue or subject).

In another aspect, the disclosure provides a method of treating a subject having an endogenous open reading frame (ORF) which comprises a premature termination codon (PTC), comprising providing a TREM composition comprising a TREM, a TREM core fragment, or a TREM fragment disclosed herein, wherein the TREM comprises an anticodon that pairs with the PTC in the ORF; contacting the subject with the composition comprising a TREM, TREM core fragment or TREM fragment in an amount and/or for a time sufficient to treat the subject, thereby treating the subject. In an embodiment, the PTC comprises UAA, UGA or UAG.

In another aspect, the disclosure provides a method of treating a subject having an disease or disorder associated with a premature termination codon (PTC), comprising providing a TREM composition comprising a TREM, a TREM core fragment, or a TREM fragment disclosed herein; contacting the subject with the composition comprising a TREM, TREM core fragment or TREM fragment in an amount and/or for a time sufficient to treat the subject, thereby treating the subject. In an embodiment, the PTC comprises UAA, UGA or UAG. In an embodiment, the disease or disorder associated with a PTC is a disease or disorder described herein, e.g., a cancer or a monogenic disease.

In an embodiment of any of the methods disclosed herein, the codon having the first sequence comprises a mutation (e.g., a point mutation, e.g., a nonsense mutation), resulting in a premature termination codon (PTC) chosen from UAA, UGA or UAG. In an embodiment, the codon having the first sequence or the PTC comprises a UAA mutation. In an embodiment, the codon having the first sequence or the PTC comprises a UGA mutation. In an embodiment, the codon having the first sequence or the PTC comprises a UAG mutation.

In another aspect, the disclosure provides a method of making a TREM, a TREM core fragment, or a TREM fragment disclosed herein, comprising linking a first nucleotide to a second nucleotide to form the TREM.

In an embodiment, the TREM, TREM core fragment or TREM fragment is non-naturally occurring (e.g., synthetic).

In an embodiment, the TREM, TREM core fragment or TREM fragment is made by cell-free solid phase synthesis.

In another aspect, the disclosure provides a method of modulating a tRNA pool in a cell comprising: providing a TREM, a TREM core fragment, or a TREM fragment disclosed herein, and contacting the cell with the TREM, TREM core fragment or TREM fragment, thereby modulating the tRNA pool in the cell.

In an aspect, the disclosure provides a method of contacting a cell, tissue, or subject with a TREM, a TREM core fragment, or a TREM fragment disclosed herein, comprising: contacting the cell, tissue or subject with the TREM, TREM core fragment or TREM fragment, thereby contacting the cell, tissue, or subject with the TREM, TREM core fragment or TREM fragment.

In another aspect, the disclosure provides a method of delivering a TREM, TREM core fragment or TREM fragment to a cell, tissue, or subject, comprising: providing a cell, tissue, or subject, and contacting the cell, tissue, or subject, a TREM, a TREM core fragment, or a TREM fragment disclosed herein.

In an aspect, the disclosure provides a method of modulating a tRNA pool in a cell comprising an endogenous open reading frame (ORF), which ORF comprises a codon having a first sequence, comprising:
optionally, acquiring knowledge of the abundance of one or both of (i) and (ii), e.g., acquiring knowledge of the relative amounts of: (i) and (ii) in the cell, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the cell;
contacting the cell with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the cell,
thereby modulating the tRNA pool in the cell.

In another aspect, the disclosure provides a method of modulating a tRNA pool in a subject having an ORF, which ORF comprises a codon having a first sequence, comprising:
optionally, acquiring knowledge of the abundance of one or both of (i) and (ii), e.g., acquiring knowledge of the relative amounts of: (i) and (ii) in the subject, wherein (i) is a tRNA moiety having an anticodon that pairs with the codon of the ORF having a first sequence (the first tRNA moiety) and (ii) is an isoacceptor tRNA moiety having an anticodon that pairs with a codon other than the codon having the first sequence (the second tRNA moiety) in the subject;
contacting the subject with a TREM, a TREM core fragment, or a TREM fragment disclosed herein, wherein the TREM, TREM core fragment or TREM fragment has an anticodon that pairs with: the codon having the first sequence; or the codon other than the codon having the first sequence, in an amount and/or for a time sufficient to modulate the relative amounts of the first tRNA moiety and the second tRNA moiety in the subject,
thereby modulating the tRNA pool in the subject.

All references and publications cited herein are hereby incorporated by reference.

### ENUMERATED EMBODIMENTS

1. A TREM entity comprising an asialoglycoprotein receptor (ASGPR) binding moiety, wherein the ASGPR binding moiety is bound to:
   a) a sugar moiety (e.g., ribose moiety);
   b) a nucleobase (e.g., A, G, C, or U); and/or
   c) a phosphate backbone
at any nucleotide position within the TREM.
2. The TREM entity of embodiment 1, wherein the asialoglycoprotein receptor binding moiety comprises a galactose (Gal), galactosaminic (GalNH₂), or N-acetylgalactosamine (GalNAc) moiety.
3. The TREM entity of embodiment 2, wherein the GalNAc moiety comprises GalNAc or an analog thereof.
4. The TREM entity of embodiment 1, wherein the ASGPR binding moiety is bound to a sugar moiety (e.g., ribose moiety) at the 2′ or 4′ position of the sugar moiety.
5. The TREM entity of embodiment 1, wherein the ASGPR binding moiety is bound to a nucleobase (e.g., A, G, C or U).
6. The TREM entity of embodiment 1, wherein the sugar moiety (e.g., ribose moiety), nucleobase, and/or the phosphate backbone are within the L1 region of a TREM sequence.
7. The TREM entity of any one of embodiments 1-6, wherein the sugar moiety (e.g., ribose moiety), nucleobase, and/or the phosphate backbone are within the ASt Domain 1 of a TREM sequence.
8. The TREM entity of any one of embodiments 1-6, wherein the sugar moiety (e.g., ribose moiety), nucleobase, and/or the phosphate backbone are within the ASt Domain 2 of a TREM sequence.
9. The TREM entity of any one of embodiments 1-6, wherein the sugar moiety (e.g., ribose moiety), nucleobase, and/or the phosphate backbone are within the L2 region of a TREM sequence.
10. The TREM entity of any one of embodiments 1-6, wherein the sugar moiety (e.g., ribose moiety), nucleobase, and/or the phosphate backbone are within the DH Domain of a TREM sequence.
11. The TREM entity of any one of embodiments 1-6, wherein the sugar moiety (e.g., ribose moiety), nucleobase, and/or the phosphate backbone are within the L3 region of a TREM sequence.
12. The TREM entity of any one of embodiments 1-6, wherein the sugar moiety (e.g., ribose moiety), nucleobase, and/or the phosphate backbone are within the ACH Domain of a TREM sequence.
13. The TREM entity of any one of embodiments 1-6, wherein the sugar moiety (e.g., ribose moiety) and/or the phosphate backbone are within the VL Domain of a TREM sequence.
14. The TREM entity of any one of embodiments 1-6, wherein the sugar moiety (e.g., ribose moiety), nucleobase, and/or the phosphate backbone are within the TH Domain of a TREM sequence.
15. The TREM entity of any one of embodiments 1-6, wherein the sugar moiety (e.g., ribose moiety), nucleobase, and/or the phosphate backbone are within the L⁴ region of a TREM sequence.
16. The TREM entity of any one of embodiments 1-15, wherein the TREM entity comprises a TREM, a TREM Core Fragment, or a TREM Fragment.
17. A TREM comprising:
   (i) a sequence of Formula A comprising:
   [L1]_{y}-[ASt Domain1]ₓ-[L2]ₓ-[DH Domain]ₓ-[L3]ₓ-[ACH Domain]ₓ-[VL Domain]_{y}-[TH Domain]ₓ-[L4]ₓ-[ASt Domain2]ₓ, (A); and
   (ii) an asialoglycoprotein receptor (ASGPR) binding moiety (e.g., a GalNAc moiety, e.g., GalNAc);
   wherein the ASGPR binding moiety is bound to:
      a) a sugar moiety (e.g., ribose moiety);
      b) a nucleobase (e.g., A, G, C, or U); and/or
      c) the phosphate backbone
   wherein y is 0 or 1 and x is 1.
18. The TREM of embodiment 17, wherein the ASGPR binding moiety is bound to a sugar moiety (e.g., a ribose moiety).
19. The TREM of embodiment 18, wherein the ASGPR binding moiety is present on the sugar moiety at the 2′ or 4′ position of the sugar moiety.
20. The TREM of embodiment 18, wherein the ASGPR binding moiety is present on a nucleobase (e.g., A, G, C, or U).
21. The TREM of embodiment 17, wherein the ASGPR binding moiety is bound to the phosphate backbone at any nucleotide position within the TREM.
22. The TREM of any one of embodiments 1-17, wherein the ASGPR binding moiety is bound to a sugar moiety (e.g., ribose moiety) within the ASt Domain 1.
23. The TREM of any one of embodiments 1-17, wherein the ASGPR binding moiety is bound to a sugar moiety (e.g., ribose moiety) at any one of positions 1-9 within the TREM.
24. The TREM of embodiment 23, wherein the ASGPR binding moiety is present on the sugar moiety (e.g., the ribose moiety) at the 2′ or 4′ position of the sugar moiety.
25. The TREM of embodiment 23, wherein the ASGPR binding moiety is present on a nucleobase (e.g., A, G, or U).
26. The TREM of any one of embodiments 1-17, wherein the ASGPR binding moiety is bound to the phosphate backbone at any one of positions 1-9 within the TREM.
27. The TREM of embodiment 17, wherein the ASGPR binding moiety is bound to a sugar moiety (e.g., ribose moiety) within the ASt Domain 2.
28. The TREM of any one of embodiments 1-27, wherein the ASGPR binding moiety is bound to a sugar moiety (e.g., ribose moiety) at any one of positions 65-76 within the TREM.
29. The TREM of embodiment 28, wherein the ASGPR binding moiety is present on the sugar moiety (e.g., the ribose moiety) at the 2′ oxygen or carbon or 4′ carbon of the sugar moiety.
30. The TREM of embodiment 28, wherein the ASGPR binding moiety is present a nucleobase (e.g., A, G, C, U).
31. The TREM of any one of embodiments 1-27, wherein the ASGPR binding moiety is bound to the phosphate backbone at any nucleotide position at any one of positions 65-76 within the TREM.
32. The TREM of any one of embodiments 1-17, wherein the ASGPR binding is bound to a sugar moiety (e.g., a ribose moiety) within the ACH Domain.
33. The TREM of embodiment 32, wherein the ASGPR binding moiety is bound to a sugar moiety (e.g., ribose moiety) at any one of positions 27-43 within the TREM.
34. The TREM of any one of embodiments 32 or 33, wherein the ASGPR binding moiety is present on the sugar moiety (e.g., the ribose moiety) at the 2′ or 4′ position of the sugar moiety.
35. The TREM of embodiment 33, wherein the ASGPR binding moiety is present on a nucleobase (e.g., A, G, C, or U).
36. The TREM of any one of embodiments 1-17, wherein the ASGPR binding moiety is bound to the phosphate backbone at any one of positions 27-43 within the TREM.
37. The TREM of any one of embodiments 1-17, wherein the ASGPR binding moiety is present within the DHD.
38. The TREM of embodiment 37, wherein the ASGPR binding moiety is bound to a sugar moiety (e.g., ribose moiety) of a nucleotide in the DHD.
39. The TREM of embodiment 38, wherein the ASGPR binding moiety is bound to a sugar moiety (e.g., ribose moiety) of a nucleotide at positions 10-26 within a TREM sequence.
40. The TREM of any one of embodiments 38 or 39, wherein the ASGPR binding moiety is present on the sugar moiety (e.g., the ribose moiety) at the 2′ or 4′ position of the sugar moiety.
41. The TREM of any one of embodiments 38 and 39, wherein the ASGPR binding moiety is present on a nucleobase (e.g., A, G, C, or U).
42. The TREM of any one of embodiments 1-17, wherein the ASGPR binding moiety is bound to the phosphate backbone at any one of positions 27-43 within the TREM.
43. The TREM of any one of embodiments 1-17, wherein the ASGPR binding moiety is present on the sugar moiety (e.g., the ribose moiety) of a nucleotide or the phosphate backbone within a linker region.
44. The TREM of embodiment 43, wherein the linker region is L1, L2, L3, and/or L4.
45. The TREM of any one of embodiments 1-44, wherein the ASGPR binding moiety (e.g., a GalNAc moiety, e.g., GalNAc) is coupled to a sugar moiety (e.g., ribose moiety) of a nucleotide of the TREM molecule via a covalent linkage (e.g., at a nitrogen or carbon atom in the sugar moiety).
46. The TREM of any one of embodiments 1-45, wherein the ASGPR binding moiety (e.g., a GalNAc moiety, e.g., GalNAc) is coupled to the phosphate backbone of a nucleotide of the TREM molecule via a covalent linkage.
47. The TREM of any one of embodiments 1-45, wherein the ASGPR binding moiety is bound to a carbon atom or an oxygen atom within the sugar moiety (e.g., ribose moiety).
48. The TREM of any one of embodiments 1-45, wherein the ASGPR binding moiety is bound to the 2′-oxygen or carbon atom within the sugar moiety (e.g., ribose moiety).
49. The TREM molecule of any one of embodiments 1-48, wherein the ASGPR binding moiety comprises a GalNAc moiety (e.g., a GalNAc or a GalNAc analog).
50. The TREM molecule of any one of embodiments 1-49, wherein the ASGPR binding moiety comprises a plurality of GalNAc moieties.
51. The TREM molecule of any one of embodiments 1-50, wherein the ASGPR binding moiety comprises a structure of Formula (I):

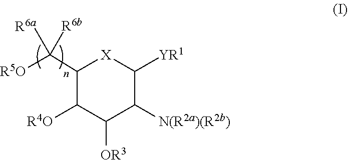

or a salt thereof, wherein:
   each of X and Y is independently O, N (R⁷), or S;
   each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸;
   or R³ and R⁴ are taken together with the oxygen atoms to which they are connected to form a heterocyclyl ring optionally substituted with one or more R⁸;
   R^{2a} is hydrogen or alkyl; R^{2b} is —C(O)alkyl (e.g., C(O)CH₃);
   each of R^{6a} and R^{6b} is hydrogen, alkyl, alkenyl, alkynyl, heteroalkyl, haloalkyl, halo, cyano, nitro,-ORA, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹;
   R⁷ is hydrogen, alkyl, or C(O)-alkyl;
   each of R⁸ and R⁹ is independently hydrogen, halo, cyano, alkyl, alkenyl, alkynyl, heteroalkyl, haloalkyl, cycloalkyl, or heterocyclyl;
   R^{A} is hydrogen, or alkyl, alkenyl, alkynyl, and n is an integer between 0 and 6, wherein the structure of Formula (I) may be connected to a linker or TREM at:
   (a) a sugar moiety (e.g., a ribose)
   (b) a nucleobase (e.g., A, G, C, or U); and/or
   (c) the phosphate backbone at any nucleotide position
   within a TREM sequence.
52. The TREM of embodiment 51, wherein the GalNAc moiety comprises a plurality of structures of Formula (I).
53. The TREM of embodiment 51 or 52, wherein the GalNAc moiety further comprises a linker.
54. The TREM of any one of embodiments 1-53, wherein the ASGPR binding moiety comprises a structure of Formula (I-a):

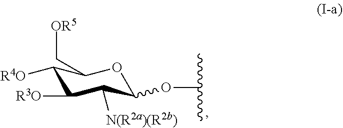

or a salt thereof, wherein:
   R^{2a} is hydrogen or alkyl;
   R^{2b} is —C(O)alkyl (e.g., C(O)CH₃);
   each of R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸; and
   R⁸ is hydrogen, halo, cyano, alkyl, alkenyl, alkynyl, heteroalkyl, haloalkyl, cycloalkyl, or heterocyclyl,
   wherein the “ [symbol] ” represents a bond in any configuration, and “ [symbol] ” represents an attachment point to a linker, nucleobase, or a sugar moiety of a TREM.
55. The TREM of any one of embodiments 1-50, wherein the ASGPR binding moiety comprises a structure of Formula (II):

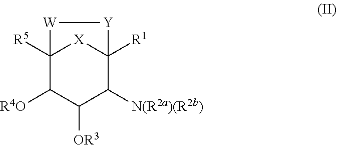

or a salt thereof, wherein:
   X is O, N (R⁷), or S;
   each of W or Y is independently O or C (R^{10a})(R^{10b}), wherein one of W and Y is O;
   each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸;
   or R³ and R⁴ are taken together with the oxygen atoms to which they are connected to form a heterocyclyl ring optionally substituted with one or more R⁸;
   R^{2a} is hydrogen or alkyl; R^{2b} is —C(O)alkyl (e.g., C(O)CH₃);
   each of R^{6a} and R^{6b} is hydrogen, alkyl, alkenyl, alkynyl, heteroalkyl, haloalkyl, halo, cyano, nitro,-ORA, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹; R⁷ is hydrogen, alkyl, or C(O)-alkyl;
   each of R⁸ and R⁹ is independently hydrogen, halo, cyano, alkyl, alkenyl, alkynyl, heteroalkyl, haloalkyl, cycloalkyl, or heterocyclyl; each of R^{10a} and R^{10b} is independently hydrogen, heteroalkyl, haloalkyl, or halo; and RA is hydrogen, or alkyl, alkenyl, alkynyl,
   wherein the structure of Formula (I) may be connected to a linker or a TREM at:
   (a) a sugar moiety;
   (b) a nucleobase; and/or
   (c) the phosphate backbone at any nucleotide position.
56. The TREM of any one of embodiments 1-50, wherein the ASGPR binding moiety comprises a structure of Formula (II):

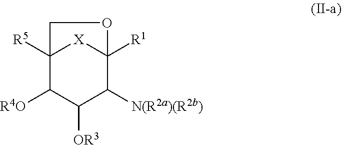

or a salt thereof, wherein X is O, N (R⁷), or S;
   each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸;
   or R³ and R⁴ are taken together with the oxygen atoms to which they are connected to form a heterocyclyl ring optionally substituted with one or more R⁸;
   R^{2a} is hydrogen or alkyl; R^{2b} is —C(O)alkyl (e.g., C(O)CH₃); each of R^{6a} and R^{6b} is hydrogen, alkyl, alkenyl, alkynyl, heteroalkyl, haloalkyl, halo, cyano, nitro,-ORA, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹;
   R⁷ is hydrogen, alkyl, or C(O)-alkyl;
   each of R⁸ and R⁹ is independently hydrogen, halo, cyano, alkyl, alkenyl, alkynyl, heteroalkyl, haloalkyl, cycloalkyl, or heterocyclyl; and RA is hydrogen, or alkyl, alkenyl, alkynyl,
   wherein the structure of Formula (I) may be connected to a linker or TREM at
   (a) a sugar moiety;
   (b) a nucleobase; and/or
   (c) the phosphate backbone at any nucleotide position.
57. The TREM of any one of embodiments 1-50, wherein the ASGPR binding moiety comprises a structure of Formula (II-b):

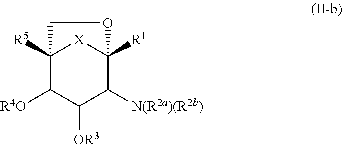

or a salt thereof, wherein:
   X is O, N (R⁷), or S;
   each of R¹, R³, R⁴, and R⁵ are independently hydrogen, alkyl, alkenyl, alkynyl, heteroalkyl, haloalkyl, aryl, heteroaryl, cycloalkyl, heterocyclyl, C(O)-alkyl, C(O)-alkenyl, C(O)-alkynyl, C(O)-heteroalkyl, C(O)-haloalkyl, C(O)-aryl, C(O)-heteroaryl, C(O)-cycloalkyl, or C(O)-heterocyclyl, wherein each alkyl, alkenyl, alkynyl, heteroalkyl, haloalkyl, aryl, heteroaryl, cycloalkyl, and heterocyclyl is optionally substituted with one or more R⁸;
   or R³ and R⁴ are taken together with the oxygen atoms to which they are connected to form a heterocyclyl ring optionally substituted with one or more R⁸;
   R^{2a} is hydrogen or alkyl; R^{2b} is —C(O)alkyl (e.g., C(O)CH₃);
   each of R^{6a} and R^{6b} is hydrogen, alkyl, alkenyl, alkynyl, heteroalkyl, haloalkyl, halo, cyano, nitro,-ORA, aryl, heteroaryl, cycloalkyl, or heterocyclyl, wherein each alkyl, alkenyl, alkynyl, heteroalkyl, haloalkyl, aryl, heteroaryl, cycloalkyl, and heterocyclyl is optionally substituted with one or more R⁹;
   R⁷ is hydrogen, alkyl, or C(O)-alkyl; each of R⁸ and R⁹ is independently hydrogen, halo, cyano, alkyl, alkenyl, alkynyl, heteroalkyl, haloalkyl, cycloalkyl, or heterocyclyl; and
   R^{A} is hydrogen, or alkyl, alkenyl, alkynyl, wherein the structure of Formula (I) may be connected to a linker or a TREM at:
   (a) a sugar moiety;
   (b) a nucleobase; and/or
   (c) the phosphate backbone at any nucleotide position.
58. The TREM of any one of embodiments 1-50, wherein the ASGPR binding moiety comprises a structure of Formula (III):

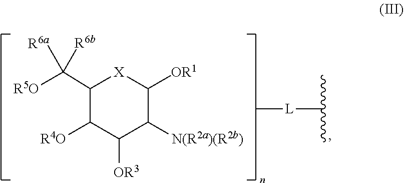

or a salt thereof, wherein each of R¹, R2a, R2b, R³, R⁴, R⁵, R^{6a}, and R^{6b} and subvariables thereof are as defined for Formula (I), L is a linker, and n is an integer between 1 and 100, wherein “ [symbol] ” represents an attachment point to a branching point, additional linker, nucleobase, or a sugar of a TREM.
59. The TREM of embodiment 58, wherein L comprises an alkylene, alkenylene, alkynylene, heteroalkylene, or haloalkylene group.
60. The TREM of embodiment 58 or 59, wherein L comprises a carbonyl, amide, amine, or ester moiety.
61. The TREM of any one of embodiments 1-50, wherein the ASGPR binding moiety comprises a structure of Formula (III-a):

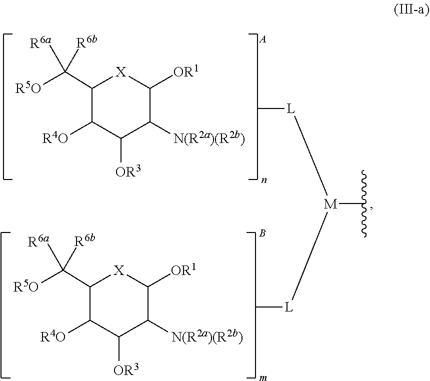

or a salt thereof, wherein:
   each of R¹, R^{2a}, R^{2b}, R³, R⁴, R⁵, R^{6a}, and R^{6b} and subvariables thereof are as defined for Formula (I), each of L¹ and L² is independently a linker, each of m and n is independently an integer between 1 and 100, and M is a linker, wherein “ [symbol] ” represents an attachment point to a branching point, additional linker, nucleobase, or a sugar of a TREM.
62. The TREM of any one of embodiments 1-50, wherein the ASGPR binding moiety comprises a structure of Formula (II-b):

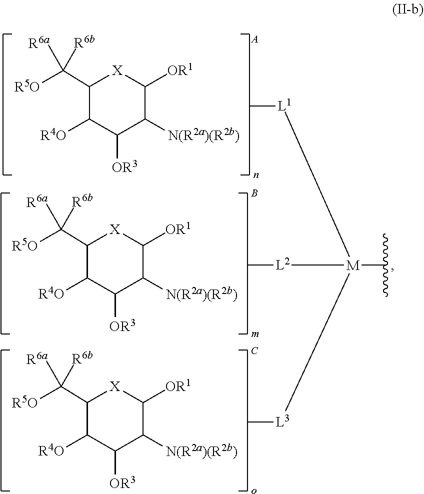

or a salt thereof, wherein:
   each of R¹, R^{2a}, R^{2b}, R³, R⁴, R⁵, R^{6a}, and R^{6b} and subvariables thereof are as defined for Formula (I);
   each of L¹, L², and L³ is independently a linker;
   each of m, n, and o is independently an integer between 1 and 100;
   and M is a branching point, wherein “ [symbol] ” represents an attachment point to a branching point, additional linker, nucleobase, or a sugar of aTREM.
63. The TREM of any one of embodiments 61 or 62, wherein each of L¹, L², and optionally L³ independently comprises an alkylene, alkenylene, alkynylene, heteroalkylene, or haloalkylene group.
64. The TREM of any one of embodiments 61-63, wherein each of L¹, L², and optionally L³ independently comprises a carbonyl, amide, amine, or ester moiety.
65. The TREM of any one of embodiments 61-64, wherein M comprises an alkylene, alkenylene, alkynylene, heteroalkylene, or haloalkylene group.
66. The TREM of any one of embodiments 61-65, wherein M comprises a carbonyl, amide, amine, or ester moiety.
67. The TREM of any one of embodiments 1-50, wherein the ASGPR binding moiety comprises a structure of Formula (II-c):

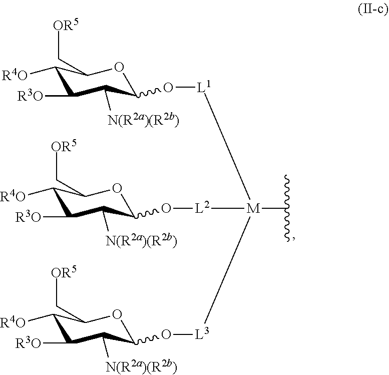

or a salt thereof, wherein:
   each of R^{2a}, R^{2b}, R³, R⁴, R⁵, and subvariables thereof are as defined for Formula (I);
   each of L¹, L², and L³ is independently a linker; and
   M is a branching point, wherein “ [symbol] ” represents an attachment point to a branching point, additional linker, nucleobase, or a sugar of a TREM.
68. The TREM of embodiment 67, each of L¹, L², and L³ independently comprises an alkylene, alkenylene, alkynylene, heteroalkylene, or haloalkylene group.
69. The TREM of embodiment 67 or 68, wherein each of L¹, L², and L³ independently comprises a carbonyl, amide, amine, or ester moiety.
70. The TREM of any one of embodiments 67-69, wherein M comprises an alkylene, alkenylene, alkynylene, heteroalkylene, or haloalkylene group.
71. The TREM of any one of embodiments 67-70, wherein M comprises a carbonyl, amide, amine, or ester moiety.
72. The TREM of any one of embodiments 1-71, wherein the TREM position bound to the ASGPR binding moiety comprises:

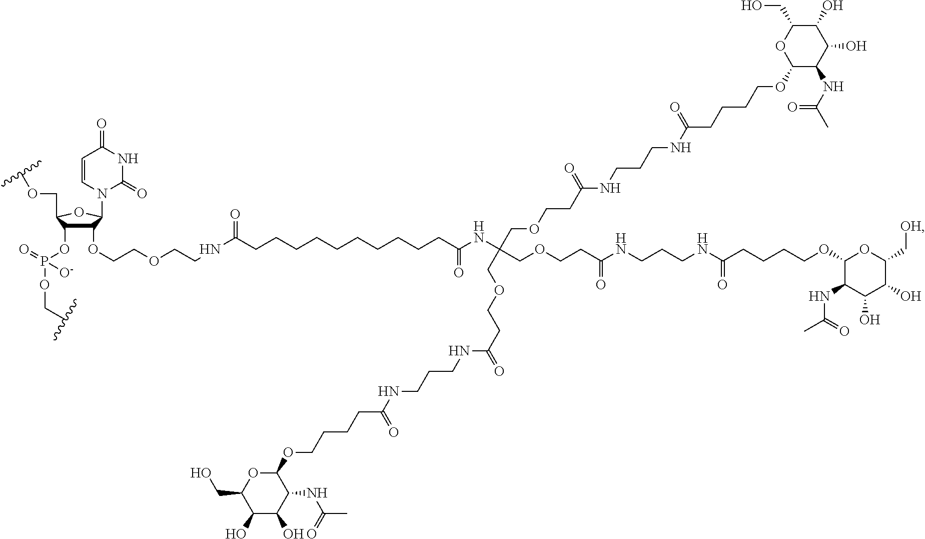

or a pharmaceutically acceptable salt thereof.
73. The TREM of any one of embodiments 1-72, wherein the ASGPR binding moiety is bound to a sugar (e.g., a ribose) at any one of TREM positions 1, 2, 3, 4, 5, 6, 7, 8, or 9.
74. The TREM of any one of embodiments 1-73, wherein the ASGPR binding moiety is bound to a sugar (e.g., a ribose) at a plurality of TREM positions selected from 1, 2, 3, 4, 5, 6, 7, 8, or 9.
75. The TREM of any one of embodiments 1-72, wherein the ASGPR binding moiety is bound to the phosphate backbone at any one of TREM positions 1, 2, 3, 4, 5, 6, 7, 8, or 9.
76. The TREM of any one of embodiments 1-75, wherein the ASGPR binding moiety is bound to the phosphate backbone at a plurality of TREM positions selected from 1, 2, 3, 4, 5, 6, 7, 8, or 9.
77. The TREM of any one of embodiments 1-76, wherein the ASGPR binding moiety is bound to a sugar (e.g., a ribose) at any one of TREM positions 65, 66, 67, 68, 69, 70, 71, 72, 73, 74, 75, or 76.
78. The TREM of any one of embodiments 1-77, wherein the ASGPR binding moiety is bound to a sugar (e.g., a ribose) at a plurality of TREM positions selected from 65, 66, 67, 68, 69, 70, 71, 72, 73, 74, 75, or 76.
79. The TREM of any one of embodiments 1-78, wherein the ASGPR binding moiety is bound to the phosphate backbone at any one of TREM positions 65, 66, 67, 68, 69, 70, 71, 72, 73, 74, 75, or 76.
80. The TREM of any one of embodiments 1-79, wherein the ASGPR binding moiety is bound to the phosphate backbone at a plurality of TREM positions selected from 65, 66, 67, 68, 69, 70, 71, 72, 73, 74, 75, or 76.
81. The TREM of any one of embodiments 1-80, wherein the ASGPR binding moiety is bound to a sugar (e.g., a ribose) at any one of TREM positions 27, 28, 29, 30, 31, 32, 33, 34, 35, 36, 37, 38, 39 40, 41, 42, or 43.
82. The TREM of any one of embodiments 1-81, wherein the ASGPR binding moiety is bound to a sugar (e.g., a ribose) at a plurality of TREM positions selected from 27, 28, 29, 30, 31, 32, 33, 34, 35, 36, 37, 38, 39 40, 41, 42, or 43.
83. The TREM of any one of embodiments 1-82, wherein the ASGPR binding moiety is bound to the phosphate backbone at any one of TREM positions 27, 28, 29, 30, 31, 32, 33, 34, 35, 36, 37, 38, 39 40, 41, 42, or 43.
84. The TREM of any one of embodiments 1-83, wherein the ASGPR binding moiety is bound to the phosphate backbone at a plurality of TREM positions selected from 27, 28, 29, 30, 31, 32, 33, 34, 35, 36, 37, 38, 39 40, 41, 42, or 43.
85. The TREM of any one of embodiments 1-84, wherein the ASGPR binding moiety is bound to a sugar moiety (e.g., ribose moiety) at any one of TREM positions 10, 11, 12, 13, 14, 15, 16, 17, 18, 19, 20, 21, 22, 23, 24, 25, or 26.
86. The TREM of any one of embodiments 1-85, wherein the ASGPR binding moiety is bound to a sugar moiety (e.g., ribose moiety) at a plurality of TREM positions selected from 10, 11, 12, 13, 14, 15, 16, 17, 18, 19, 20, 21, 22, 23, 24, 25, or 26.
87. The TREM of any one of embodiments 1-86, wherein the ASGPR binding moiety is bound to the phosphate backbone at any one of TREM positions 10, 11, 12, 13, 14, 15, 16, 17, 18, 19, 20, 21, 22, 23, 24, 25, or 26.
88. The TREM of any one of embodiments 1-87, wherein the TREM retains the ability to support protein synthesis, e.g., relative to a TREM that does not comprise an ASGPR binding moiety or a naturally occurring tRNA.
89. The TREM of any one of embodiments 1-88, wherein the TREM retains the ability to be charged by a synthetase, e.g., relative to a TREM that does not comprise an ASGPR binding moiety or a naturally occurring tRNA.
90. The TREM of any one of embodiments 1-89, wherein the TREM retains the ability to be bound by an elongation factor, e.g., relative to a TREM that does not comprise an ASGPR binding moiety or a naturally occurring tRNA.
91. The TREM of any one of embodiments 1-90, wherein the TREM retains the ability to introduce an amino acid into a peptide chain, e.g., relative to a TREM that does not comprise an ASGPR binding moiety or a naturally occurring tRNA.
92. The TREM of any one of embodiments 1-91, wherein the TREM retains the ability to support elongation or support initiation, e.g., relative to a TREM that does not comprise an ASGPR binding moiety or a naturally occurring tRNA.
93. The TREM of any one of embodiments 1-92, wherein the TREM has a binding affinity to an ASGPR of between 0.01 nM and 100 mM.

### EXAMPLES

The following examples are provided to further illustrate some embodiments of the present disclosure, but are not intended to limit the scope of the invention; it will be understood by their exemplary nature that other procedures, methodologies, or techniques known to those skilled in the art may alternatively be used.

Table of Contents for Examples

| | |
|---|---|
| Example 1 | Preparation of Selected ASGPR Binding Moieties |
| Example 2 | Preparation of Selected Nucleotides |
| Example 3 | Synthesis of a TREM |
| Example 4 | Synthesis of TREMs with a terminal amino linker |
| Example 5 | Synthesis of TREMs comprising an ASGPR binding moiety |
| Example 6 | Analysis of GalNAc-TREMs via HPLC |
| Example 7 | Analysis of GalNAc-TREMs via mass spectrometry |
| Example 8 | In vitro delivery of GalNAc-TREMs to cells expressing the ASGPR |
| Example 9 | In vitro delivery of GalNAc-TREMs to primary human hepatocytes |
| Example 10 | Readthrough of a premature termination codon (PTC) in a reporter protein via |
| | administration of TREMs comprising an ASGPR binding moiety through |
| | transfection |
| Example 11 | Readthrough of a premature termination codon (PTC) in a reporter protein via |
| | administration of a TREM comprising an ASGPR binding moiety in cells |
| | expressing the ASGPR |
| Example 12 | Readthrough of a premature termination codon (PTC) in the alpha- |
| | galactosidase (GLA) ORF through administration of a TREM comprising an |
| | ASGPR binding moiety |
| Example 13 | Readthrough of a premature termination codon (PTC) in the alpha- |
| | galactosidase (GLA) ORF to produce a functional GLA protein through |
| | administration of a TREM comprising an ASGPR binding moiety |

### Example 1: Preparation of Selected ASGPR Binding Moieties

Compound 100: 11-(((2R,3R,4R,5R,6R)-3-acetamido-4,5-diacetoxy-6-(acetoxymethyl)tetrahydro-2H-pyran-2-yl)oxy)-16,16-bis ((3-((3-(5-(((2R,3R,4R,5R,6R)-3-acetamido-4,5-diacetoxy-6-(acetoxymethyl)tetrahydro-2H-pyran-2-yl)oxy) pentanamido)-propyl) amino)-3-oxopropoxy)methyl)-5,11,18-trioxo-14-oxa-6,10,17-triazanonacosan-29-oic acid (Compound 100) may be prepared according to the procedures provided by Nair K. et al. (2014) J. Am. Chem. Soc, 134 (49), 16958-16961.

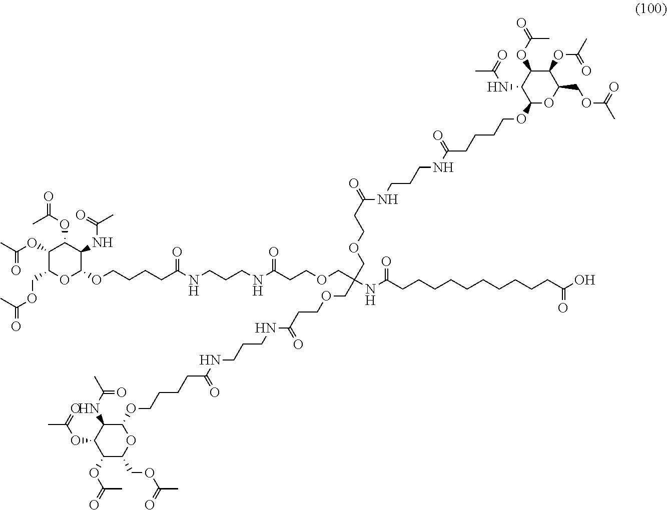 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCCCCCCCCC(=O)O)O[C@H](COC(C)=O)[C@H](OC(C)=O)[C@@H]1OC(C)=O]

Compound 101: Trebler GalNAc azide (N—(N-propargyldodecanoylamido)-tris{2-oxa-6,10-diaza-5,11-dioxo-15-[3,4,6-tri-O-acetyl-2-acetamido-2-deoxy-β-D-glucopyranosyloxy] pentadecyl}methane) is commercially available (e.g., from Primetich; catalog #0079).

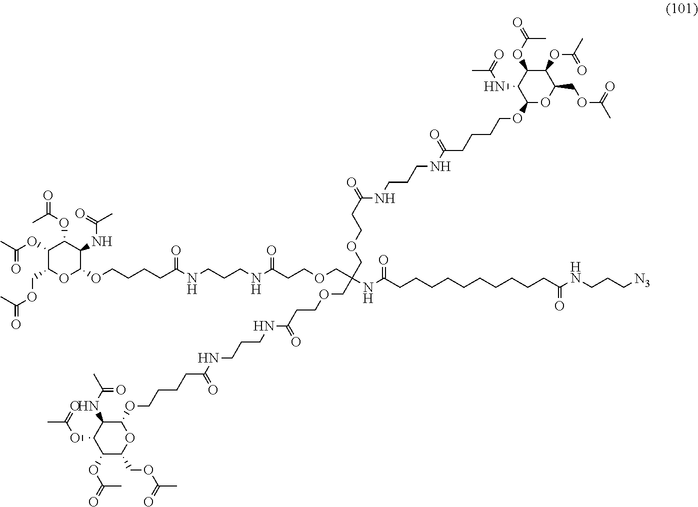 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCCCCCCCCC(=O)NCCCN=[N+]=[N-])O[C@H](COC(C)=O)[C@H](OC(C)=O)[C@@H]1OC(C)=O]

Compound 200: 11-(((2R,3R,4R,5R,6R)-3-acetamido-4,5-diacetoxy-6-(acetoxymethyl)tetrahydro-2H-pyran-2-yl)oxy)-16,16-bis ((3-((3-(5-(((2R,3R,4R,5R,6R)-3-acetamido-4,5-diacetoxy-6-(acetoxymethyl)tetrahydro-2H-pyran-2-yl)oxy)pentanamido)-propyl)amino)-3-oxopropoxy)methyl)-5,11,18-trioxo-14-oxa-6,10,17-triazanonacosan-29-oic acid (Compound 100) may be prepared according to the procedures provided by Nair K. et al. (2014) J. Am. Chem. Soc, 134 (49), 16958-16961, which is incorporated herein by reference in its entirety.

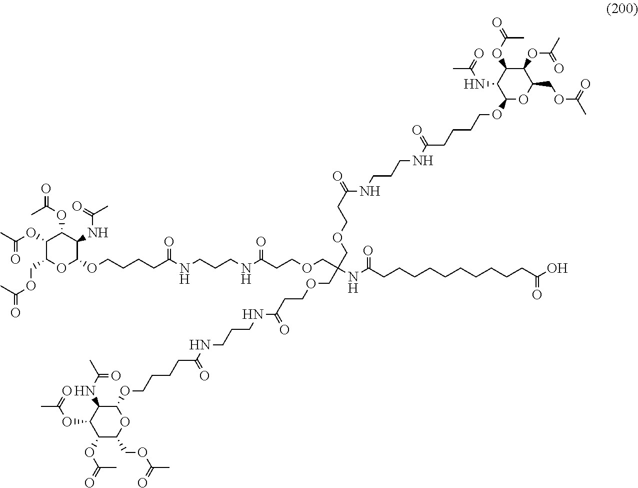 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCCCCCCCCC(=O)O)O[C@H](COC(C)=O)[C@H](OC(C)=O)[C@@H]1OC(C)=O]

Compound 201: Trebler GalNAc azide (N-(N-propargyldodecanoylamido)-tris {2-oxa-6,10-diaza-5,11-dioxo-15-[3,4,6-tri-O-acetyl-2-acetamido-2-deoxy-β-D-glucopyranosyloxy]pentadecyl}methane) is commercially available (e.g., from Primetich; catalog #0079).

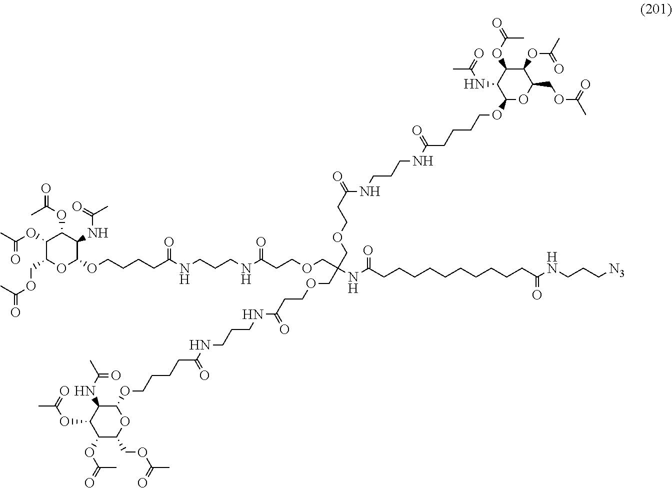 [SMILES: CC(=O)N[C@H]1[C@H](OCCCCC(=O)NCCCNC(=O)CCOCC(COCCC(=O)NCCCNC(=O)CCCCO[C@@H]2O[C@H](COC(C)=O)[C@H](OC(C)=O)[C@H](OC(C)=O)[C@H]2NC(C)=O)(COCCC(=O)NCCCNC(=O)CCCCO[C@@H]2O[C@H](COC(C)=O)[C@H](OC(C)=O)[C@H](OC(C)=O)[C@H]2NC(C)=O)NC(=O)CCCCCCCCCCC(=O)NCCCN=[N+]=[N-])O[C@H](COC(C)=O)[C@H](OC(C)=O)[C@@H]1OC(C)=O]

Compound 202: 1-(((2R,3R,4R,5R,6R)-3-acetamido-4,5-dihydroxy-6-(hydroxymethyl)tetrahydro-2H-pyran-2-yl)oxy)-18,18-bis(17-(((2R,3R,4R,5R,6R)-3-acetamido-4,5-dihydroxy-6-(hydroxymethyl)tetrahydro-2H-pyran-2-yl)oxy)-5-oxo-2,9,12,15-tetraoxa-6-azaheptadecyl)-13,20-dioxo-3,6,9,16-tetraoxa-12,19-diazahentriacontan-31-oic acid

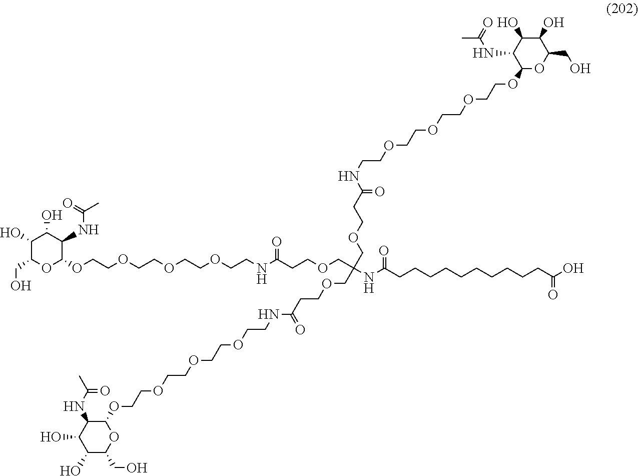 [SMILES: CC(=O)N[C@H]1[C@H](OCCOCCOCCOCCNC(=O)CCOCC(COCCC(=O)NCCOCCOCCOCCO[C@@H]2O[C@H](CO)[C@H](O)[C@H](O)[C@H]2NC(C)=O)(COCCC(=O)NCCOCCOCCOCCO[C@@H]2O[C@H](CO)[C@H](O)[C@H](O)[C@H]2NC(C)=O)NC(=O)CCCCCCCCCCC(=O)O)O[C@H](CO)[C@H](O)[C@@H]1O]

Compound 203: (17S,20S)-1-(((2R,3R,4R,5R,6R)-3-acetamido-4,5-diacetoxy-6-(acetoxymethyl)tetrahydro-2H-pyran-2-yl)oxy)-20-(1-(((2R,3R,4R,5R,6R)-3-acetamido-4,5-diacetoxy-6-(acetoxymethyl)tetrahydro-2H-pyran-2-yl)oxy)-11-oxo-3,6,9-trioxa-12-azahexadecan-16-yl)-17-(2-(2-(2-(2-(((2R,3R,4R,5R,6R)-3-acetamido-4,5-diacetoxy-6-(acetoxymethyl)tetrahydro-2H-pyran-2-yl)oxy) ethoxy) ethoxy) ethoxy) acetamido)-11,18-dioxo-3,6,9-trioxa-12,19-diazahenicosan-21-oic acid was prepared according the procedures outlined in U.S. Pat. No. 9,796,756, which is incorporated herein by reference in its entirety.

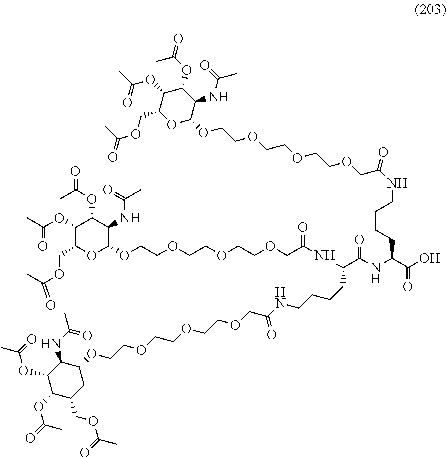 [SMILES: CC(=O)N[C@@H]1[C@@H](OC(C)=O)[C@@H](OC(C)=O)[C@@H](COC(C)=O)C[C@H]1OCCOCCOCCOCC(=O)NCCCC[C@H](NC(=O)COCCOCCOCCO[C@@H]1O[C@H](COC(C)=O)[C@H](OC(C)=O)[C@H](OC(C)=O)[C@H]1NC(C)=O)C(=O)N[C@@H](CCCCNC(=O)COCCOCCOCCO[C@@H]1O[C@H](COC(C)=O)[C@H](OC(C)=O)[C@H](OC(C)=O)[C@H]1NC(C)=O)C(=O)O]

### Example 2: Preparation of Selected Nucleotides

Amino Ribose 1: Modified nucleotides comprising an amino handle at the ribose sugar, such as AR1 (2′-O-aminolinker U phosphoramidite (3′-O-[(diisopropylamino) (2-cyanoethoxy) phosphino]-5′-O-(4,4′-dimethoxytrityl)-2′-O-2-[2-(trifluoroacetamido)-ethoxy]ethyluridine)), may be purchased from Berry & Associates; catalog #BA 0281). Briefly, O-aminolinker U phosphoramidite may be purchased with the primary amine protected trifluoroacetate and incorporated into a TREM to afford the amino ribose AR1.

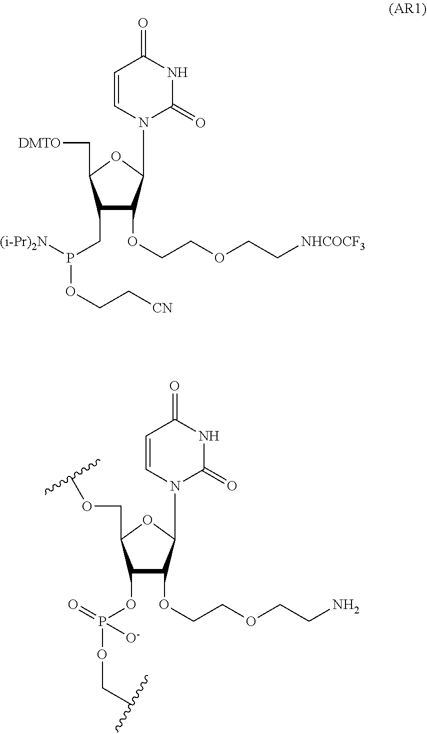

Alkyne Ribose 2: Modified nucleotides comprising an alkyne handle on the ribose, such as AR2 (5′-O-DMT-2′-O-propynyluridine 3′-CE phosphoramidite (5′-O-[Bis (4-methoxyphenyl)-phenylmethyl]-2′-O-2-propyn-1-yl-uridine 3′-[2-cyanoethyl N,N-bis(1-methylethyl)-phosphoramidite]; 5′-O-DMT-2′-O-propargyluridine 3′-CE phosphoramidite)) may be purchased from Biosynth-Carbosynth; catalog #PD139176. 5′-O-DMT-2′-O-propynyluridine 3′-CE phosphoramidite may be incorporated into TREM molecules via standard phosphoramidite chemistry to afford the alkyne ribose AR2.

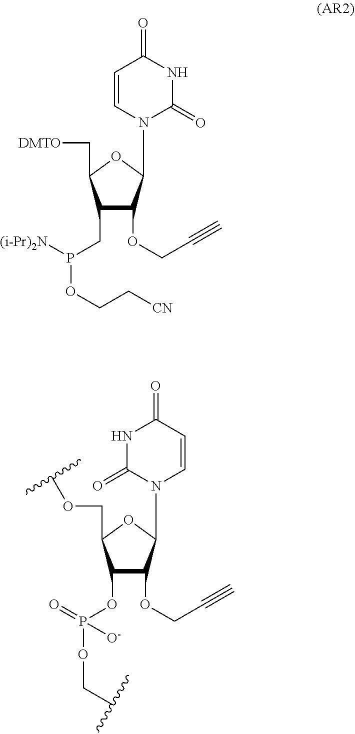

Alkyne Phosphoramidiate 1: (2R,3S,5R)-2-((bis(4-methoxyphenyl) (phenyl) methoxy)methyl)-5-(5-methyl-2,4-dioxo-3,4-dihydropyrimidin-1 (2H)-yl) tetrahydrofuran-3-yl (3-(2-nitro-5-(prop-2-yn-1-yloxy)phenyl) propyl) Diisopropylphosphoramidite may be prepared and purified according to previously published procedures (Saneyoshi H. et al. (2017) Bioorg. Med. Chem, 25, 3350-3356).

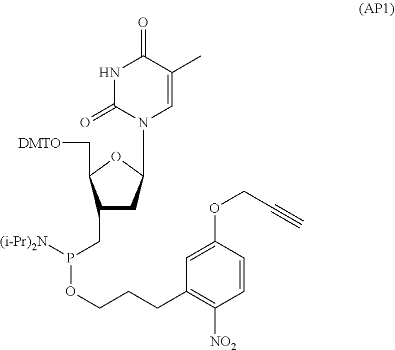

Alkyne Phosphate 1: Modified nucleotides comprising an alkyne handle on a phosphate may be prepared, starting from AP1, using standard phosphoramidite chemistry.

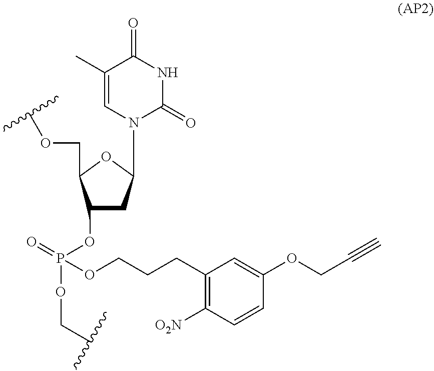 [SMILES: *COP(=O)(OCCCc1cc(OCC#C)ccc1[N+](=O)[O-])O[C@@H]1C[C@H](n2cc(C)c(=O)[nH]c2=O)O[C@@H]1CO*]

Amino Nucleobase 1: Modified nucleotides comprising an amine handle at the nucleobase, such as AN1 (C6-U phosphoramidite (5′-Dimethoxytrityl-5-[N-(trifluoroacetylaminohexyl)-3-acrylimido]-Uridine, 2′-O-triisopropylsilyloxymethyl-3′-[(2-cyanoethyl)-(N,N-diisopropyl)]-phosphoramidite)), may be purchased from Glen Research; catalog #10-3039. Briefly, Amino-Modifier C6-U phosphoramidite may be purchased with the primary amine protected as trifluoroacetate and incorporated into a TREM to afford the amino nucleobase AN1.

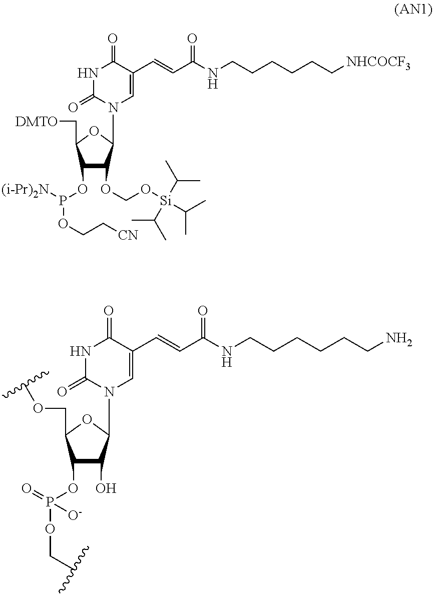

Alkyne Nucleobase 2: Modified nucleotides comprising an alkyne handle at the nucleobase, such as AN2 (C8-alkync-dT-CE phosphoramidite (5′-dimethoxytrityl-5-(octa-1,7-diynyl)-2′-deoxyuridine, 3′-[(2-cyanoethyl)-(N,N-diisopropyl)]-phosphoramidite)) may be purchased from Glen Research; catalog #10-1540. C8-Alkyne-dT-CE Phosphoramidite is incorporated into TREM molecules via standard phosphoramidite chemistry to afford the amino nucleobase AN2.

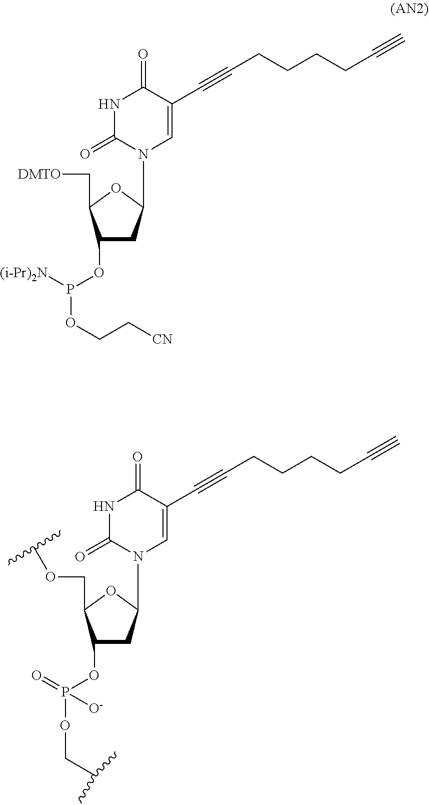

### Example 3: Synthesis of a TREM

The example describes the synthesis of exemplary TREMs. The TREMs may be chemically synthesized and purified by HPLC according to standard solid phase synthesis methods and phosphoramidite chemistry. (see, e.g., Scaringe S. et al. (2004) Curr Protoc Nucleic Acid Chem, 2.10.1-2.10.16; Usman N. et al. (1987) J. Am. Chem. Soc, 109, 7845-7854). More specifically, an arginine non-cognate TREM molecule named as TREM-Arg-TGA contains the sequence of ARG-UCU-TREM body but with the anticodon sequence corresponding to UCA instead of UCU. Exemplary nucleotide phosphoramidites to be used in the syntheses include 5′-O-dimethoxytrityl-N6-(benzoyl)-2′-O-t-butyldimethylsilyl-adenosine-3′-O-(2-cyanoethyl-N,N-diisopropylamino) phosphoramidite, 5′-O-dimethoxytrityl-N4-(acetyl)-2′-O-t-butyldimethylsilyl-cytidine-3′-O-(2-cyanoethyl-N,N-diisopropylamino) phosphoramidite, 5′-O-dimethoxytrityl-N2-(isobutyryl)-2′-O-t-butyldimethylsilyl-guanosine-3′-O-(2-cyanoethyl-N,N-diisopropylamino) phosphoramidite, and 5′-O-dimethoxytrityl-2′-O-t-butyldimethylsilyl-uridine-3′-O-(2-cyanoethyl-N,N-diisopropylamino) phosphoramidite.

A large number of TREMs may be synthesized in this manner, including, inter alia, (1) an arginine non-cognate TREM (e.g., TREM-Arg-TGA) that contains the sequence of ARG-UCU-TREM but with the anticodon sequence corresponding to UCA instead of UCU (i.e., SEQ ID NO: 622); (2) a serine non-cognate TREM named TREM-Ser-TAG that contains the sequence of SER-GCU-TREM but with the anticodon sequence corresponding to CUA instead of GCU (i.e., SEQ ID NO: 653); and (3) a glutamine non-cognate TREM named TREM-Gln-TAA that contains the sequence of GLN-CUG-TREM but with the anticodon sequence corresponding to UUA instead of CUG (i.e., SEQ ID NO: 650).

### Example 4: Synthesis of TREMs with a Terminal Amino Linker

This example describes the synthesis of TREM molecules with an amino linker at the 5′ terminus. The amino linker is added to the 5′ end of the oligonucleotides via phosphoramidite chemistry on a synthesizer. For example, TFA-amino C6 CED phosphoramidite may be incorporated at the 5′ end of oligonucleotide. Similar chemistry may be employed to couple the amino linker to the 3′ terminus.

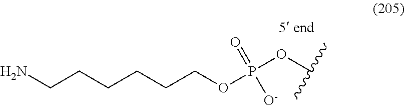 [SMILES: *OP(=O)([O-])OCCCCCCN]

Additionally, the amino linker may be incorporated into the TREM sequence by using a phosphoramidite comprising an aminohexyl linker. In these cases, a compound such as 6-(4-monomethoxytritylamino) hexyl-(2-cyanoethyl)-(N,N-diisopropyl) phosphoramidite may be used, which is commercially available from ChemGenes; catalog #CLP-1563.

### Example 5: Synthesis of TREMs Comprising an ASGPR Binding Moiety

The example describes the synthesis of an exemplary TREM comprising an ASGPR binding moiety. Several methods of coupling the ASGPR binding moieties to the TREM may be used, including employing amide formation and triazole-based click chemistry may be used. For example, a carboxylic acid triantennary GalNAc molecule may be coupled with oligonucleotides bearing amino linkers via an amide bond formation reaction. Briefly, a solution of Compound 100 (2 equivalents), HATU (1.8 equivalents) and diisopropylethylamine (8 equivalents) in dry acetonitrile (or dry DMF) is vortexed for 2 minutes. To this solution is added an aqueous solution of a TREM bearing an amino linker (1 equivalent), such as the TREM bearing an amino linker outlined in Example 4. The reaction mixture is vortexed for 2 minutes and kept at room temperature for 60 minutes, at which point the solvent is removed under vacuum, diluted with water and purified by reversed phase column chromatography or ion exchange chromatography. In cases where GalNAc moieties are protected, the protecting groups may then be removed by appropriate treatment. For example, when the free hydroxyl groups in the GalNAc moieties are protected with acetyl groups, ammonium hydroxide treatment is performed for 6 h at room temperature, followed by purification to afford the final GalNAc-TREM conjugate (106).

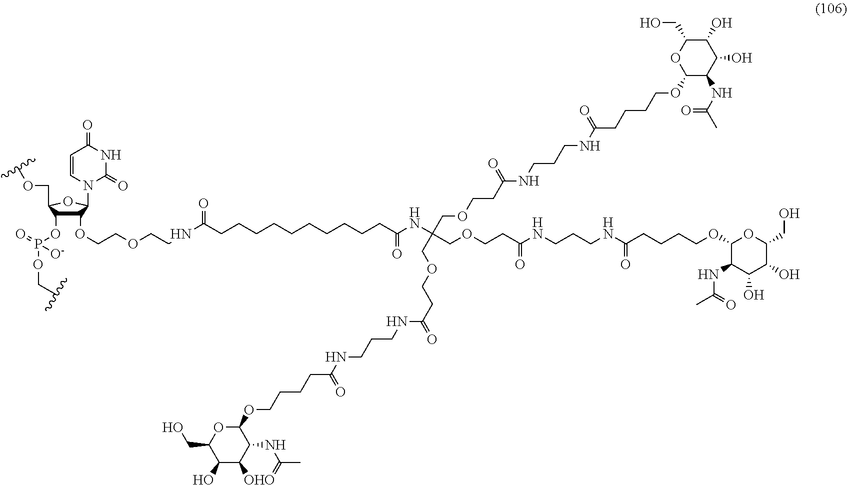

Alternatively, TREM molecules bearing an alkyne group can be conjugated to ASGPR binding moieties bearing an azide group, such as Trebler GalNAc azide. The reaction may be carried out via copper catalyzed azide-alkyne cycloaddition (Saneyoshi H. et al. (2017) Bioorg. Med. Chem, 25, 3350-3356), and purified using standard techniques to yield triazolyl-containing moieties such as Compound 107 below.

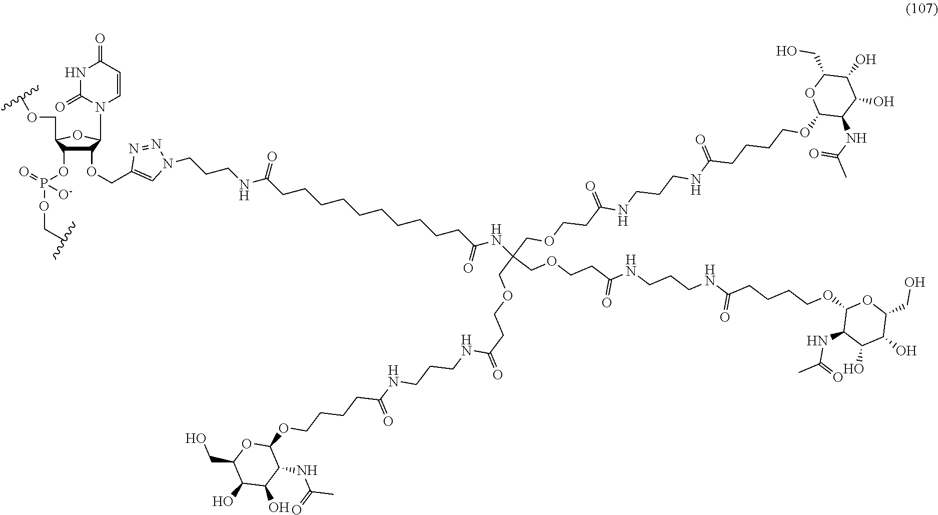

For example, a carboxylic acid triantennary GalNAc molecule may be coupled with oligonucleotides bearing amino linkers via an amide bond formation reaction. Briefly, a solution of Compound 200 (2 equivalents), HATU (1.8 equivalents) and diisopropylethylamine (8 equivalents) in dry acetonitrile (or dry DMF) can be vortexed for 2 minutes. To this solution an aqueous solution of a TREM bearing an amino linker (1 equivalent) can be added, such as the TREM bearing an amino linker outlined in Example 4. The reaction mixture will be vortexed for 2 minutes and kept at room temperature for 60 minutes, at which point the solvent can be removed under vacuum, diluted with water, and purified by reversed phase column chromatography or ion exchange chromatography. In cases where GalNAc moieties contain protecting groups, these protecting groups can be removed by appropriate treatment. For example, when the free hydroxyl groups in the GalNAc moieties are protected with acetyl groups, ammonium hydroxide treatment are performed for 6 h at room temperature, followed by purification to afford the final GalNAc-TREM conjugate (206).

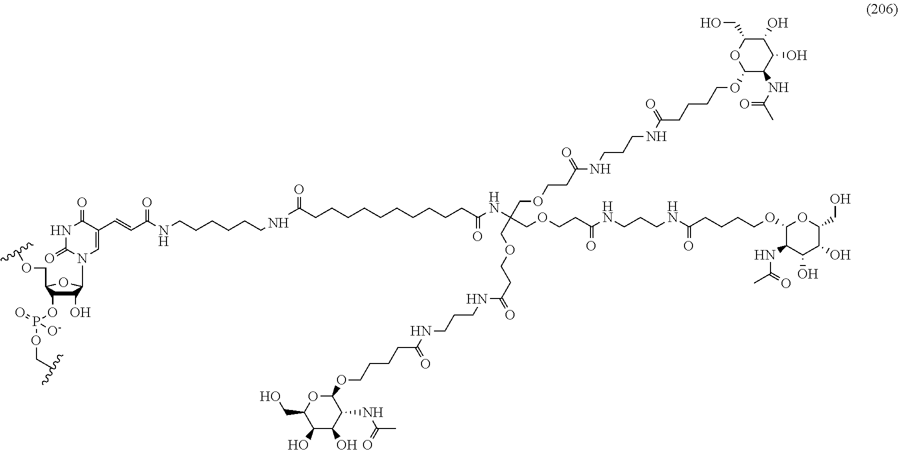

ASGPR binding moieties bearing a free carboxylate may be also first activated to pentafluorophenyl esters (PFPs), followed by coupling a free amine on the TREM, either at the 3′ or 5′ terminus or internally on a nucleobase amine (for example, a linker on a nucleobase).

Additionally, TREMs were coupled to various ASGPR binding moieties by converting certain ASGPR binding moieties bearing free carboxylates, such as Compounds 200, 202, and 203, to N-hydroxysuccinimide (NHS)-activated compounds. Briefly, the carboxylate-bearing ASGPR binding moieties were dissolved in dimethylformamide (DMF) and N-hydroxysuccinimide (NHS, 1.1 equiv) and N,N-diisopropylcarbodiimide (1.1 equiv) were added. The solution was stirred at room temperature for 18 hours and coupled directly to a TREM without further purification. A TREM bearing a free amine group, such as a TREM with a terminal amino linker or a TREM bearing a modified nucleotide (e.g., AN1 or AN2), was dissolved in mixture of 50 mM sodium carbonate/bicarbonate buffer pH 9.6 and dimethylsulfoxide (DMSO) 4:6 v/v. To this solution was added 1.2 molar equivalents of the NHS ester-activated ASGPR binding moiety solution in DMF. The reaction was carried out at room temperature for 1 hour, after which another 1.2 molar equivalent of the NHS ester-activated ASGPR binding moiety in DMF was added. After 1 hour, the reaction was diluted 15-fold with water, filtered through a 1.2 μm filter, and purified by reversed-phase HPLC (Xbridge C18 Prep 19×50 mm, using a 100 mM triethylamine acetate pH 7/95% acetonitrile buffer system). Any protecting groups on the ASGPR binding moieties were then removed, for example, by treatment with 3M sodium acetate pH 5.2 and 80% ethanol.

Alternatively, TREM molecules bearing an alkyne group were conjugated to ASGPR binding moieties bearing an azide group, such as Trebler GalNAc azide (Compound 201). The reaction was carried out via copper catalyzed azide-alkyne cycloaddition (Saneyoshi H. et al. (2017) Bioorg. Med. Chem, 25, 3350-3356; incorporated herein by reference in its entirety), and purified using standard techniques to yield triazolyl-containing moieties such as Compound 207 below.

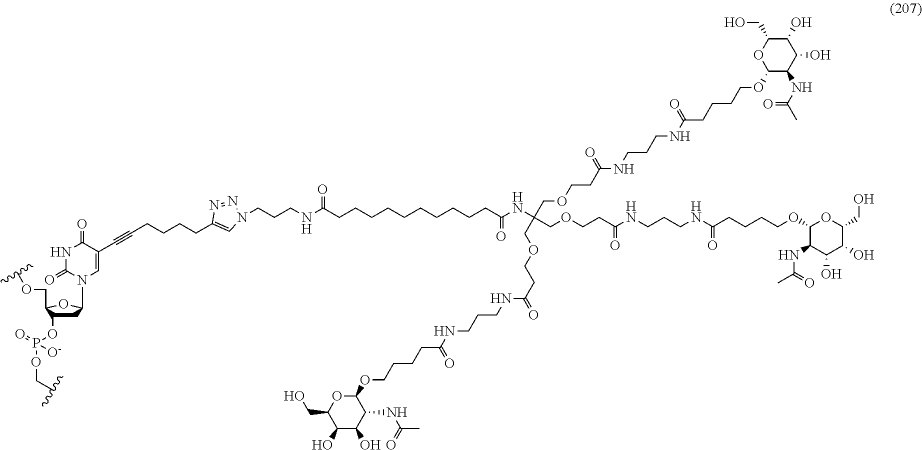

Table 12 summarizes a list of TREMs prepared containing an ASGPR binding moiety, according to the protocols provided herein. Each TREM in the sequence is either unconjugated (e.g., a control) or conjugated to either i) a ASGPR binding moiety described herein (abbreviated as “GalNAc” in the table); ii) a fluorophore such as Cy3; and/or iii) a linker (abbreviated as “5-LC-N” in the table. The molecular weight of each TREM was confirmed by LC-MS, wherein the determined molecular weight was found to be within +/−0.04% of the calculated molecular weight for each TREM.

TABLE 12
Exemplary TREMs comprising an ASGPR binding moiety

| Compound | SEQ | | | Calculated |
|---|---|---|---|---|
| No. | ID NO. | Descriptor | Sequence | MW |
| 99 | 622 | Arg-TGA | GGCUCCGUGGCGCAAUGGAUAG | |
| | | (Unconjugated) | CGCAUUGGACUUCAAAUUCAAA | |
| | | | GGUUCCGGGUUCGAGUCCCGGC | |
| | | | GGAGUCGCCA | |
| 100 | 623 | Arg-TGA (1-G- | [GalNAc]-[TE]- | 26,355.7 |
| | | GalNAc- | GGGCUCCGUGGCGCAAUGGAUA | |
| | | linkage on | GCGCAUUGGACUUCAAAUUCAA | |
| | | 5′terminus) | AGGUUCCGGGUUCGAGUCCCGG | |
| | | | CGGAGUCGCCA | |
| 101 | 624 | Arg-TGA (16-U- | GGCUCCGUGGCGCAAU[GalNAc] | 26,343.8 |
| | | GalNAc) | GGAUAGCGCAUUGGACUUCAAA | |
| | | | UUCAAAGGUUCCGGGUUCGAGU | |
| | | | CCCGGCGGAGUCGCCA | |
| 102 | 625 | Arg-TGA (20-U- | GGCUCCGUGGCGCAAUGGAU | 26,343.8 |
| | | GalNAc) | [GalNAc]AGCGCAUUGGACUUCA | |
| | | | AAUUCAAAGGUUCCGGGUUCGA | |
| | | | GUCCCGGCGGAGUCGCCA | |
| 103 | 626 | Arg-TGA (47-U- | GGCUCCGUGGCGCAAUGGAUAG | 26,343.8 |
| | | GalNAc) | CGCAUUGGACUUCAAAUUCAAA | |
| | | | GGU[GalNAc]UCCGGGUUCGAG | |
| | | | UCCCGGCGGAGUCGCCA | |
| 104 | 627 | Arg-TGA (16-U- | [Cy3]GGCUCCGUGGCGCAAU | 26,851.0 |
| | | GalNAc) & 5′- | [GalNAc]GGAUAGCGCAUUGGA | |
| | | Cy3 | CUUCAAAUUCAAAGGUUCCGGGU | |
| | | | UCGAGUCCCGGCGGAGUCGCCA | |
| 105 | 628 | Arg-TGA (20-U- | [Cy3]GGCUCCGUGGCGCAAUGGA | 26,851.0 |
| | | GalNAc) & 5′- | U[GalNAc]AGCGCAUUGGACUUCA | |
| | | Cy3 | AAUUCAAAGGUUCCGGGUUCGA | |
| | | | GUCCCGGCGGAGUCGCCA | |
| 106 | 629 | Arg-TGA (47-U- | [Cy3]GGCUCCGUGGCGCAAUGGA | 26,851.0 |
| | | GalNAc) & 5′- | UAGCGCAUUGGACUUCAAAUUC | |
| | | Cy3 | AAAGGU[GalNAc]UCCGGGUUCGA | |
| | | | GUCCCGGCGGAGUCGCCA | |
| 107 | 630 | Arg-TGA (16- | GGCUCCGUGGCGCAAU[5-LC-N] | 24,676.9 |
| | | linker) | GGAUAGCGCAUUGGACUUCAAA | |
| | | | UUCAAAGGUUCCGGGUUCGAGU | |
| | | | CCCGGCGGAGUCGCCA | |
| 108 | 631 | Arg-TGA (20- | GGCUCCGUGGCGCAAUGGAU[5- | 24,676.9 |
| | | linker) | LC-N]AGCGCAUUGGACUUCAAA | |
| | | | UUCAAAGGUUCCGGGUUCGAGU | |
| | | | CCCGGCGGAGUCGCCA | |
| 109 | 632 | Arg-TGA (47- | GGCUCCGUGGCGCAAUGGAUAG | 24,676.9 |
| | | linker) | CGCAUUGGACUUCAAAUUCAAA | |
| | | | GGU[5-LC-N] | |
| | | | UCCGGGUUCGAGUCCC | |
| | | | GGCGGAGUCGCCA | |
| 110 | 633 | Gln-TAA (16- | GGUUCCAUGGUGUAAU[GalNAc]G | 25,890.5 |
| | | U-GalNAc) | GUAAGCACUCUGGACUUUAAAU | |
| | | | CCAGCGAUCCGAGUUCGAGUCUC | |
| | | | GGUGGAACCUCCA | |
| 111 | 634 | Gln-TAA (19- | GGUUCCAUGGUGUAAUGGU | 25,890.5 |
| | | U-GalNAc) | [GalNAc]AAGCACUCUGGACUU | |
| | | | UAAAUCCAGCGAUCCGAGUUCGA | |
| | | | GUCUCGGUGGAACCUCCA | |
| 112 | 635 | Gln-TAA (16- | [Cy3]GGUUCCAUGGUGUAAU | 26,397.7 |
| | | U-GalNAc) & | [GalNAc]GGUAAGCACUCUGGA | |
| | | 5′-Cy3 | CUUUAAAUCCAGCGAUCCGAGUU | |
| | | | CGAGUCUCGGUGGAACCUCCA | |
| 113 | 636 | Gln-TAA (19- | [Cy3]GGUUCCAUGGUGUAAUGGU | 26,397.7 |
| | | U-GalNAc) & | [GalNAc]AAGCACUCUGGACUUUA | |
| | | 5′-Cy3 | AAUCCAGCGAUCCGAGUUCGAG | |
| | | | UCUCGGUGGAACCUCCA | |
| 114 | 637 | Gln-TAA (47- | [Cy3]GGUUCCAUGGUGUAAUGGU | 26,397.7 |
| | | U-GalNAc) & | AAGCACUCUGGACUUUAAAUCC | |
| | | 5′-Cy3 | AGCGAU[GalNAc]CCGAGUUCGAG | |
| | | | UCUCGGUGGAACCUCCA | |
| 115 | 638 | Gln-TAA (16- | GGUUCCAUGGUGUAAU[5-LC- | 24,223.6 |
| | | linker) | N]GGUAAGCACUCUGGACUUUAA | |
| | | | AUCCAGCGAUCCGAGUUCGAGU | |
| | | | CUCGGUGGAACCUCCA | |
| 116 | 639 | Gln-TAA (19- | GGUUCCAUGGUGUAAUGGU[5-LC- | 24,223.6 |
| | | linker) | N]AAGCACUCUGGACUUUAAAUC | |
| | | | CAGCGAUCCGAGUUCGAGUCUC | |
| | | | GGUGGAACCUCCA | |
| 117 | 640 | Gln-TAA (47- | GGUUCCAUGGUGUAAUGGUAAG | 24,223.6 |
| | | linker) | CACUCUGGACUUUAAAUCCAGC | |
| | | | GA U[5-LC-N]CCGAGUUCGAGU | |
| | | | CUCGGUGGAACCUCCA | |
| 118 | 641 | Ser-TAG (1-G- | [GalNAc]-[TE]- | 29,170.4 |
| | | GalNAc- | GACGAGGUGGCCGAGUGGUUAA | |
| | | linkage on | GGCGAUGGACUCUAAAUCCAUU | |
| | | 5′terminus) | GUGCUCUGCACGCGUGGGUUCG | |
| | | | AAUCCCAUCCUCGUCGCCA | |
| 119 | 642 | Ser-TAG (16- | GACGAGGUGGCCGAGU[GalNAc]G | 29,158.4 |
| | | U-GalNAc) | GUUAAGGCGAUGGACUCUAAAU | |
| | | | CCAUUGUGCUCUGCACGCGUGG | |
| | | | GUUCGAAUCCCAUCCUCGUCGCC | |
| | | | A | |
| 120 | 643 | Ser-TAG (20- | GACGAGGUGGCCGAGUGGUU | 29,158.4 |
| | | U-GalNAc) | [GalNAc]AAGGCGAUGGACUCU | |
| | | | AAAUCCAUUGUGCUCUGCACGCG | |
| | | | UGGGUUCGAAUCCCAUCCUCGUC | |
| | | | GCCA | |
| 121 | 644 | Ser-TAG (46- | GACGAGGUGGCCGAGUGGUUAA | 29,158.4 |
| | | U-GalNAc) | GGCGAUGGACUCUAAAUCCAUU | |
| | | | GU[GalNAc]GCUCUGCACGCGUGG | |
| | | | GUUCGAAUCCCAUCCUCGUCGCC | |
| | | | A | |
| 122 | 645 | Ser-TAG (20- | [Cy3]GACGAGGUGGCCGAGUGGU | 29,665.7 |
| | | U-GalNAc) & | U[GalNAc]AAGGCGAUGGACUCUA | |
| | | 5′-Cy3 | AAUCCAUUGUGCUCUGCACGCG | |
| | | | UGGGUUCGAAUCCCAUCCUCGUC | |
| | | | GCCA | |
| 123 | 646 | Ser-TAG (46- | [Cy3]GACGAGGUGGCCGAGUGGU | 29,665.7 |
| | | U-GalNAc) & | UAAGGCGAUGGACUCUAAAUCC | |
| | | 5′-Cy3 | AUUGU[GalNAc]GCUCUGCACGCG | |
| | | | UGGGUUCGAAUCCCAUCCUCGUC | |
| | | | GCCA | |
| 124 | 647 | Ser-TAG (16- | GACGAGGUGGCCGAGU[5-LC- | 27,491.6 |
| | | linker) | N]GGUUAAGGCGAUGGACUCUAA | |
| | | | AUCCAUUGUGCUCUGCACGCGU | |
| | | | GGGUUCGAAUCCCAUCCUCGUCG | |
| | | | CCA | |
| 125 | 648 | Ser-TAG (20- | GACGAGGUGGCCGAGUGGUU[5- | 27,491.6 |
| | | linker) | LC-N]AAGGCGAUGGACUCUAA | |
| | | | AUCCAUUGUGCUCUGCACGCGU | |
| | | | GGGUUCGAAUCCCAUCCUCGUCG | |
| | | | CCA | |
| 126 | 649 | Ser-TAG (46- | GACGAGGUGGCCGAGUGGUUAA | 27,491.6 |
| | | linker) | GGCGAUGGACUCUAAAUCCAUU | |
| | | | GU[5-LC-N] | |
| | | | GCUCUGCACGCGUGGGU | |
| | | | UCGAAUCCCAUCCUCGUCGCCA | |
| 127 | 650 | Gln-TAA | GGUUCCAUGGUGUAAUGGUAAG | |
| | | (Unconjugated) | CACUCUGGACUUUAAAUCCAGC | |
| | | | GAUCCGAGUUCGAGUCUCGGUG | |
| | | | GAACCUCCA | |
| 128 | 651 | Gln-TAA-1-G- | [GalNAc]-[TE]- | |
| | | GalNAc | GGUUCCAUGGUGUAAUGGUAAG | |
| | | | CACUCUGGACUUUAAAUCCAGC | |
| | | | GAUCCGAGUUCGAGUCUCGGUG | |
| | | | GAACCUCCA | |
| 129 | 652 | Gln-TAA-47- | GGUUCCAUGGUGUAAUGGUAAG | |
| | | U-GalNAc | CACUCUGGACUUUAAAUCCAGC | |
| | | | GAU[GalNAc]CCGAGUUCGAGUCU | |
| | | | CGGUGGAACCUCCA | |
| 130 | 653 | Ser-TAG | GACGAGGUGGCCGAGUGGUUAA | |
| | | (Unconjugated) | GGCGAUGGACUCUAAAUCCAUU | |
| | | | GUGCUCUGCACGCGUGGGUUCG | |
| | | | AAUCCCAUCCUCGUCGCCA | |
| 131 | 654 | Ser-TAG (16- | [Cy3]GACGAGGUGGCCGAGU | |
| | | U-GalNAc) & | [GalNAc]GGUUAAGGCGAUGGA | |
| | | 5′-Cy3 | CUCUAAAUCCAUUGUGCUCUGCA | |
| | | | CGCGUGGGUUCGAAUCCCAUCCU | |
| | | | CGUCGCCA | |

### Example 6: Analysis of GalNAc-TREMs Via HPLC

The example describes the analysis of GalNAc-TREM molecules via HPLC. GalNAc-TREM molecules may be analyzed by HPLC, for example, to evaluate the purity and homogeneity of the compositions. A Waters Aquity UPLC system using a Waters BEH C18 column (2.1 mm×50 mm×1.7 μm) may be used for this analysis. Samples may be prepared by dissolving 0.5 nmol of the oligonucleotide in 75 μL of water and injecting 2 μL of the solution. The buffers used may be 50 mM dimethylhexylammonium acetate with 10% CH₃CN (acetonitrile) as buffer A and 50 mM dimethylhexylammonium acetate with 75% CH₃CN as buffer B (gradient 25-75% buffer B over 5 mins), with a flow rate of 0.5 mL/min at 60° C.

### Example 7: Analysis of GalNAc-TREMs Via Mass Spectrometry

The example describes the mass spectrometry analysis of the GalNAc-TREM molecules. ESI-LCMS data for the oligonucleotides may be acquired on a Thermo Ultimate 3000-LTQ-XL mass spectrometer. Samples may be prepared by dissolving 0.5 nmol of the oligonucleotide in 75 μL of water and injecting 10 μL of the solution directly onto a Novatia C18 (HTCS-HTC1-4) trap column. Following injection into the trap column, the sample may be eluted directly onto the LTQ-MS with 85% CH₃CN, 50 mM HFIP (hexafluoro-2-propanol), 10 μM EDTA (ethylenediaminetetraacetic acid), 0.35% DIPEA (N,N-diisopropylethylamine) and the mass to charge ratio (m/z) is determined.

### Example 8. In Vitro Delivery of GalNAc-TREMs to Cells Expressing the ASGPR

This example describes the in vitro delivery of exemplary GalNAc-conjugated TREMs into U2OS cells expressing the ASGPR under gymnotic conditions (without a transfection agent). The methods described in this example can be adopted for evaluating the levels of GalNAc-TREMs in ASGR-expressing cells after delivery.

### Host Cell Modification

A U2OS cell line engineered to stably express the ASGP receptor (ASGPR) can be generated using plasmid transfection and selection. Briefly, the cells will be co-transfected with a plasmid encoding the ASGPRI gene and a puromycin selection cassette. The next day, cells are selected with puromycin. The remaining cells are expanded and tested for ASGPR expression.

### Delivery of GalNAc-TREMs Under Gymnotic Conditions

The ASGPR engineered U2OS cells will be harvested and diluted to 4×10⁴ cells/mL in complete growth medium, and 100 uL of the diluted cell suspension will be added in a 96-well plate (3904, Corning, USA). The plate will be placed in a 37° C. 5% CO₂ incubator for cell attachment to the well bottom. After 20-24 hours, various GalNAc-TREMs modified with a fluorophore at the 5′ terminus (Cy3) will be diluted to a 10-fold concentration (e.g. 1000 nM) into the RNase-free water and added to the well at a 1:10 dilution. The plate will be placed in the 37° C. 5% CO2 incubator for 20-24h before the tRNA quantification assay to determine the intracellular levels of the GalNAc-TREM.

Quantitative tRNA Delivery Using Live Imaging

At 20-24h post tRNA delivery, the plate will be taken out of the incubator. After aspiration, the culture medium (Hoechest 33342; Thermofisher, USA) will be diluted to 1:10,000 in the full growth medium and added to the cells. The plate will be incubated at room temperature (˜25° C.) for 10 min, then washed with 1×DPBS for 6 times. After the last wash, full growth medium (100 uL per/well) will be added to the plate. The plate will be imaged under ImageXpress Pico Micrscope (Molecular Device, USA) with three channels (Cy3/DAPI/Brightfield) at 20× magnification. The average intensity of Cy3 channel will be quantified by the “Cell scoring” function from the microscope software. Free uptake by the ASGPR1-expressing U2OS cells of Gln-TAA conjugated with GalNAc along the TREM will be detected by visualizing the Cy3 tag with fluorescent microscopy. The negative control cells will be exposed to unconjugated Gln-TAA TREMs while the positive control will be exposed to GalNAc-modified Gln-TAA TREMs with RNAiMAX transfection reagent. Quantification of the average intensity of the microscopy results will be calculated, comparing free uptake of the TREM and transfection-facilitated uptake of the TREM. Other modified TREMs (e.g., Arg-TGA or Ser-TAG) will also be tested.

### Example 9. In Vitro Delivery of GalNAc-TREMs to Primary Human Hepatocytes

This example describes the in vitro delivery of a GalNAc-conjugated TREM into primary human hepatocytes under gymnotic conditions (without a transfection agent). The methods described in this example can be adopted for evaluating the levels of GalNAc-TREMs in the hepatocytes after delivery.

One cryo-vial of Liverpool, 10 donor human cryoplateable hepatocytes (X008001-P, BioIVT, USA), is carefully thawed and diluted in pre-warmed INVITROGRO CP Medium at 37° C. The total cell count and the number of viable cells are determined using a cell counter. A >70% viability is expected with a successful thawing procedure. The cells are further diluted to 7×105 viable cells/mL, and 70 uL of the diluted cell suspension is seeded in a collagen-coated 96-well plate (354649, Corning, USA). The plate is shaken gently in a back-and-forth and side-to-side manner to evenly distribute the cells. The plate is placed in a 37° C. 5% CO₂ incubator. After 2 hours, the plate is carefully washed with INVITROGRO CP Medium. GalNAc-TREMs are diluted to a working concentration (e.g. 100 nM) into the growth medium and added to the well. The plate is placed in the 37° C. 5% CO₂ incubator for 20-24 h before the tRNA quantification assay to determine the intracellular levels of the GalNAc-TREM.

Quantitative tRNA Profiling

The intracellular levels of GalNAc-TREM may be determined using next generation sequencing, as previously described in Pinkard et al., Nat Comm (2020) 11, 4104. Briefly, the hepatocytes treated under gymnotic conditions with GalNAc-TREM as described above may be lysed and total RNA purified using a method such as phenol chloroform extraction. RNAs smaller than 200 nucleotides are separated from the lysate using a small RNA isolation kit per manufacturer's instructions to generate a small RNA (sRNA) fraction.

The sRNA fraction is deacylated using 100 mM Tris-HCl (pH 9.0) at 37° C. for 45 minutes. The solution is neutralized by the addition of an equal volume of 100 mM Na-acetate/acetic acid (pH 4.8) and 100 mM NaCl, followed by ethanol precipitation. Deacylated sRNA is splint ligated in a reaction with 3′ adapter, a mix of 4 splint strands and annealing buffer at 37° C. for 15 minutes followed by addition of a RNL2 ligase reaction buffer mix at 37° C. for 1 h and then at 4° C. for 1 hr. The deacylated and splint ligated sRNA is precipitated using a method such as phenol chloroform extraction.

The deacylated and splint ligated sRNA is then reverse transcribed using an RT enzyme such as Superscript IV at 55° C. for 1 hr. The reaction product is desalted in a micro Bio-Spin P30 (BioRad cat #7326250) according to manufacturer directions, and the sample is run on a denaturing polyacrylamide gel. Gel bands from 65-200 nt are excised, and sRNA is extracted. The sRNA is circularized using a circligase and purified. The purified circularized RNA is PCR amplified and product run on a e-gel ex. Bands from 100-250 nt are excised and purified using a commercial kit (e.g., Qiaquick gel extraction kit) according to manufacturer directions, and RNA is precipitated. Next generation sequencing may then be performed on the libraries and the sequences mapped to a genomic database, in this example to the genomic tRNA database, GtRNAdb.

Quantitative tRNA Delivery Using Cy3 Live Imaging

At 20-24h post tRNA delivery, the plate will be taken out of the incubator. After aspirating the culture medium, Hoechest 33342 (62249, Thermofisher, USA) will be diluted to 1:10,000 in the INVITROGRO CP Medium and added to the cells. The plate will be incubated at room temperature (˜25° C.) for 10 min, then washed with 1×DPBS for 6 times. After the last wash, INVITROGRO CP medium (100 uL per/well) will be added to the plate. The plate will be imaged using ImageXpress Pico Microscope (Molecular Device, USA) with three channels (Cy3/DAPI/Brightfield) at 20× magnification. The average intensity of Cy3 channel will be quantified by the “Cell scoring” function from the microscope software. Free uptake of TREMs and transfection of TREMs using a reagent will be compared.

### Example 10. Readthrough of a Premature Termination Codon (PTC) in a Reporter Protein Via Administration of TREMs Comprising an ASGPR Binding Moiety Through Transfection

This example describes an assay to test the ability of non-cognate TREMs bearing an ASGPR binding moiety (“GalNAc-TREMs”) to readthrough a PTC in a cell expressing a protein having a PTC. This Example describes an arginine non-cognate TREM, though a non-cognate TREM specifying any one of the other 19 amino acids can also be used. Host cell modification

A cell line engineered to stably express the NanoLuc reporter construct containing a premature termination codon (PTC) may be generated using the FlpIn system according to the manufacturer's instructions.

### Synthesis and Preparation of Non-Cognate GalNAc-TREM

In this example, the arginine non-cognate GalNAc-TREM, may be produced such that it contains the sequence of the ARG-UCU-TREM body but with the anticodon sequence corresponding to UCA instead of UCU, and is conjugated to the GalNAc moiety. The arginine non-cognate GalNAc-TREM is synthesized as described previously, and its quality controlled using methods as described in Examples 6-7. To ensure proper folding, the TREM may be heated at 85° C. for 2 minutes and then snap cooled at 4° C. for 5 minutes.

Delivery of Non-Cognate GalNAc-TREM into Host Cells Through Transfection

To deliver the GalNAc-TREM into the NanoLuc reporter cells, a reverse transfection reaction is performed on the NanoLuc reporter cells using lipofectamine RNAiMAX (ThermoFisher Scientific, USA) according to manufacturer instructions. Briefly, 5 uL of a 2.5 uM solution of GalNAc-TREMs are diluted in a 20 uL RNAiMAX/OptiMEM mixture. After 30 min gentle mixing at room temperature, the 25 uL GalNAc-TREM/transfection mixture is added to a 96-well plate and kept still for 20-30 min before adding the cells. The NanoLuc reporter cells are harvested and diluted to 4×10⁵ cells/mL in complete growth medium, and 100 L of the diluted cell suspension is added and mixed to the plate containing the GalNAc-TREM. After 24 h, 100 uL complete growth medium is added to the 96-well plate for cell health.

### Translation Suppression Assay

To monitor the efficacy of the GalNAc-TREM to readthrough the PTC in the reporter construct 48 hours after GalNAc-TREM delivery into cells, a NanoGlo bioluminescent assay (Promega, USA) may be performed according to manufacturer instruction. Briefly, cell media is replaced and allowed to equilibrate to room temperature. NanoGlo reagent is prepared by mixing the buffer with substrate in a 50:1 ratio. 50 uL of mixed NanoGlo reagent is added to the 96-well plate and mixed on the shaker at 600 rpm for 10 min. After 2 min, the plate is centrifuged at 1000 g, followed by a 5 min incubation step at room temperature before measuring sample bioluminescence. As a positive control, a host cell expressing the NanoLuc reporter construct without a PTC is used. As a negative control, a host cell expressing the NanoLuc reporter construct with a PTC is used, but no GalNAc-TREM is transfected. The efficacy of the GalNAc-TREMs is measured as a ratio of the NanoLuc luminescence in the experimental sample to the NanoLuc luminescence of the positive control or as a ratio of the NanoLuc luminescence in the experimental sample to the NanoLuc luminescence of the negative control. It is expected that if the arginine non-cognate TREM is functional, it may be able to read-through the stop mutation in the NanoLuc reporter and produce a luminescent reading higher than the luminescent reading measured in the negative control. If the arginine non-cognate TREM is not functional, the stop mutation is not rescued, and luminescence less or equal to the negative control is detected.

The impacts of including ASGPR binding moieties in the TREM sequence will be evaluated. The data for each modified TREM will be provided as log 2 fold changes compared with the mock sample, wherein “1” indicates less than a 4.00 log 2 fold change; “2” indicates a log 2 fold change greater than or equal to 4.01 and less than 7.00 log 2 fold change; and “3” indicates greater than or equal to 7.01 log 2 fold change. The results will show if the ASGPR binding moieties and other modifications can be tolerated at many positions, and if particular sites are sensitive to modification or exhibit improved activity when modified.

### Example 11: Readthrough of a Premature Termination Codon (PTC) in a Reporter Protein Via Administration of a TREM Comprising an ASGPR Binding Moiety in Cells Expressing the ASGPR

This example describes an assay to test the ability of a non-cognate GalNAc-TREM to readthrough a PTC in a cell line expressing a reporter protein having a PTC. This Example describes an arginine non-cognate TREM though a non-cognate TREM specifying any one of the other 19 amino acids can be used.

### Host Cell Modification

A cell line engineered to stably express the ASGPR and a NanoLuc reporter construct containing a premature termination codon (PTC) may be generated using the FlpIn system according to manufacturer's instructions. Briefly, HEK293T (293T ATCC® CRL-3216) cells are co-transfected with an expression vector containing a Nanoluc reporter with a PTC, such as pcDNA5/FRT-NanoLuc-TAA and a pOG44 Flp-Recombinase expression vector using Lipofectamine2000 according to manufacturer's instructions. After 24 hours, the media is replaced with fresh media. The next day, the cells are split 1:2 and selected with 100 ug/mL hygromycin for 5 days. The remaining cells are expanded and tested for reporter construct expression. Following that expansion step, the cells are co-transfected with a plasmid encoding the ASGRI gene and selection cassette, such as a puromycin cassette. The next day, cells are selected with puromycin. The remaining cells are expanded and tested for ASGPR expression.

### Synthesis and Preparation of Non-Cognate GalNAc-TREM

In this example, the arginine non-cognate GalNAc-TREM, is produced such that it contains the sequence of the ARG-UCU-TREM body but with the anticodon sequence corresponding to UCA instead of UCU and is conjugated to the GalNAc moiety. The arginine non-cognate GalNAc-TREM may be synthesized as described previously and its quality controlled using methods as described herein. To ensure proper folding, the TREM is heated at 85° C. for 2 minutes and then snap cooled at 4° C. for 5 minutes.

Delivery of Non-Cognate GalNAc-TREM into Host Cells

100 nM of the arginine non-cognate GalNAc-TREM may be delivered to mammalian cells gymnotically or using transfection reagents, as described herein.

### Translation Suppression Assay

To monitor the efficacy of the arginine non-cognate GalNAc-TREM to readthrough the PTC in the reporter construct, the cells are evaluated roughly 24-48 hours after TREM delivery. The cell media is replaced and the cells are allowed to equilibrate to room temperature. An equal volume to the cell media of ONE-Glo™ EX Reagent is added to the well and mixed on the orbital shaker at 500 rpm for 3 min followed by addition of an equal volume of cell media of NanoDLR™ Stop & Glo, followed by and mixing on the orbital shaker at 500 rpm for 3 min. The reaction is incubated at room temperature for 10 min and NanoLuc activity is detected by reading the luminescence in a plate reader. As a positive control, a host cell expressing the NanoLuc reporter construct without a PTC is used. As a negative control, a host cell expressing the NanoLuc reporter construct with a PTC is used but no GalNAc-TREM is transfected. The efficacy of the GalNAc-TREM may be measured as a ratio of the NanoLuc luminescence in the experimental sample to the NanoLuc luminescence of the positive control. It is expected that if the arginine non-cognate TREM is functional, read-through the stop mutation in the NanoLuc reporter may occur and produce a luminescent reading higher than the luminescent reading measured in the negative control. If the arginine non-cognate TREM is not functional, the stop mutation may not be not rescued, and luminescence less or equal to the negative control is detected.

### Example 12. Readthrough of a Premature Termination Codon (PTC) in the Alpha-Galactosidase (GLA) ORF Through Administration of a TREM Comprising an ASGPR Binding Moiety

This example describes an assay to test the ability of a non-cognate GalNAc-TREM to readthrough a PTC, such as R220X, in the alpha-galactosidase (GLA) open reading frame (ORF) in hepatocytes differentiated from reprogrammed Fabry disease patient-derived cell line. This Example describes an arginine non-cognate GalNAc-TREM, though a non-cognate TREM specifying any one of the other 19 amino acids can be used.

### Patient-Derived Cells

Fibroblast cells derived from a patient with Fabry disease having a PTC in the alpha-galactosidase (GLA) open reading frame (ORF), such as R220X, may be obtained from a center or an organization, such as the Coriell Institute (catalog #s GM00881 and GM02769). The patient-derived fibroblast cells are reprogrammed into iPSCs and differentiated into hepatocytes as previously shown (Takahashi, K. & Yamanaka, S. (2006) Cell 126, 663-676 (2006); Park I. et al. (2008) Nature 451, 141-146); Jia, B. et al. (2014) Life Sci. 108, 22-29).

### Synthesis and Preparation of Non-Cognate GalNAc-TREM

In this example, the arginine non-cognate GalNAc-TREM is produced such that it contains the sequence of the ARG-UCU-TREM body but with the anticodon sequence corresponding to UCA instead of UCU and is conjugated to the GalNAc moiety. The arginine non-cognate GalNAc-TREM is synthesized as described previously and its quality controlled using methods as described in Examples 10-11. To ensure proper folding, the TREM is heated at 85° C. for 2 minutes and then snap cooled at 4° C. for 5 minutes.

Delivery of Non-Cognate GalNAc-TREM into Hepatocytes

100 nM of the arginine non-cognate GalNAc-TREM may be delivered gymnotically, to iPSC-derived hepatocytes cells originating from Fabry patient-derived fibroblasts.

### Translation Suppression Assay

To monitor the efficacy of the arginine non-cognate GalNAc-TREM to readthrough the PTC in the GLA ORF, 24-48 hours after transfection, cell media is replaced, and cells are lysed. Using Western blot detection, the non-cognate GalNAc-TREM efficacy is measured as the level of full-length protein expression, in this example of GLA enzyme, in the reprogrammed hepatocyte cells dosed with the Arg non-cognate TREM, in comparison to the GLA expression levels found in control hepatocyte cells not receiving the TREM. For example, as a control, cells of a person unaffected by the disease (i.e. cells having an ORF with a WT GLA transcript) may be used. It is expected that if the non-cognate GalNAc-TREM is functional, it can readthrough the PTC and the full-length protein level will be detected at higher levels than that found in reprogrammed hepatocyte cells which have not been administered the non-cognate GalNAc-TREM. If the non-cognate GalNAc-TREM is not functional, the full-length protein level will be detected at a similar level as detected in patient-derived fibroblast cells or reprogrammed hepatocyte cells which have not been administered the non-cognate GalNAc-TREM.

### Example 13: Readthrough of a Premature Termination Codon (PTC) in the Alpha-Galactosidase (GLA) ORF to Produce a Functional GLA Protein Through Administration of a TREM Comprising an ASGPR Binding Moiety

This example describes an assay to test the ability of a non-cognate GalNAc-TREM to readthrough a PTC, such as R220X, in the alpha-galactosidase (GLA) open reading frame (ORF) in hepatocytes differentiated from reprogrammed Fabry disease patient-derived cell line to generate the production of a functional GLA protein. This Example describes an arginine non-cognate GalNAc-TREM, though a non-cognate TREM specifying any one of the other 19 amino acids can be used. Fibroblast cells derived from a patient with Fabry disease having a PTC in the alpha-galactosidase (GLA) open reading frame (ORF), such as R220X, may be obtained from a center or an organization, such as the Coriell Institute (catalog #s GM00881 and GM02769). The cells can be reprogrammed and differentiated according to the exemplary protocols provided in Example 12.

To monitor the functionality of the GLA protein produced as a result of arginine non-cognate GalNAc-TREM-mediated PTC readthrough, a GLA protein activity assay may be performed using the Alpha Galactosidase Activity Assay Kit (Abcam) according to manufacturer instructions. Alternatively, GLA activity may be determined using the artificial substrate 4-methylumbelliferyl-α-D-galactoside as described previously in Desnick R J, et al. J Lab Clin Med. 1973; 81:157-71.

1. A tRNA-based effector molecule (TREM) comprising an asialoglycoprotein receptor (ASGPR) binding moiety, wherein the ASGPR binding moiety is bound to a sugar moiety (e.g., a ribose moiety), nucleobase, or the internucleotide linkage (e.g., the phosphate backbone) of a nucleotide within a TREM sequence, wherein the TREM comprises:
(i) a sequence of Formula A comprising:
[L1]_{y}-[ASt Domain1]ₓ-[L2]ₓ-[DH Domain]ₓ-[L3]ₓ-[ACH Domain]ₓ-[VL Domain]_{y}-[TH Domain]ₓ-[L4]ₓ-[ASt Domain2]ₓ, (A); and
(ii) an asialoglycoprotein receptor (ASGPR) binding moiety (e.g., a GalNAc moiety, e.g., GalNAc); and
wherein y is 0 or 1 and x is 1.

2. The TREM of claim 1, wherein the asialoglycoprotein receptor binding moiety comprises a galactose (Gal) moiety, galactosamine (GalNH₂) moiety, or N-acetylgalactosamine (GalNAc) moiety.

3. The TREM of claim 2, wherein the GalNAc moiety comprises GalNAc or an analog thereof (e.g., a triantennary GalNAc or an analog thereof).

4. The TREM of any one of claims 1-3, wherein the TREM comprises a full length TREM, a TREM Core Fragment, or a TREM Fragment.

5. The TREM of claim 1, wherein the sequence of Formula A:
(i) a sequence of Formula A comprising:
[L1]_{y}-[ASt Domain1]ₓ-[L2]ₓ-[DH Domain]ₓ-[L3]ₓ-[ACH Domain]ₓ-[VL Domain]_{y}-[TH Domain]ₓ-[L4]ₓ-[ASt Domain2]ₓ, (A); and
(ii) an asialoglycoprotein receptor (ASGPR) binding moiety (e.g., a GalNAc moiety, e.g., GalNAc);
wherein the ASGPR binding moiety is bound to a sugar moiety (e.g., a ribose moiety) or the internucleotide linkage (e.g., the phosphate backbone),
wherein y is 0 or 1 and x is 1.

6. The TREM of claim 5, wherein the ASGPR binding moiety is present on the sugar moiety (e.g., a ribose moiety) at the 2′ position of the sugar moiety.

7. The TREM of claim 6, wherein the ASGPR binding moiety is present on the sugar moiety (e.g., a ribose moiety) at the 2′ oxygen or carbon of the sugar moiety.

8. The TREM of claim 6, wherein the ASGPR binding moiety is present on a sugar moiety (e.g., a ribose moiety) at the 4′ position of the sugar moiety.

9. The TREM of claim 5, wherein the ASGPR binding moiety is bound to the phosphate backbone at any nucleotide position within the TREM.

10. The TREM of any one of claims 5-9, wherein the ASGPR binding moiety is present within a linker region.

11. The TREM of claim 10, wherein the linker region comprises L1, L2, L3, and/or L4.

12. The TREM of any one of the preceding claims, wherein the ASGPR binding moiety (e.g., a GalNAc moiety, e.g., GalNAc) is bound to the sugar moiety (e.g., ribose moiety) of a nucleotide of the TREM molecule.

13. The TREM of any one of the preceding claims, wherein the ASGPR binding moiety (e.g., a GalNAc moiety, e.g., GalNAc) is bound to the sugar (e.g., ribose) moiety of a nucleotide of the TREM via a covalent linkage (e.g., at a nitrogen or carbon atom in the sugar moiety).

14. The TREM of any one of the preceding claims, wherein the ASGPR binding moiety (e.g., a GalNAc moiety, e.g., GalNAc) is bound to a nucleobase within a nucleotide of the TREM.

15. The TREM of any one of the preceding claims, wherein the ASGPR binding moiety (e.g., a GalNAc moiety, e.g., GalNAc) is bound to a nucleobase within a nucleotide of the TREM via covalent linkage.

16. The TREM of any one of the preceding claims, wherein the ASGPR binding moiety (e.g., a GalNAc moiety, e.g., GalNAc) is bound to an adenine, uracil, cytosine, or guanosine.

17. The TREM of any one of the preceding claims, wherein the ASGPR binding moiety (e.g., a GalNAc moiety, e.g., GalNAc) is bound to a uracil.

18. The TREM of any one of the preceding claims, wherein the ASGPR binding moiety (e.g., a GalNAc moiety, e.g., GalNAc) is bound to an atom in the internucleotide linkage of the TREM.

19. The TREM of any one of claims 5-18, wherein the ASGPR binding moiety is present on a sugar moiety (e.g., ribose moiety) of a nucleotide within the AStD.

20. The TREM of any one of claims 5-19, wherein the ASGPR binding moiety is present within the ASt Domain 1 (e.g., positions 1-9).

21. The TREM of any one of claims 5-20, wherein the ASGPR binding moiety is present within the ASt Domain 2 (e.g., positions 65-76).

22. The TREM of any one of claims 5-21, wherein the ASGPR binding moiety is present within the ACHD (e.g., positions 27-43).

23. The TREM of any one of claims 5-22, wherein the ASGPR binding moiety is present within the DHD (e.g., positions 10-26).

24. The TREM of any one of claims 5-23, wherein the ASGPR binding moiety is present within the THD (e.g., positions 50-64).

25. The TREM of any one of the preceding claims, wherein the ASGPR binding moiety comprises a GalNAc moiety (e.g., a GalNAc or a GalNAc analog).

26. The TREM of any one of the preceding claims, wherein the TREM retains the ability to support protein synthesis, e.g., relative to a TREM that does not comprise an ASGPR binding moiety or a naturally occurring tRNA.

27. The TREM of any of claims 1-21, wherein the TREM retains the ability to be charged by a synthetase, e.g., relative to a TREM that does not comprise an ASGPR binding moiety or a naturally occurring tRNA.

28. The TREM of any of claims 1-22, wherein the TREM retains the ability to be bound by an elongation factor, e.g., relative to a TREM that does not comprise an ASGPR binding moiety or a naturally occurring tRNA.

29. The TREM of any of claims 1-23, wherein the TREM retains the ability to introduce an amino acid into a peptide chain, e.g., relative to a TREM that does not comprise an ASGPR binding moiety or a naturally occurring tRNA.

30. The TREM of any of claims 1-24, wherein the TREM retains the ability to support elongation or support initiation, e.g., relative to a TREM that does not comprise an ASGPR binding moiety or a naturally occurring tRNA.

31. The TREM of any one of claims 1-25, wherein the TREM has a binding affinity to an ASGPR of between 0.01 nM and 100 mM.

32. The TREM of claim 1, wherein the non-naturally occurring modification is present on the 2′-position of a nucleotide sugar or within the internucleotide region (e.g., a backbone modification).

33. The TREM of any one of the preceding claims, wherein the TREM comprises a non-naturally occurring modification.

34. The TREM of claim 33, wherein the non-naturally occurring modification is selected from a 2′-O-methyl (2-OMe), 2′-halo (e.g., 2′F or 2′Cl), 2′-O-methoxyethyl (2′MOE), or 2′deoxy modification.

35. The TREM of any one of claims 33-34, wherein the non-naturally occurring modification is a phosphorothioate modification.

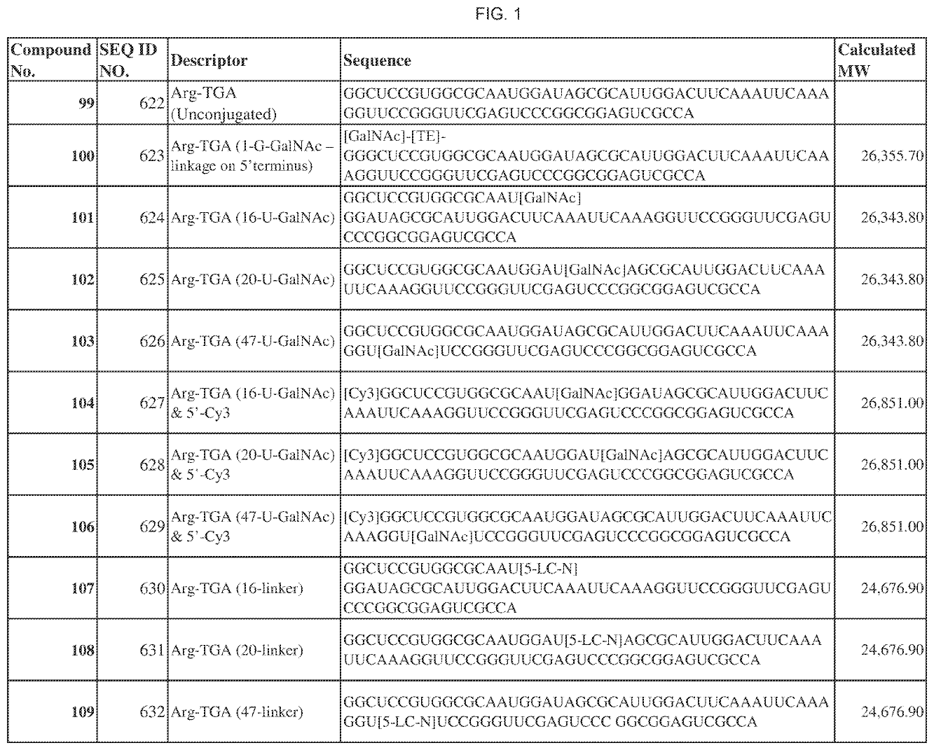

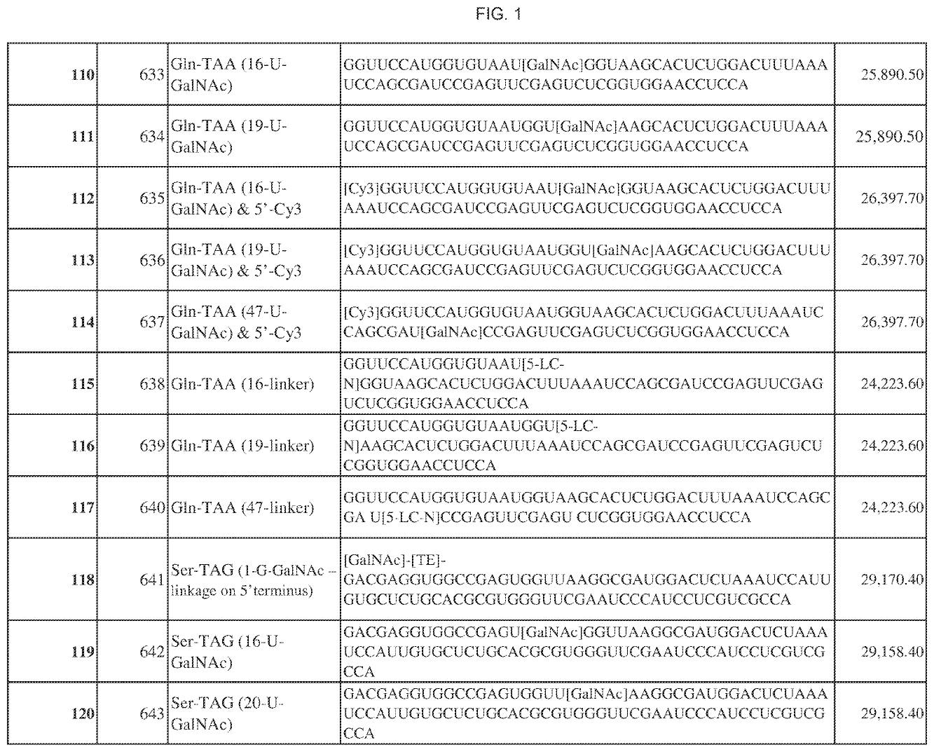

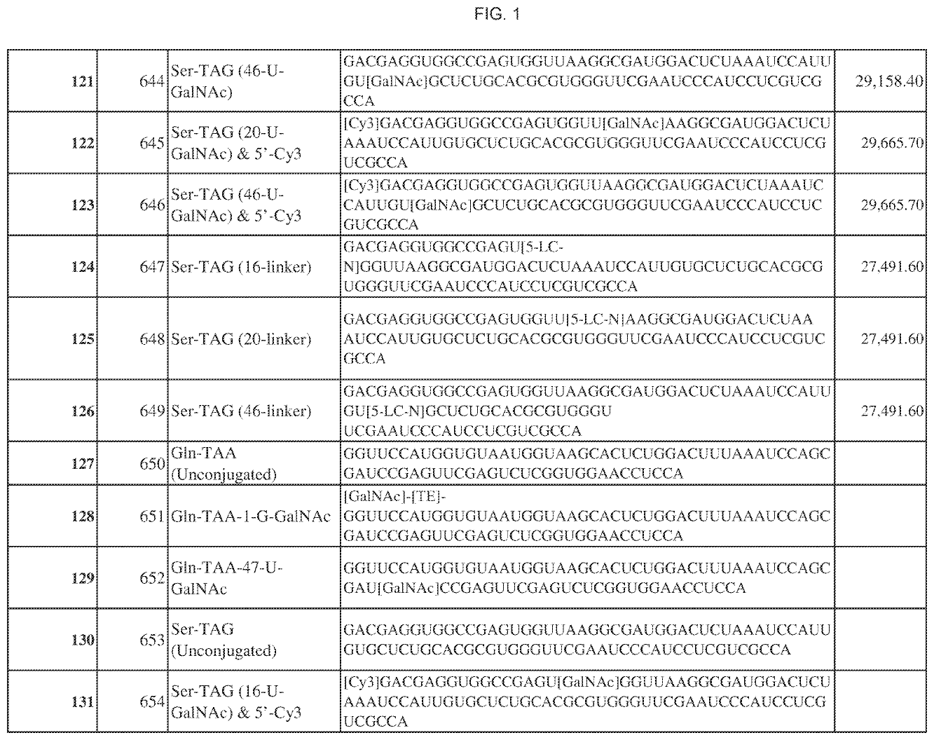

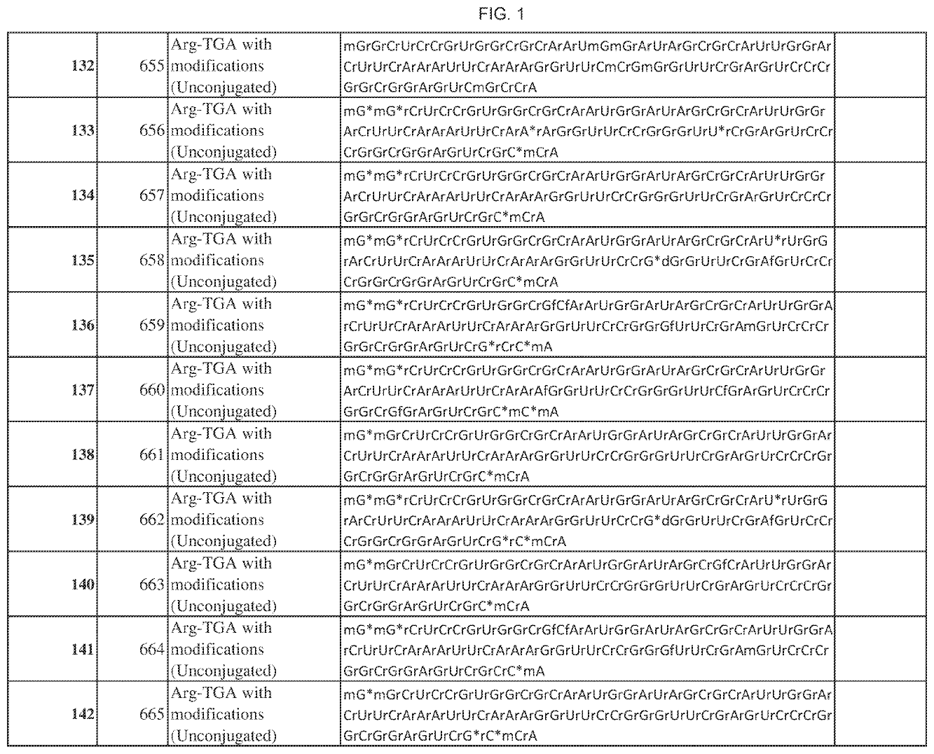

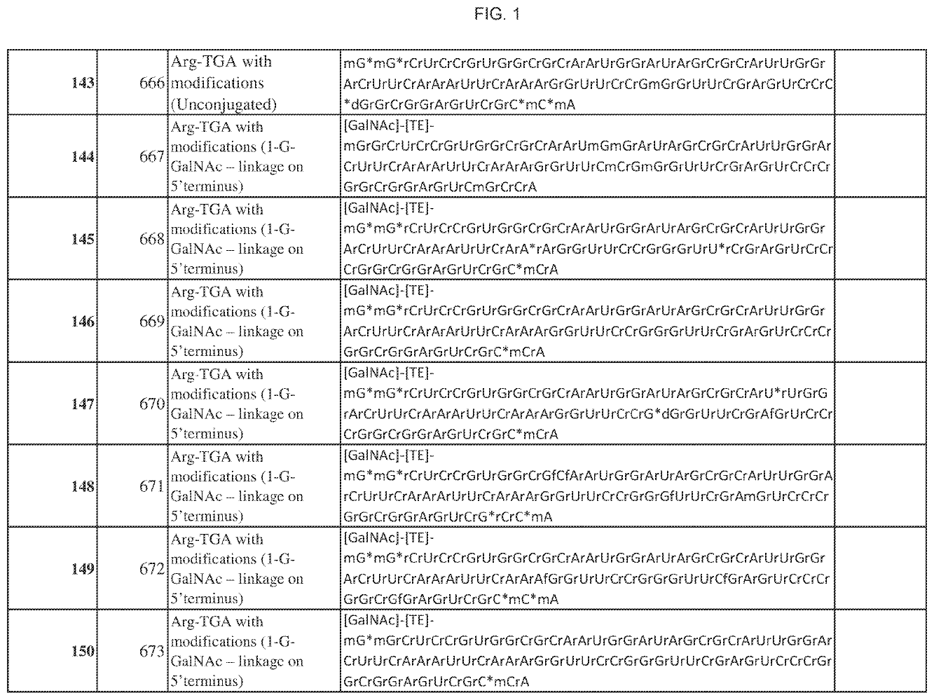

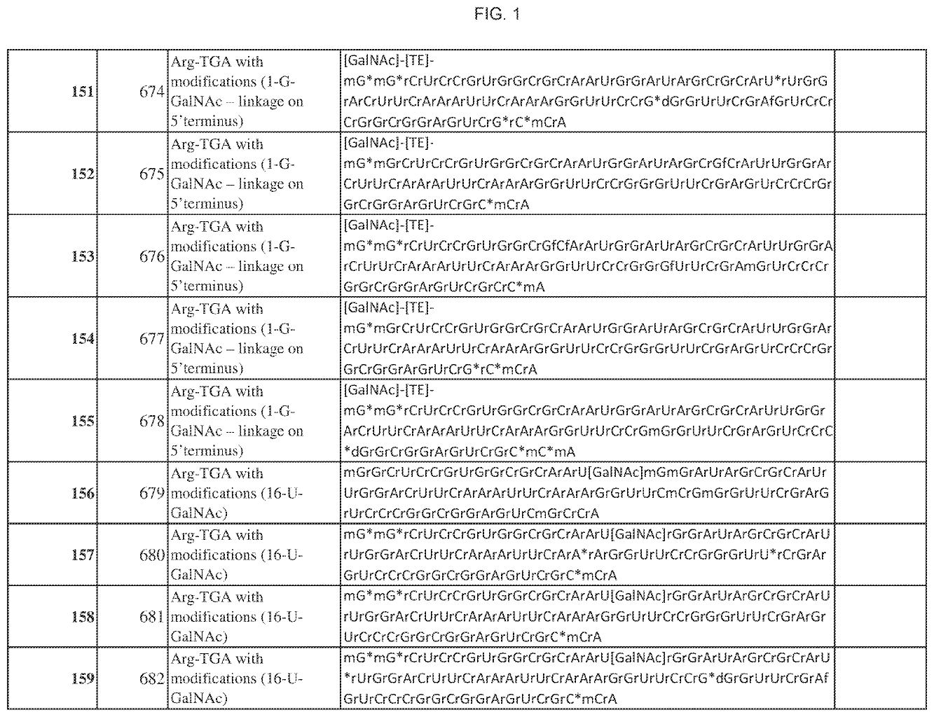

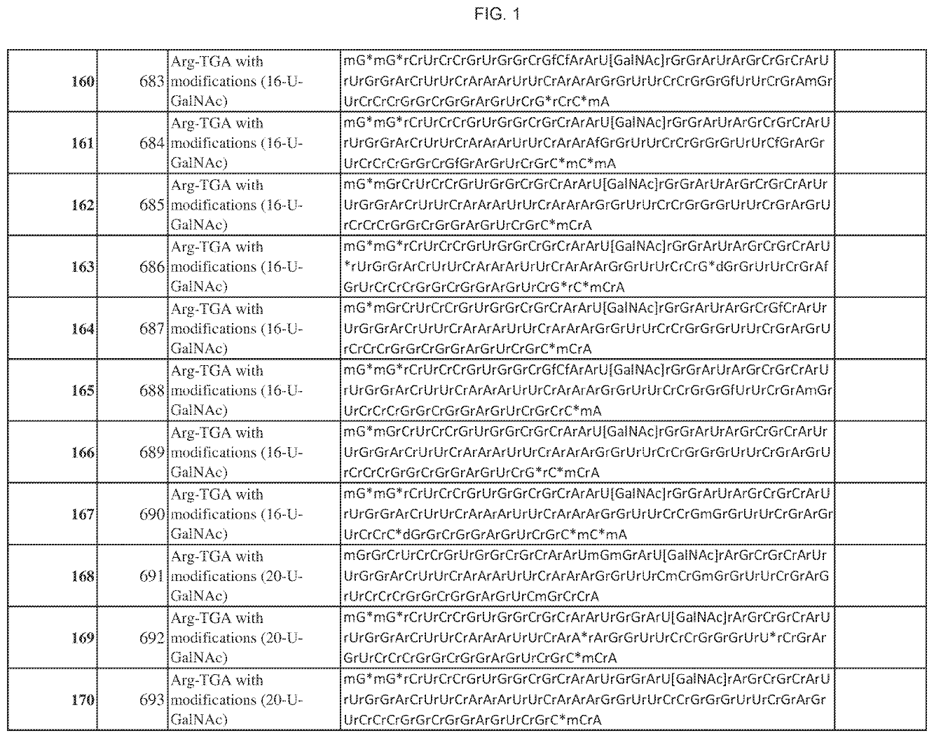

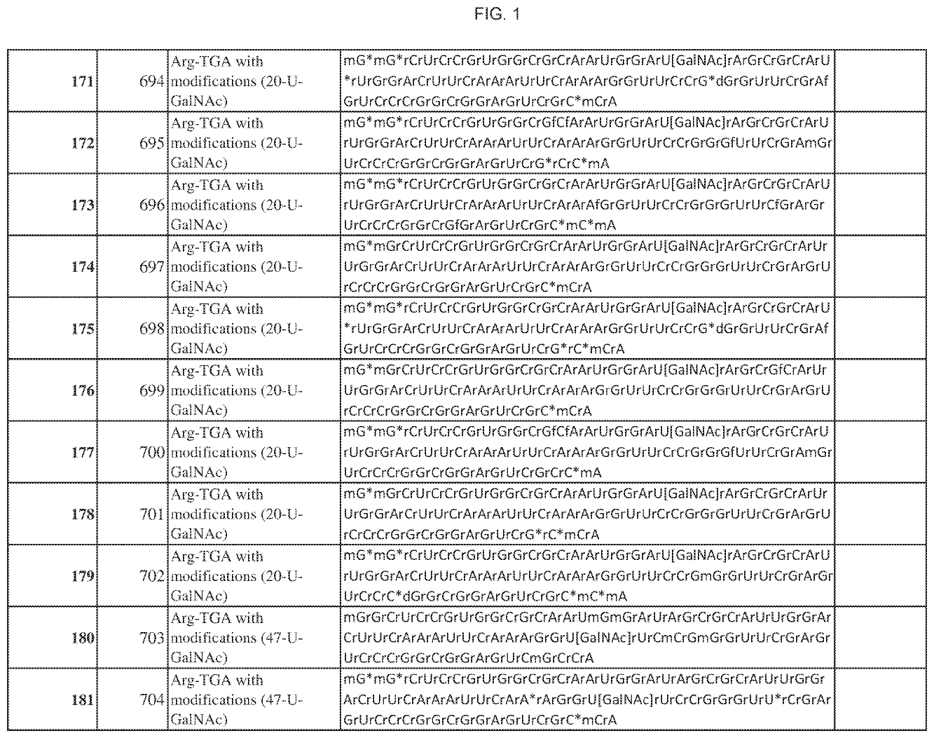

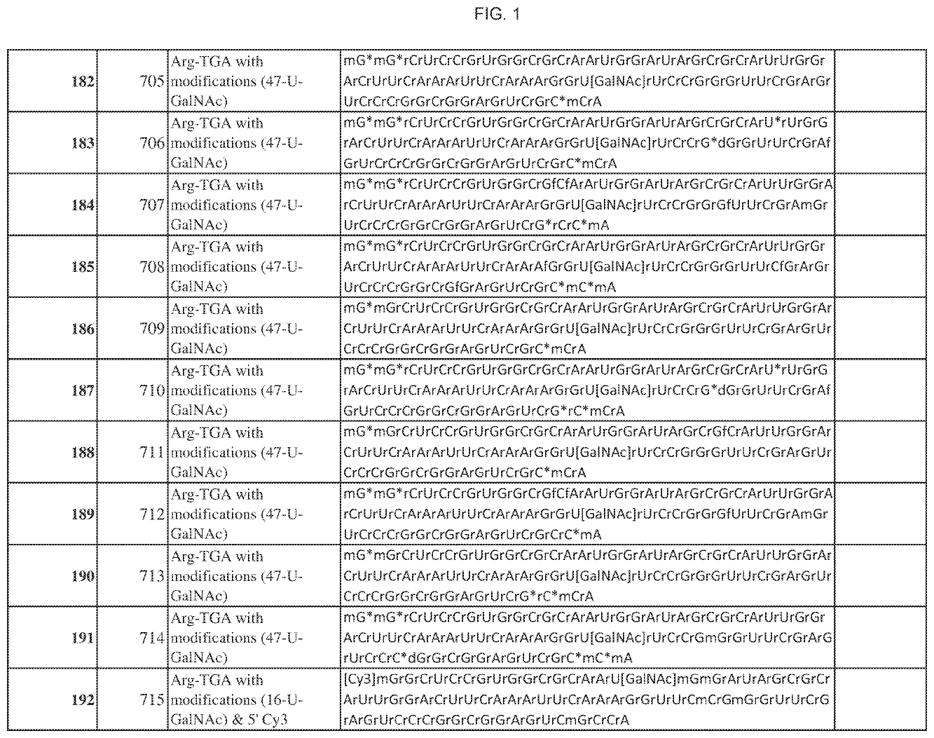

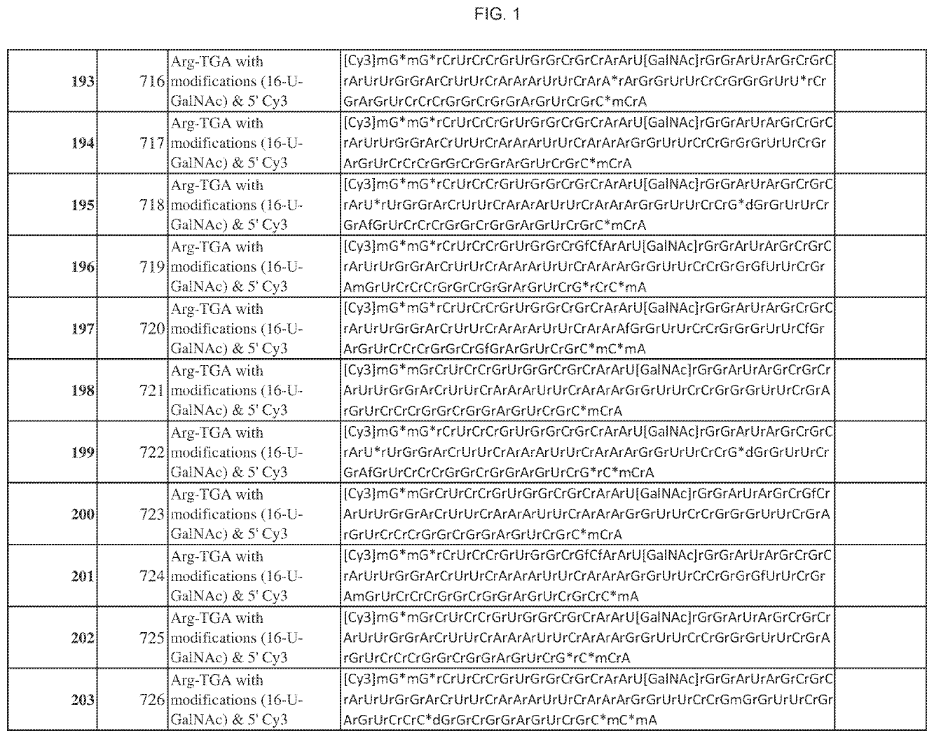

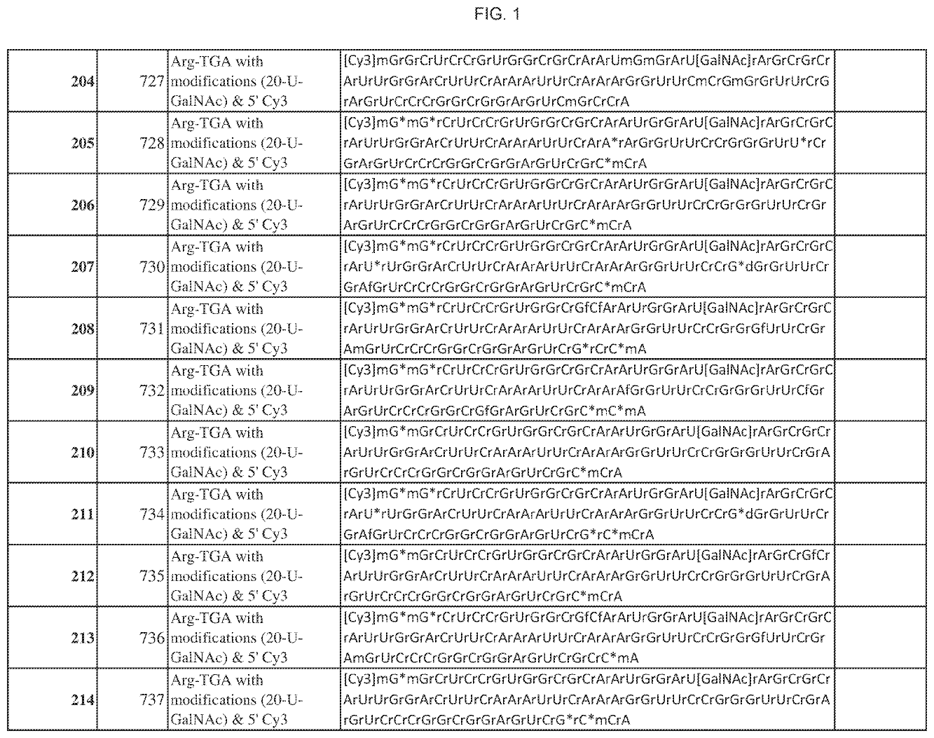

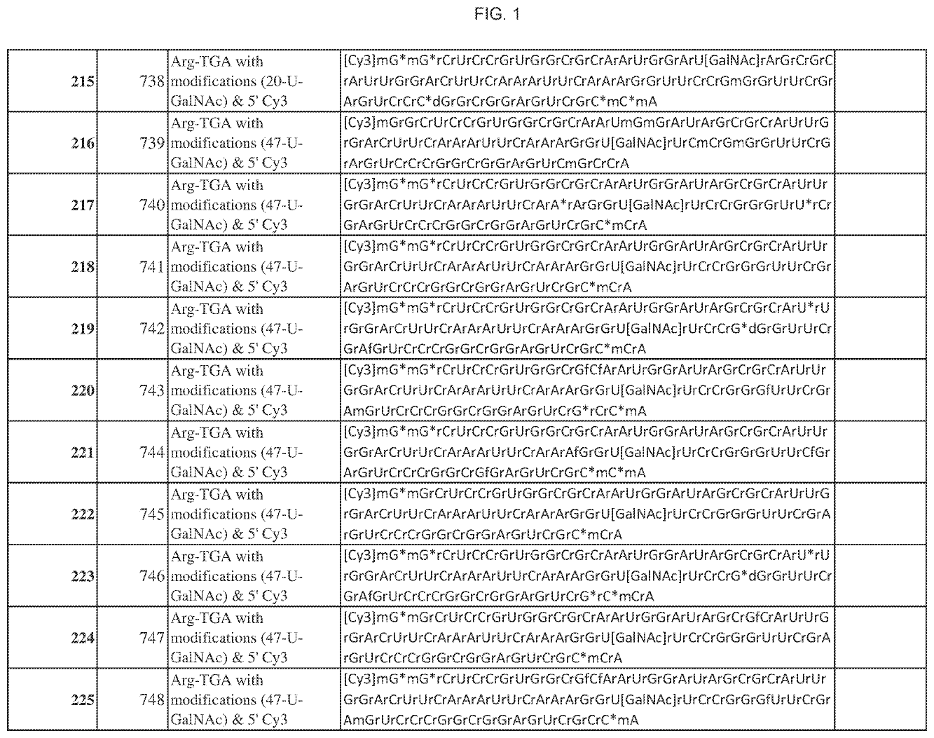

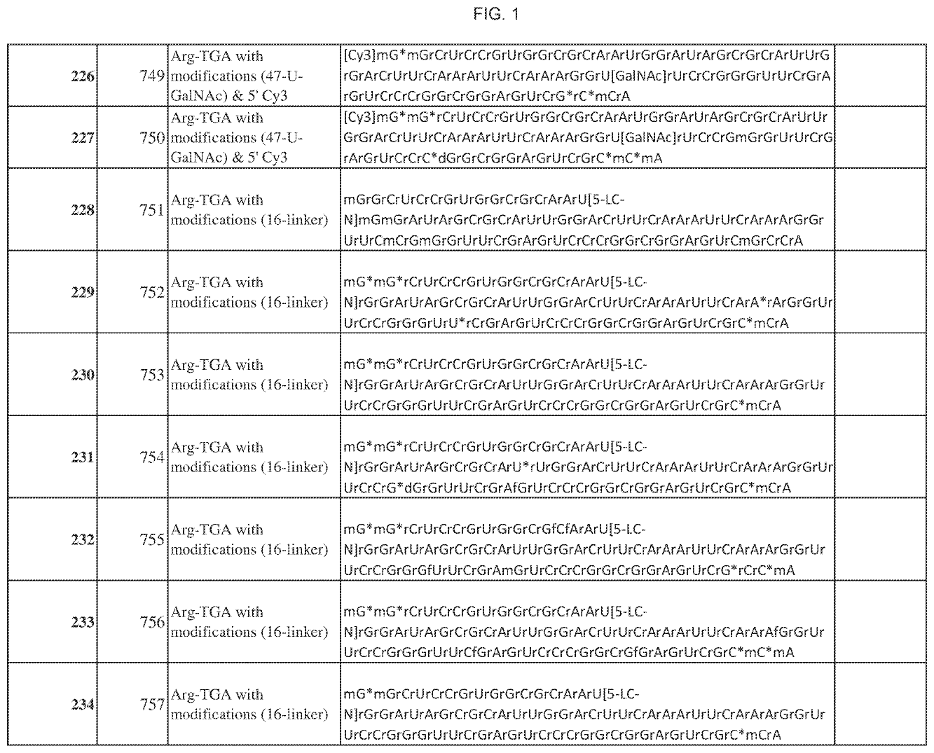

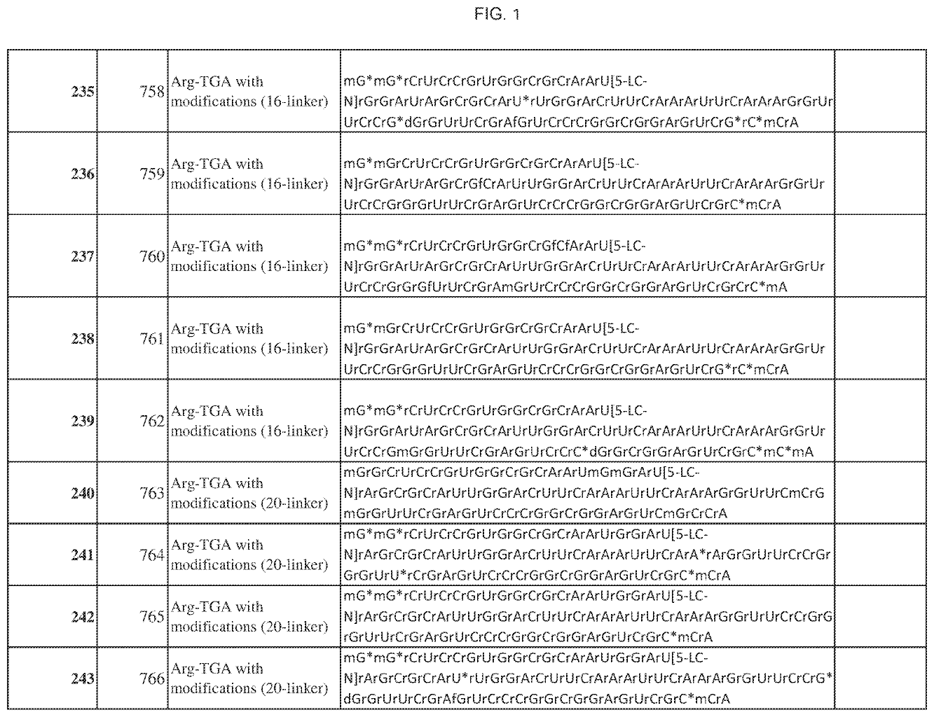

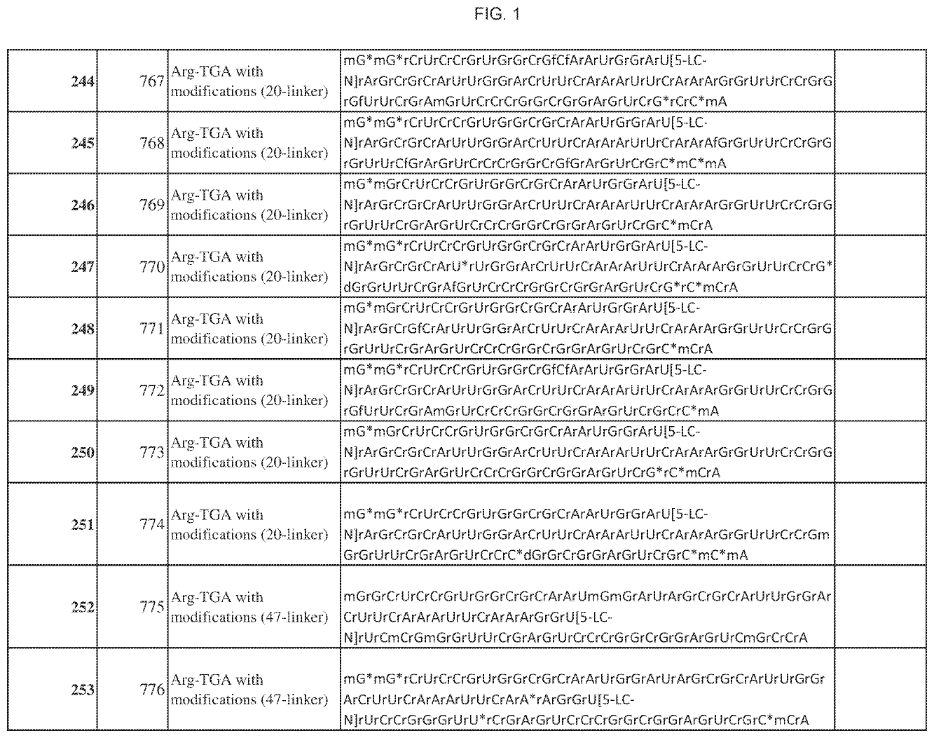

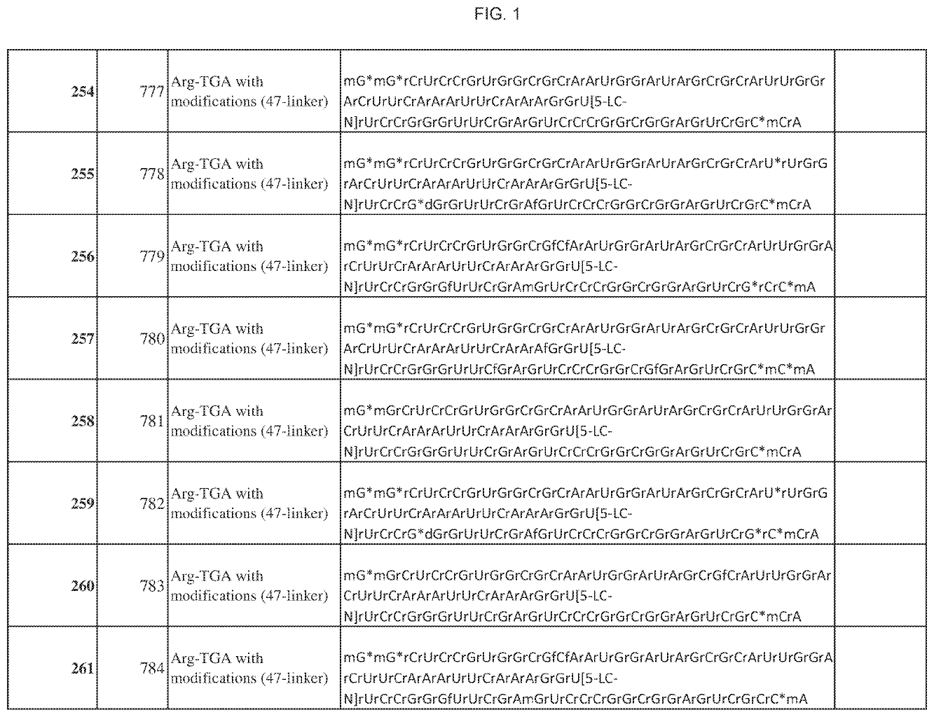

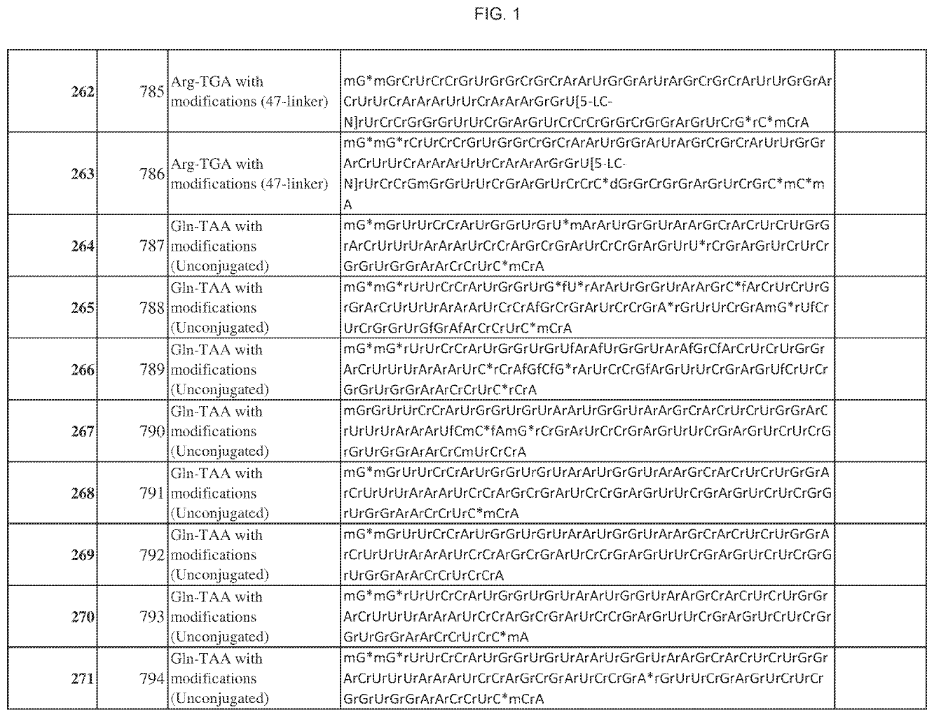

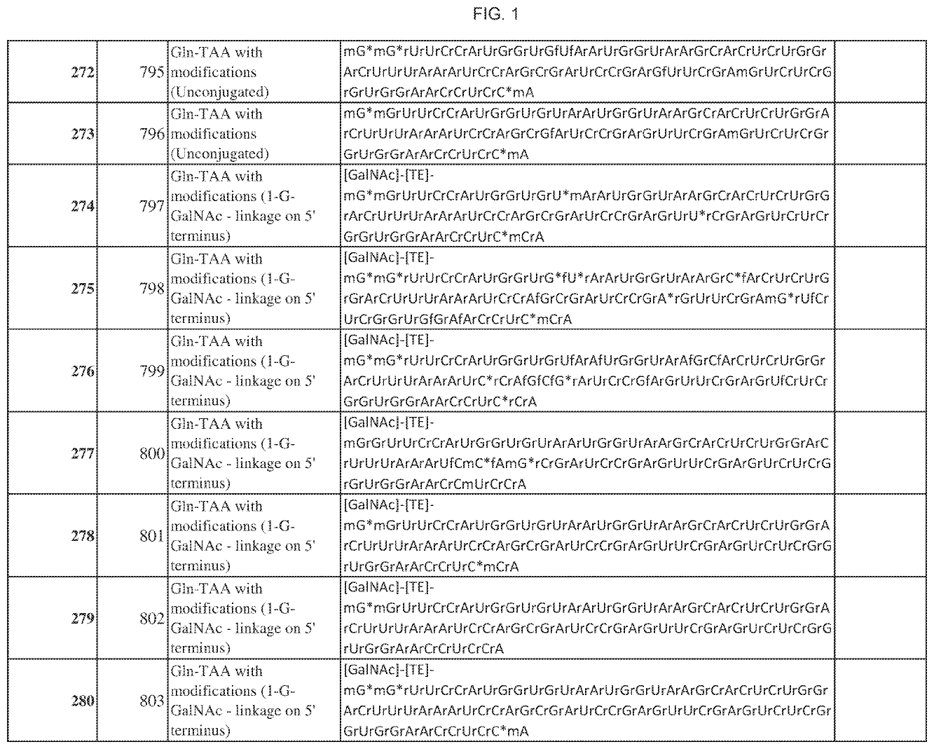

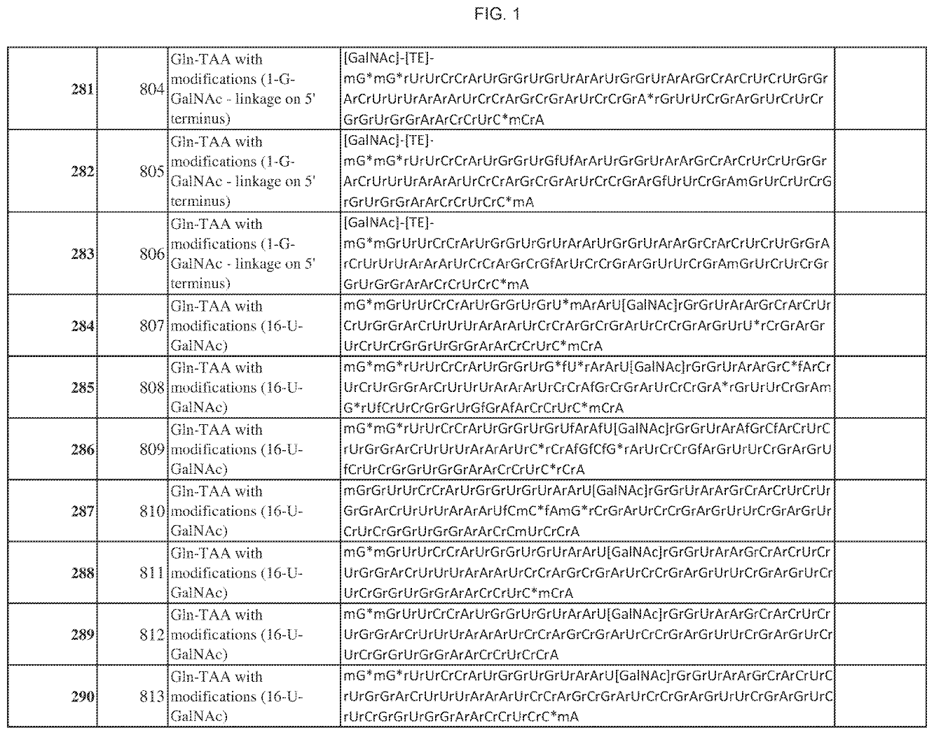

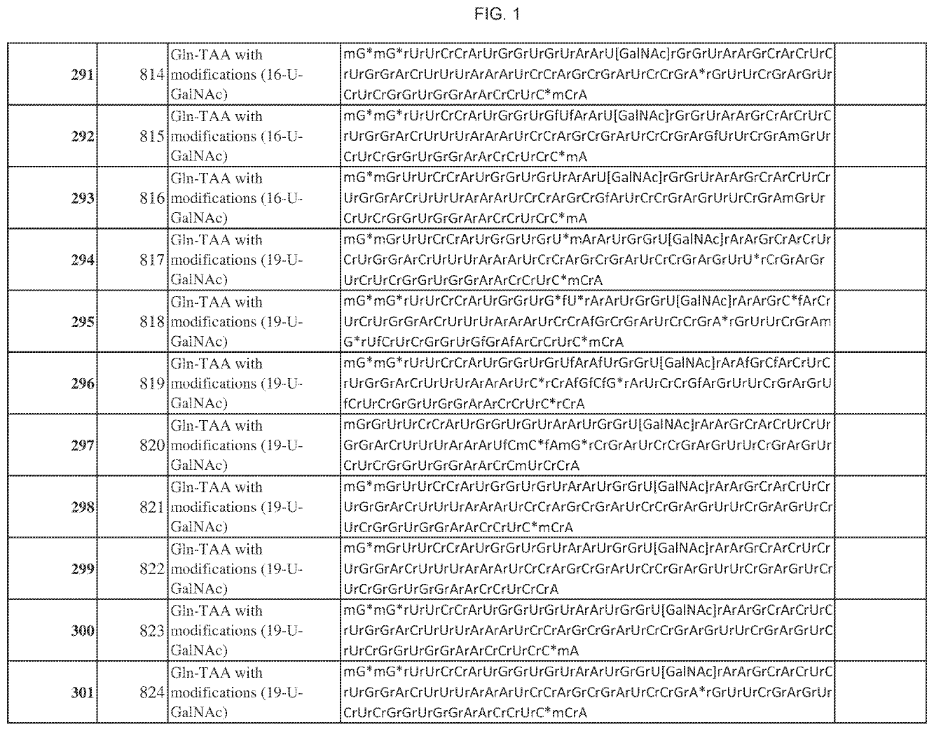

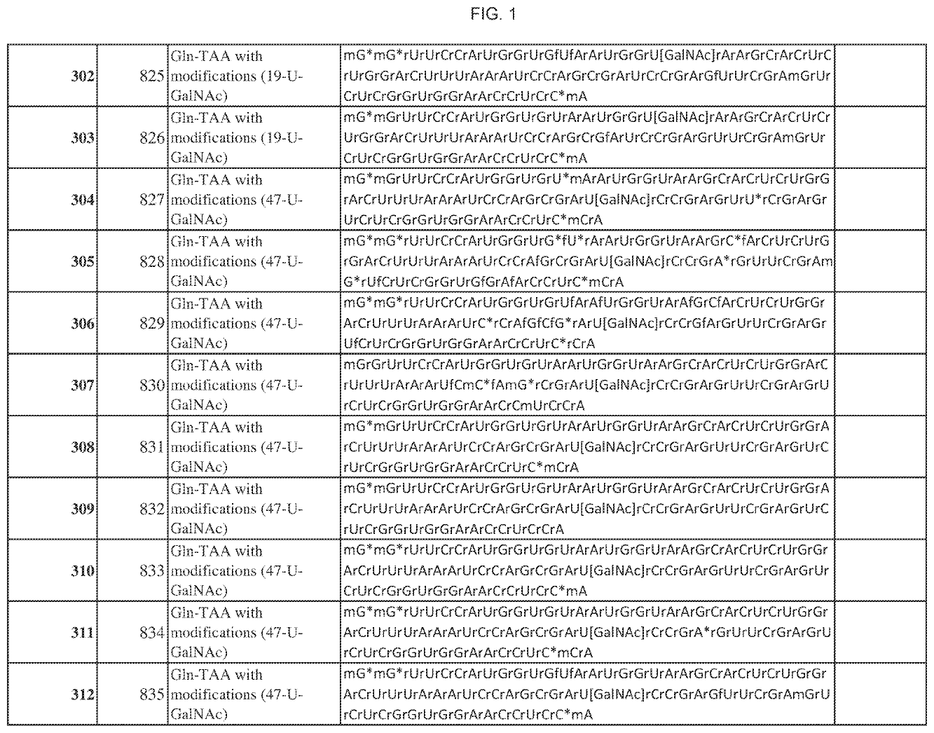

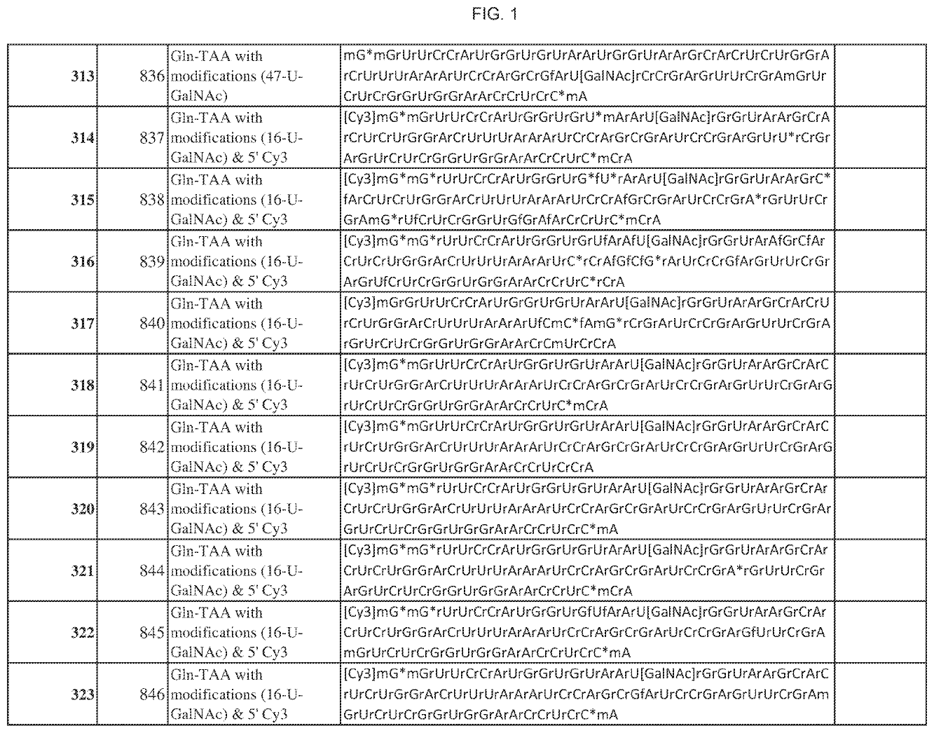

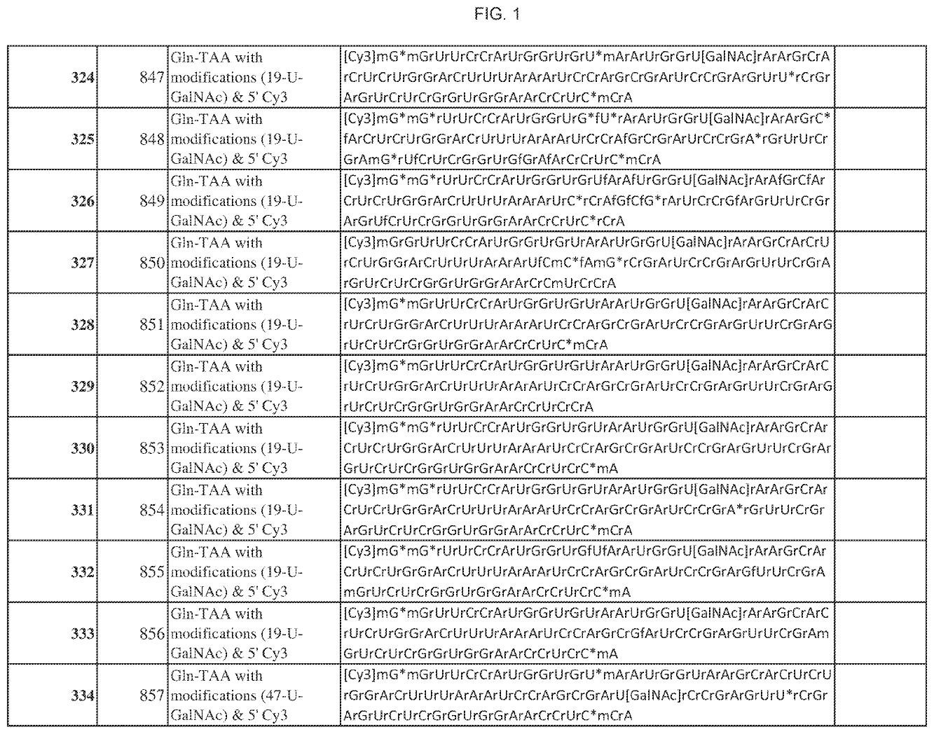

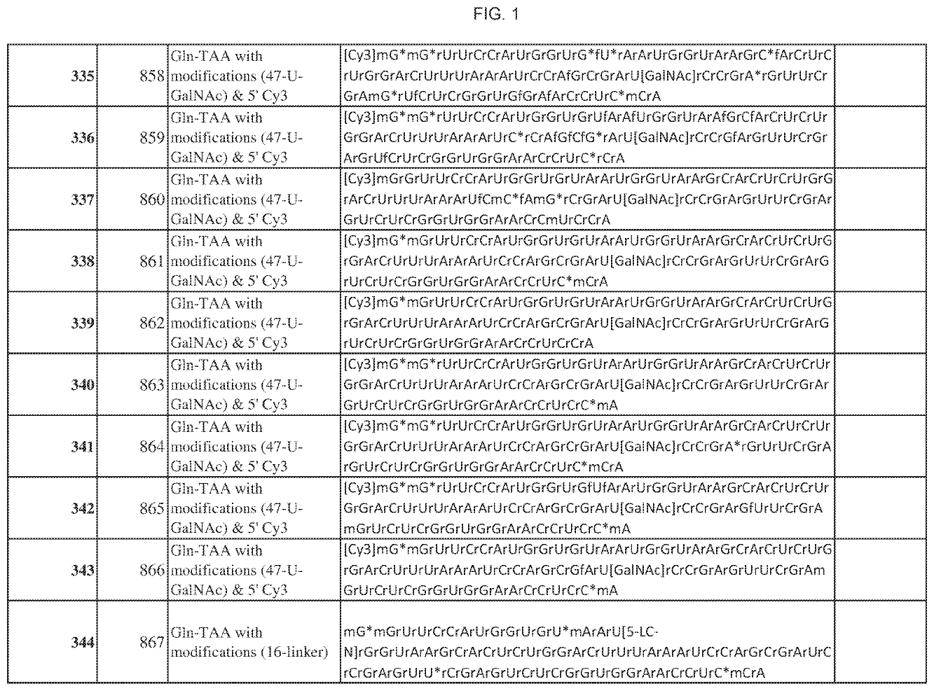

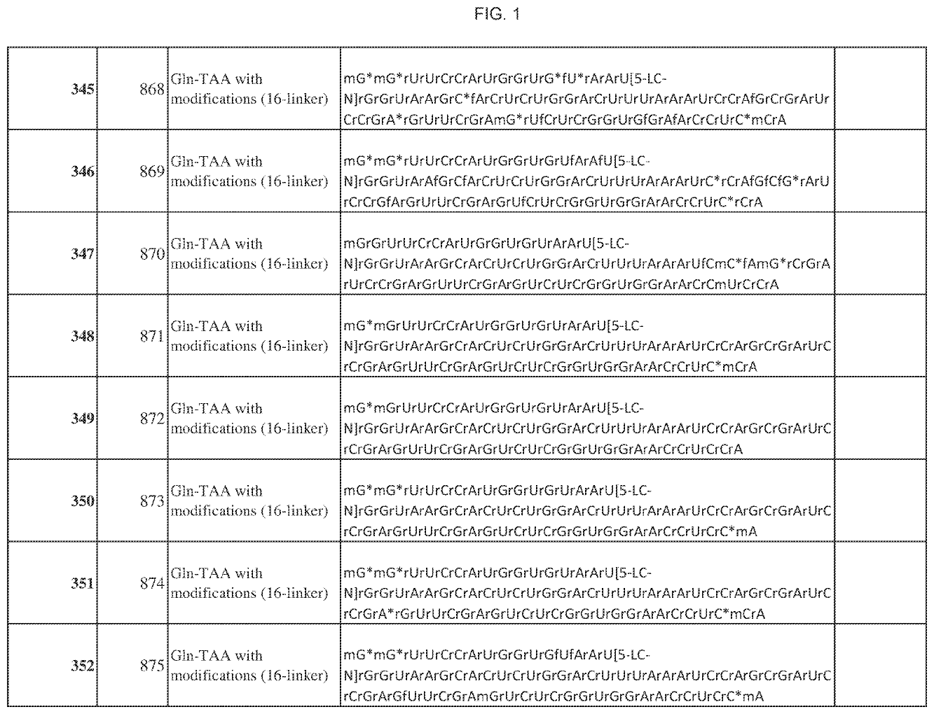

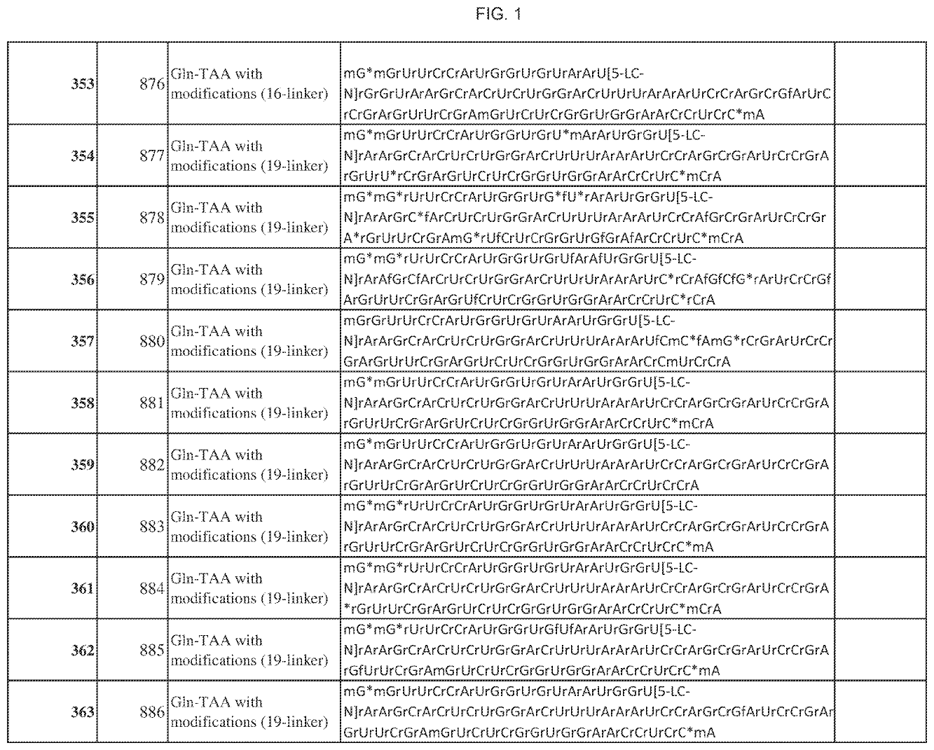

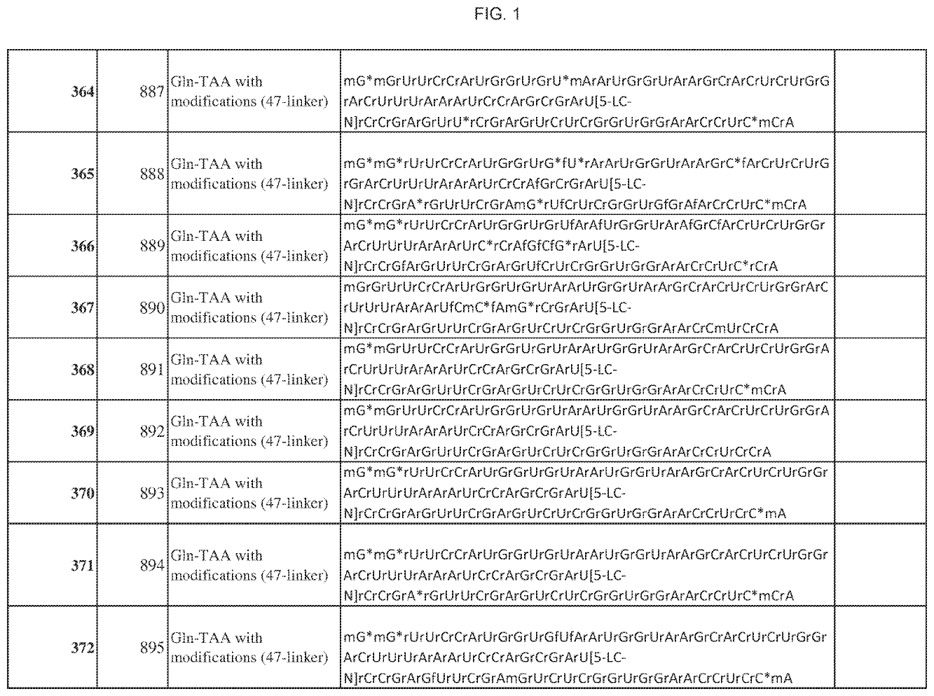

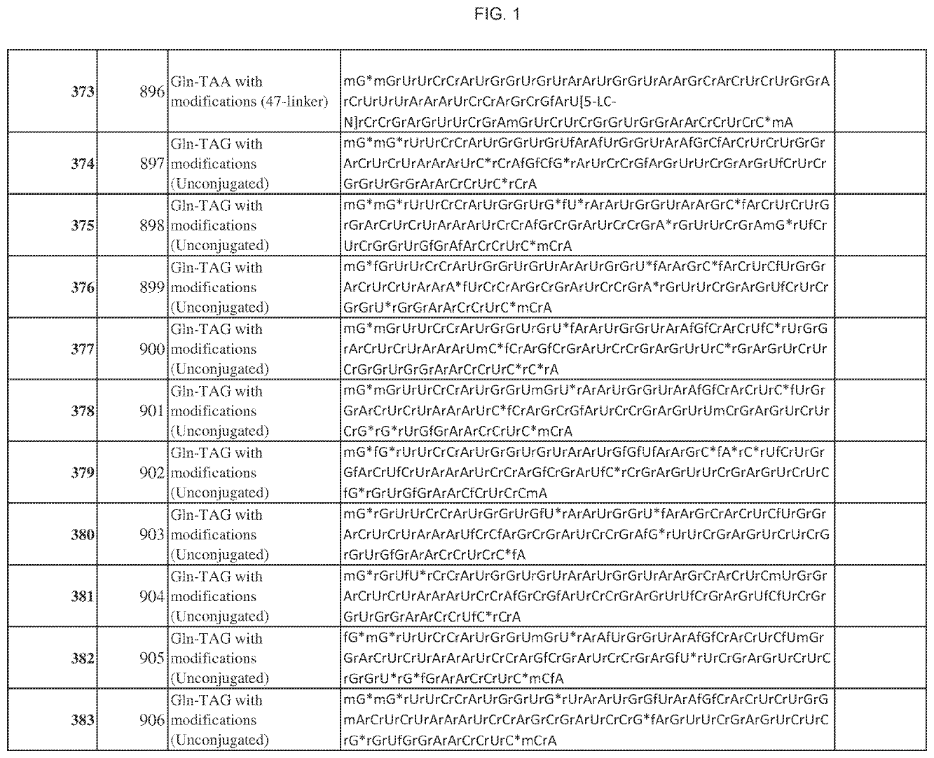

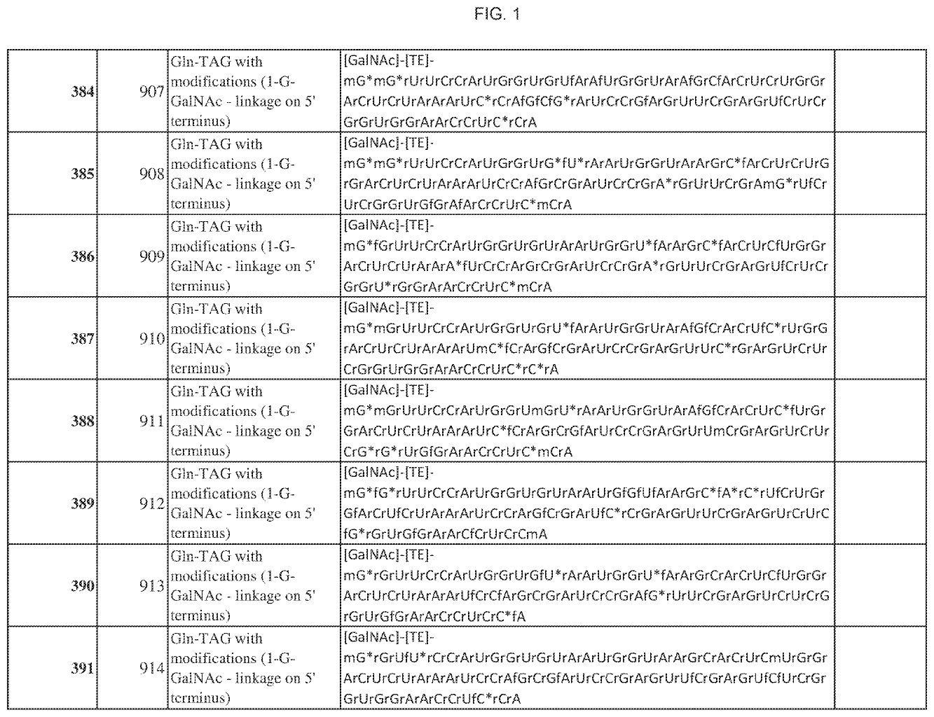

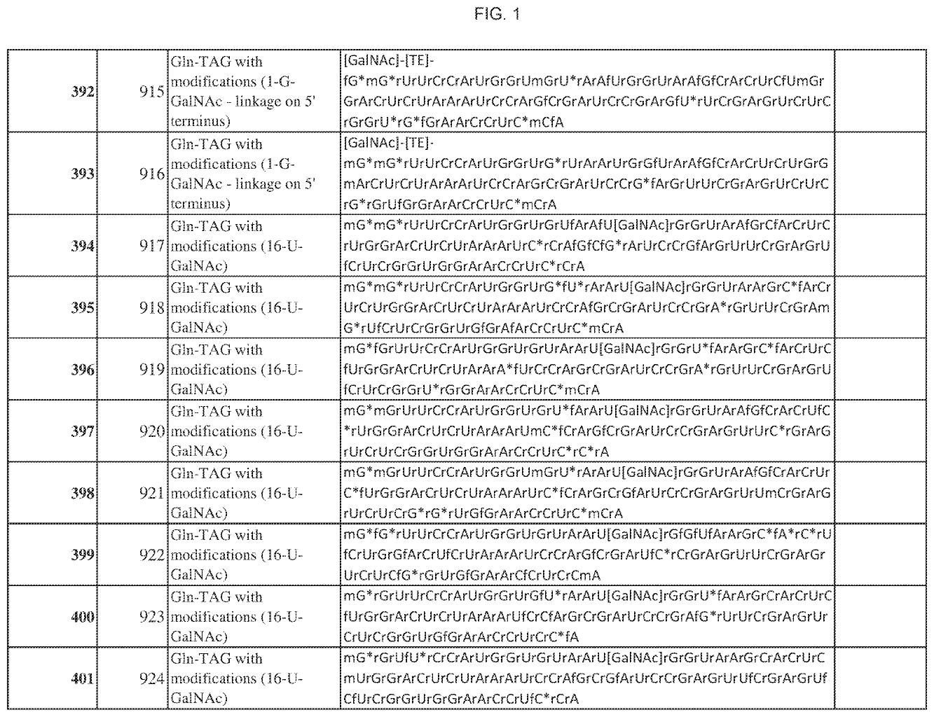

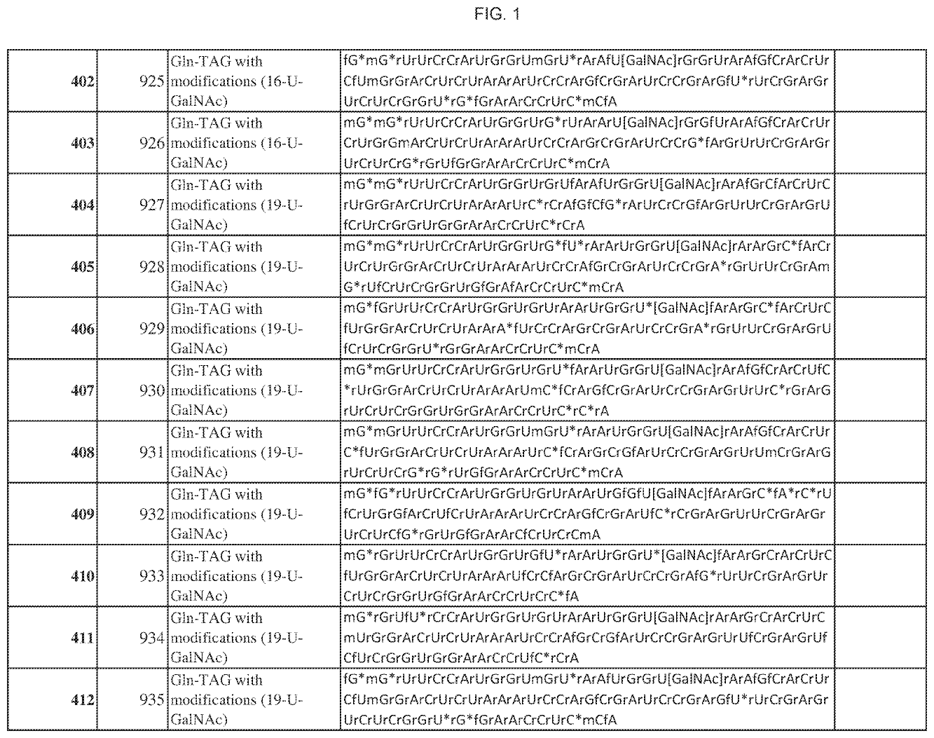

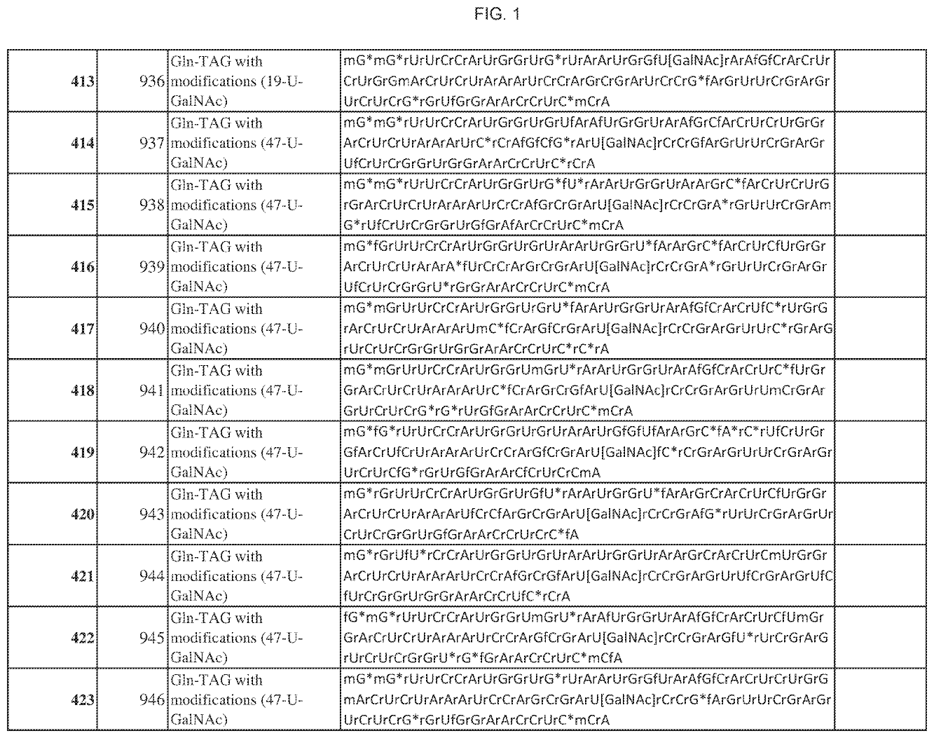

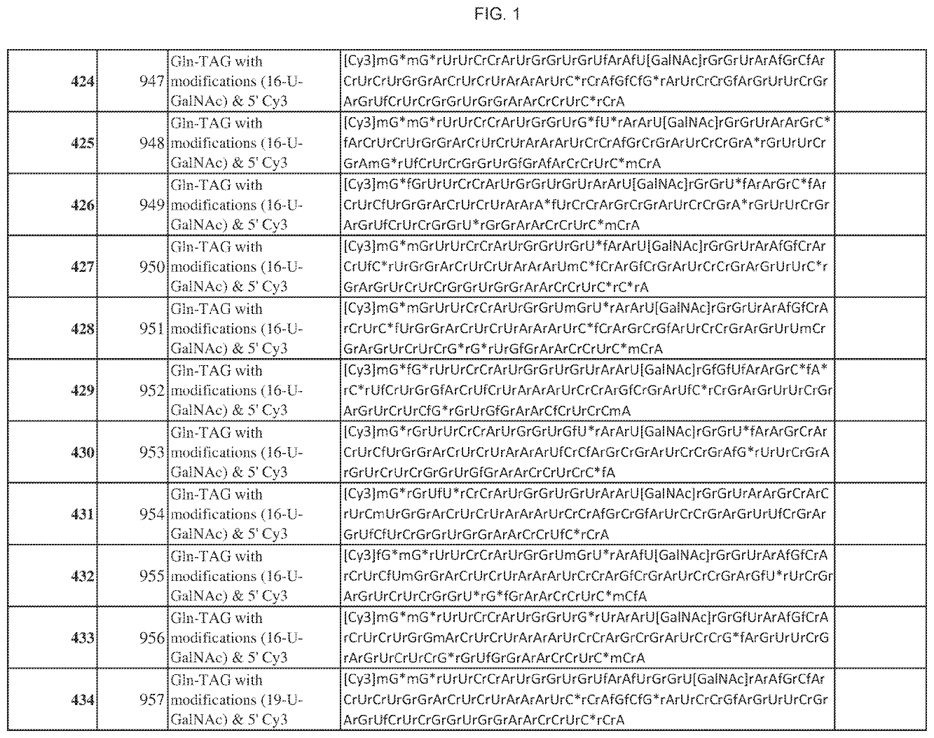

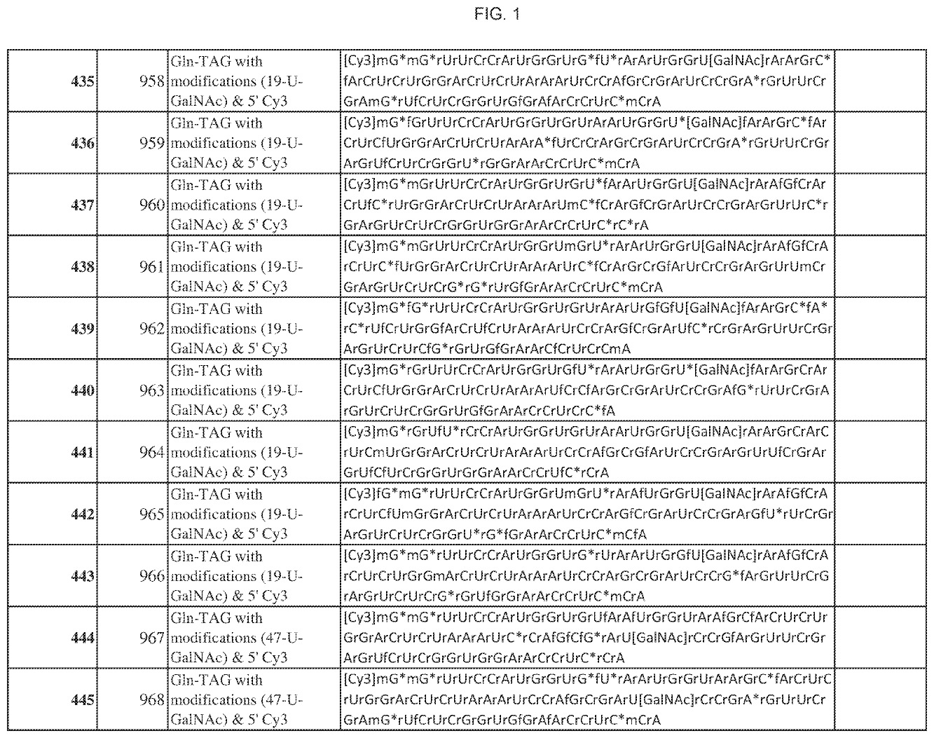

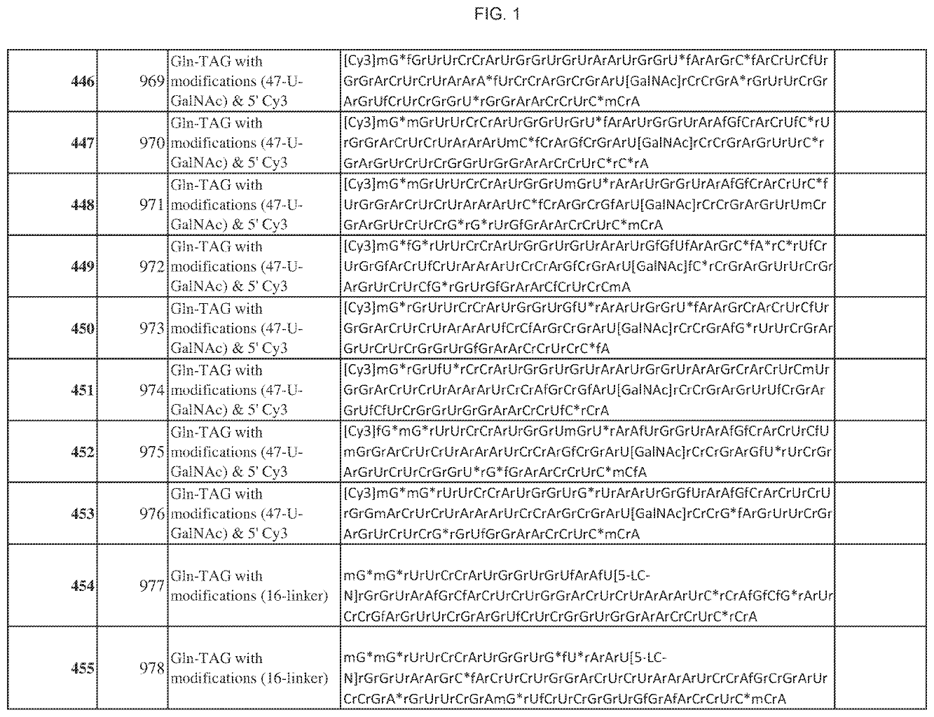

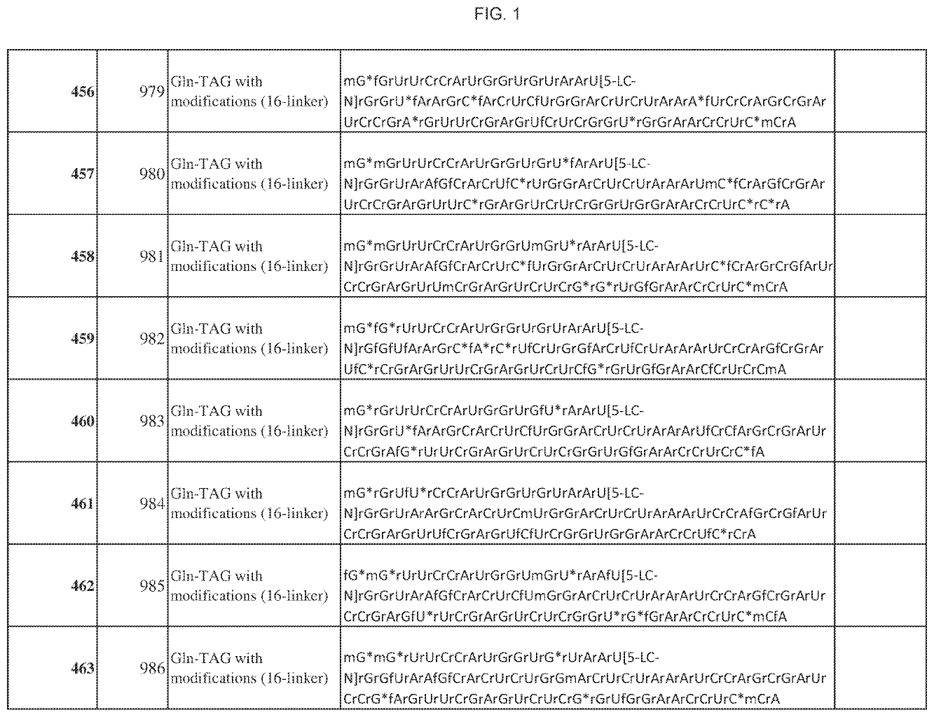

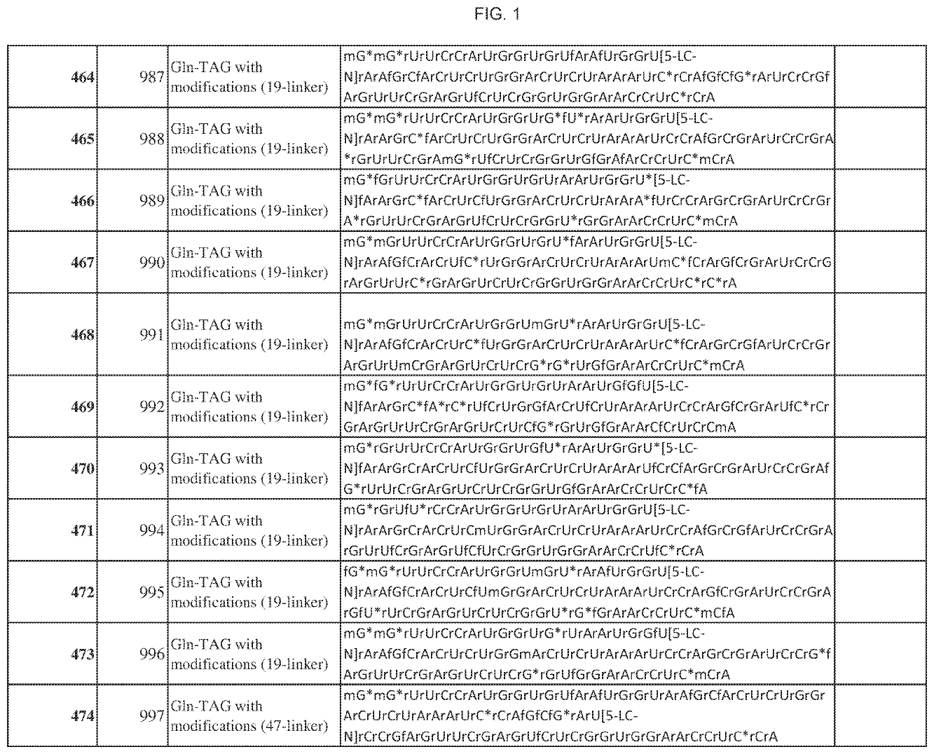

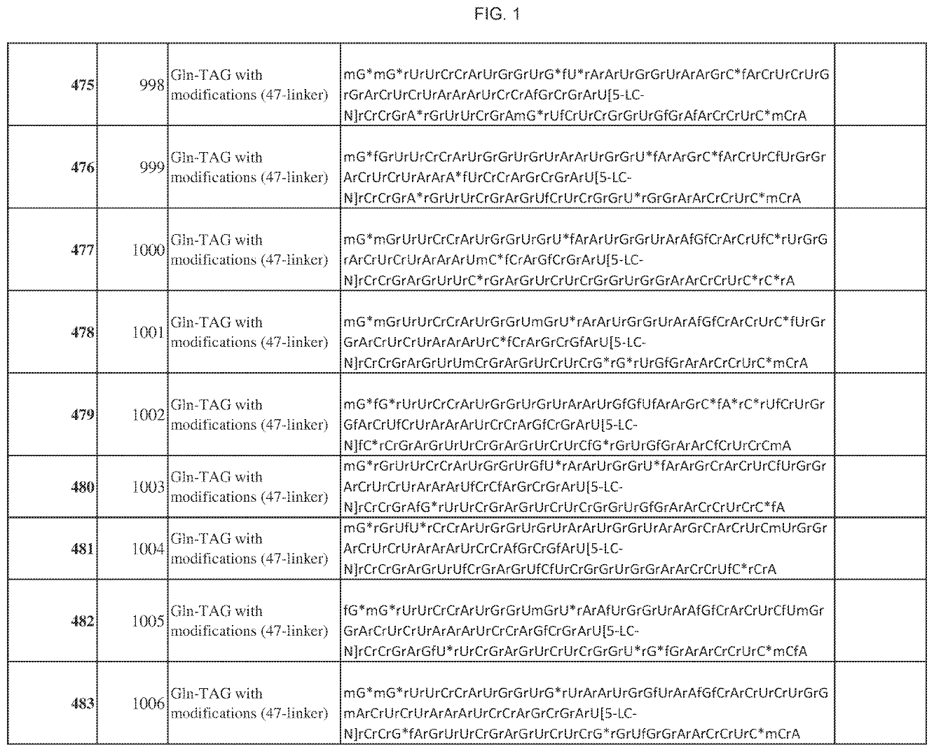

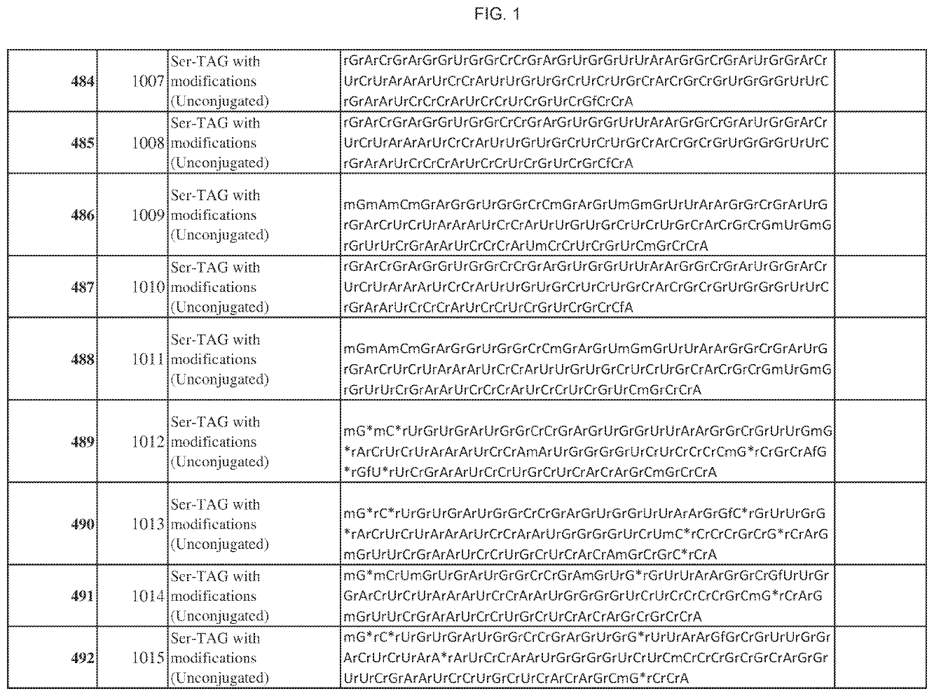

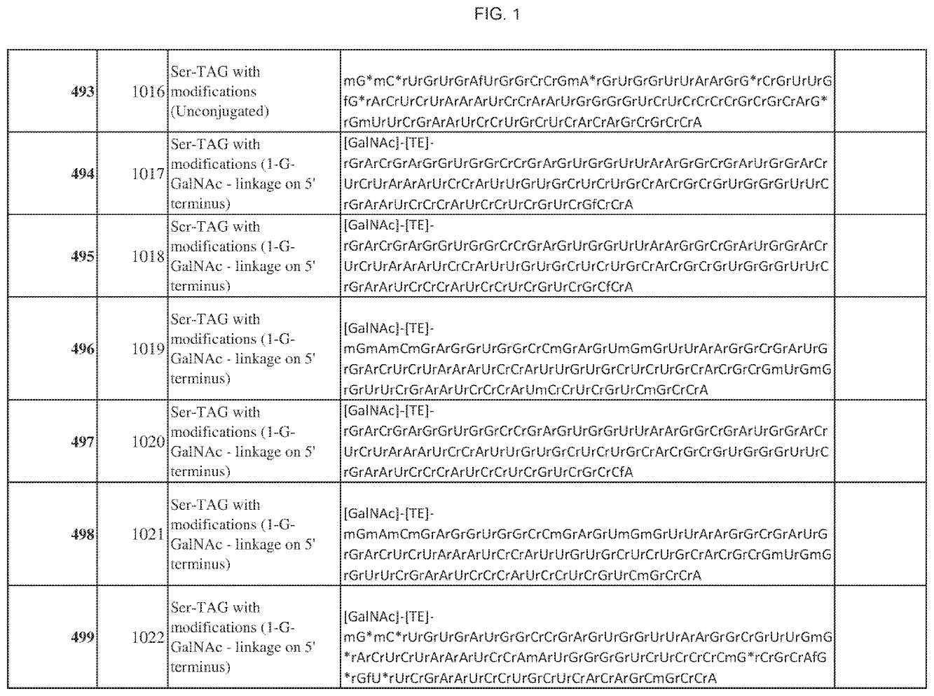

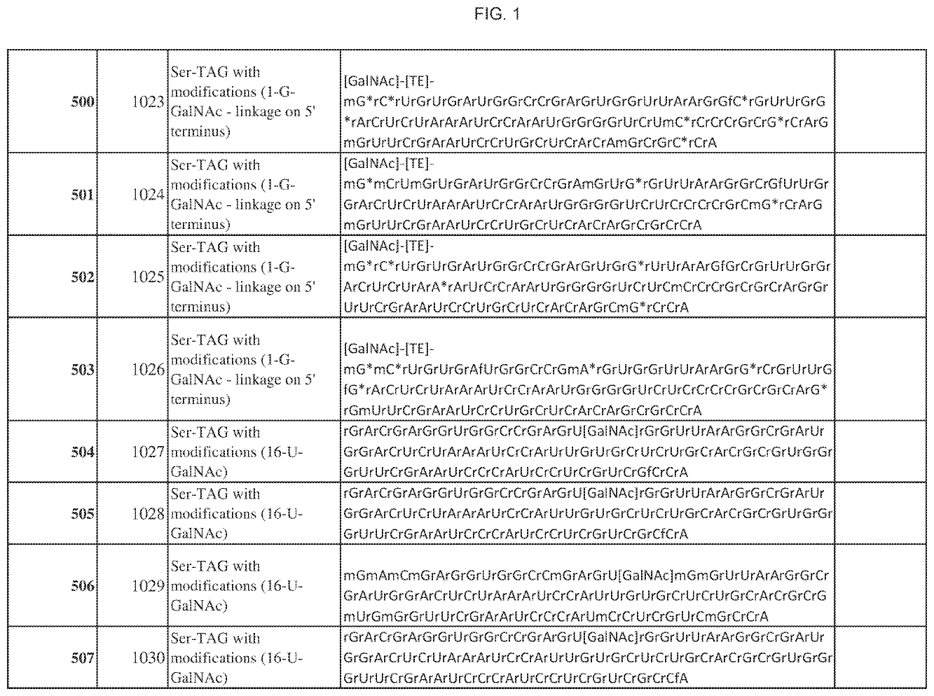

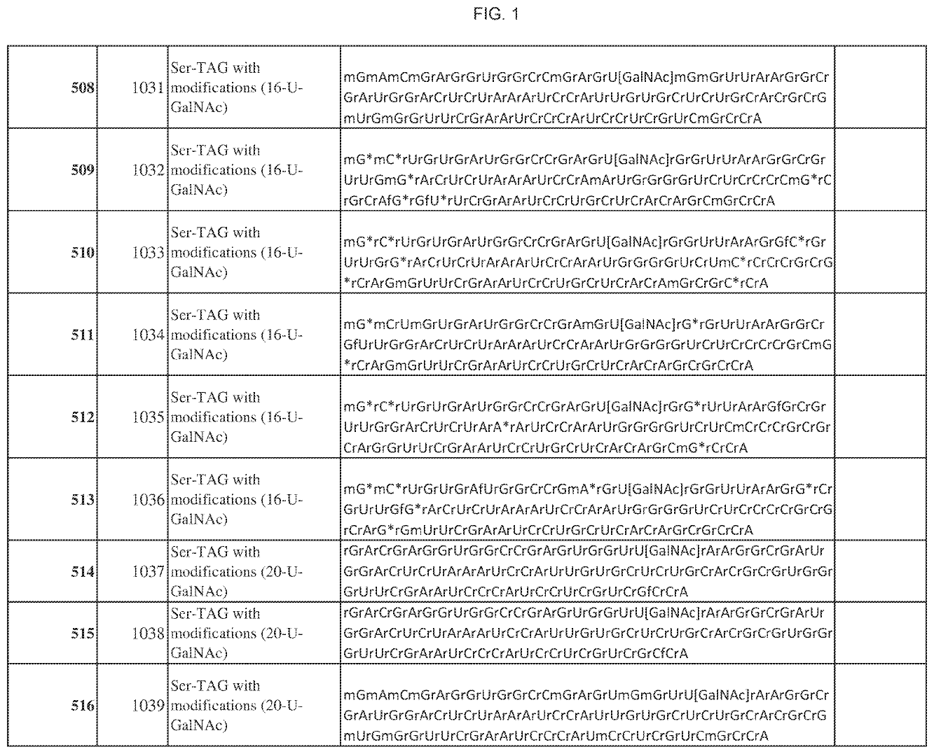

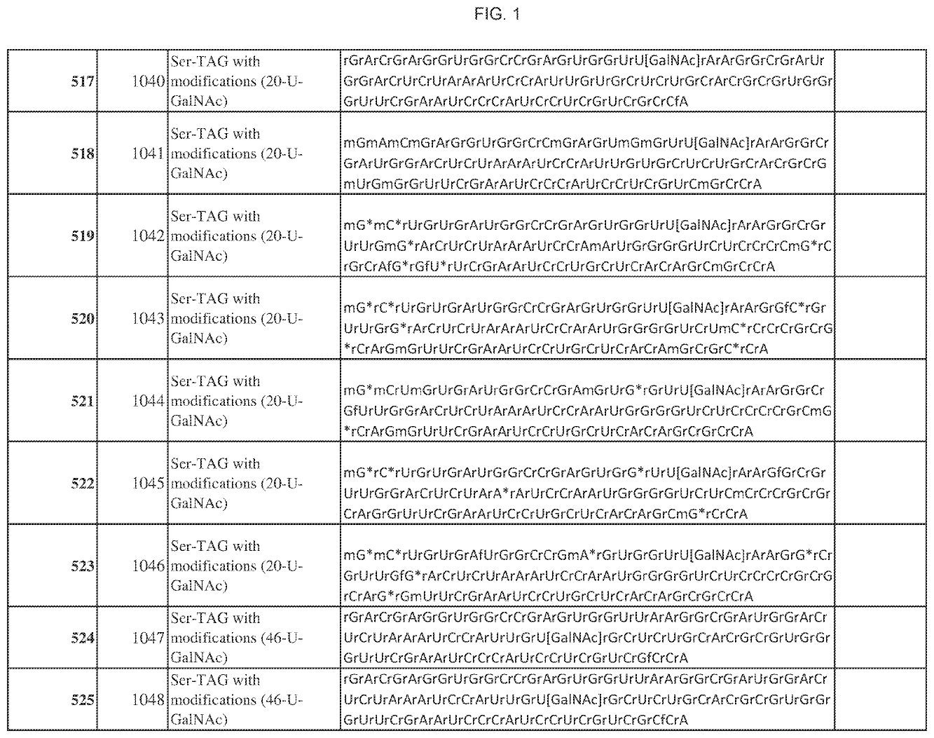

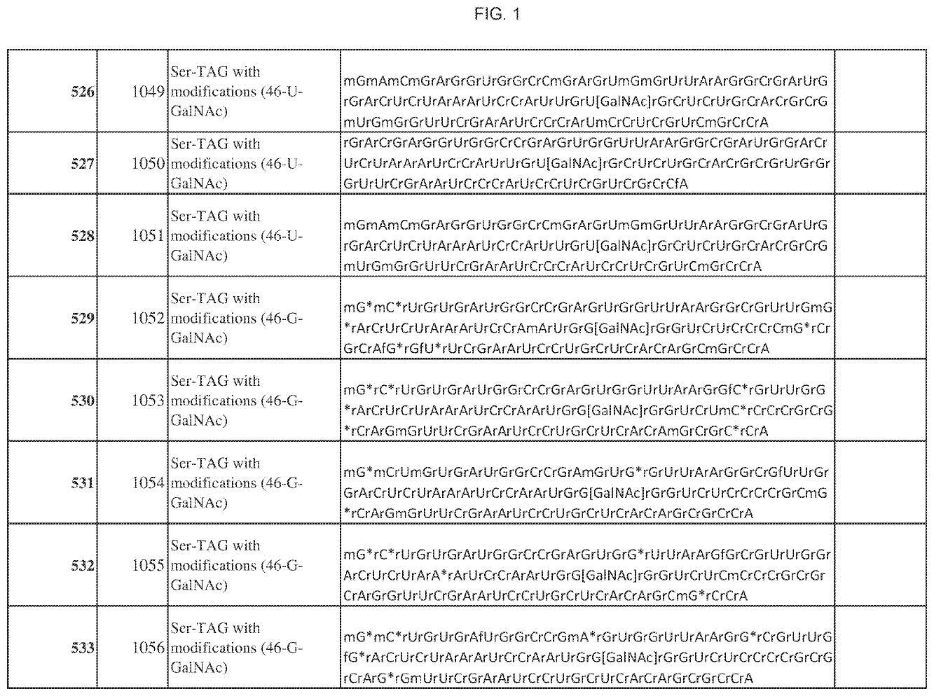

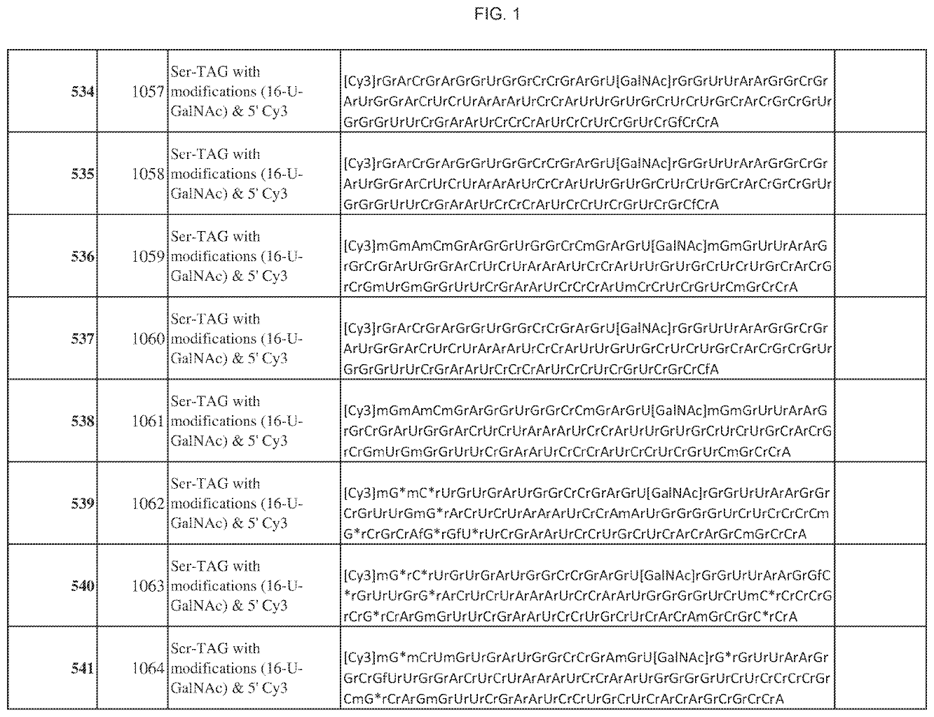

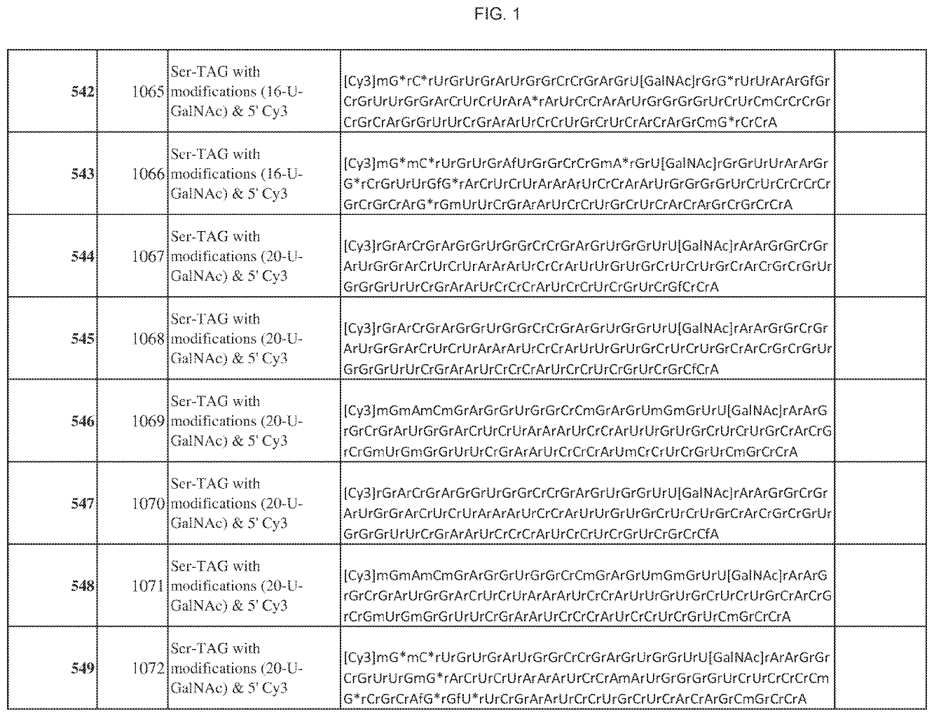

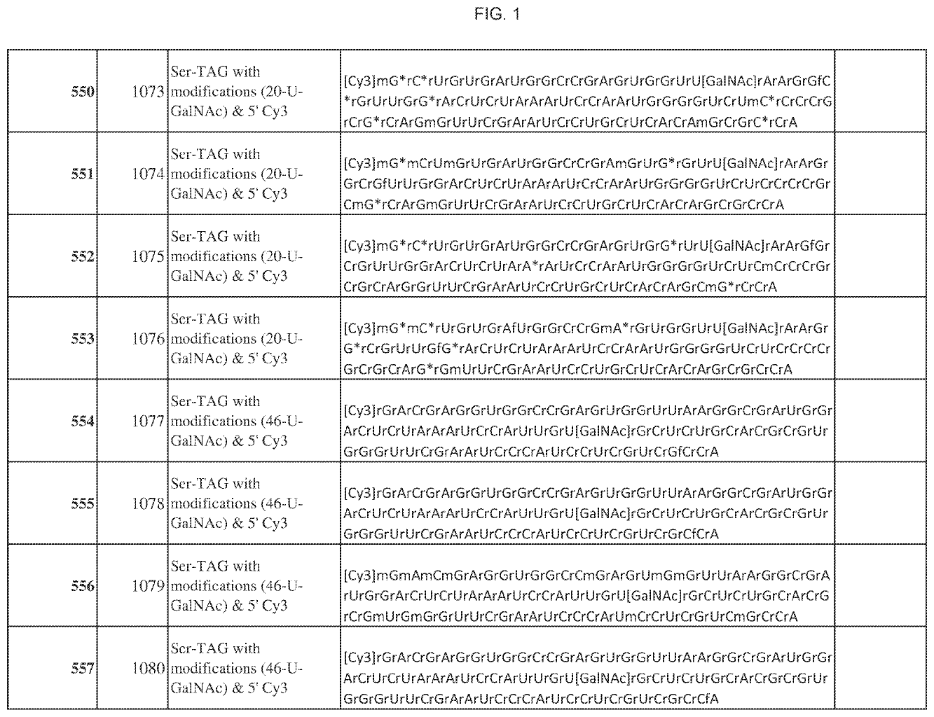

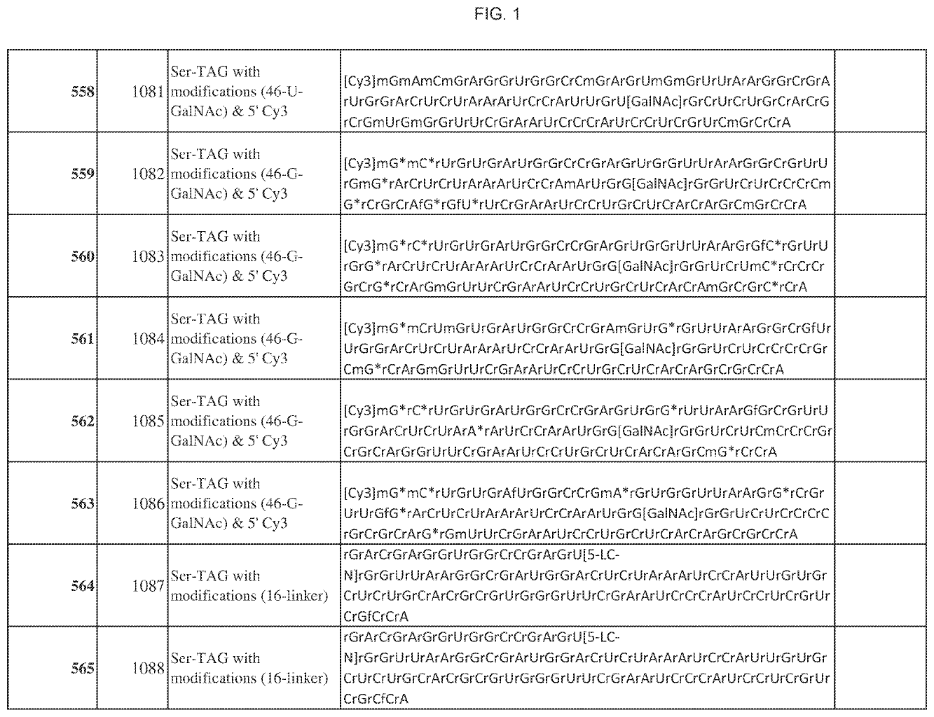

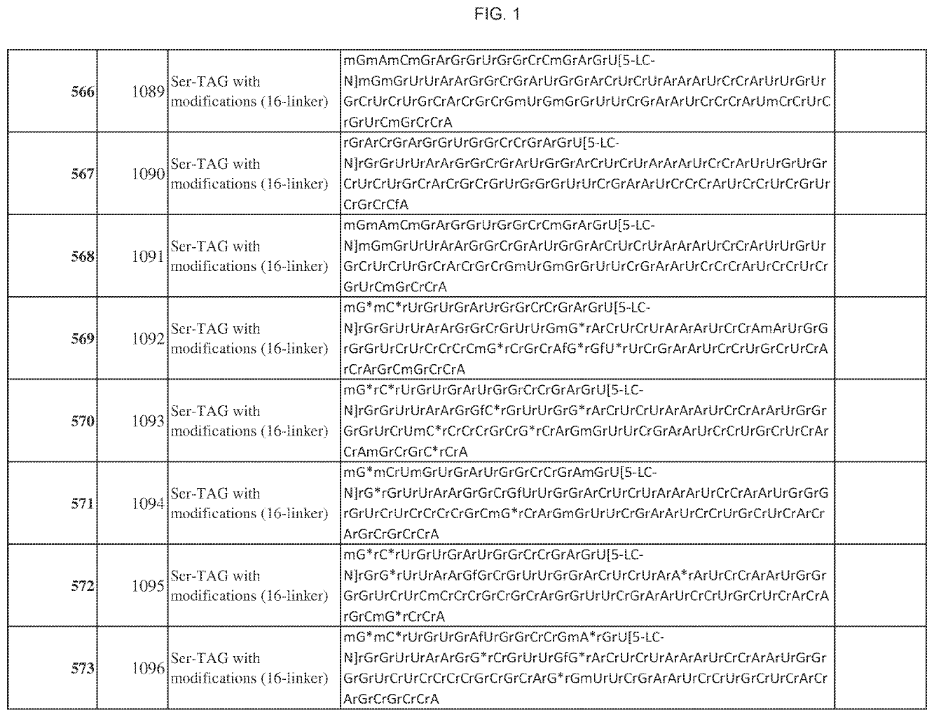

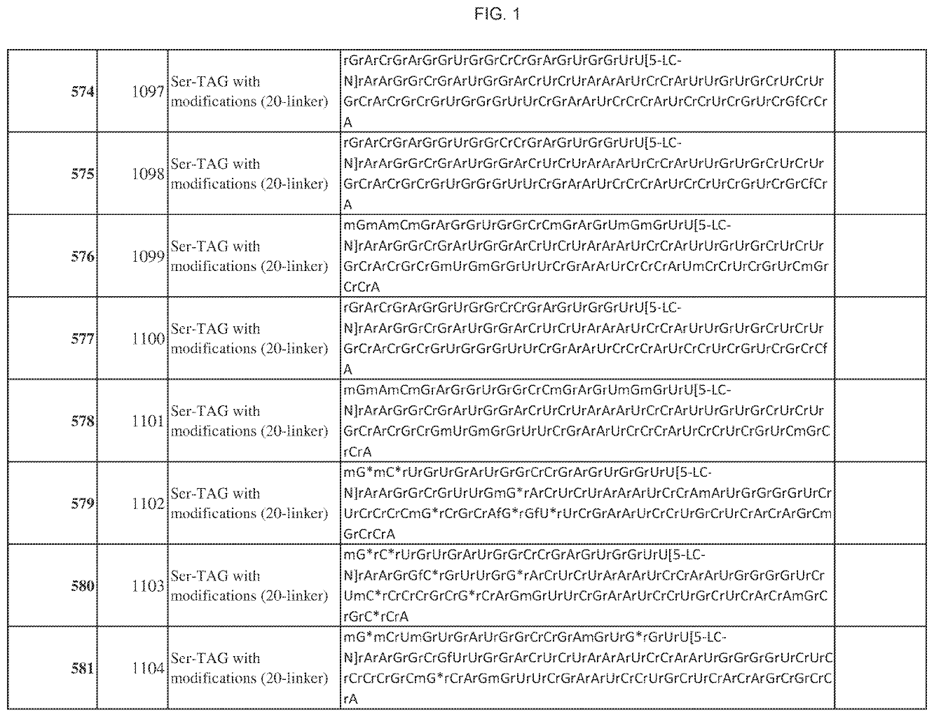

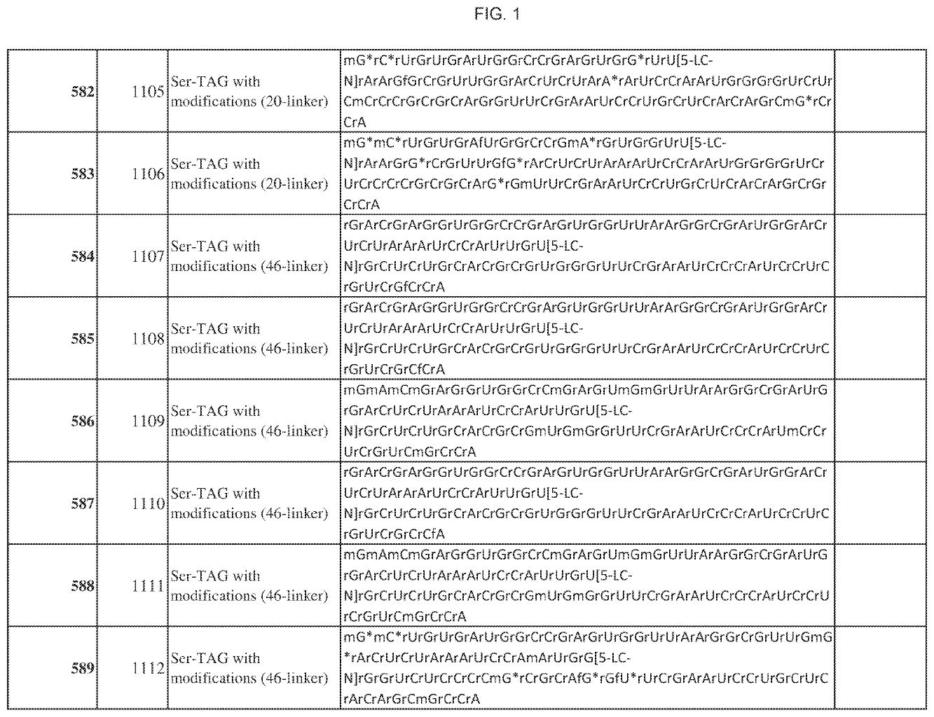

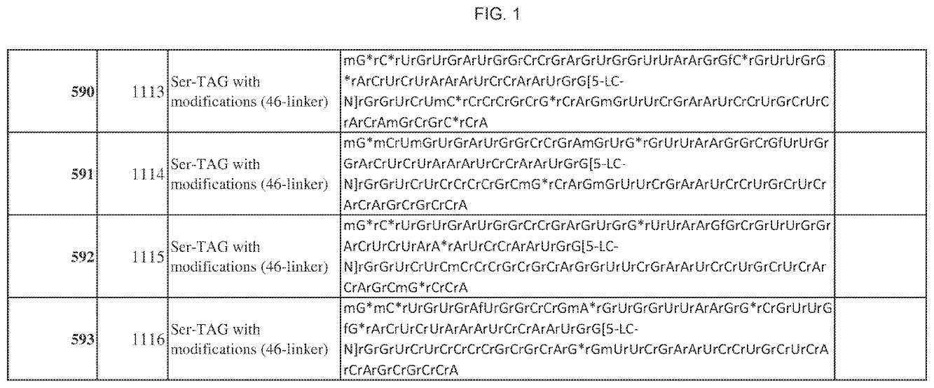

36. The TREM of any one of claims 33-35, wherein the TREM comprises 1, 2, 3, 4, 5, 6, 7, 8, 9, or 10 additional non-naturally occurring modifications compared with a TREM provided in FIG. 1 (e.g., 2′-ribose modifications or an internucleotide modification, e.g., 2′OMe, 2′-halo, 2′-MOE, 2′-deoxy, or phosphorothiorate modifications).

37. The TREM of any one of the preceding claims, wherein the TREM has a sequence selected from a sequence provided in FIG. 1.

38. The TREM of any one of the preceding claims, wherein the TREM is a TREM provided in Table 12.

39. The TREM of any one of the preceding claims, wherein the TREM comprises a TREM having at least 70%, 75%, 80%, 85%, 90%, 95%, or 99% sequence identity with a TREM provided in FIG. 1.

40. The TREM of any one of the preceding claims, wherein the TREM comprises a nucleotide sequence that differs by 2, 3, 4, 5, 6, 7, 8, 9, 10, 11, 12, 13, 14, 15, 16, 17, 18, 19, or 20 nucleotides from the nucleotide sequence of a TREM provided in FIG. 1.

41. The TREM of any one of the preceding claims, wherein the TREM comprises a nucleotide sequence that differs by 10, 15, 20, 25, 30, 35 or 40 nucleotides from the nucleotide sequence of a TREM provided in FIG. 1.

42. The TREM of any one of the preceding claims, wherein the TREM comprises a nucleotide sequence that differs by more than 5 nucleotides from the nucleotide sequence of a TREM provided in FIG. 1.

43. The TREM of any one of the preceding claims, wherein the TREM comprises a nucleotide sequence that differs by more than 10 nucleotides from the nucleotide sequence of a TREM provided in FIG. 1.

44. The TREM of any one of the preceding claims, wherein the TREM comprises a sequence that differs by no more than 1, 2, 3, 4, 5, 6, 7, 8, 9, or 10 nucleotides from the nucleotide sequence of a TREM provided in FIG. 1.

45. The TREM of any one of the preceding claims, wherein the TREM comprises a nucleotide sequence that differs by no more than 10, 15, 20, 25, 30, 35 or 40 nucleotides from the nucleotide sequence of a TREM provided in FIG. 1.

46. The TREM of any one of the preceding claims, wherein the TREM comprises a nucleotide sequence that differs by no more than 1 nucleotide from the nucleotide sequence of a TREM provided in FIG. 1.

47. The TREM of any one of the preceding claims, wherein the TREM comprises a nucleotide sequence that differs by no more than 5 nucleotides from the nucleotide sequence of a TREM provided in FIG. 1.

48. The TREM of any one of the preceding claims, wherein the TREM comprises a nucleotide sequence that differs by no more than 10 nucleotides from the nucleotide sequence of a TREM provided in FIG. 1.

49. The TREM of any one of the preceding claims, wherein the TREM is selected from SEQ NOs. 622-1116 in FIG. 1.

50. A pharmaceutical composition comprising a TREM entity (e.g., a TREM) of any one of claims 1-49.

51. A lipid nanoparticle comprising a TREM of any one of claims 1-49 or a pharmaceutical composition of claim 50.

52. A method of making a TREM entity (e.g., a TREM) of any one of claims 1-49.

53. A method of treating a subject having a disease or disorder associated with a PTC, comprising administering to the subject a TREM comprising an ASGPR binding moiety described herein (e.g., a TREM of any one of claims 1-49) or a pharmaceutical composition of claim 50 or a lipid nanoparticle formulation of claim 51, thereby treating the subject having the disease or disorder.

54. The method of claim 53, wherein the subject is a human.

55. A composition for use in treating a subject having a disease or disorder associated with a PTC, wherein the composition for use comprises a comprising an ASGPR binding moiety described herein (e.g., a TREM of any one of claims 1-49) or a pharmaceutical composition of claim 50 or a lipid nanoparticle formulation of claim 51.
